(19) United States
(12) Patent Application Publication
(10) Pub. No.: US 2022/0307053 A1
(43) Pub. Date: Sep. 29, 2022

(54) REGULATABLE EXPRESSION SYSTEMS

(71) Applicant: Novartis AG, Basel (CH)

(72) Inventors: Martin BEIBEL, Freiburg (DE); Caroline Gubser KELLER, Basel (CH); Dmitriy LUKASHEV, Ashland, MA (US); Nicole RENAUD, Somerville, MA (US); Nikita RUDINSKIY, Charlestown, MA (US); Rajeev SIVASANKARAN, Acton, MA (US)

(21) Appl. No.: 17/629,286

(22) PCT Filed: Jul. 24, 2020
(86) PCT No.: PCT/IB2020/057038

§ 371 Date: Jan. 21, 2022

Related U.S. Application Data
(60) Provisional application No. 63/013,283, filed on Apr. 21, 2020.
(60) Provisional application No. 62/878,514, filed on Jul. 25, 2019.

(51) Int. Cl. C12N 15/86; C12N 15/113; C12N 15/67; C12N 5/079; C12N 5/0793

(52) U.S. Cl. CPC C12N 15/86; C12N 15/113; C12N 15/67; C12N 5/0622; C12N 5/0619; C12N 2510/00; C12N 2750/14121; C12N 2750/14143; C12N 2830/42; C12N 2800/60

(57) ABSTRACT

Provided herein are compositions comprising minigenes comprising splice modulator binding sequences, for regulatable gene expression, and systems and methods of use thereof.

### SEQUENCE LISTING

The instant application contains a Sequence Listing which has been submitted electronically in ASCII format and is hereby incorporated by reference in its entirety. Said ASCII copy, created on May 10, 2022, is named PAT058643-WO-PCT_SL.txt and is 108,491 bytes in size.

### FIELD OF THE DISCLOSURE

Disclosed herein are compositions comprising minigenes for regulatable gene expression and systems and methods of use thereof.

### BACKGROUND

Gene therapy methods that deliver genetic material (e.g., heterologous nucleic acids) into target cells in order to increase the expression of desired gene products may support this therapeutic objective. Viruses have evolved to become highly efficient at nucleic acid delivery to specific cell types while avoiding immunosurveillance by an infected host. Robbins et al., (1998) Pharmacol. Ther., 80(1):35-47. These properties make viruses attractive as delivery vehicles, or vectors, for gene therapy. Several types of viruses, including retrovirus, adenovirus, adeno-associated virus (AAV), and herpes simplex virus, have been modified in the laboratory for use in gene therapy applications. Lunstrom et al., (2018) Diseases, 6(2): 42. In particular, vectors derived from Adeno-Associated Viruses (AAVs) may effectively deliver genetic material because (i) they are able to infect (transduce) a wide variety of non-dividing and dividing cell types including muscle fibers and neurons; (ii) they are devoid of the virus structural genes, thereby eliminating the natural host cell responses to virus infection, e.g., interferon-mediated responses; (iii) wild-type viruses have never been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response (see ii), thus granting long-term expression of, e.g., therapeutic heterologous nucleic acid(s) (provided their gene products are not rejected). Wold et al., (2013) Curr. Gene Ther., 13(6):421-33; Lee et al., (2017) Genes Dis., 4(2): 43-63.

AAV is a member of the parvoviridae family. The AAV genome comprises a linear single-stranded DNA molecule which typically contains approximately 4.7 kilobases (kb) and two major open reading frames encoding the non-structural Rep (replication) and structural Cap (capsid) proteins. Flanking the AAV coding regions are two cis-acting inverted terminal repeat (ITR) sequences, which are typically approximately 145 nucleotides in length and have interrupted palindromic sequences that can fold into hairpin structures that function as primers during initiation of DNA replication. In addition to their role in DNA replication, the ITR sequences have been shown to contribute to viral integration, rescue from the host genome, and encapsidation of viral nucleic acid into mature virions. Muzyczka et al., (1992) Curr. Top. Micro. Immunol., 158:97-129.

Many proteins have been developed which are important scientific research tools or medications for preventing or treating diseases. While viral vectors such as AAVs are desirable for their ability to transduce a variety of cell types and deliver the heterologous nucleic acids encoding these proteins to a variety of target tissue types, side effects can occur upon expression of the proteins, varying from, for example, a loss of drug efficacy to serious toxicities. It is desirable to develop strategies to modulate the expression level of the therapeutic proteins, e.g., to modulate the timing or location of expression of therapeutic proteins and/or levels of the therapeutic proteins to increase efficacy and/or decrease side effects.

Accordingly, the present disclosure provides, in part, minigene nucleotide sequences that are useful to control expression of proteins using a small-molecule to turn off or turn on expression of the protein of interest. The disclosure also provides vectors, recombinant viruses and pharmaceutical compositions comprising such minigene sequences, and contemplates their use in methods regulating gene expression.

### SUMMARY

In a first aspect, provided is a nucleic acid molecule including a minigene linked to a transgene encoding a protein of interest, wherein the minigene includes: A first exon; A first intron; A second exon; A second intron; and A third exon; wherein said second exon includes a splice modulator binding sequence and wherein, in the presence of a splice modulator, said second exon is included in an mRNA product of the nucleic acid, and in the absence of said splice modulator, said second exon is not included in an mRNA product of the nucleic acid.

In embodiments, the third exon includes a stop codon that is in frame in the mRNA product of the nucleic acid produced in the absence of the splice modulator and which is not in frame in the mRNA product of the nucleic acid produced in the presence of the splice modulator.

In embodiments, the second exon includes a stop codon that is in frame in the mRNA product of the nucleic acid produced in the presence of the splice modulator.

In embodiments, the first exon and the third exon do not comprise a start codon. In some embodiments, the second exon comprises a start codon.

In embodiments of any of the aforementioned aspects and embodiments, the nucleic acid includes a sequence encoding a protease cleavage site disposed between the minigene and the transgene.

In embodiments said protease cleavage site is cleaved by a mammalian protease.

In embodiments the mammalian protease is furin, PCSK1, PCSK5, PCSK6, PCSK7, cathepsin B, Granzyme B, Factor XA, Enterokinase, genenase, sortase, precission protease, thrombin, TEV protease, or elastase 1.

In embodiments of any of the aforementioned aspects and embodiments, the protease cleavage site includes a polypeptide having an cleavage motif selected from the group consisting of RX(K/R)R consensus motif, RXXX[KR]R consensus motif, RRX consensus motif, RNRR (SEQ ID NO: 39), I-E-P-D-X consensus motif (SEQ ID NO: 35), Glu/Asp-Gly-Arg, Asp-Asp-Asp-Asp-Lys (SEQ ID NO: 36), Pro-Gly-Ala-Ala-His-Tyr (SEQ ID NO: 37), LPXTG/A consensus motif, Leu-Glu-Val-Phe-Gln-Gly-Pro (SEQ ID NO: 38), Leu-Val-Pro-Arg-Gly-Ser (SEQ ID NO: 40), E-N-L-Y-F-Q-G (SEQ ID NO: 41), and [AGSV]-x (SEQ ID NO: 42). In embodiments said cleavage site is cleaved by furin. In embodiments, the protease cleavage site cleaved by furin is RNRR (SEQ ID NO: 39); RTKR (SEQ ID NO: 43); GTGAEDPRPSRKRRSLGDVG (SEQ ID NO: 45); GTGAEDPRPSRKRR (SEQ ID NO: 47); LQWLEQQVAKRRTKR (SEQ ID NO: 49); GTGAEDPRPSRKRRSLGG (SEQ ID NO: 51); GTGAEDPRPSRKRRSLG (SEQ ID NO: 53); SLNLTESHNSRKKR (SEQ ID NO: 55); or CKINGYPKRGRKRR (SEQ ID NO: 57). In embodiments the protease cleavage site cleaved by furin includes RNRR (SEQ ID NO: 39). In embodiments the sequence encoding the protease cleave site includes, e.g., consists of, CGCAACCGCCGC (SEQ ID NO: 19).

In embodiments including in any of the aforementioned aspects and embodiments the nucleic acid includes a sequence encoding a self-cleaving peptide disposed between the minigene and the transgene, optionally wherein the self-cleaving peptide cleaves within 1, 2, 3, 4, 5, 6, 7, 8, 9, or 10 amino acids of the N-terminus of the protein of interest. In embodiments, the self-cleaving peptide is a 2A peptide, optionally selected from a T2A peptide, a P2A peptide, a E2A peptide and a F2A peptide, e.g., includes a T2A peptide, e.g., wherein the self-cleaving peptide includes EGRGSLLTCGDVEENPGP (SEQ ID NO: 61), optionally wherein the self-cleaving peptide includes (GSG)EGRGSLLTCGDVEENPGP (SEQ ID NO: 59).

In embodiments including in any of the aforementioned aspects and embodiments the splice modulator binding sequence is located at the 3′ terminus of the second exon.

In embodiments including in any of the aforementioned aspects and embodiments the splice modulator binding sequence includes, e.g., consists of, AGA and the splice modulator is 5-(1H-Pyrazol-4-yl)-2-(6-((2,2,6,6-tetramethylpiperidin-4-yl)oxy)pyridazin-3-yl)phenol (LMI070).

In embodiments including in any of the aforementioned aspects and embodiments the second exon includes, e.g., consists of a sequence selected from:

(SEQ ID NO: 1)
CCTTGCTATCCCTGTCTTCTGTAGCTATTCTGAAACCATCAACAAAGGAG
CACACCATTCCATCAGCAAAAGA;
(SEQ ID NO: 2)
GTAATTAGCTGAGAAGGAAGATCTGAAGGTTTAACGAGAGAGGGCGAGAG
ATACAAAATATCTGCTAGGAGA;
(SEQ ID NO: 3)
GGATTGTTTGTATTCCTGCCAATGATTTGTGAGACAGTCTGTTCCCCACA
TCCTCGTCAACAGA;
(SEQ ID NO: 4)
CTTTCTGACATCTTAACGAGGCAATACAGAGAGACGAATTTTCATCAGTT
TGTTCAGGGAGACACATATAACAAAAGA;
(SEQ ID NO: 5)
ATCCATACATACTTAATGCTGAAATGTGAAGGGCTGAGAAAAAAGAAAAG
A;
(SEQ ID NO: 6)
AATTGGAAACATCGAGGGAAAATGGGCTTTTTATTATTAAAACAAAACCT
CAGTATTATCACTTAGAAACCTGAAATTGAACTCCAAAAGCCAAAGA;
(SEQ ID NO: 7)
AAGAATGTTCCTTTTGTGAAGAATGACTTAAGGAAGATTCATGATGACTG
AGTGTGCCCGTGTGGAACTTTAGGACATAGATGCACTCCTACAGA;
(SEQ ID NO: 8)
TTGTCCTTCACTCCGTACTCCAGTTGGCCAAGCATAGGTCGCATGCCAGG
GTCAAGGAGACTAAGGGAGA;
(SEQ ID NO: 9)
GACATACAGACATGGCAGCCCCTAGCATGTGTATCCTAAGA;
(SEQ ID NO: 10)
ACATACAGACATGGCAGCCCCTAGCATGTGTATCCTAAGA;
(SEQ ID NO: 80)
AGTTTGCAAAGGAAGGAAAGGAGCAGAGACTTGAATGAGCAGAAAATCAT
TTCAGGGCCTGTTCTCTATGTCCTTGCTATCCCTGTCTTCTGTAGCTATT
CTGAAACCATCAACAAAGGAGCACACCATTCCATCAGCAAAAGA

and

A fragment or mutant of any of (a) to (k) having at least 90%, at least 95% at least 96%, at least 97%, at least 98% or at least 99% identity thereto.

In embodiments including in any of the aforementioned aspects and embodiments the second exon includes a sequence derived from an exon of SNX7, optionally wherein the sequence is derived a cryptic exon of SNX7.

In embodiments including in any of the aforementioned aspects and embodiments the second exon includes, e.g., consists of,

(SEQ ID NO: 16)
AGTTTGCAAAGGAAGGAAAGGAGCAGAGACTTGATTGAGCAGAAAATCAT
TTCAGGGCCTGTTCTCTATTGTCCTTGCTATCCTGTCTTCTGTAGCTATC
TGAAACCATCAACAAAGGAGCACACCATTCCATCAGCAAAAGA;

a fragment of SEQ ID NO: 16; or
a mutant sequence of SEQ ID NO: 16 or a fragment thereof having at least 90%, at least 95% at least 96%, at least 97%, at least 98% or at least 99% identity thereto.

In embodiments including in any of the aforementioned aspects and embodiments the second exon includes, e.g., consists of,

(SEQ ID NO: 98)
AGTTTGCAAAGGAAGGAAAGGAGCAGAGACTTGATTGAGCAGAAAATCAT
TTCAGGGCCTGTTCTCTATTGTCCTTGCTATCCTGTCTTCTGTAGCTATC
TGAAACCATCAACAAAGGAGCACACCATGGCATCAGCAAAAGA;

a fragment of SEQ ID NO: 98; or
a mutant sequence of SEQ ID NO: 98 or a fragment thereof having at least 90%, at least 95% at least 96%, at least 97%, at least 98% or at least 99% identity thereto.

In embodiments including in any of the aforementioned aspects and embodiments the second exon consists of 3n−1 nucleotides, where n is an integer.

In embodiments including in any of the aforementioned aspects and embodiments the first exon includes: One or more, e.g., three, GAA repeats (SEQ ID NO: 69) (for example, includes GAAGAAGAA (SEQ ID NO: 69));

A Kozak sequence (e.g., a Kozak sequence including GCCACC (SEQ ID NO: 70)); or

Both (a) and (b).

In embodiments including in any of the aforementioned aspects and embodiments the first exon includes, e.g., consists of,

(SEQ ID NO: 96)
GAAGAAGAAGATATCAAGTTAGCATTTACAGATTTGGCTGAGGAGAAGAA
CAG;

a fragment of SEQ ID NO: 96; or
a mutant sequence of SEQ ID NO: 96 or a fragment thereof having at least 90%, at least 95% at least 96%, at least 97%, at least 98% or at least 99% identity thereto.

In embodiments including in any of the aforementioned aspects and embodiments the first intron includes, e.g., consists of,

(SEQ ID NO: 97)
GTAATTAGTGTTGTTTGATATTGCTTCATTTTAAAGTTATTTGCTCATTT
AGCATTTGATATTGCTTTCTATTGATTGTCCTAACTACTCCTCTTTCCTC
TCCCTTCTCCATTTTTGAAG;

a fragment of SEQ ID NO: 97; or
a mutant sequence of SEQ ID NO: 97 or a fragment thereof having at least 90%, at least 95% at least 96%, at least 97%, at least 98% or at least 99% identity thereto.

In embodiments including in any of the aforementioned aspects and embodiments the minigene has been modified to:

Remove or mutate all but a single start codon, e.g., an ATG start codon;

Remove or mutate all cryptic splice donor and splice acceptor sequences other than those at the termini of the first exon, the second exon and the third exon.

In embodiments, the minigene has a single start codon disposed within the first exon. In embodiments, the minigene has a single start codon disposed within the second exon.

In embodiments including in any of the aforementioned aspects and embodiments the minigene includes fewer than 2000, fewer than 1900, fewer than 1800, fewer than 1700, fewer than 1600, fewer than 1500, fewer than 1400, fewer than 1300, fewer than 1200, fewer than 1100, or fewer than 1000, fewer than 900, fewer than 800, fewer than 700, fewer than 600, fewer than 500 nucleotides.

In embodiments including in any of the aforementioned aspects and embodiments the minigene includes between about 2500 and about 500 nucleotides, e.g., between about 2000 and about 600 nucleotides, e.g., between about 1500 and about 700 nucleotides, e.g., between about 1200 and about 800 nucleotides, between about 1100 and about 900 nucleotides, between about 800 and about 500 nucleotides, between about 800 and about 600 nucleotides.

In embodiments including in any of the aforementioned aspects and embodiments the minigene includes, e.g., consists of, SEQ ID NO: 71 or SEQ ID NO: 94, or a sequence with at least 90, 91, 92, 93, 94, 95, 96, 97, 98, or 99% identity thereto, or a functional fragment thereof.

In an aspect, disclosed herein is a nucleic acid molecule, including (a) a transgene encoding a protein of interest, and (b) a minigene including, e.g., consisting of, SEQ ID NO: 71 or SEQ ID NO: 94, or a sequence with at least 90, 91, 92, 93, 94, 95, 96, 97, 98, or 99% identity thereto, or a functional fragment thereof.

In embodiments including in any of the aforementioned aspects and embodiments, the nucleic acid molecule further includes a sequence encoding a furin cleavage site, said sequence including SEQ ID NO: 19, and a sequence encoding a self-cleaving peptide, said sequence including SEQ ID NO: 20, optionally wherein the minigene is disposed 5′ to the sequence encoding the furin cleavage site (e.g., immediately 5′ to the sequence encoding the furin cleavage site), the sequence encoding the furin cleavage site is disposed 5′ to the sequence encoding the self-cleaving peptide (e.g., immediately 5′ to the sequence encoding the self-cleaving peptide), and the sequence encoding the self-cleaving peptide is disposed 5′ to the transgene (e.g., immediately 5′ to the transgene).

In embodiments including in any of the aforementioned aspects and embodiments, the nucleic acid molecule further including a promoter operably linked to the minigene and transgene, optionally wherein said promoter is disposed 5′ to the minigene.

In embodiments including in any of the aforementioned aspects and embodiments the promoter is a JeT promoter, a CBA promoter, a PGK promoter, or a synapsin promoter, or any promoter that does not include an intron.

In embodiments including in any of the aforementioned aspects and embodiments, the nucleic acid molecule further includes a post-transcriptional regulatory element.

In embodiments including in any of the aforementioned aspects and embodiments the post-transcriptional regulatory element (PRE) includes a PRE derived from hepatitis B (HPRE), bat (BPRE), ground squirrel (GSPRE), arctic squirrel (ASPRE), duck (DPRE), chimpanzee (CPRE) and wooly monkey (WMPRE) or woodchuck (WPRE), optionally wherein said post-transcriptional regulatory element is disposed 3′ to the transgene.

In embodiments including in any of the aforementioned aspects and embodiments the post-transcriptional regulatory element includes SEQ ID NO: 72, SEQ ID NO: 73 or SEQ ID NO:88.

In embodiments including in any of the aforementioned aspects and embodiments, the nucleic acid molecule further includes a polyadenylation signal (polyA), optionally wherein said polyA is disposed 3′ to the transgene.

In embodiments including in any of the aforementioned aspects and embodiments the poly A signal is an SV40 polyA, human growth hormone (HGH) polyA, or bovine growth hormone (BGH) polyA, a beta-globin polyA, an alpha-globin polyA, an ovalbumin polyA, a kappa-light chain polyA, and a synthetic polyA.

In embodiments including in any of the aforementioned aspects and embodiments the polyA includes, e.g., consists of, SEQ ID NO: 22.

In another aspect, disclosed herein is a vector including a nucleic acid according to any one of the previous aspects and embodiments. In embodiments, the vector is a DNA vector, optionally a circular vector, optionally a plasmid. In embodiments, the vector is double stranded or single stranded, e.g., is double stranded.

In embodiments, the vector is a viral vector. In embodiments, the viral vector is an adeno-associated viral (AAV) vector, chimeric AAV vector, adenoviral vector, retroviral vector, lentiviral vector, DNA viral vector, herpes simplex viral vector, baculoviral vector, or any mutant or derivative thereof. In embodiments, the viral vector is a recombinant AAV vector, optionally a self-complementary AAV (scAAV) vector. In embodiments, the viral vector is a recombinant AAV vector, optionally a single-stranded AAV (ssAAV) vector. In embodiments, the recombinant AAV vector includes one or more inverted terminal repeats (ITRs), optionally wherein the ITRs are AAV2 ITRs, optionally wherein the AAV vector includes two ITRs, optionally wherein the two ITRs include SEQ ID NO: 12 and SEQ ID NO: 23.

In embodiments, including in any of the previous aspects and embodiments, the vector includes, e.g. from 5′ to 3′:

an ITR, optionally an AAV2 ITR, optionally, wherein the ITR has been modified to include a deletion of a terminal resolution site, optionally including SEQ ID NO: 12;
a promoter, optionally a JeT promoter including or consisting of SEQ ID NO: 13;
a nucleic acid molecule of any one of aspects 1-28;
a polyA signal, optionally including or consisting of SEQ ID NO: 22; and
an ITR, optionally an AAV2 ITR, optionally including or consisting of SEQ ID NO: 23.

In an aspect, provided herein is a recombinant virus including the nucleic acid or vector of any of the previous aspects and embodiments. In embodiments, the recombinant virus is an adeno-associated virus (AAV), chimeric AAV, adenovirus, retrovirus, lentivirus, DNA virus, herpes simplex virus, baculovirus, or any mutant or derivative thereof. In embodiments, the virus is an AAV. In embodiments, the AAV includes one or more of an AAV1, AAV2, AAV3, AAV4, AAV5, AAV6, AAV7, AAV 8, AAV9, AAV10, and AAV11, AAV12, AAVrh8, AAVrh10, AAVrh36, AAVrh37, AAV-DJ, AAV-DJ/8, AAV.Anc80, AAV.Anc80L65, AAV-PHP.B, AAV-PHP.B2, AAV-PHP.B3, AAV-PHP.A, AAV-PHP.eB, and AAV-PHP.S capsid serotype, or a variant thereof, e.g., a combination of capsids from more than one AAV serotype. In embodiments, the AAV includes an AAV9 capsid serotype or any mutant or derivative thereof. In embodiments, the virus includes AAV9 capsid proteins VP1, VP2, and VP3, e.g., as encoded by SEQ ID NO: 74, SEQ ID NO: 75, and SEQ ID NO: 76, respectively, or including an amino acid sequence of SEQ ID NO: 77, SEQ ID NO: 78, SEQ and ID NO: 79, respectively. In embodiments, the AAV includes a self-complementary AAV (scAAV) vector. In embodiments, the AAV includes a single-stranded AAV (ssAAV) vector.

In another aspect, provided herein is a cell including the nucleic acid molecule, the vector, or the recombinant virus of any one the previous aspects and embodiments. In embodiments, the cell is a human cell. In embodiments, the cell is a neuron or astrocyte.

In an aspect, provided herein is a cell, including a cell of any previous cell aspect and embodiments, wherein when the cell includes a splice modulator, e.g., LMI070, the level of expression of the protein of interest is greater, e.g., 2, 3, 4, 5, 6, 7, 8, 9, 10, 20, 30, 50 or 100 fold greater, than the level of expression of the protein of interest when the cell does not include said splice modulator, optionally wherein the level of expression when the cell does not include said splice modulator is undetectable.

In an aspect, provided herein is a cell, including a cell of any previous cell aspect and embodiments, wherein when the cell does not include a splice modulator, e.g., LMI070, the level of expression of the protein of interest is greater, e.g., 2, 3, 4, 5, 6, 7, 8, 9, 10, 20, 30, 50 or 100 fold greater, than the level of expression of the protein of interest when the cell includes said splice modulator, optionally wherein the level of expression when the cell includes said splice modulator is undetectable.

In an aspect, provided herein is a method of conditionally expressing a protein of interest, said method including: contacting an expression system (e.g. a cell, e.g., a cell of any one of the previous aspects and embodiments) including the nucleic acid molecule, the vector, or the recombinant virus of any previous aspect and embodiment, with a splice modulator, e.g., LMI070, wherein:
in the presence of said splice modulator, expression of said protein of interest is increased, e.g., 2, 3, 4, 5, 6, 7, 8, 9, 10, 20, 30, 50 or 100 fold greater, relative to the level of expression of said protein of interest in the absence of said splice modulator; and
   in the absence of said splice modulator, expression of said protein of interest is substantially decreased, e.g., e.g., 2, 3, 4, 5, 6, 7, 8, 9, 10, 20, 30, 50 or 100 fold less, relative to the level of expression of said protein of interest in the presence of the splice modulator.

In an aspect, provided herein is a method of conditionally expressing a protein of interest, said method including: contacting an expression system (e.g. a cell, e.g., a cell of any one of the previous aspects and embodiments) including the nucleic acid molecule, the vector, or the recombinant virus of any previous aspect and embodiment, with a splice modulator, e.g., LMI070, wherein:

in the absence of said splice modulator, expression of said protein of interest is increased, e.g., 2, 3, 4, 5, 6, 7, 8, 9, 10, 20, 30, 50 or 100 fold greater, relative to the level of expression of said protein of interest in the presence of said splice modulator; and
in the presence of said splice modulator, expression of said protein of interest is substantially decreased, e.g., e.g., 2, 3, 4, 5, 6, 7, 8, 9, 10, 20, 30, 50 or 100 fold less, relative to the level of expression of said protein of interest in the absence of the splice modulator.

In an aspect, provided herein is a pharmaceutical composition including a nucleic acid molecule, a vector, a recombinant virus, or a cell of any of the previous aspects and embodiments.

In an aspect, provided herein is a method of treating a subject in need of a gene therapy, said method including administering to said subject a nucleic acid molecule, a vector, a recombinant virus, a cell or a pharmaceutical composition of any of the previous aspects and embodiments. In embodiments, the method further includes administering to the subject an amount of a splice modulator, e.g., LMI070, effective to cause at least a 2, 3, 4, 5, 6, 7, 8, 9, 10, 20, 30, 50 or 100 fold increase or decrease in expression of the protein of interest, relative to the expression level of the protein of interest in the absence of the splice modulator.

In an aspect, provided herein is a kit including a nucleic acid molecule, a vector, a recombinant virus, a cell or a pharmaceutical composition of any of the previous aspects and embodiments; and a splice modulator.

In an aspect, provided herein is a nucleic acid molecule, a vector, a recombinant virus, a cell or a pharmaceutical composition of any of the previous aspects and embodiments, for use in a method of conditionally expressing a protein of interest, said method including: contacting an expression system (e.g. a cell, e.g., a cell of any one of aspects 53-57) including the nucleic acid molecule of any one of aspects 1-2 and 4-36, the vector of any one of aspects 37-45 or the recombinant virus of any one of aspects 46-52, with a splice modulator, e.g., LMI070, wherein:

in the presence of said splice modulator, expression of said protein of interest is increased, e.g., is at least 2, 3, 4, 5, 6, 7, 8, 9, 10, 20, 30, 50 or 100 fold greater, relative to the level of expression of said protein of interest in the absence of said splice modulator; and
in the absence of said splice modulator, expression of said protein of interest is substantially decreased, e.g., is at least 2, 3, 4, 5, 6, 7, 8, 9, 10, 20, 30, 50 or 100 fold less, relative to the level of expression of said protein of interest in the presence of the splice modulator.

In an aspect, provided herein is a nucleic acid molecule, a vector, a recombinant virus, a cell or a pharmaceutical composition of any of the previous aspects and embodiments, for use in a method of conditionally expressing a protein of interest, said method including: contacting an expression system (e.g. a cell, e.g., a cell of any one of aspects 53-57) including the nucleic acid molecule of any one of aspects 1 or 3-36, the vector of any one of aspects 37-45 or the recombinant virus of any one of aspects 46-52, with a splice modulator, e.g., LMI070, wherein:

in the absence of said splice modulator, expression of said protein of interest is increased, e.g., is at least 2, 3, 4, 5, 6, 7, 8, 9, 10, 20, 30, 50 or 100 fold greater, relative to the level of expression of said protein of interest in the presence of said splice modulator; and
in the presence of said splice modulator, expression of said protein of interest is substantially decreased, e.g., is at least 2, 3, 4, 5, 6, 7, 8, 9, 10, 20, 30, 50 or 100 fold less, relative to the level of expression of said protein of interest in the absence of the splice modulator.

In an aspect, provided herein is a nucleic acid molecule, a vector, a recombinant virus, a cell or a pharmaceutical composition of any of the previous aspects and embodiments, for use in a method of treating a subject in need of a gene therapy.

In an aspect, provided herein is a nucleic acid molecule, a vector, a recombinant virus, a cell or a pharmaceutical composition of any of the previous aspects and embodiments, or the nucleic acid, vector, recombinant virus, cell, or pharmaceutical composition for use according to any one of aspects 64-66, wherein the transgene encodes a protein of a genome editing system (for example, an RNA-guided nuclease such as a Cas9 protein, a zinc finger nuclease or a TALEN), an antibody or antibody fragment, or a therapeutic protein (for example, protein selected from progranulin, SMN, MeCP2, CLN2, CLN3, CLN4, CLN5, CLN6, CLN7, CLN8).

### BRIEF DESCRIPTION OF THE FIGURES

FIG. 1A. Describes the concept of a splice modulator-mediated “ON-switch”. In an ON-switch system, exon C contains a premature termination (stop) codon that is in frame with the coding sequence initiated by the start codon located in exon A when exon B is excluded. When a splice modulator such as LMI070 is included the transcript now includes frame-shifting exon B, thereby restoring an uninterrupted open reading frame which leads to transgene expression.

FIG. 1B. Describes the concept of a splice modulator-mediated “OFF-switch”. In an OFF-switch system, exon A is spliced to exon C, which leads to transgene expression. When a splice modulator such as LMI070 is present, exon B, which contains a premature termination (stop) codon, is included, resulting in termination of translation.

FIG. 2A. Design of AAV vector with SNX7 minigene-based switch. FIG. 2A shows a schematic diagram of the SNX7 locus containing a splice modulator (LMI070) exonic target binding site at chromosome: GRCh37:1:99204216:99204359:1 (AGTTTGCAAAGGAAGGAAAGGAGCAGAGACTTGAATGAGCAGAAAATCATTTCAGGGCCTGTTCTC TATGTCCTTGCTATCCCTGTCTTCTGTAGCTATTCTGAAACCATCAACAAAGGAGCACACCATTCCAT CAGCAAAAGA (SEQ ID NO: 80)), as well as an intronic sequence downstream of exon 8 at chromosome:GRCh37:1:99203793:99203946:1 (CTTCCAGAGGAGATTGGAAAACTTGAAGATAAAGTGGAATGTGCTAATAATGCCCTGAAAGCAGATT GGGAGAGATGGAAACAAAATATGCAAAATGATATCAAGTTAGCATTTACAGATATGGCTGAGGAGAA TATCCATTATTATGAACAG (SEQ ID NO: 99)), and 21,251 nucleotides upstream of exon 9 at chromosome:GRCh37:1:99225610:99225687:1 (TGCCTTGCTACGTGGGAGTCATTCCTTACATCACAGACCAACCTTCACTTGGAAGAAGCCTCTGAAG ATAAACCTTAA (SEQ ID NO: 100))

FIG. 2B. Design of AAV vector with SNX7 minigene-based switch. FIG. 2B shows the construction of the non-naturally occurring SNX7 minigene using exon 8 (called exon A), a 270 nucleotide intron (AB), an exon comprising a splice modulator (e.g., LMI070) binding site at its 3′ end (called exon B), a 407 nucleotide intron fragment (shortened from 21,251 nt; BC), and exon 9 (called exon C). Additional modifications were made to the minigene to improve its performance, such as: 1) a Kozak consensus sequence and ATG codon (GCCACCATG) was inserted at position 65 in exon A; 2) All other ATG sequences in the minigene were replaced with TTG; 3) a TA at position 20 of exon A was replaced with AG to make GAAGAAGAA sequence (SEQ ID NO: 69); 4) 1 nt was removed from exon B to create frame shift (number of nucleotides=3n−1) in ORF; 5) T was inserted at position 4 of exon C to create frame shift in ORF resulting in multiple stop codons; 6) TAC at position 9 of exon C was changed to TAA to create earlier termination codon; 7) CAG at position 34 of exon C was changed to ACC to mutate a potential cryptic splice site; 8) CTCT at position 60 of exon C was changed to TAGC to create a Nhe I restriction site; and 9) TAA at the end of exon C was removed to create continuous ORF.

FIG. 2C shows the construction of a scAAV vector comprising the SNX7 minigene ON switch. The scAAV was created by combining, AAV2 ITR containing a deletion of trs, followed by a JeT promoter, followed by the SNX7 minigene (see above, FIG. 2B), followed by a coding sequence for a furin cleavage site (RNRR (SEQ ID NO: 39)) added to the end of exon C, followed by coding sequence for a T2A peptide, followed by a transgene sequence (here, a coding sequence for EGFP without the first ATG); followed by a SV40 late polyadenylation signal, followed by an AAV2 ITR.

FIG. 3 shows the regulation of GFP expression using SNX7 minigene-based ON-switch (FIG. 3A) and OFF-switch (FIG. 3B), and the mRNA expression products in the absence of splice modulator (“no LMI070”) and in the presence of splice modulator (“Plus LMI070”). Figure discloses SEQ ID NOS 108-111, respectively, in order of appearance.

FIG. 4. Regulation of GFP expression by SNX7 switch in HEK293 cells. FIG. 4A shows GFP expression in HEK293 cells transfected with pSNX7-GFP (vector comprising an ON-switch) at various concentrations of splice modulator (LMI070). FIG. 4B plots GFP expression measured by mean fluorescence intensity as a function of LMI070 concentration. FIG. 4C plots quantitation of mRNA transcripts containing exon B or having direct exon A-to-exon C splicing at various concentrations of splice modulator.

FIG. 5. Regulation of GFP expression by SNX7 switch in rat cortical neurons. FIG. 5A shows GFP expression levels in primary rat neurons transfected with pSNX7-GFP (vector comprising an ON-switch) at various concentrations of splice modulator (LMI070). FIG. 5B plots quantitation of mRNA transcripts containing exon B or having direct exon A-to-exon C splicing at various concentrations of splice modulator in rat cortical neurons.

FIG. 6. AAV vectors comprising a human progranulin (PRGN) transgene under the control of SNX7 ON-switch. FIG. 6A shows 1) schematic diagram ssAAV vector comprising a neuron-specific promoter (human Synapsin promoter) and containing an SNX7 ON-switch minigene. FIG. 6B shows hPRGN expression in primary rat neurons transfected with the vectors described in FIG. 6A (Syn_SNX) in the presence or absence of splice modulator, compared with hPRGN expression levels from vectors which do not comprise the SNX7-based switch (“Syn”). FIG. 6C shows mRNA expression levels for mRNA that includes exon B and mRNA that has direct exon A to exon C splicing, in the presence and absence of splice modulator.

FIG. 7A. depicts study plan of timecourse in vivo testing AAV vector containing SNX7 switch (version 1). Single stranded AAV9 containing hPGRN expression cassette under control of synapsin promoter with SNX7 switch was injected ICV in P0 neonatal mice. After 4 weeks, mice received oral administration of 30 mg/kg LMI070 and mice were taken down at different time points starting 24 hours post administration. FIG. 7B. demonstrates that oral administration of LMI070 switches on transgene expression in mouse brain in time-dependent manner in mice previously administered the AAV factor described in FIG. 7A. Graph demonstrates TR-FRET measurement of hPGRN expression in brain after indicated times post LMI070 delivery.

FIG. 8A. depicts study plan of dose-response in vivo testing AAV vector containing SNX7 switch (version 1). Single stranded AAV9 containing hPGRN expression cassette under control of synapsin promoter with SNX7 switch was injected ICV in P0 neonatal mice. After 4 weeks, mice received oral administration of different doses LMI070 and mice were taken down at different time points starting 12 hours post administration. FIG. 8B demonstrates that oral administration of LMI070 switches on transgene expression in mouse brain in dose-dependent mannerin mice previously administered the AAV vector described in FIG. 8A. Graph demonstrates TR-FRET measurement of hPGRN expression in brain upon indicated doses of LMI070 and after indicated times post LMI070 delivery.

FIG. 9 shows comparison of the first version of SNX7 minigene and the modified SNX7 minigene (version 2), which has reduced size and reduced peptide expression in the absence of LMI070. Figure discloses SEQ ID NOS 108 and 112-113, respectively, in order of appearance.

FIG. 10 shows that the modified SNX7 minigene (version 2) is more sensitive than the previous version of SNX7 minigene in response to LMI070.

### DETAILED DESCRIPTION

The disclosed compositions and methods may be understood more readily by reference to the following detailed description taken in connection with the accompanying figures, which form a part of this disclosure.

Throughout this text, the descriptions refer to compositions and methods of using the compositions. Where the disclosure discloses or claims a feature or embodiment associated with a composition, such a feature or embodiment is equally applicable to the methods of using, or uses of the composition. Likewise, where the disclosure discloses or claims a feature or embodiment associated with a method of using a composition, such a feature or embodiment is equally applicable to the composition. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise. All references cited herein are incorporated by reference for any purpose. Where a reference and the specification conflict, the specification will control. It is to be appreciated that certain features of the disclosed compositions and methods, which are, for clarity, disclosed herein in the context of separate embodiments, may also be provided in combination in a single embodiment. Conversely, various features of the disclosed compositions and methods that are, for brevity, disclosed in the context of a single embodiment, may also be provided separately or in any sub-combination.

### Definitions

As used herein, the singular forms “a,” “an,” and “the” include plural forms unless the context clearly dictates otherwise. The term “about” or “approximately,” when used in the context of numerical values and ranges, refers to values or ranges that approximate or are close to the recited values or ranges such that the embodiment may perform as intended, as is apparent to the skilled person from the teachings contained herein. In some embodiments, about means plus or minus 10% of a numerical amount.

The terms “polynucleotide” and “nucleic acid” are used interchangeably herein and refer to a polymeric form of nucleotides of any length. They may include one or more of ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases, e.g. locked nucleic acids (LNA), peptide nucleic acids (PNA).

The terms “peptide,” “polypeptide,” and “protein” are used interchangeably, and refer to a compound comprised of amino acid residues covalently linked by peptide bonds. A protein or peptide typically contains at least two amino acids or amino acid variants, and no limitation is placed on the maximum number of amino acids that can comprise a protein's or peptide's sequence. Polypeptides include any peptide or protein comprising two or more amino acids or variants joined to each other by peptide bonds. The terms include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “sequence identity” and “sequence homology” are used interchangeably herein, and as used in connection with a polynucleotide or polypeptide, refers to the percentage of bases or amino acids that are the same, and are in the same relative position, when comparing or aligning two sequences of polynucleotides of polypeptides. Sequence identity can be determined in a number of different manners. For instance, sequences may be aligned using various methods and computer programs (e.g., BLAST, T-COFFEE, MUSCLE, MAFFT, etc.). See, e.g., Altschul et al., (1990) J. Mol. Bioi., 215:403-10.

The term “isolated” in reference to a nucleic acid or protein discussed herein refers to a nucleic acid or protein that has been separated from one or more of the components normally found associated with it in the natural environment. The separation may comprise removal from a larger nucleic acid (e.g., from a gene or chromosome) or from other proteins or molecules normally in contact with the nucleic acid or protein. The term encompasses but does not require complete isolation.

As used herein, an isolated nucleic acid comprising a “heterologous nucleic acid sequence” refers to an isolated nucleic acid comprising a portion (i.e., the heterologous nucleic acid portion) that is not normally found operably linked to one or more other components of the isolated nucleic acid in a natural context. For instance, the heterologous nucleic acid may comprise a nucleic acid sequence not originally found in a cell, bacterial cell, virus, or organism from which other components of the isolated nucleic acid (e.g., the promoter) naturally derive or where the other components of the isolated nucleic acid (e.g., the promoter) are not naturally found operatively linked with the heterologous nucleic acid in the cell, bacterial cell, virus, or organism. In some embodiments the heterologous nucleic acid includes a transgene. As used herein, a “transgene” is a nucleic acid sequence that encodes a molecule of interest (for example, a therapeutic protein, a reporter protein or a therapeutic RNA molecule) that is not originally associated with one or more components of the nucleic acid molecule. In some embodiments, the heterologous nucleic acid sequence encodes a human protein. In some embodiments, the heterologous nucleic acid sequence encodes an RNA sequence, e.g., a shRNA.

A DNA sequence or DNA polynucleotide sequence that “encodes” a particular RNA is a sequence of DNA that is capable of being transcribed into RNA. A DNA polynucleotide may encode an RNA (mRNA) that is translated into protein, or a DNA polynucleotide may encode an RNA that is not translated into protein (e.g. tRNA, rRNA, or a guide RNA; also called “non-coding” RNA or “ncRNA”). A DNA sequence or DNA polynucleotide sequence may also “encode” a particular polypeptide or protein sequence, wherein, for example, the DNA directly encodes an mRNA that can be translated into the polypeptide or protein sequence. A “protein coding sequence” or a sequence that encodes a particular protein or polypeptide is a nucleic acid sequence that is capable of being transcribed into mRNA (in the case of DNA) and translated (in the case of mRNA) into a polypeptide in vitro or in vivo when placed under the control of appropriate regulatory sequences. The boundaries of the coding sequence may be determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The term “promoter” or “promoter sequence” as used herein is a DNA regulatory sequence capable of facilitating transcription (e.g., capable of causing detectable levels of transcription and/or increasing the detectable level of transcription over the level provided in the absence of the promoter) of an operatively linked coding or non-coding sequence, e.g., of a downstream (3′ direction) coding or non-coding sequence, e.g., through binding RNA polymerase. In some embodiments, the promoter sequence is bounded at its 3′ terminus by the transcription initiation site and extends upstream (5′ direction) to include the minimum number of bases or elements to initiate transcription at levels detectable above background. In some embodiments, a promoter sequence may comprise a transcription initiation site, as well as protein binding domains responsible for the binding of RNA polymerase. In addition to sequences sufficient to initiate transcription, a promoter may also include sequences of other regulatory elements that are involved in modulating transcription (e.g., enhancers, Kozak sequences and introns). Various promoters, including inducible promoters and constitutive promoters, may be used to drive the vectors disclosed herein. Examples of promoters known in the art that may be used in some embodiments, e.g., in viral vectors disclosed herein, include the CMV promoter, CBA promoter, smCBA promoter and those promoters derived from an immunoglobulin gene, SV40, or other tissue specific genes (e.g: RLBP1, RPE, VMD2). In addition, standard techniques are known in the art for creating functional promoters by mixing and matching known regulatory elements. Fragments of promoters, e.g., those that retain at least minimum number of bases or elements to initiate transcription at levels detectable above background, may also be used.

In some embodiments, a promoter can be a constitutively active promoter (i.e., a promoter that constitutively drives expression in any cell type and/or under any conditions). In other embodiments, a promoter can be a constitutively active promoter in a particular tissue context, e.g., in neurons, in cardiac cells, etc. In other embodiments, a promoter can be an inducible promoter (i.e., a promoter whose activity is controlled by an external stimulus, e.g., the presence of a particular temperature, compound, or protein). In some embodiments, a promoter may be a spatially restricted promoter that can drive activity or not depending on the physical context in which the promoter is found. Non-limiting examples of spatially restricted promoters include tissue specific promoter, cell type specific promoter, etc. In some embodiments, a promoter may be a temporally restricted promoter that drives expression depending on the temporal context in which the promoter is found. For example, a temporally restricted promoter may drive expression only at specific stages of embryonic development or during specific stages of a biological process. Non-limiting examples of temporally restricted promoters include hair follicle cycle promoters in mice.

In some embodiments, the promoter is tissue-specific such that, in a multi-cellular organism, the promoter drives expression only in a subset of specific cells. For example, tissue-specific promoters include, but are not limited to, neuron-specific promoters, adipocyte-specific promoters, cardiomyocyte-specific promoters, smooth muscle-specific promoters, photoreceptor-specific promoters, etc. A neuron-specific promoter refers to a promoter that, when administered e.g., peripherally, directly into the central nervous system (CNS), or delivered to neuronal cells, including in vitro, ex vivo, or in vivo, preferentially drives or regulates expression of an operatively-linked heterologous nucleic acid, e.g., one encoding a protein or peptide or shRNA of interest, in neurons as compared to expression in non-neuronal cells.

The terms “DNA regulatory sequences,” “control elements,” and “regulatory elements,” used interchangeably herein, refer to transcriptional and translational control sequences, such as promoters, enhancers, silencers, polyadenylation signals, terminators, protein degradation signals, and the like, that provide for and/or regulate transcription of a non-coding sequence (e.g., a short hairpin RNA) or a coding sequence (e.g., PGRN) and/or regulate translation of an encoded polypeptide.

The terms “polyadenylation (polyA) signal sequence” and “polyadenylation sequence” refer to a regulatory element that provides a signal for transcription termination and addition of an adenosine homopolymeric chain to the 3′ end of an RNA transcript. The polyadenylation signal may comprise a termination signal (e.g., an AAUAAA sequence or other non-canonical sequences) and optionally flanking auxiliary elements (e.g., a GU-rich element) and/or other elements associated with efficient cleavage and polyadenylation. The polyadenylation sequence may comprise a series of adenosines attached by polyadenylation to the 3′ end of an mRNA. Specific polyA signal sequences may include the poly(A) signal of SEQ ID NO:22 or of SEQ ID NO: 89. In some embodiments, DNA regulatory sequences or control elements are tissue-specific regulatory sequences.

The term “post-transcriptional regulatory element” (“PRE”) refers to one or more regulatory elements that, when transcribed into mRNA, regulate gene expression at the level of the mRNA transcript. Examples of such post-transcriptional regulatory elements may include sequences that encode micro-RNA binding sites, RNA binding protein binding sites, etc. Examples of post-transcriptional regulatory element that may be used with the nucleic acid molecules and vectors disclosed herein include the woodchuck hepatitis post-transcriptional regulatory element (WPRE), the hepatitis post-transcriptional regulatory element (HPRE). Exemplary PREs may also include the PRE disclosed as SEQ ID NO: 88. Examples PREs may also include the PRE disclosed as SEQ ID NO: 72 or the PRE disclosed as SEQ ID NO: 73.

The term “intron” refers to nucleic acid sequence(s), e.g., those within an open reading frame, that are noncoding for one or more amino acids of a polypeptide transcript (e.g., protein of interest) expressed from the nucleic acid. Intronic sequences may be transcribed from DNA into RNA (i.e., may be present in the pre-mRNA), but may be removed before the protein is expressed from the mature mRNA, e.g., through splicing.

The term “exon” refers to nucleic acid sequence(s), e.g., those within an open reading frame, that are coding for one or more amino acids of a transcript (e.g., a protein of interest) expressed from a nucleic acid. Exonic sequences may be transcribed from DNA into RNA (i.e., may be present in the pre-mRNA), and also may be present in a mature mRNA (i.e., the processed form of RNA (e.g., after splicing)) that is translated to a polypeptide.

As used herein, processes conducted “in vitro” refer to processes which are performed outside of the normal biological environment, for example, studies performed in a test tube, a flask, a petri dish, in artificial culture medium. Processes conducted “in vivo” refer to processes performed within living organisms or cells. for example, studies performed in cell cultures or in mice. Studies performed “ex vivo” refer to studies done in or on tissue from an organism in an external environment, e.g., with minimal alteration of natural conditions, e.g., allowing for manipulation of an organism's cells or tissues under more controlled conditions than may be possible in in vivo experiments.

The term “naturally-occurring” or “unmodified” as used herein as applied to, e.g., a nucleic acid, a polypeptide, a cell, or an organism, is one found in nature. For example, a polypeptide or polynucleotide sequence that is present in an organism (such as a virus) is naturally occurring whether present in that organism or isolated from one or more components of the organism.

In some embodiments, a “vector” is any genetic element (e.g., DNA, RNA, or a mixture thereof) that contains a nucleic acid of interest (e.g., a transgene) that is capable of being expressed in a host cell, e.g., a nucleic acid of interest within a larger nucleic acid sequence or structure suitable for delivery to a cell, tissue, and/or organism, such as a plasmid, phage, transposon, cosmid, chromosome, virus, virion, etc. For instance, a vector may comprise an insert (e.g., a heterologous nucleic acid comprising a transgene encoding a gene to be expressed or an open reading frame of that gene) and one or more additional elements, e.g., a minigene as described herein and/or elements suitable for delivering or controlling expression of the insert. The vector may be capable of replication and/or expression, e.g., when associated with the proper control elements, and it may be capable of transferring genetic information between cells. In some embodiments, a vector may be a vector suitable for expression in a host cell, e.g, an AAV vector. In some embodiments, a vector may be a plasmid suitable for expression and/or replication, e.g., in a cell or bioreactor. In some embodiments, vectors designed specifically for the expression of a heterologous nucleic acid sequence, e.g., a transgene encoding a protein of interest, shRNA, and the like, in the target cell may be referred to as expression vectors, and generally have a promoter sequence that drives expression of the transgene. In other embodiments, vectors, e.g., transcription vectors, may be capable of being transcribed but not translated: they can be replicated in a target cell but not expressed. Transcription vectors may be used to amplify their insert.

The term “expression vector” refers to a vector comprising a polynucleotide comprising expression control sequences operatively linked to a nucleotide sequence to be expressed. An expression vector may comprise sufficient cis-acting elements for expression, alone or in combination with other elements for expression supplied by the host cell or in an in vitro expression system.

Expression vectors include, e.g., cosmids, plasmids (e.g., naked or contained in liposomes) and viruses (e.g., lentiviruses, retroviruses, adenoviruses, and adeno-associated viruses) that incorporate the recombinant polynucleotide.

The term “plasmid” refers to a nonchromosomal (and typically double-stranded) DNA sequence comprising an intact “replicon” such that the plasmid is replicated in a host cell. A plasmid may be a circular nucleic acid. When the plasmid is placed within a unicellular organism, the characteristics of that organism are changed or transformed as a result of the DNA of the plasmid. For example, a plasmid carrying the gene for tetracycline resistance (TcR) transforms a cell previously sensitive to tetracycline into one which is resistant to it. Exemplary plasmids useful in some embodiments for the viral vectors disclosed herein include SEQ ID NO: 92.

The term “recombinant virus” as used herein is intended to refer to a non-wild-type and/or artificially produced recombinant virus (e.g., a parvovirus, adenovirus, lentivirus or adeno-associated virus etc.) that comprises a transgene or other heterologous nucleic acid. The recombinant virus may comprise a recombinant viral genome (e.g. comprising a minigene as described herein and a transgene) packaged within a viral (e.g.: AAV) capsid. A specific type of recombinant virus may be a “recombinant adeno-associated virus”, or “rAAV”. The recombinant viral genome packaged in the viral capsid may be a viral vector. In some embodiments, the recombinant viruses disclosed herein comprise viral vectors (e.g., comprising a minigene and transgene of interest, e.g., as described herein). Examples of viral vectors include but are not limited to an adeno-associated viral (AAV) vector, a chimeric AAV vector, an adenoviral vector, a retroviral vector, a lentiviral vector, a DNA viral vector, a herpes simplex viral vector, a baculoviral vector, or any mutant or derivative thereof.

In another embodiment, the term “transfection” is used to refer to the uptake of foreign DNA by a cell, such that the cell has been “transfected” once the exogenous DNA has been introduced inside the cell membrane. See, e.g., Graham et al., (1973) Virology, 52:456; Sambrook et al., (1989) Molecular Cloning, a laboratory manual, Cold Spring Harbor Laboratories, New York; Davis et al., (1986) Basic Methods in Molecular Biology, Elsevier; Chu et al., (1981) Gene, 13:197. Such techniques can be used to introduce one or more exogenous DNA moieties into suitable host cells. In some embodiments, the term “transduction” is used to refer to the uptake of foreign DNA by a cell, where the foreign DNA is provided by a virus or a viral vector. Consequently, a cell has been “transduced” when exogenous DNA has been introduced inside the cell membrane. In some embodiments, the term “transformation” is used to refer to the uptake of foreign DNA by bacterial cells.

As used herein, the term “cell line” refers to a population of cells capable of continuous or prolonged growth and division in vitro. In certain circumstances, spontaneous or induced changes can occur in karyotype during storage or transfer of such clonal populations. Therefore, cells derived from the cell line referred to may not be precisely identical to the ancestral cells or cultures, and the cell line referred to includes such variants.

The term “operably linked” refers to a functional relationship between two or more polynucleotide (e.g., DNA) segments. Typically, the term refers to the functional relationship of a transcriptional regulatory sequence and a sequence to be transcribed. For example, a promoter or enhancer sequence is operably linked to a coding sequence if it, e.g., stimulates or modulates the transcription of the coding sequence in an appropriate host cell or other expression system. Generally, promoter transcriptional regulatory sequences that are operably linked to a sequence are contiguous to that sequence or are separated by short spacer sequences, i.e., they are cis-acting. However, some transcriptional regulatory sequences, such as enhancers, need not be physically contiguous or located in close proximity to the coding sequences whose transcription they enhance.

As used herein, the term “AAV vector” refers to a vector derived from or comprising one or more nucleic acid sequences derived from an adeno-associated virus serotype, including without limitation, an AAV-1, AAV-2, AAV-3, AAV-4, AAV-5, AAV-6, AAV-7, AAV-8 or AAV-9 viral vector. AAV vectors may have one or more of the AAV wild-type genes deleted in whole or part, e.g., the rep and/or cap genes, while retaining, e.g., functional flanking inverted terminal repeat (“ITR”) sequences. In some embodiments, an AAV vector may be packaged in a protein shell or capsid, e.g., comprising one or more AAV capsid proteins, which may provide a vehicle for delivery of vector nucleic acid to the nucleus of target cells. In some embodiments, an AAV vector comprises one or more AAV ITR sequences (e.g., AAV2 ITR sequences). In some embodiments, an AAV vector comprises one or more AAV ITR sequences (e.g., AAV2 ITR sequences) but does not contain any additional viral nucleic acid sequence. In some embodiments, the AAV vector components (e.g., ITRs) are derived from a different serotype virus than the rAAV capsid (for example, the AAV vector may comprise ITRs derived from AAV2 and the AAV vector may be packaged into an AAV9 capsid). Embodiments of these vector constructs are provided, e.g., in WO/2019/094253 (PCT/US2018/058744), which is incorporated herein by reference in its entirety.

In some embodiments, an “scAAV” is a self-complementary adeno-associated virus (scAAV). scAAV is termed “self-complementary” because at least a portion of the vector (e.g., at least a portion of the coding region) of the scAAV forms an intra-molecular double-stranded DNA. In some embodiments, the rAAV is an scAAV. In some embodiments, a viral vector is engineered from a naturally occurring adeno-associated virus (AAV) to provide an scAAV for use in gene therapy. Embodiments of these vector constructs and methods of preparing and purifying them are provided, e.g., in WO/2019/094253 (PCT/US2018/058744), which is incorporated herein by reference in its entirety.

In some embodiments, an “ssAAV” is a single-stranded adeno-associated virus (ssAAV). ssAAV is termed “single-stranded” because at least a portion of the vector (e.g., at least a portion of the coding region) of the ssAAV is single-stranded DNA. In some embodiments, the rAAV is an ssAAV. In some embodiments, a viral vector is engineered from a naturally occurring adeno-associated virus (AAV) to provide an ssAAV for use in gene therapy.

As used herein, an “virus” or “virion” indicates a viral particle, comprising a viral vector, e.g., alone or in combination with one or more additional components such as one or more viral capsids. For instance, an AAV virus may comprise, e.g., a linear, single-stranded AAV nucleic acid genome associated with an AAV capsid protein coat.

In some embodiments, terms such as “virus,” “virion,” “AAV virus,” “recombinant AAV virion,” “rAAV virion,” “AAV vector particle,” “full capsids,” “full particles,” and the like refer to infectious, replication-defective virus, e.g., those comprising an AAV protein shell encapsidating a heterologous nucleotide sequence of interest, e.g., in a viral vector which is flanked on one or both sides by AAV ITRs. A rAAV virion may be produced in a suitable host cell which comprises sequences, e.g., one or more plasmids, specifying an AAV vector, alone or in combination with nucleic acids encoding AAV helper functions and accessory functions (such as cap genes), e.g., on the same or additional plasmids. In some embodiments, the host cell is rendered capable of encoding AAV polypeptides that provide for packaging the AAV vector (containing a recombinant nucleotide sequence of interest) into infectious recombinant virion particles for subsequent gene delivery.

The terms “inverted terminal repeat” or “ITR” refer to a stretch of nucleotide sequences that can form a T-shaped palindromic structure, e.g., in adeno-associated viruses (AAV) and/or recombinant adeno-associated viral vectors (rAAV). Muzyczka et al., (2001) Fields Virology, Chapter 29, Lippincott Williams & Wilkins. In recombinant AAV vectors, these sequences may play a functional role in genome packaging and in second-strand synthesis.

The term “host cell” denotes a cell comprising an exogenous nucleic acid of interest, for example, one or more microorganism, yeast cell, insect cell, or mammalian cell. For instance, the host cell may comprise an AAV helper construct, an AAV vector plasmid, an accessory function vector, and/or other transfer DNA. The term includes the progeny of the original cell which has been transfected. The progeny of a single parental cell may not necessarily be completely identical in morphology or in genomic or total DNA complement as the original parent, due to natural, accidental, or deliberate mutation.

The term “AAV helper function” refers to an AAV-derived coding sequences which can be expressed to provide AAV gene products, e.g., those that function in trans for productive AAV replication. For instance, AAV helper functions may include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions may be used herein to complement AAV functions in trans that are missing from AAV vectors.

The term “AAV helper construct” refers generally to a nucleic acid molecule that includes nucleotide sequences providing or encoding proteins or nucleic acids that provide AAV functions deleted from an AAV vector, e.g. a vector for delivery of a nucleotide sequence of interest to a target cell or tissue. AAV helper constructs are commonly used to provide transient expression of AAV rep and/or cap genes to complement missing AAV functions for AAV replication. Typically, helper constructs lack AAV ITRs and can neither replicate nor package themselves. AAV helper constructs may be in the form of a plasmid, phage, transposon, cosmid, virus, or virion. A number of AAV helper constructs have been disclosed, such as the commonly used plasmids pAAV/Ad and pIM29+45 which encode both Rep and Cap expression products. See, e.g., Samulski et al., (1989) J. Virol., 63:3822-3828; McCarty et al., (1991) J. Virol., 65:2936-2945. A number of other vectors have been disclosed which encode Rep and/or Cap expression products. See, e.g., U.S. Pat. Nos. 5,139,941 and 6,376,237. Embodiments of these vector constructs and methods of preparing and purifying them are provided, e.g., in WO/2019/094253 (PCT/US2018/058744), which is incorporated herein by reference in its entirety.

A “minigene” as the term is used herein refers to a nucleic acid sequence comprising a plurality of introns and exons and at least one splice modulator binding site. In embodiments, the presence or absence of the splice modulator during expression from the heterologous nucleic acid sequence modulates the number of exons present in the mature mRNA. Minigenes are described more fully herein.

A “splice modulator” is a molecule which binds to a splice modulator binding site and modulates the splicing of a pre-mRNA molecule, for example, a pre-mRNA molecule produced from a nucleic acid molecule described herein. In embodiments, the splice modulator increases the inclusion of an exon in the mature mRNA molecule. In other embodiments, the splice modulator decreases the inclusion of an exon in the mature mRNA molecule.

A “splice modulator binding sequence” is a sequence of nucleic acids which is recognized by a splice modulator. The term should be understood to encompass both the sequence found in a pre-mRNA as well as the sequence found in the DNA from which the pre-mRNA was produced. In exemplary embodiments, the splice modulator is a compound described herein, e.g., LMI070, and the splice modulator binding site includes the sequence AGA. In embodiments, the splice modulator binding site is disposed at or near, e.g., at, the 3′ end of an exon of a minigene described herein.

A “pre-mRNA” is the first form of RNA created through transcription of DNA (e.g., of a nucleic acid molecule described herein) that has not yet undergone further processing, such as, for example, splicing. Thus, a pre-mRNA can include both introns and exons. Pre-mRNA molecules are further processed, e.g., through splicing, to from the “mature-RNA” or “mRNA.”

The nucleic acid sequences, minigenes, vectors, and methods disclosed herein relate to minigenes and regulatable expression systems comprising said minigenes, uses of splice modulators in combination with such minigenes and expression systems to control transgene expression, other uses therefore, and combinations thereof, for example, those that (1) drive expression of a transgene sequence in the presence of a splice modulator and reduce expression of the transgene sequence in the absence of a splice modulator (ON-switch) and (2) drive expression of a transgene sequence in the absence of a splice modulator and reduce expression of the transgene sequence in the presence of a splice modulator (OFF-switch). For instance, the nucleic acid sequences, vectors, and methods disclosed herein may drive expression of human PGRN or other therapeutic protein sequence in a splice-modulator-dependent fashion.

### Nucleic Acid Molecules

1. Disclosed herein are nucleic acid molecules comprising a transgene encoding a molecule of interest (e.g., a protein of interest) wherein the transgene is operably linked to a minigene, e.g., as described hererin.

### Minigenes

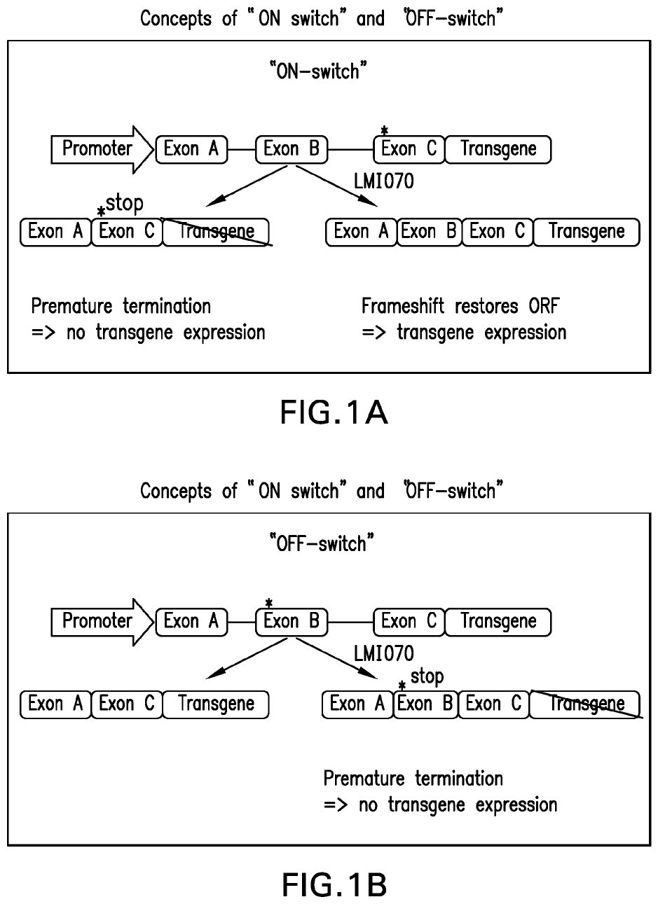

The nucleic acid molecules and other aspects disclosed herein include a minigene. Exemplary minigenes of the invention are depicted in FIG. 1A (on switch) and FIG. 1B (off switch). Disclosed herein are minigenes which are nucleic acid sequences comprising a plurality of introns and exons and at least one splice modulator binding site. In embodiments, the minigene is operably linked to a transgene. Minigenes as described herein are used in conjunction with one or more splice modulators to control (e.g., turn on or turn off) expression of a molecule of interest from a transgene that is associated with the minigene.

In aspects, a minigene comprises: a first exon; a first intron; a second exon; a second intron; and a third exon; wherein said second exon comprises a splice modulator binding sequence and wherein, in the presence of a splice modulator, said second exon is included in an mRNA product of the nucleic acid, and in the absence of said splice modulator, said second exon is not included in an mRNA product of the nucleic acid.

In aspects, the third exon of the minigene includes a stop codon that is in frame in the mRNA product of the nucleic acid produced in the absence of the splice modulator and which is not in frame in the mRNA product of the nucleic acid produced in the presence of the splice modulator. Thus, in the absence of a splice modulator, translation of a sequence encoding a molecule of interest (e.g., a protein of interest) disposed downstream of the minigene is reduced, for example, due to premature termination of translation by inclusion of the exon comprising the in-frame stop codon, whereas in the presence of the splice modulator, the stop codon is out of frame and translation of the molecule of interest is increased. Such aspects are thus referred to herein as “on-switch” minigenes since the presence of the splice modulator turns “on” (e.g., increases) expression of the molecule of interest.

In other embodiments, the second exon comprises a stop codon that is in frame in the mRNA product of the nucleic acid produced in the presence of the splice modulator. Thus, in the presence of a splice modulator, the exon comprising the stop codon is included in the transcript, and translation of a sequence encoding a molecule of interest (e.g., a protein of interest) disposed downstream of the minigene is decreased, whereas in the absence of the splice modulator, the exon comprising the stop codon is not present in the mRNA and expression of the molecule of interest is increased. Such aspects are thus referred to herein as “off-switch” minigenes since the presence of the splice modulator turns “off” (e.g., decreases) expression of the molecule of interest.

Without being bound by theory, it is recognized herein that vectors may have limited coding capacity (i.e., in order to be functional, their size may be limited). Thus, contemplated herein are minigenes which comprises fewer than 2000, fewer than 1900, fewer than 1800, fewer than 1700, fewer than 1600, fewer than 1500, fewer than 1400, fewer than 1300, fewer than 1200, fewer than 1100, fewer than 1000, fewer than 900, fewer than 800, fewer than 700, fewer than 600, or fewer than 500 nucleotides. Also contemplated herein are minigenes which comprise between about 2500 and about 500 nucleotides, e.g., between about 2000 and about 600 nucleotides, e.g., between about 1500 and about 700 nucleotides, e.g., between about 1200 and about 800 nucleotides, e.g. between about 1100 and about 900 nucleotides. Without being bound by theory, minigenes having such length can be included by a vector comprising a transgene and the resulting vector is of appropriate size to be functional, e.g., in a host cell. In embodiments, the sequences of the minigene are of human origin or are derived from sequences of human origin. Where the reference sequences of human origin which are identified as comprising a slice modulator binding sequence are longer than the lengths contemplated herein, such sequences may be shortened such as, for example, by deleting intronic or exonic sequence.

In embodiments, the minigenes described herein may be further modified. Such modifications are designed to improve one or more properties of the minigene. For example, a sequence derived from a human genome sequence may be included in a minigene may be further modified to mutate or remove one or more start codons (e.g., ATG sequences); remove or mutate all unwanted potential splice acceptor or splice donor sequences; include 1 or more, e.g., 2, 3, 4, 5, or 6 GAA repeats (SEQ ID NO: 101) (e.g., include GAAGAAGAA: SEQ ID NO: 69); include a Kozak sequence (e.g., a Kozak sequence of GCCACC: SEQ ID NO: 70); or any combination of modifications thereof.

### Splice Modulator Binding Sequences

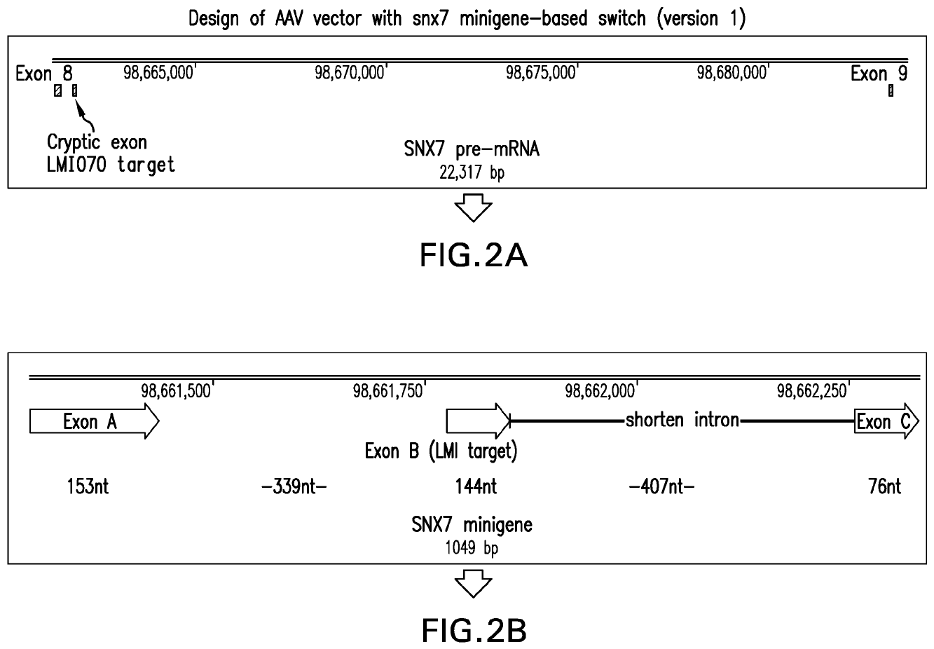

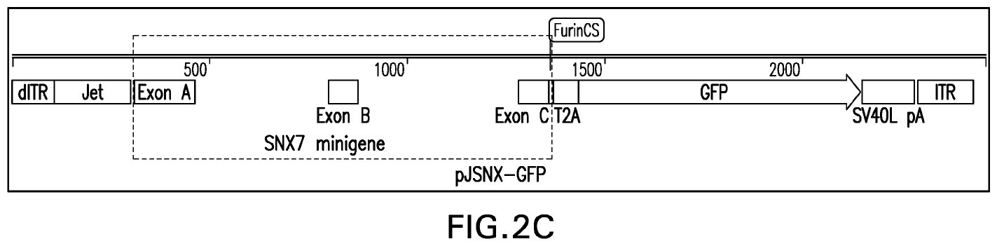

The aspects of the invention include minigenes comprising at least one exon comprising a splice modulator binding sequence. In aspects, the splice modulator binding sequence is disposed at or near the ′3 end of an exon of the minigene. In aspects, the splice modulator binding sequence is disposed at the ′3 end of an exon of the minigene. In aspects, the splice modulator binding site is derived from a sequence of the human genome. The methods described herein, e.g., in the Examples, are used to identify splice modulator binding sites recognized by splice modulators. Table 1 below, lists exemplary sequences of exons comprising a splice modulator binding site (e.g., the sequence AGA) at the ′3 end of exon. Such splice modulator binding sequences are recognized by splice modulators described herein such as LMI070. FIG. 2 shows the design of a minigene derived from SNX7.

TABLE 1
Sequences of top 10 exonic targets of LMI070 (e.g., comprising a sequence -AGA at or
near the 3′ end of the exon) as identified by RNAseq.

| | | | | | EXON | | SEQ ID |
|---|---|---|---|---|---|---|---|
| SYMBOL | CHR. | START | END | STRAND | LENGTH | SEQUENCE | NO: |
| ARSJ | CHR4 | 114894796 | 114894867 | − | 72 | GTAATTAGCTGAGAAGGAAGATCTG | 2 |
| | | | | | | AAGGTTTAACGAGAGAGGGCGAGAG | |
| | | | | | | ATACAAAATATCTGCTAGGAGA | |
| GXYLT1 | CHR12 | 42488953 | 42489016 | − | 64 | GGATTGTTTGTATTCCTGCCAATGAT | 3 |
| | | | | | | TTGTGAGACAGTCTGTTCCCCACATC | |
| | | | | | | CTCGTCAACAGA | |
| HSD17B4 | CHR5 | 118792986 | 118793063 | + | 78 | CTTTCTGACATCTTAACGAGGCAATA | 4 |
| | | | | | | CAGAGAGACGAATTTTCATCAGTTTG | |
| | | | | | | TTCAGGGAGACACATATAACAAAAG | |
| | | | | | | A | |
| IFT57 | CHR3 | 107911323 | 107911373 | − | 51 | ATCCATACATACTTAATGCTGAAATG | 5 |
| | | | | | | TGAAGGGCTGAGAAAAAAGAAAAGA | |
| MARCH7 | CHR2 | 160619771 | 160619867 | + | 97 | AATTGGAAACATCGAGGGAAAATGG | 6 |
| | | | | | | GCTTTTTATTATTAAAACAAAACCTCA | |
| | | | | | | GTATTATCACTTAGAAACCTGAAATT | |
| | | | | | | GAACTCCAAAAGCCAAAGA | |
| SNX24 | CHR5 | 122233837 | 122233931 | + | 95 | AAGAATGTTCCTTTTGTGAAGAATGA | 7 |
| | | | | | | CTTAAGGAAGATTCATGATGACTGA | |
| | | | | | | GTGTGCCCGTGTGGAACTTTAGGAC | |
| | | | | | | ATAGATGCACTCCTACAGA | |
| SNX7 | CHR1 | 99204287 | 99204359 | + | 73 | CCTTGCTATCCCTGTCTTCTGTAGCT | 1 |
| | | | | | | ATTCTGAAACCATCAACAAAGGAGCA | |
| | | | | | | CACCATTCCATCAGCAAAAGA | |
| STRADB | CHR2 | 202335765 | 202335834 | + | 70 | TTGTCCTTCACTCCGTACTCCAGTTG | 8 |
| | | | | | | GCCAAGCATAGGTCGCATGCCAGG | |
| | | | | | | GTCAAGGAGACTAAGGGAGA | |
| VDAC2 | CHR10 | 76990168 | 76990208 | + | 41 | GACATACAGACATGGCAGCCCCTAG | 9 |
| | | | | | | CATGTGTATCCTAAGA | |
| VDAC2 | CHR10 | 76990169 | 76990208 | + | 40 | ACATACAGACATGGCAGCCCCTAGC | 10 |
| | | | | | | ATGTGTATCCTAAGA | |

SEQ ID NO: 80 is the full genomic sequence (144 nt) of the cryptic exon comprising a splice modulator binding site, located between exon 8 and 9 of the snx7 locus comprising SEQ ID NO: 1.

(SEQ ID NO: 80)
AGTTTGCAAAGGAAGGAAAGGAGCAGAGACTTGAATGAGCAGAAAATCAT
TTCAGGGCCTGTTCTCTATGTCCTTGCTATCCCTGTCTTCTGTAGCTATT
CTGAAACCATCAACAAAGGAGCACACCATTCCATCAGCAAAAGA.

SEQ ID NO:16 is derived from SEQ ID NO: 80, with the modifications to create frameshift in ORF and removed start codons to avoid leaking expression.

(SEQ ID NO: 16)
AGTTTGCAAAGGAAGGAAAGGAGCAGAGACTTGATTGAGCAGAAAATCAT
TTCAGGGCCTGTTCTCTATTGTCCTTGCTATCCTGTCTTCTGTAGCTATC

TGAAACCATCAACAAAGGAGCACACCATTCCATCAGCAAAAGA.

In embodiments, the minigene comprises an exon sequence, e.g., a second exon sequence, derived from any one of SEQ ID NO:1 to SEQ ID NO: 10 or SEQ ID NO: 80. In embodiments, an exon of the minigene, e.g., the second exon, includes or consists of any one of SEQ ID NO:1 to SEQ ID NO: 10 or SEQ ID NO: 80. In some embodiments, the minigenes described herein include an exon, e.g., a second exon, comprising or consisting of SEQ ID NO: 1, or a sequence having at least 90%, 95%, 97%, 98%, or 99% identity thereto; or a fragment of SEQ ID NO: 1 comprising at least 50%, at least 60%, at least 70%, at least 80%, at least 90%, 95%, 97%, 98%, or 99% of the nucleotides of the sequence. In some embodiments, the minigenes described herein include an exon, e.g., a second exon, comprising or consisting of SEQ ID NO: 2, or a sequence having at least 90%, 95%, 97%, 98%, or 99% identity thereto; or a fragment of SEQ ID NO: 2 comprising at least 50%, at least 60%, at least 70%, at least 80%, at least 90%, 95%, 97%, 98%, or 99% of the nucleotides of the sequence. In some embodiments, the minigenes described herein include an exon, e.g., a second exon, comprising or consisting of SEQ ID NO: 3, or a sequence having at least 90%, 95%, 97%, 98%, or 99% identity thereto; or a fragment of SEQ ID NO: 3 comprising at least 50%, at least 60%, at least 70%, at least 80%, at least 90%, 95%, 97%, 98%, or 99% of the nucleotides of the sequence. In some embodiments, the minigenes described herein include an exon, e.g., a second exon, comprising or consisting of SEQ ID NO: 4, or a sequence having at least 90%, 95%, 97%, 98%, or 99% identity thereto; or a fragment of SEQ ID NO: 4 comprising at least 50%, at least 60%, at least 70%, at least 80%, at least 90%, 95%, 97%, 98%, or 99% of the nucleotides of the sequence. In some embodiments, the minigenes described herein include an exon, e.g., a second exon, comprising or consisting of SEQ ID NO: 5, or a sequence having at least 90%, 95%, 97%, 98%, or 99% identity thereto; or a fragment of SEQ ID NO: 5 comprising at least 50%, at least 60%, at least 70%, at least 80%, at least 90%, 95%, 97%, 98%, or 99% of the nucleotides of the sequence. In some embodiments, the minigenes described herein include an exon, e.g., a second exon, comprising or consisting of SEQ ID NO: 6, or a sequence having at least 90%, 95%, 97%, 98%, or 99% identity thereto; or a fragment of SEQ ID NO: 6 comprising at least 50%, at least 60%, at least 70%, at least 80%, at least 90%, 95%, 97%, 98%, or 99% of the nucleotides of the sequence. In some embodiments, the minigenes described herein include an exon, e.g., a second exon, comprising or consisting of SEQ ID NO: 7, or a sequence having at least 90%, 95%, 97%, 98%, or 99% identity thereto; or a fragment of SEQ ID NO: 7 comprising at least 50%, at least 60%, at least 70%, at least 80%, at least 90%, 95%, 97%, 98%, or 99% of the nucleotides of the sequence. In some embodiments, the minigenes described herein include an exon, e.g., a second exon, comprising or consisting of SEQ ID NO: 8, or a sequence having at least 90%, 95%, 97%, 98%, or 99% identity thereto; or a fragment of SEQ ID NO: 8 comprising at least 50%, at least 60%, at least 70%, at least 80%, at least 90%, 95%, 97%, 98%, or 99% of the nucleotides of the sequence. In some embodiments, the minigenes described herein include an exon, e.g., a second exon, comprising or consisting of SEQ ID NO: 9, or a sequence having at least 90%, 95%, 97%, 98%, or 99% identity thereto; or a fragment of SEQ ID NO: 9 comprising at least 50%, at least 60%, at least 70%, at least 80%, at least 90%, 95%, 97%, 98%, or 99% of the nucleotides of the sequence. In some embodiments, the minigenes described herein include an exon, e.g., a second exon, comprising or consisting of SEQ ID NO: 10, or a sequence having at least 90%, 95%, 97%, 98%, or 99% identity thereto; or a fragment of SEQ ID NO: 10 comprising at least 50%, at least 60%, at least 70%, at least 80%, at least 90%, 95%, 97%, 98%, or 99% of the nucleotides of the sequence. In some embodiments, the minigenes described herein include an exon, e.g., a second exon, comprising or consisting of SEQ ID NO: 80, or a sequence having at least 90%, 95%, 97%, 98%, or 99% identity thereto; or a fragment of SEQ ID NO: 80 comprising at least 50%, at least 60%, at least 70%, at least 80%, at least 90%, 95%, 97%, 98%, or 99% of the nucleotides of the sequence. In some embodiments, the minigenes described herein include an exon, e.g., a second exon, comprising or consisting of SEQ ID NO: 16, or a sequence having at least 90%, 95%, 97%, 98%, or 99% identity thereto; or a fragment of SEQ ID NO: 16 comprising at least 50%, at least 60%, at least 70%, at least 80%, at least 90%, 95%, 97%, 98%, or 99% of the nucleotides of the sequence. In embodiments, the second exon consists of SEQ ID NO: 16.

In some embodiments, the second exon is modified to consist of 3n−1 nucleotides, where n is any integer, such that inclusion of the second exon in the mRNA results in a frame shift relative to the mRNA which does not include the second exon.

Splice Modulators

A “splice modulator” as term is used herein refers to a compound which is capable of mediating alternative splicing. In exemplary embodiments the splice modulator modulates (e.g., increases) the inclusion of an exon in an mRNA product. In exemplary embodiments, the splice modulator modulates (e.g., increases) the inclusion of an exon in an mRNA product by biding to a splice modulator binding sequence (e.g., the sequence AGA, e.g., the sequence AGA at the 3′ end of the exon that is modulated).

In aspects of the invention, the splice modulator is a compound described herein. In a first splice modulator aspect, the splice modulator is a compound according to Formula (I):

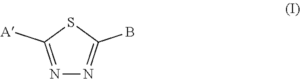

or pharmaceutically acceptable salts thereof, wherein A′ is phenyl which is substituted with 0, 1, 2, or 3 substituents independently selected from C₁-C₄alkyl, wherein 2 C₁-C₄alkyl groups can combine with the atoms to which they are bound to form a 5-6 membered ring and is substituted with 0 or 1 substituents selected from oxo, oxime and, hydroxy, haloC₁-C₄alkyl, dihaloC₁-C₄alkyl, trihaloC₁-C₄alkyl, C₁-C₄alkoxy, C₁-C₄alkoxy-C₃-C₇cycloalkyl, haloC₁-C₄alkoxy, dihaloC₁-C₄alkoxy, trihaloC₁-C₄alkoxy, hydroxy, cyano, halogen, amino, mono- and di-C₁-C₄alkylamino, heteroaryl, C₁-C₄alkyl substituted with hydroxy, C₁-C₄alkoxy substituted with aryl, amino, —C(O)NH C₁-C₄alkyl-heteroaryl, —NHC(O)—C₁-C₄alkyl-heteroaryl, C₁-C₄alkyl C(O)NH— heteroaryl, C₁-C₄alkyl NHC(O)-heteroaryl, 3-7 membered cycloalkyl, 5-7 membered cycloalkenyl or 5, 6 or 9 membered heterocycle containing 1 or 2 heteroatoms, independently, selected from S, O and N, wherein heteroaryl has 5, 6 or 9 ring atoms, 1, 2 or 3 ring heteroatoms selected from N, O and S and substituted with 0, 1, or 2 substituents independently selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC1-C₄alkylamino, hydroxyC₁-C₄alkyl, 4-7 member heterocycleC₁-C₄alkyl, aminoC₁-C₄alkyl and mono- and di-C₁-C₄alkylaminoC₁-C₄alkyl; or A′ is 6 member heteroaryl having 1-3 ring nitrogen atoms, which 6 member heteroaryl is substituted by phenyl or a heteroaryl having 5 or 6 ring atoms, 1 or 2 ring heteroatoms independently selected from N, O and S and substituted with 0, 1, or 2 substituents independently selected from C₁-C₄alkyl, mono- and di-C₁-C₄alkylamino, hydroxyC₁-C₄alkylamino, hydroxyC₁-C₄alkyl, aminoC₁-C₄alkyl and mono- and di-C₁-C₄alkylaminoC₁-C₄alkyl; or A′ is bicyclic heteroaryl having 9 to 10 ring atoms and 1, 2, or 3 ring heteroatoms independently selected from N, O or S, which bicyclic heteroaryl is substituted with 0, 1, or 2 substituents independently selected from oxo, cyano, halogen, hydroxy, C₁-C₄alkyl, C₂-C₄alkenyl, C₂-C₄alkynyl, C₁-C₄alkoxy and C₁-C₄alkoxy substituted with hydroxy, C₁-C₄alkoxy, amino and mono- and di-C₁-C₄alkylamino; B is a group of the formula:

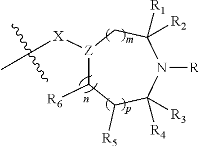

wherein m, n and p are independently selected from 0 or 1; R, R₁, R₂, R₃, and R₄ are independently selected from the group consisting of hydrogen, C₁-C₄alkyl, which alkyl is optionally substituted with hydroxy, amino or mono- and di-C₁-C₄akylamino; R₅ and R₆ are independently selected from hydrogen and fluorine; or R and R₃, taken in combination form a fused 5 or 6 member heterocyclic ring having 0 or 1 additional ring heteroatoms selected from N, O or S; R₁ and R₃, taken in combination form a C₁-C₃alkylene group; R₁ and R₅, taken in combination form a C₁-C₃alkylene group; R₃ and R₄, taken in combination with the carbon atom to which they attach, form a spirocyclicC₃-C₆cycloalkyl; X is CR_{A′}R_{B′}, NR₇ or a bond; R₇ is hydrogen, or C₁-C₄alkyl; R_{A′} and R_{B′} are independently selected from hydrogen and C₁-C₄alkyl, or R_{A′} and R_{B′}, taken in combination, form a divalent C₂-C₅alkylene group; Z is CR₈ or N; when Z is N, X is a bond; R₈ is hydrogen or taken in combination with R₆ form a double bond; or B is a group of the formula:

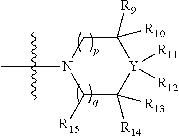

wherein Y is C or O and when Y is O R₁₁ and R₁₂ are both absent; p and q are independently selected from the group consisting of 0, 1, and 2; R₉ and R₁₃ are independently selected from hydrogen and C₁-C₄alkyl; R₁₀ and R₁₄ are independently selected from hydrogen, amino, mono- and di-C₁-C₄akylamino and C₁-C₄alkyl, which alkyl is optionally substituted with hydroxy, amino or mono- and di-C₁-C₄akylamino; R₁₁ is hydrogen, C₁-C₄alkyl, amino or mono- and di-C₁-C₄akylamino; R₁₂ is hydrogen or C₁-C₄alkyl; or R₉ and R₁₁, taken in combination form a saturated azacycle having 4 to 7 ring atoms which is optionally substituted with 1-3 C₁-C₄alkyl groups; or R₁₁ and R₁₂, taken in combination form a saturated azacycle having 4 to 7 ring atoms which is optionally substituted with 1-3 C₁-C₄alkyl groups.

In a second splice modulator aspect, the splice modulator is a compound or pharmaceutically acceptable salt thereof, according to the first splice modulator aspect wherein A′ is selected from:

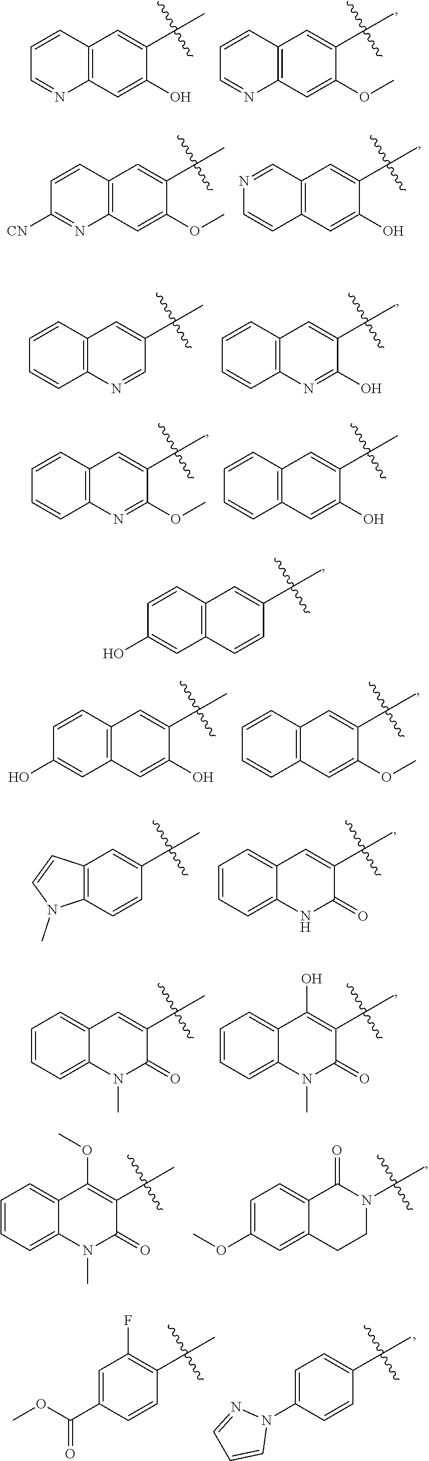

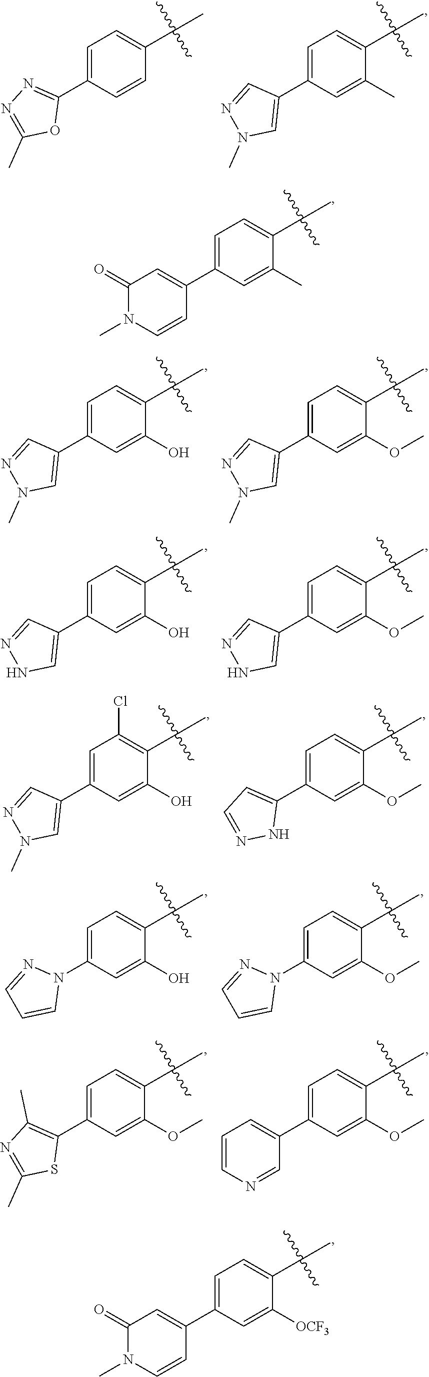

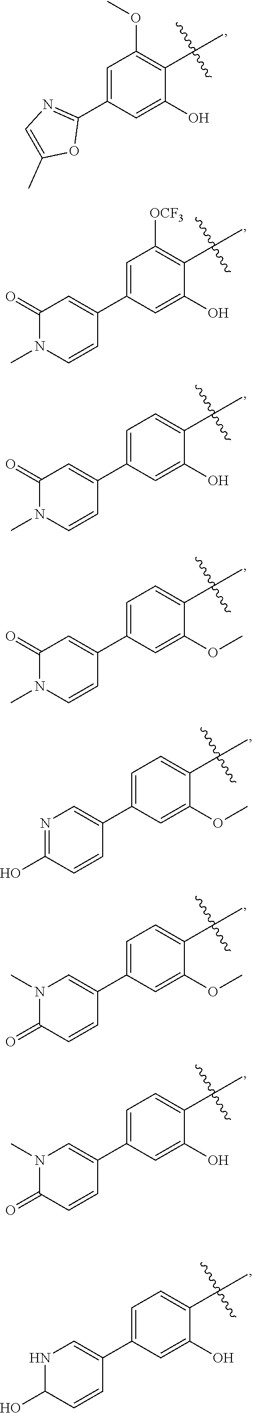

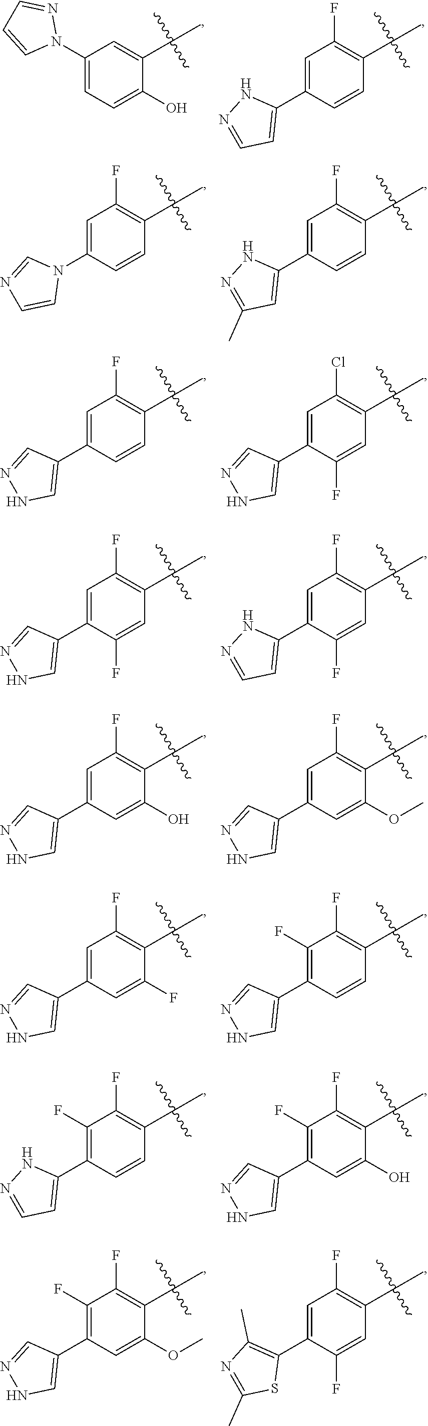

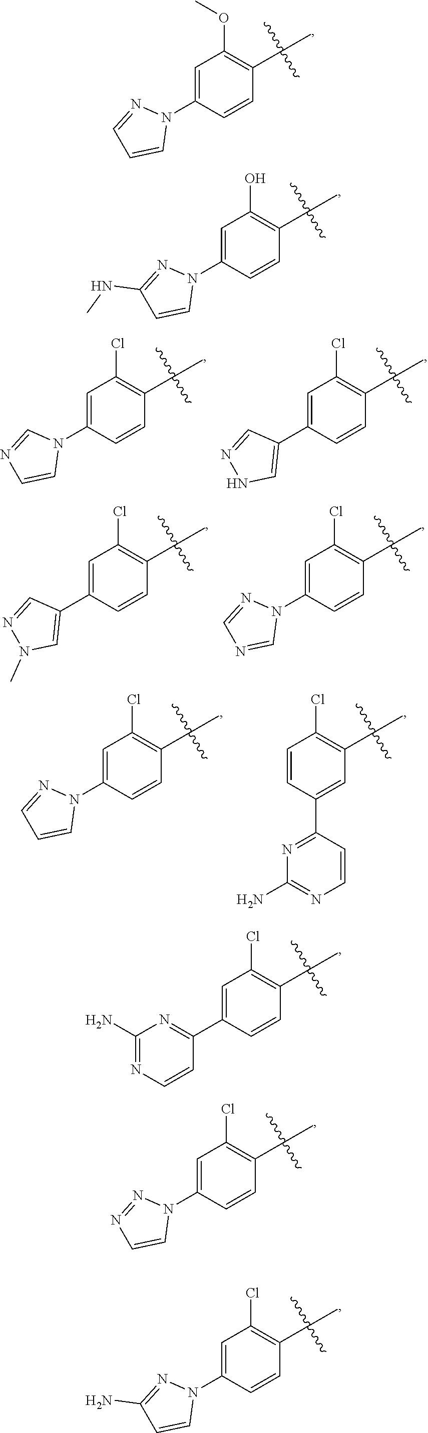

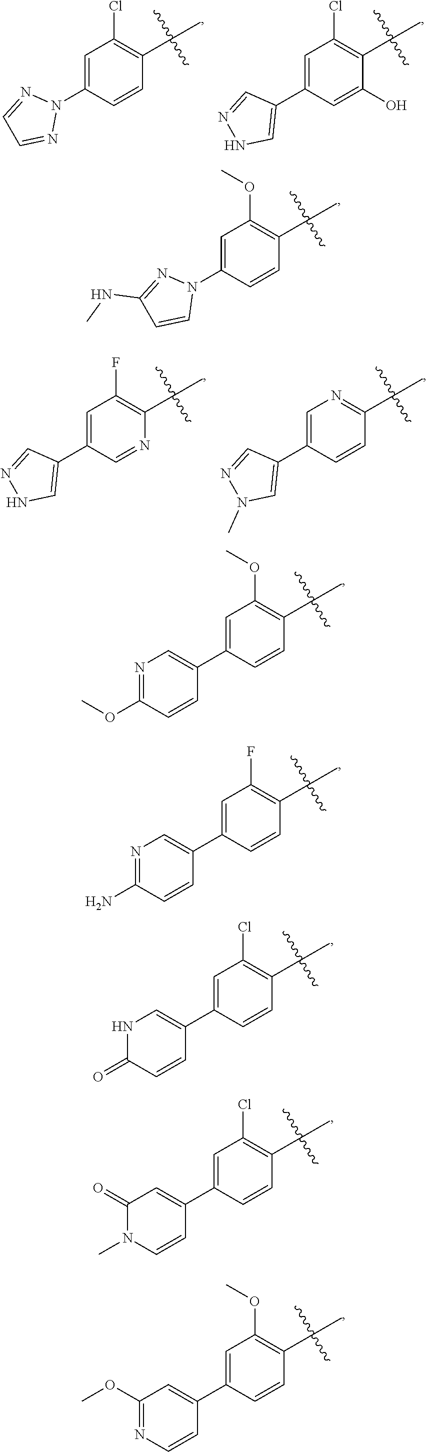

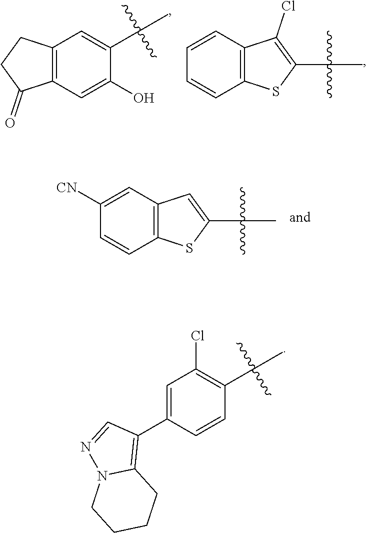

In a third splice modulator aspect, the splice modulator is a compound according to Formula (II):

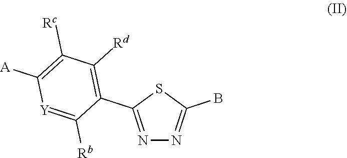

or pharmaceutically acceptable salts thereof, wherein Y is N or C—R^{a}; R^{a} is hydrogen or C₁-C₄alkyl; R^{b} is hydrogen, C₁-C₄alkyl, C₁-C₄alkoxy, hydroxy, cyano, halogen, trihalo C₁-C₄alkyl or trihalo C₁-C₄alkoxy; R^{c} and R^{d} are each, independently, hydrogen, C₁-C₄alkyl, C₁-C₄alkoxy, hydroxy, trihalo C₁-C₄alkyl, trihalo C₁-C₄alkoxy or heteroaryl; A is 6 member heteroaryl having 1-3 ring nitrogen atoms, which 6 member heteroaryl is substituted with 0, 1, or 2 substituents independently selected from oxo, C₁-C₄alkyl, mono- and di-C₁-C₄alkylamino, hydroxyC₁-C₄alkylamino, hydroxyC₁-C₄alkyl, aminoC₁-C₄alkyl and mono- and di-C₁-C₄alkylaminoC₁-C₄alkyl; or A is 5 member heteroaryl having 1-3 ring heteroatoms independently selected from N, O and S and substituted with 0, 1, or 2 substituents independently selected from C₁-C₄alkyl, hydroxyl, mono- and di-C₁-C₄alkylamino, hydroxyC₁-C₄alkylamino, hydroxyC₁-C₄alkyl, aminoC₁-C₄alkyl and mono- and di-C₁-C₄alkylaminoC₁-C₄alkyl; or A and R^{c}, together with the atoms to which they are bound, form a 6 member aryl with 0, 1, or 2 substituents independently selected from cyano, halogen, hydroxy, C₁-C₄alkyl, C₂-C₄alkenyl, C₂-C₄alkynyl, C₁-C₄alkoxy and C₁-C₄alkoxy substituted with hydroxy, C₁-C₄alkoxy, amino and mono- and di-C₁-C₄alkylamino; B is a group of the formula:

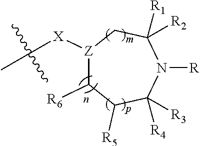

wherein m, n and p are independently selected from 0 or 1; R, R₁, R₂, R₃, and R₄ are independently selected from the group consisting of hydrogen, C₁-C₄alkyl, which alkyl is optionally substituted with hydroxy, amino or mono- and di-C₁-C₄akylamino; R₅ and R₆ are independently selected from hydrogen and fluorine; or R and R₃, taken in combination form a fused 5 or 6 member heterocyclic ring having 0 or 1 additional ring heteroatoms selected from N, O or S; R₁ and R₃, taken in combination form a C₁-C₃alkylene group; R₁ and R₅, taken in combination form a C₁-C₃alkylene group; R₃ and R₄, taken in combination with the carbon atom to which they attach, form a spirocyclicC₃-C₆cycloalkyl; X is CR_{A′}R_{B′}, NR₇ or a bond; R₇ is hydrogen, or C₁-C₄alkyl; R_{A′} and R_{B′} are independently selected from hydrogen and C₁-C₄alkyl, or R_{A′} and R_{B′}, taken in combination, form a divalent C₂-C₅alkylene group; Z is CR₈ or N; when Z is N, X is a bond; R₈ is hydrogen or taken in combination with R₆ form a double bond; or B is a group of the formula:

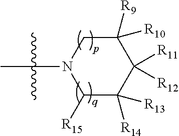

wherein p and q are independently selected from the group consisting of 0, 1, and 2; R₉ and R₁₃ are independently selected from hydrogen and C₁-C₄alkyl; R₁₀ and R₁₄ are independently selected from hydrogen, amino, mono- and di-C₁-C₄akylamino and C₁-C₄alkyl, which alkyl is optionally substituted with hydroxy, amino or mono- and di-C₁-C₄akylamino; R₁₁ is hydrogen, C₁-C₄alkyl, amino or mono- and di-C₁-C₄akylamino; R₁₂ is hydrogen or C₁-C₄alkyl; or R₉ and R₁₁, taken in combination form a saturated azacycle having 4 to 7 ring atoms which is optionally substituted with 1-3 C₁-C₄alkyl groups; or R₁₁ and R₁₂, taken in combination form a saturated azacycle having 4 to 7 ring atoms which is optionally substituted with 1-3 C₁-C₄alkyl groups.

In a fourth splice modulator aspect, the splice modulator is a compound or pharmaceutically acceptable salt thereof, according to the third splice modulator aspect, wherein A is 6 member heteroaryl having 1-3 ring nitrogen atoms, which 6 member heteroaryl is substituted with 0, 1, or 2 substituents independently selected from oxo, C₁-C₄alkyl, mono- and di-C₁-C₄alkylamino, hydroxyC₁-C₄alkylamino, hydroxyC₁-C₄alkyl, aminoC₁-C₄alkyl and mono- and di-C₁-C₄alkylaminoC₁-C₄alkyl.

In a fifth splice modulator aspect, the splice modulator is a compound or pharmaceutically acceptable salt thereof, according to any one of the third or fourth splice modulator aspects, wherein A is selected from:

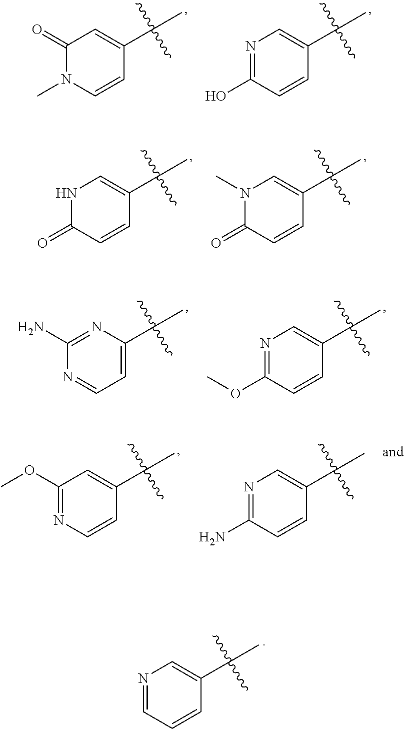

In a sixth splice modulator aspect, the splice modulator is a compound or pharmaceutically acceptable salt thereof, according to the third splice modulator aspect, wherein A is 5 member heteroaryl having 1-3 ring heteroatoms independently selected from N, O and S and substituted with 0, 1, or 2 substituents independently selected from C₁-C₄alkyl, hydroxyl, mono- and di-C₁-C₄alkylamino, hydroxyC₁-C₄alkylamino, hydroxyC₁-C₄alkyl, aminoC₁-C₄alkyl and mono- and di-C₁-C₄alkylaminoC₁-C₄alkyl.

In a seventh splice modulator aspect, the splice modulator is a compound or pharmaceutically acceptable salt thereof, according to any one of the third or sixth splice modulator aspects, wherein A is selected from:

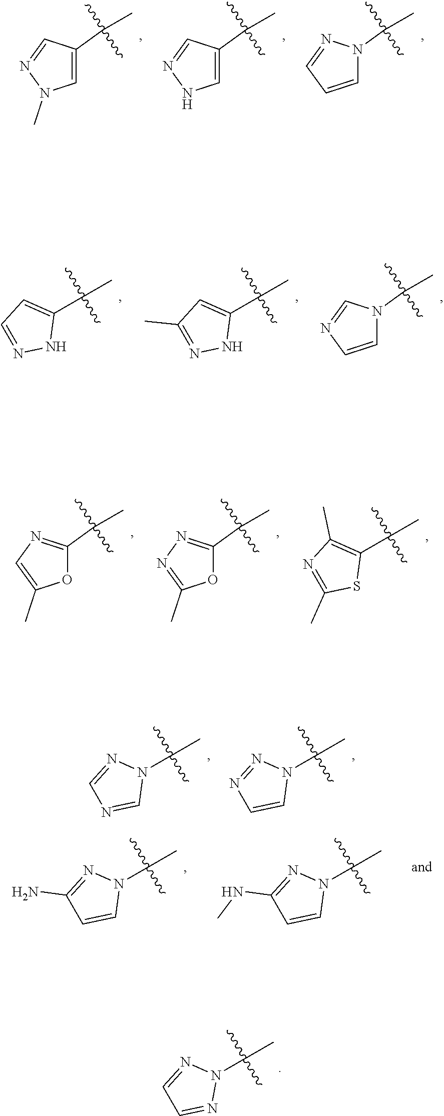

In an eighth splice modulator aspect, the splice modulator is a compound or pharmaceutically acceptable salt thereof, according to any one of the first through seventh splice modulator aspects, wherein B is a group of the formula:

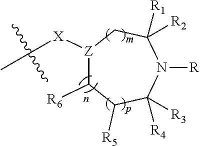

wherein m, n and p are independently selected from 0 or 1; R, R₁, R₂, R₃, and R₄ are independently selected from the group consisting of hydrogen, C₁-C₄alkyl, which alkyl is optionally substituted with hydroxy, amino or mono- and di-C₁-C₄akylamino; R₅ and R₆ are hydrogen; or R and R₃, taken in combination form a fused 5 or 6 member heterocyclic ring having 0 or 1 additional ring heteroatoms selected from N, O or S; R₁ and R₃, taken in combination form a C₁-C₃alkylene group; R₁ and R₅, taken in combination form a C₁-C₃alkylene group; R₃ and R₄, taken in combination with the carbon atom to which they attach, form a spirocyclicC₃-C₆cycloalkyl; X is CR_{A′}R_{B′}, O, NR₇ or a bond; R_{A′} and R_{B′} are independently selected from hydrogen and C₁-C₄alkyl, or R_{A′} and R_{B′}, taken in combination, form a divalent C₂-C₅alkylene group; Z is CR₈ or N; when Z is N, X is a bond; R₈ is hydrogen or taken in combination with R₆ form a double bond.

In a ninth splice modulator aspect, the splice modulator is a compound or pharmaceutically acceptable salt thereof, according to any one of the first through seventh splice modulator aspects, wherein B is a group of the formula:

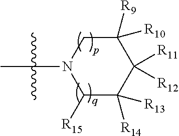

wherein p and q are independently selected from the group consisting of 0, 1, and 2; R₉ and R₁₃ are independently selected from hydrogen and C₁-C₄alkyl; R₁₀ and R₁₄ are independently selected from hydrogen, amino, mono- and di-C₁-C₄akylamino and C₁-C₄alkyl, which alkyl is optionally substituted with hydroxy, amino or mono- and di-C₁-C₄akylamino; R₁₁ is hydrogen, C₁-C₄alkyl, amino or mono- and di-C₁-C₄akylamino; R₁₂ is hydrogen or C₁-C₄alkyl; or R₉ and R₁₁, taken in combination form a saturated azacycle having 4 to 7 ring atoms which is optionally substituted with 1-3 C₁-C₄alkyl groups; or R₁₁ and R₁₂, taken in combination form a saturated azacycle having 4 to 7 ring atoms which is optionally substituted with 1-3 C₁-C₄alkyl groups.

In a tenth splice modulator aspect, the splice modulator is a compound according to Formula (III):

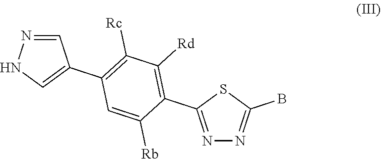

or pharmaceutically acceptable salt thereof, wherein R^{b} is hydrogen or hydroxy; R^{c} is hydrogen or halogen; and R^{d} is halogen.

In an eleventh splice modulator aspect, the splice modulator is a compound according to Formula (IV):

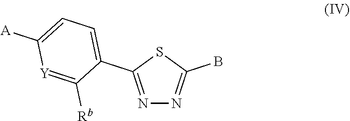

or pharmaceutically acceptable salt thereof, wherein R^{b} is hydroxyl, methoxy, trifluoromethyl or trifluoromethoxy.

In a twelfth splice modulator aspect, the splice modulator is a compound according to Formula (V):

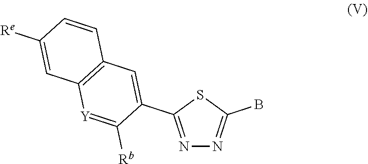

or pharmaceutically acceptable salt thereof, wherein R^{b} is hydroxyl, methoxy, trifluoromethyl or trifluoromethoxy; and R^{e} is hydrogen, hydroxy or methoxy.

In a thirteenth splice modulator aspect, the splice modulator is a compound or pharmaceutically acceptable salt thereof, according to any one of the third through ninth or eleventh through twelfth splice modulator aspects, wherein Y is N.

In a fourteenth splice modulator aspect, the splice modulator is a compound or pharmaceutically acceptable salt thereof, according to any one of the third through ninth or eleventh through twelfth splice modulator aspects, wherein Y is CH.

In a fifteenth splice modulator aspect, the splice modulator is a compound or pharmaceutically acceptable salt thereof, according of any one of the first through eighth or tenth through fourteenth splice modulator aspects, wherein B is selected from

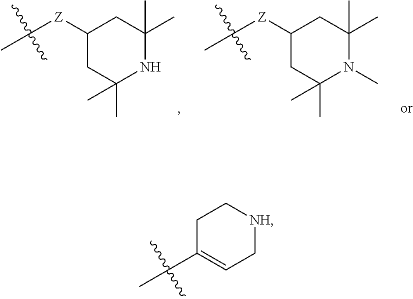

wherein Z is NH or N(Me).

In a sixteenth splice modulator aspect, the splice modulator is a compound or pharmaceutically acceptable salts thereof, according of any one of the first through eighth or tenth through fifteenth splice modulator aspects, wherein B is

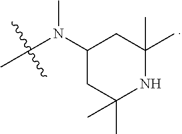 [SMILES: *N(C)C1CC(C)(C)NC(C)(C)C1]

In a seventeenth splice modulator aspect, the splice modulator is a compound or pharmaceutically acceptable salt thereof, according of any one of the first through seventh or ninth through fourteenth splice modulator aspects, wherein B is selected from

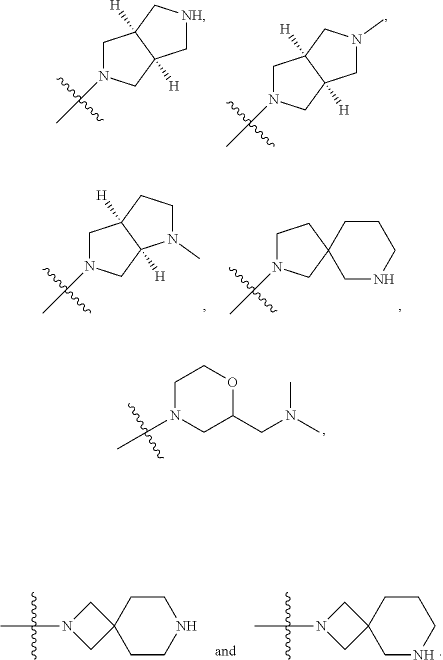

In an eighteenth splice modulator aspect, the splice modulator is a compound or pharmaceutically acceptable salt thereof, according of any one of the first through seventh, ninth through fourteenth or seventeenth splice modulator aspects wherein B is

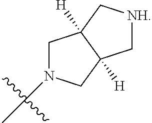 [SMILES: *N1C[C@H]2CNC[C@H]2C1]

In a nineteenth splice modulator aspect, the splice modulator is a compound according to Formula (VI):

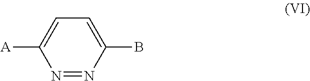

or pharmaceutically acceptable salt thereof, wherein A is bicyclic heteroaryl or heterocyle having 9 or 10 ring atoms and 1 or 2 ring N atoms and 0 or 1 O atoms, which bicyclic heteroaryl or heterocycle is substituted with 0, 1, 2, 3, 4 or 5 substituents independently selected from —C(O)NH₂, —C(O)O—C₁-C₄alkyl, aryl, oxo, cyano, halogen, hydroxy, C₁-C₄alkyl, C₂-C₄alkenyl, C₂-C₄alkynyl, C₁-C₄alkoxy, C₃-C₇cycloalkyl, heterocyclyl, heteroaryl, heterocyclyl C₁-C₄alkyl, C₁-C₄alkyl aryl, C₁-C₄alkyl heterocyclyl, C₁-C₄alkyl heteroaryl, C₁-C₄alkoxy aryl, C₁-C₄alkoxy heterocyclyl, C₁-C₄alkoxy heteroaryl, C₁-C₄alkoxy substituted with hydroxy, C₁-C₄alkoxy, amino and mono- and di-C₁-C₄alkylamino; and B is a group of the formula:

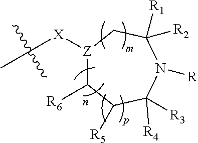

wherein m, n and p are independently selected from 0 or 1; R, R₁, R₂, R₃, and R₄ are independently selected from the group consisting of hydrogen, C₁-C₄alkyl, which alkyl is optionally substituted with hydroxy, amino or mono- and di-C₁-C₄akylamino; R₅ and R₆ are independently selected from hydrogen and fluorine; or R and R₃, taken in combination form a fused 5 or 6 member heterocyclic ring having 0 or 1 additional ring heteroatoms selected from N, O or S; R₁ and R₃, taken in combination form a C₁-C₃alkylene group; R₁ and R₅, taken in combination form a C₁-C₃alkylene group; R₃ and R₄, taken in combination with the carbon atom to which they attach, form a spirocyclicC₃-C₆cycloalkyl; X is CR_{A}R_{B}, O, NR₇ or a bond; R₇ is hydrogen, or C₁-C₄alkyl; R_{A} and R_{B} are independently selected from hydrogen and C₁-C₄alkyl, or R_{A} and R_{B}, taken in combination, form a divalent C₂-C₅alkylene group; Z is CR₈ or N; when Z is N, X is a bond; R₈ is hydrogen or taken in combination with R₆ form a double bond; or B is a group of the formula:

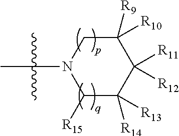

wherein p and q are independently selected from the group consisting of 0, 1, and 2; R₉ and R₁₃ are independently selected from hydrogen and C₁-C₄alkyl; R₁₀ and R₁₄ are independently selected from hydrogen, amino, mono- and di-C₁-C₄akylamino and C₁-C₄alkyl, which alkyl is optionally substituted with hydroxy, amino or mono- and di-C₁-C₄akylamino; R₁₁ is hydrogen, C₁-C₄alkyl, amino or mono- and di-C₁-C₄akylamino; R₁₂ is hydrogen or C₁-C₄alkyl; or R₉ and R₁₁, taken in combination form a saturated azacycle having 4 to 7 ring atoms which is optionally substituted with 1-3 C₁-C₄alkyl groups; or R₁₁ and R₁₂, taken in combination form a saturated azacycle having 4 to 7 ring atoms which is optionally substituted with 1-3 C₁-C₄alkyl groups.

In a twentieth splice modulator aspect, the splice modulator is a compound according to Formula (VII):

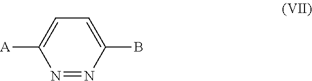

or pharmaceutically acceptable salt thereof, wherein A is bicyclic heteroaryl having 10 ring atoms and 1 or 2 ring N atoms, which bicyclic heteroaryl is substituted with 0, 1, or 2 substituents independently selected from oxo, cyano, halogen, hydroxy, C₁-C₄alkyl, C₂-C₄alkenyl, C₂-C₄alkynyl, C₁-C₄alkoxy, C₃-C₇cycloalkyl, heterocyclyl, heteroaryl, heterocyclyl C₁-C₄alkyl, C₁-C₄alkyl aryl, C₁-C₄alkyl heterocyclyl, C₁-C₄alkyl heteroaryl, C₁-C₄alkoxy aryl, C₁-C₄alkoxy heterocyclyl, C₁-C₄alkoxy heteroaryl, C₁-C₄alkoxy substituted with hydroxy, C₁-C₄alkoxy, amino and mono- and di-C₁-C₄alkylamino; and B is a group of the formula:

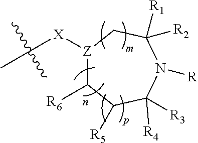

wherein m, n and p are independently selected from 0 or 1; R, R₁, R₂, R₃, and R₄ are independently selected from the group consisting of hydrogen, C₁-C₄alkyl, which alkyl is optionally substituted with hydroxy, amino or mono- and di-C₁-C₄akylamino; R₅ and R₆ are independently selected from hydrogen and fluorine; or R and R₃, taken in combination form a fused 5 or 6 member heterocyclic ring having 0 or 1 additional ring heteroatoms selected from N, O or S; R₁ and R₃, taken in combination form a C₁-C₃alkylene group; R₁ and R₅, taken in combination form a C₁-C₃alkylene group; R₃ and R₄, taken in combination with the carbon atom to which they attach, form a spirocyclicC₃-C₆cycloalkyl; X is CR_{A}R_{B}, O, NR₇ or a bond; R₇ is hydrogen, or C₁-C₄alkyl; R_{A} and R_{B} are independently selected from hydrogen and C₁-C₄alkyl, or R_{A} and R_{B}, taken in combination, form a divalent C₂-C₅alkylene group; Z is CR₈ or N; when Z is N, X is a bond; R₈ is hydrogen or taken in combination with R₆ form a double bond; or B is a group of the formula:

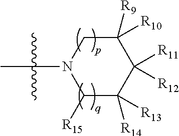

wherein p and q are independently selected from the group consisting of 0, 1, and 2; R₉ and R₁₃ are independently selected from hydrogen and C₁-C₄alkyl; R₁₀ and R₁₄ are independently selected from hydrogen, amino, mono- and di-C₁-C₄akylamino and C₁-C₄alkyl, which alkyl is optionally substituted with hydroxy, amino or mono- and di-C₁-C₄akylamino; R₁₁ is hydrogen, C₁-C₄alkyl, amino or mono- and di-C₁-C₄akylamino; R₁₂ is hydrogen or C₁-C₄alkyl; or R₉ and R₁₁, taken in combination form a saturated azacycle having 4 to 7 ring atoms which is optionally substituted with 1-3 C₁-C₄alkyl groups; or R₁₁ and R₁₂, taken in combination form a saturated azacycle having 4 to 7 ring atoms which is optionally substituted with 1-3 C₁-C₄alkyl groups.

In a twenty-first splice modulator aspect, the splice modulator is a compound or pharmaceutically acceptable salt thereof, according to any one of the nineteenth or twentieth splice modulator aspects, wherein A is selected from:

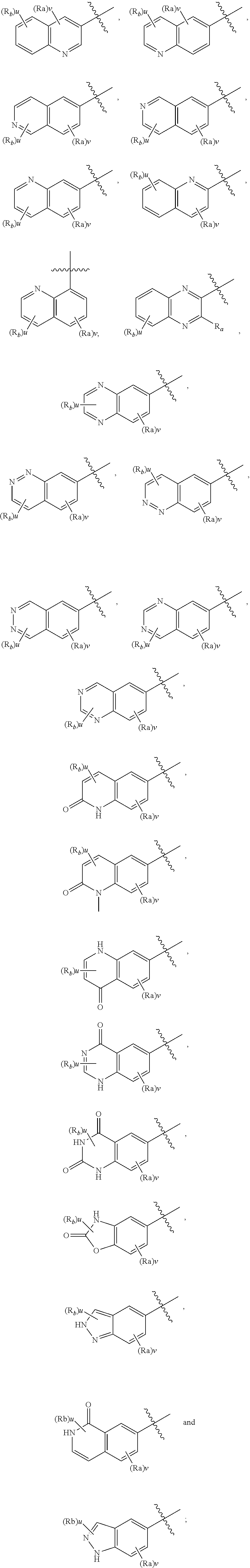

wherein u and v are each, independently, 0, 1, 2 or 3; and each Rₐ and R_{b} are, independently, selected from cyano, halogen, hydroxy, C₁-C₄alkyl, C₂-C₄alkenyl, C₂-C₄alkynyl, C₁-C₄alkoxy, C₃-C₇cycloalkyl, heterocyclyl, heteroaryl, heterocyclyl C₁-C₄alkyl, C₁-C₄alkyl aryl, C₁-C₄alkyl heterocyclyl, C₁-C₄alkyl heteroaryl, C₁-C₄alkoxy aryl, C₁-C₄alkoxy heterocyclyl, C₁-C₄alkoxy heteroaryl, and C₁-C₄alkoxy substituted with hydroxy, C₁-C₄alkoxy, amino and mono- and di-C₁-C₄alkylamino.

In a twenty-second splice modulator aspect, the splice modulator is a compound or pharmaceutically acceptable salt thereof, according to any one of the nineteenth through twenty-first splice modulator aspect, wherein A is selected from:

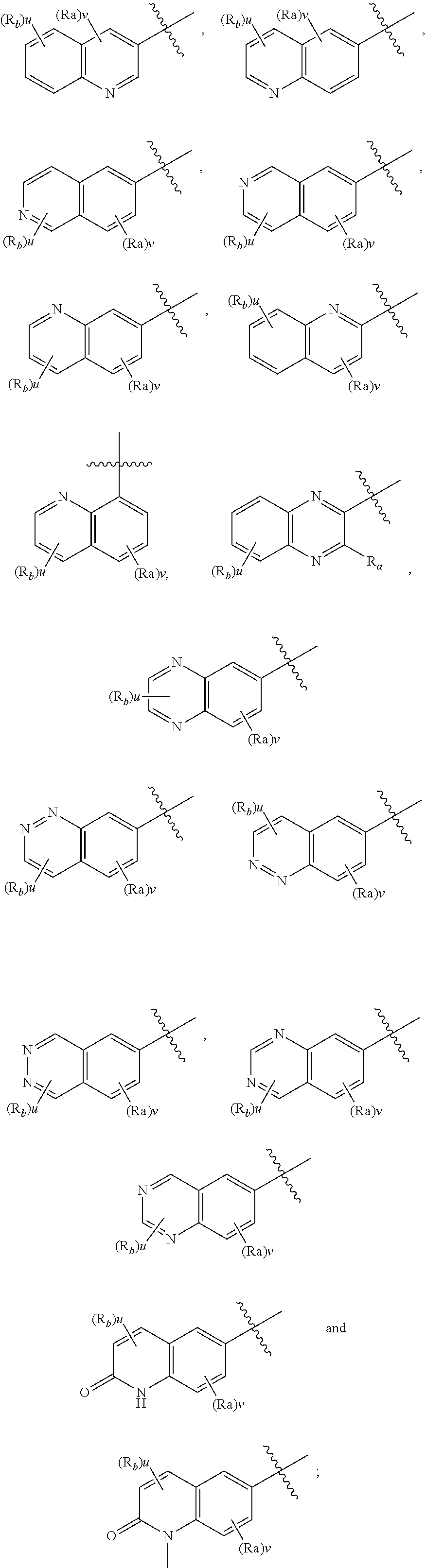

wherein u and v are each, independently, 0, 1, 2 or 3; and each Rₐ and R_{b} are, independently, selected from, cyano, halogen, hydroxy, C₁-C₄alkyl, C₂-C₄alkenyl, C₂-C₄alkynyl, C₁-C₄alkoxy, C₃-C₇cycloalkyl, heterocyclyl, heteroaryl, heterocyclyl C₁-C₄alkyl, C₁-C₄alkyl aryl, C₁-C₄alkyl heterocyclyl, C₁-C₄alkyl heteroaryl, C₁-C₄alkoxy aryl, C₁-C₄alkoxy heterocyclyl, C₁-C₄alkoxy heteroaryl, and C₁-C₄alkoxy substituted with hydroxy, C₁-C₄alkoxy, amino and mono- and di-C₁-C₄alkylamino.

In another splice modulator aspect, provided herein are compounds or pharmaceutically acceptable salts thereof, according to any one of the nineteenth through twenty-second splice modulator aspects, wherein A is substituted in the ortho position with a hydroxyl group.

In a twenty-third splice modulator aspect, the splice modulator is a compound or pharmaceutically acceptable salt thereof, according to any one of the nineteenth through twenty-second splice modulator aspects, wherein A is selected from:

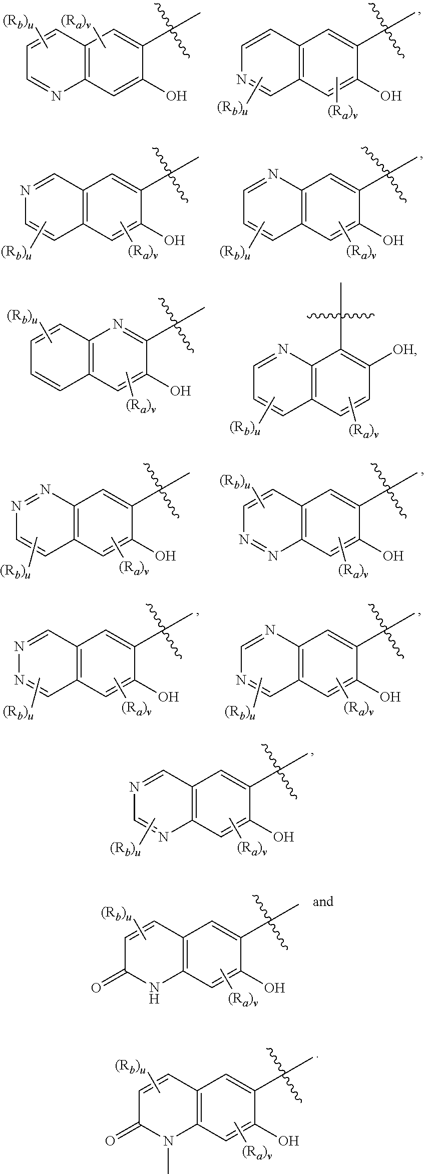

In a twenty-fourth splice modulator aspect, the splice modulator is a compound or pharmaceutically acceptable salt thereof, according to any one of the nineteenth through twenty-third splice modulator aspect, wherein A has a single N atom.

In a twenty-fifth splice modulator aspect, the splice modulator is a compound according to Formula (VIII):

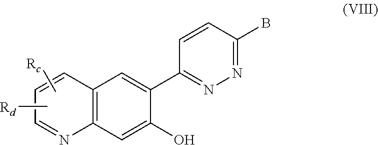

or pharmaceutically acceptable salt thereof, wherein R_{c} and R_{d} are each, independently, selected from hydrogen, cyano, halogen, hydroxy, C₁-C₄alkyl, C₂-C₄alkenyl, C₂-C₄alkynyl, C₁-C₄alkoxy, C₃-C₇cycloalkyl, heterocyclyl, heteroaryl, heterocyclyl C₁-C₄alkyl, C₁-C₄alkyl aryl, C₁-C₄alkyl heterocyclyl, C₁-C₄alkyl heteroaryl, C₁-C₄alkoxy aryl, C₁-C₄alkoxy heterocyclyl, C₁-C₄alkoxy heteroaryl, C₁-C₄alkoxy substituted with hydroxy, C₁-C₄alkoxy, amino and mono- and di-C₁-C₄alkylamino.

In a twenty-sixth splice modulator aspect, the splice modulator is a compound according to Formula (IX):

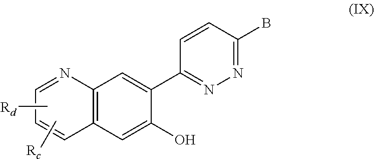

or pharmaceutically acceptable salt thereof, wherein R_{c} and R_{d} are each, independently, selected from hydrogen, cyano, halogen, hydroxy, C₁-C₄alkyl, C₂-C₄alkenyl, C₂-C₄alkynyl, C₁-C₄alkoxy, C₃-C₇cycloalkyl, heterocyclyl, heteroaryl, heterocyclyl C₁-C₄alkyl, C₁-C₄alkyl aryl, C₁-C₄alkyl heterocyclyl, C₁-C₄alkyl heteroaryl, C₁-C₄alkoxy aryl, C₁-C₄alkoxy heterocyclyl, C₁-C₄alkoxy heteroaryl, C₁-C₄alkoxy substituted with hydroxy, C₁-C₄alkoxy, amino and mono- and di-C₁-C₄alkylamino.

In a twenty-seventh splice modulator aspect, the splice modulator is a compound according to Formula (X):

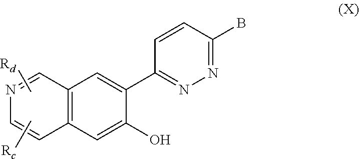

or pharmaceutically acceptable salt thereof, wherein R_{c} and R_{d} are each, independently, selected from hydrogen, cyano, halogen, hydroxy, C₁-C₄alkyl, C₂-C₄alkenyl, C₂-C₄alkynyl, C₁-C₄alkoxy, C₃-C₇cycloalkyl, heterocyclyl, heteroaryl, heterocyclyl C₁-C₄alkyl, C₁-C₄alkyl aryl, C₁-C₄alkyl heterocyclyl, C₁-C₄alkyl heteroaryl, C₁-C₄alkoxy aryl, C₁-C₄alkoxy heterocyclyl, C₁-C₄alkoxy heteroaryl, C₁-C₄alkoxy substituted with hydroxy, C₁-C₄alkoxy, amino and mono- and di-C₁-C₄alkylamino.

In a twenty-eighth splice modulator aspect, the splice modulator is a compound according to Formula (XI):

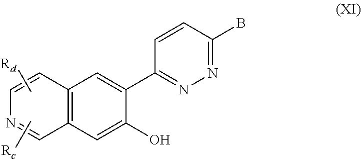

or pharmaceutically acceptable salt thereof, wherein R_{c} and R_{d} are each, independently, selected from hydrogen, cyano, halogen, hydroxy, C₁-C₄alkyl, C₂-C₄alkenyl, C₂-C₄alkynyl, C₁-C₄alkoxy, C₃-C₇cycloalkyl, heterocyclyl, heteroaryl, heterocyclyl C₁-C₄alkyl, C₁-C₄alkyl aryl, C₁-C₄alkyl heterocyclyl, C₁-C₄alkyl heteroaryl, C₁-C₄alkoxy aryl, C₁-C₄alkoxy heterocyclyl, C₁-C₄alkoxy heteroaryl, C₁-C₄alkoxy substituted with hydroxy, C₁-C₄alkoxy, amino and mono- and di-C₁-C₄alkylamino.

In a twenty-ninth splice modulator aspect, the splice modulator is a compound according to Formula (XII):

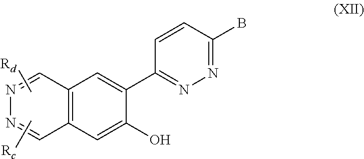

or pharmaceutically acceptable salt thereof, wherein R_{c} and R_{d} are each, independently, selected from hydrogen, cyano, halogen, hydroxy, C₁-C₄alkyl, C₂-C₄alkenyl, C₂-C₄alkynyl, C₁-C₄alkoxy, C₃-C₇cycloalkyl, heterocyclyl, heteroaryl, heterocyclyl C₁-C₄alkyl, C₁-C₄alkyl aryl, C₁-C₄alkyl heterocyclyl, C₁-C₄alkyl heteroaryl, C₁-C₄alkoxy aryl, C₁-C₄alkoxy heterocyclyl, C₁-C₄alkoxy heteroaryl, C₁-C₄alkoxy substituted with hydroxy, C₁-C₄alkoxy, amino and mono- and di-C₁-C₄alkylamino.

In a thirtieth splice modulator aspect, the splice modulator is a compound according to Formula (XIII):

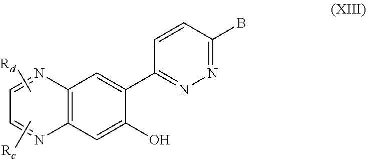

or pharmaceutically acceptable salt thereof, wherein R_{c} and R_{d} are each, independently, selected from hydrogen, cyano, halogen, hydroxy, C₁-C₄alkyl, C₂-C₄alkenyl, C₂-C₄alkynyl, C₁-C₄alkoxy, C₃-C₇cycloalkyl, heterocyclyl, heteroaryl, heterocyclyl C₁-C₄alkyl, C₁-C₄alkyl aryl, C₁-C₄alkyl heterocyclyl, C₁-C₄alkyl heteroaryl, C₁-C₄alkoxy aryl, C₁-C₄alkoxy heterocyclyl, C₁-C₄alkoxy heteroaryl, C₁-C₄alkoxy substituted with hydroxy, C₁-C₄alkoxy, amino and mono- and di-C₁-C₄alkylamino.

In a thirty-first splice modulator aspect, the splice modulator is a compound according to Formula (XIV):

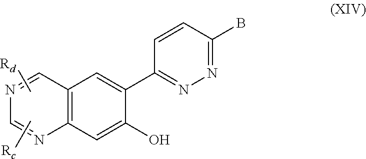

or pharmaceutically acceptable salt thereof, wherein R_{c} and R_{d} are each, independently, selected from hydrogen, cyano, halogen, hydroxy, C₁-C₄alkyl, C₂-C₄alkenyl, C₂-C₄alkynyl, C₁-C₄alkoxy, C₃-C₇cycloalkyl, heterocyclyl, heteroaryl, heterocyclyl C₁-C₄alkyl, C₁-C₄alkyl aryl, C₁-C₄alkyl heterocyclyl, C₁-C₄alkyl heteroaryl, C₁-C₄alkoxy aryl, C₁-C₄alkoxy heterocyclyl, C₁-C₄alkoxy heteroaryl, C₁-C₄alkoxy substituted with hydroxy, C₁-C₄alkoxy, amino and mono- and di-C₁-C₄alkylamino.

In a thirty-second splice modulator aspect, the splice modulator is a compound or pharmaceutically acceptable salt thereof, according to any one of the nineteenth through thirty-first splice modulator aspects, wherein B is a group of the formula:

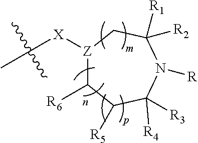

wherein m, n and p are independently selected from 0 or 1; R, R₁, R₂, R₃, and R₄ are independently selected from the group consisting of hydrogen, C₁-C₄alkyl, which alkyl is optionally substituted with hydroxy, amino or mono- and di-C₁-C₄akylamino; R₅ and R₆ are hydrogen; or R and R₃, taken in combination form a fused 5 or 6 member heterocyclic ring having 0 or 1 additional ring heteroatoms selected from N, O or S; R₁ and R₃, taken in combination form a C₁-C₃alkylene group; R₁ and R₅, taken in combination form a C₁-C₃alkylene group; R₃ and R₄, taken in combination with the carbon atom to which they attach, form a spirocyclicC₃-C₆cycloalkyl; X is CR_{A}R_{B}, O, NR₇ or a bond; R_{A} and R_{B} are independently selected from hydrogen and C₁-C₄alkyl, or R_{A} and R_{B}, taken in combination, form a divalent C₂-C₅alkylene group; Z is CR₈ or N; when Z is N, X is a bond; R₈ is hydrogen or taken in combination with R₆ form a double bond.

In a thirty-third splice modulator aspect, the splice modulator is a compound or pharmaceutically acceptable salt thereof, according to any one of the nineteenth through thirty-second splice modulator aspects, wherein B is a group of the formula:

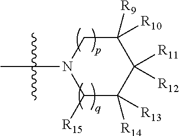

wherein p and q are independently selected from the group consisting of 0, 1, and 2; R₉ and R₁₃ are independently selected from hydrogen and C₁-C₄alkyl; R₁₀ and R₁₄ are independently selected from hydrogen, amino, mono- and di-C₁-C₄akylamino and C₁-C₄alkyl, which alkyl is optionally substituted with hydroxy, amino or mono- and di-C₁-C₄akylamino; R₁₁ is hydrogen, C₁-C₄alkyl, amino or mono- and di-C₁-C₄akylamino; R₁₂ is hydrogen or C₁-C₄alkyl; or R₉ and R₁₁, taken in combination form a saturated azacycle having 4 to 7 ring atoms which is optionally substituted with 1-3 C₁-C₄alkyl groups; or R₁₁ and R₁₂, taken in combination form a saturated azacycle having 4 to 7 ring atoms which is optionally substituted with 1-3 C₁-C₄alkyl groups.

In a thirty-fourth splice modulator aspect, the splice modulator is a compound or pharmaceutically acceptable salt thereof, according to any one of the nineteenth through thirty-third splice modulator aspects, wherein B is selected from the group consisting of:

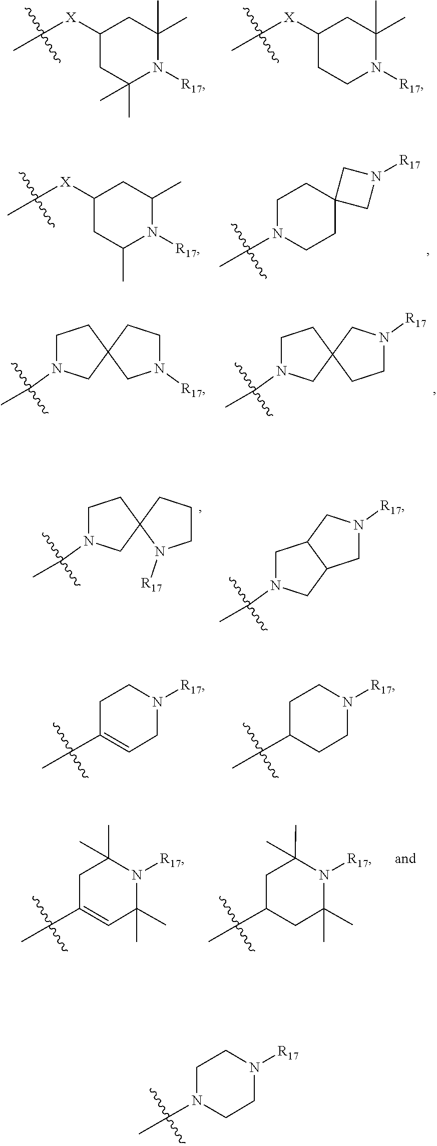

wherein X is O or N(Me) or NH; and R₁₇ is hydrogen or methyl.

In a thirty-fifth splice modulator aspect, the splice modulator is a compound or pharmaceutically acceptable salt thereof, according to any one of the nineteenth through thirty-fourth splice modulator aspects, wherein B is:

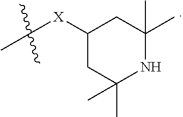

In a thirty-sixth splice modulator aspect, the splice modulator is a compound or pharmaceutically acceptable salt thereof, according to any one of the nineteenth through thirty-fifth splice modulator aspects, wherein X is —O—.

In a thirty-seventh splice modulator aspect, the splice modulator is a compound or pharmaceutically acceptable salt thereof, according to any one of the nineteenth through thirty-sixth splice modulator aspects, wherein X is N(Me).

In a thirty-eighth splice modulator aspect, the splice modulator is a compounds according to Formula (XV):

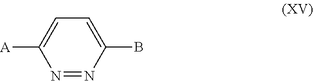

or pharmaceutically acceptable salt thereof, wherein A is 2-hydroxy-phenyl which is substituted with 0, 1, 2, or 3 substituents independently selected from C₁-C₄alkyl, wherein 2 C₁-C₄alkyl groups can combine with the atoms to which they are bound to form a 5-6 membered ring and is substituted with 0 or 1 substituents selected from oxo, oxime and hydroxy, haloC₁-C₄alkyl, dihaloC₁-C₄alkyl, trihaloC₁-C₄alkyl, C₁-C₄alkoxy, C₁-C₄alkoxy-C₃-C₇cycloalkyl, haloC₁-C₄alkoxy, dihaloC₁-C₄alkoxy, trihaloC₁-C₄alkoxy, hydroxy, cyano, halogen, amino, mono- and di-C₁-C₄alkylamino, heteroaryl, C₁-C₄alkyl substituted with hydroxy, C₁-C₄alkoxy substituted with aryl, amino, —C(O)NH C₁-C₄alkyl-heteroaryl, —NHC(O)—C₁-C₄alkyl-heteroaryl, C₁-C₄alkyl C(O)NH— heteroaryl, C₁-C₄alkyl NHC(O)-heteroaryl, 3-7 membered cycloalkyl, 5-7 membered cycloalkenyl or 5, 6 or 9 membered heterocycle containing 1 or 2 heteroatoms, independently, selected from S, O and N, wherein heteroaryl has 5, 6 or 9 ring atoms, 1, 2 or 3 ring heteroatoms selected from N, O and S and substituted with 0, 1, or 2 substituents independently selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC₁-C₄alkylamino, hydroxyC₁-C₄alkyl, 4-7 member heterocycleC₁-C₄alkyl, aminoC₁-C₄alkyl and mono- and di-C₁-C₄alkylaminoC₁-C₄alkyl; or A is 2-naphthyl optionally substituted at the 3 position with hydroxy and additionally substituted with 0, 1, or 2 substituents selected from hydroxy, cyano, halogen, C₁-C₄alkyl, C₂-C₄alkenyl, C₁-C₅alkoxy, wherein the alkoxy is unsubstituted or substituted with hydroxy, C₁-C₄alkoxy, amino, N(H)C(O)C₁-C₄alkyl, N(H)C(O)₂ C₁-C₄alkyl, alkylene 4 to 7 member heterocycle, 4 to 7 member heterocycle and mono- and di-C₁-C₄alkylamino; or A is 6 member heteroaryl having 1-3 ring nitrogen atoms, which 6 member heteroaryl is substituted by phenyl or a heteroaryl having 5 or 6 ring atoms, 1 or 2 ring heteroatoms independently selected from N, O and S and substituted with 0, 1, or 2 substituents independently selected from C₁-C₄alkyl, mono- and di-C₁-C₄alkylamino, hydroxyC₁-C₄alkylamino, hydroxyC₁-C₄alkyl, aminoC₁-C₄alkyl and mono- and di-C₁-C₄alkylaminoC₁-C₄alkyl; or A is bicyclic heteroaryl having 9 to 10 ring atoms and 1, 2, or 3 ring heteroatoms independently selected from N, O or S, which bicyclic heteroaryl is substituted with 0, 1, or 2 substituents independently selected from cyano, halogen, hydroxy, C₁-C₄alkyl, C₂-C₄alkenyl, C₂-C₄alkynyl, C₁-C₄alkoxy and C₁-C₄alkoxy substituted with hydroxy, C₁-C₄alkoxy, amino and mono- and di-C₁-C₄alkylamino; or A is tricyclic heteroaryl having 12 or 13 ring atoms and 1, 2, or 3 ring heteroatoms independently selected from N, O or S, which tricyclic heteroaryl is substituted with 0, 1, or 2 substituents independently selected from cyano, halogen, hydroxy, C₁-C₄alkyl, C₂-C₄alkenyl, C₂-C₄alkynyl, C₁-C₄alkoxy, C₁-C₄alkoxy substituted with hydroxy, C₁-C₄alkoxy, amino, mono- and di-C₁-C₄alkylamino and heteroaryl, wherein said heteroaryl has 5, 6 or 9 ring atoms, 1, 2 or 3 ring heteroatoms selected from N, O and S and substituted with 0, 1, or 2 substituents independently selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC₁-C₄alkylamino, hydroxyC₁-C₄alkyl, 4-7 member heterocycleC₁-C₄alkyl, aminoC₁-C₄alkyl and mono- and di-C₁-C₄alkylaminoC₁-C₄alkyl; B is a group of the formula:

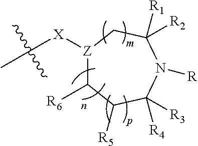

wherein m, n and p are independently selected from 0 or 1; R, R₁, R₂, R₃, and R₄ are independently selected from the group consisting of hydrogen, C₁-C₄alkyl, which alkyl is optionally substituted with hydroxy, amino or mono- and di-C₁-C₄akylamino; R₅ and R₆ are independently selected from hydrogen and fluorine; or R and R₃, taken in combination form a fused 5 or 6 member heterocyclic ring having 0 or 1 additional ring heteroatoms selected from N, O or S; R₁ and R₃, taken in combination form a C₁-C₃alkylene group; R₁ and R₅, taken in combination form a C₁-C₃alkylene group; R₃ and R₄, taken in combination with the carbon atom to which they attach, form a spirocyclicC₃-C₆cycloalkyl; X is CR_{A}R_{B}, O, NR₇ or a bond; R₇ is hydrogen, or C₁-C₄alkyl; R_{A} and R_{B} are independently selected from hydrogen and C₁-C₄alkyl, or R_{A} and R_{B}, taken in combination, form a divalent C₂-C₅alkylene group; Z is CR₈ or N; when Z is N, X is a bond; R₈ is hydrogen or taken in combination with R₆ form a double bond; or B is a group of the formula:

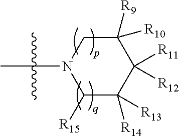

wherein p and q are independently selected from the group consisting of 0, 1, and 2; R₉ and R₁₃ are independently selected from hydrogen and C₁-C₄alkyl; R₁₀ and R₁₄ are independently selected from hydrogen, amino, mono- and di-C₁-C₄akylamino and C₁-C₄alkyl, which alkyl is optionally substituted with hydroxy, amino or mono- and di-C₁-C₄akylamino; R₁₁ is hydrogen, C₁-C₄alkyl, amino or mono- and di-C₁-C₄akylamino; R₁₂ is hydrogen or C₁-C₄alkyl; or R₉ and R₁₁, taken in combination form a saturated azacycle having 4 to 7 ring atoms which is optionally substituted with 1-3 C₁-C₄alkyl groups; or R₁₁ and R₁₂, taken in combination form a saturated azacycle having 4 to 7 ring atoms which is optionally substituted with 1-3 C₁-C₄alkyl groups.

In a thirty-ninth splice modulator aspect, the splice modulator is a compound or pharmaceutically acceptable salt thereof, according to the thirty-eighth splice modulator aspect, wherein A is 6 member heteroaryl having 1-3 ring nitrogen atoms, which 6 member heteroaryl is substituted by phenyl or a heteroaryl having 5 or 6 ring atoms, 1 or 2 ring heteroatoms independently selected from N, O and S and substituted with 0, 1, or 2 substituents independently selected from C₁-C₄alkyl, mono- and di-C₁-C₄alkylamino, hydroxyC₁-C₄alkylamino, hydroxyC₁-C₄alkyl, aminoC₁-C₄alkyl and mono- and di-C₁-C₄alkylaminoC₁-C₄alkyl; or A is bicyclic heteroaryl having 9 to 10 ring atoms and 1, 2, or 3 ring heteroatoms independently selected from N, O or S, which heteroaryl is substituted with 0, 1, or 2 substituents independently selected from cyano, halogen, hydroxy, C₁-C₄alkyl, C₂-C₄alkenyl, C₂-C₄alkynyl, C₁-C₄alkoxy and C₁-C₄alkoxy substituted with hydroxy, C₁-C₄alkoxy, amino and mono- and di-C₁-C₄alkylamino.

In a fortieth splice modulator aspect, the splice modulator is a compound or pharmaceutically acceptable salt thereof, according to the thirty-eighth splice modulator aspect, wherein A is 2-hydroxy-phenyl which is substituted with 0, 1, 2, or 3 substituents independently selected from C₁-C₄alkyl, haloC₁-C₄alkyl C₁-C₄alkoxy, hydroxy, cyano, halogen, amino, mono- and di-C₁-C₄alkylamino, heteroaryl and C₁-C₄alkyl substituted with hydroxy or amino, which heteroaryl has 5 or 6 ring atoms, 1 or 2 ring heteroatoms selected from N, O and S and substituted with 0, 1, or 2 substituents independently selected from C₁-C₄alkyl, mono- and di-C₁-C₄alkylamino, hydroxyC₁-C₄alkylamino, hydroxyC₁-C₄alkyl, 4-7 member heterocycleC₁-C₄alkyl, aminoC₁-C₄alkyl and mono- and di-C₁-C₄alkylaminoC₁-C₄alkyl.

In a forty-first splice modulator aspect, the splice modulator is a compound or pharmaceutically acceptable salt thereof, according to the thirty-eighth splice modulator aspect, wherein A is 2-naphthyl optionally substituted at the 3 position with hydroxy and additionally substituted with 0, 1, or 2 substituents selected from hydroxy, cyano, halogen, C₁-C₄alkyl, C₂-C₄alkenyl, C₁-C₄alkoxy, wherein the alkoxy is unsubstituted or substituted with hydroxy, C₁-C₄alkoxy, amino, N(H)C(O)C₁-C₄alkyl, N(H)C(O)₂ C₁-C₄alkyl, 4 to 7 member heterocycle and mono- and di-C₁-C₄alkylamino; or

In a forty-second splice modulator aspect, the splice modulator is a compound or pharmaceutically acceptable salt thereof, according to the thirty-eighth through forty-first splice modulator aspects, wherein B is a group of the formula:

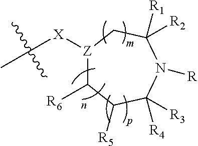

wherein m, n and p are independently selected from 0 or 1; R, R₁, R₂, R₃, and R₄ are independently selected from the group consisting of hydrogen, C₁-C₄alkyl, which alkyl is optionally substituted with hydroxy, amino or mono- and di-C₁-C₄akylamino; R₅ and R₆ are hydrogen; or R and R₃, taken in combination form a fused 5 or 6 member heterocyclic ring having 0 or 1 additional ring heteroatoms selected from N, O or S; R₁ and R₃, taken in combination form a C₁-C₃alkylene group; R₁ and R₅, taken in combination form a C₁-C₃alkylene group; R₃ and R₄, taken in combination with the carbon atom to which they attach, form a spirocyclicC₃-C₆cycloalkyl; X is CR_{A}R_{B}, O, NR₇ or a bond; R_{A} and R_{B} are independently selected from hydrogen and C₁-C₄alkyl, or R_{A} and R_{B}, taken in combination, form a divalent C₂-C₅alkylene group; Z is CR₈ or N; when Z is N, X is a bond; R₈ is hydrogen or taken in combination with R₆ form a double bond.

In a forty-third splice modulator aspect, the splice modulator is a compound or pharmaceutically acceptable salt thereof, according to the thirty-eighth through forty-first splice modulator aspects, wherein B is a group of the formula:

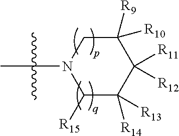

wherein p and q are independently selected from the group consisting of 0, 1, and 2; R₉ and R₁₃ are independently selected from hydrogen and C₁-C₄alkyl; R₁₀ and R₁₄ are independently selected from hydrogen, amino, mono- and di-C₁-C₄akylamino and C₁-C₄alkyl, which alkyl is optionally substituted with hydroxy, amino or mono- and di-C₁-C₄akylamino; R₁₁ is hydrogen, C₁-C₄alkyl, amino or mono- and di-C₁-C₄akylamino; R₁₂ is hydrogen or C₁-C₄alkyl; or R₉ and R₁₁, taken in combination form a saturated azacycle having 4 to 7 ring atoms which is optionally substituted with 1-3 C₁-C₄alkyl groups; or R₁₁ and R₁₂, taken in combination form a saturated azacycle having 4 to 7 ring atoms which is optionally substituted with 1-3 C₁-C₄alkyl groups.

In a forty-fourth splice modulator aspect, the splice modulator is a compound according to Formula (XVI):

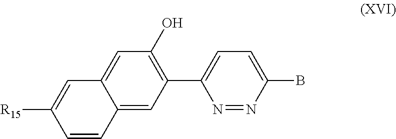

or pharmaceutically acceptable salt thereof, wherein R₁₅ is hydrogen, hydroxyl, C₁-C₄alkoxy, which alkoxy is optionally substituted with hydroxy, methoxy, amino, mono- and di-methylamino or morpholine.

In a forty-fifth splice modulator aspect, the splice modulator is a compound according to Formula (XVII):

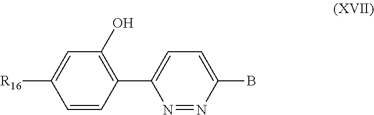

or pharmaceutically acceptable salt thereof, wherein R₁₆ is a 5 member heteroaryl having one ring nitrogen atom and 0 or 1 additional ring heteroatom selected from N, O or S, wherein the heteroaryl is optionally substituted with C₁-C₄alkyl.

In a forty-sixth splice modulator aspect, the splice modulator is a compound according to of the thirty-eighth through forty-first, forty-fourth and forty-fifth splice modulator aspects, wherein B is selected from the group consisting of

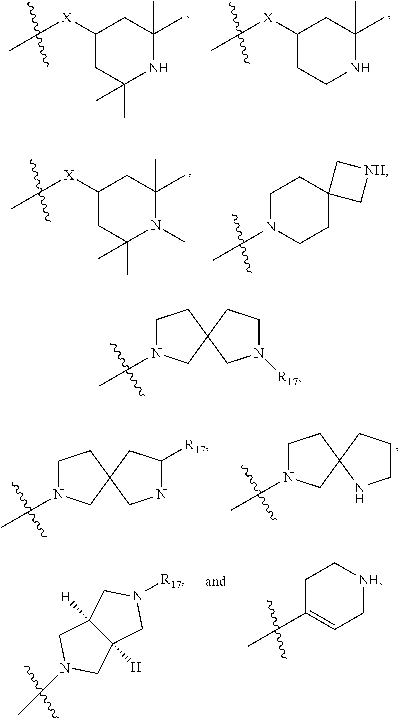

wherein X is O or N(Me); and R₁₇ is hydrogen or methyl.

In a forty-seventh splice modulator aspect, the splice modulator is a compound according to the thirty-eighth through forty-second and forty-fourth through forty-fifth splice modulator aspects, wherein X is —O—.

In a forty-eighth splice modulator aspect, the splice modulator is a compound according to the thirty-eighth through forty-second and forty-fourth through forty-fifth splice modulator aspects, wherein B is:

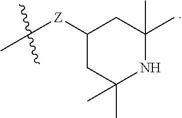

In a forty-ninth splice modulator aspect, the splice modulator is a compound according to the forty-fifth through forty-eighth splice modulator aspects, wherein R₁₆ is:

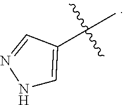 [SMILES: *c1cn[nH]c1]

In a fiftieth splice modulator aspect, the splice modulator is a compound according to Formula (XVIII):

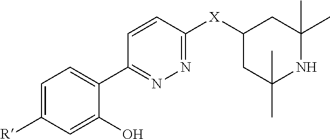

or pharmaceutically acceptable salt thereof, wherein X is —O— or

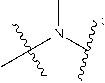 [SMILES: *N(*)C]

R′ is a 5-membered heteroaryl optionally substituted with 0, 1, or 2 groups selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC₁-C₄alkylamino, hydroxyC₁-C₄alkyl, 4-7 member heterocycleC₁-C₄alkyl, aminoC₁-C₄alkyl and mono- and di-C₁-C₄alkylaminoC₁-C₄alkyl.

In certain embodiments, the splice modulator is 5-(1H-Pyrazol-4-yl)-2-(6-((2,2,6,6-tetramethylpiperidin-4-yl)oxy)pyridazin-3-yl)phenol (LMI070; branaplam) having the following structure,

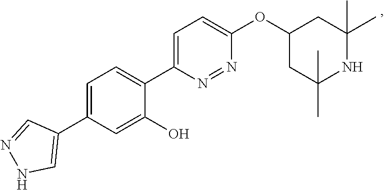

or a pharmaceutically acceptable salt thereof.

In certain embodiments, the splice modulator is splice modulator 2, wherein the compound is 7-(6-(methyl(2,2,6,6-tetramethylpiperidin-4-yl)amino)pyridazin-3-yl)isoquinolin-6-ol having the following structure,

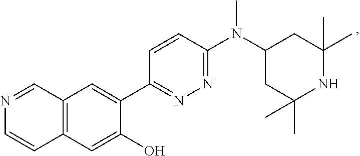 [SMILES: CN(c1ccc(-c2cc3cnccc3cc2O)nn1)C1CC(C)(C)NC(C)(C)C1]

or a pharmaceutically acceptable salt thereof.

Additional splice modulators and splice modulator binding sequences bound by those modulators are described in, for example, patent application publications US2012/0083495, WO2014/028459, WO2015/017589, WO2014/116845, WO2017/100726, WO2018/098446, WO2018/226622, WO2019/005993, WO2019/005980, and WO2019028440, the contents of which are hereby incorporated herein by reference in their entireties, and the splice modulators and splice modulator binding sequences described therein are contemplated for use in the methods, minigenes and other aspects and embodiments described herein.

### Cleavage Sites

In aspects, the nucleic acid molecule of the invention includes one or more sequences encoding a cleavage site, which serves the function of cleaving the sequence (e.g., all the sequence or substantially all the sequence) encoded by the minigene from the sequence (e.g., protein of interest) encoded by the transgene. In aspects, the cleavage site can either be a self-cleavage site, a protease cleavage site or any combination thereof. The cleavage site can be designed to be cleaved by any site-specific protease that is expressed in a cell of interest (either through recombinant expression or endogenous expression) at adequate levels to cleave off the sequence encoded by the one or more exons of the minigene from the protein of interest. In important aspects of the invention, the protease cleavage site is chosen to correspond to a protease natively (or by virtue of cell engineering) to be present in a cellular compartment relevant to the expression of the protein of interest. I.e., the intracellular trafficking of the protease should overlap or partially overlap with the intracellular trafficking of the protein of interest. For example, if the protein of interest is located at the cell surface, the enzyme to cleave it can be added exogenous to the cell.

If the protein of interest resides in or passes through the endosomal/lysosomal system a protease cleavage site for an enzyme resident in those compartments can be used. Such protease/consensus motifs include, e.g.,

Furin:
RX(K/R)R consensus motif 1
Furin:
(SEQ ID NO: 39)
RNRR
PCSK1:
RX(K/R)R consensus motif
PCSK5:
RX(K/R)R consensus motif
PCSK6:
RX(K/R)R consensus motif
PCSK7:
RXXX[KR]R consensus motif
Cathepsin B:
RRX
Granzyme B:
(SEQ ID NO: 35)
I-E-P-D-X
Factor XA:
Ile-Glu/Asp-Gly-Arg
Enterokinase:
(SEQ ID NO: 36)
Asp-Asp-Asp-Asp-Lys
Genenase:
(SEQ ID NO: 37)
Pro-Gly-Ala-Ala-His-Tyr
Sortase:
LPXTG/A
PreScission protease:
(SEQ ID NO: 38)
Leu-Glu-Val-Phe-Gln-Gly-Pro
Thrombin:
(SEQ ID NO: 40)
Leu-Val-Pro-Arg-Gly-Ser
TEV protease:
(SEQ ID NO: 41)
E-N-L-Y-F-Q-G
Elastase 1
(SEQ ID NO: 42)
[AGSV]-x

In some embodiments, the nucleic acid described herein includes a sequence encoding a furin cleavage site. In some embodiments, the nucleic acids described herein include a sequence encoding any one of the furin cleavage sites listed in Table 20. In embodiments, the furin cleavage site is SEQ ID NO: 39. In some embodiments, the nucleic acids described herein include a sequence encoding a furin cleavage site that includes or consists of SEQ ID NO: 39, for example, the sequence encoding a cleavage site includes or consists of SEQ ID NO: 19.

In some embodiments, the nucleic acids described herein include a sequence encoding a furin cleavage site selected from RNRR (SEQ ID NO: 39) or a sequence having at least 90%, 95%, 97%, 98%, or 99% identity thereto; RTKR (SEQ ID NO: 43) or a sequence having at least 90%, 95%, 97%, 98%, or 99% identity thereto; GTGAEDPRPSRKRRSLGDVG (SEQ ID NO: 45) or a sequence having at least 90%, 95%, 97%, 98%, or 99% identity thereto; GTGAEDPRPSRKRR (SEQ ID NO: 47) or a sequence having at least 90%, 95%, 97%, 98%, or 99% identity thereto; LQWLEQQVAKRRTKR (SEQ ID NO: 49) or a sequence having at least 90%, 95%, 97%, 98%, or 99% identity thereto; GTGAEDPRPSRKRRSLGG (SEQ ID NO: 51) or a sequence having at least 90%, 95%, 97%, 98%, or 99% identity thereto; GTGAEDPRPSRKRRSLG (SEQ ID NO: 53) or a sequence having at least 90%, 95%, 97%, 98%, or 99% identity thereto; SLNLTESHNSRKKR (SEQ ID NO: 55) or a sequence having at least 90%, 95%, 97%, 98%, or 99% identity thereto; or CKINGYPKRGRKRR (SEQ ID NO: 57) or a sequence having at least 90%, 95%, 97%, 98%, or 99% identity thereto.

In some embodiments, the nucleic acids described herein include a sequence encoding a furin cleavage site selected from RNRR (SEQ ID NO: 39); RTKR (SEQ ID NO: 43); GTGAEDPRPSRKRRSLGDVG (SEQ ID NO: 45); GTGAEDPRPSRKRR (SEQ ID NO: 47); LQWLEQQVAKRRTKR (SEQ ID NO: 49); GTGAEDPRPSRKRRSLGG (SEQ ID NO: 51); GTGAEDPRPSRKRRSLG (SEQ ID NO: 53); SLNLTESHNSRKKR (SEQ ID NO: 55); and CKINGYPKRGRKRR (SEQ ID NO: 57).

In some embodiments, the nucleic acids described herein include a sequence encoding a furin cleavage site selected from RNRR (SEQ ID NO: 39) or a sequence having at least 90%, 95%, 97%, 98%, or 99% identity thereto. In some embodiments, the nucleic acids described herein include SEQ ID NO: 19, or a sequence having at least 90%, 95%, 97%, 98%, or 99% identity thereto. In some embodiments, the nucleic acids described herein include SEQ ID NO: 19.

In some embodiments, the nucleic acids described herein include a sequence encoding a furin cleavage site selected from GTGAEDPRPSRKRRSLGDVG (SEQ ID NO: 45) or a sequence having at least 90%, 95%, 97%, 98%, or 99% identity thereto, or GTGAEDPRPSRKRR (SEQ ID NO: 47) or a sequence having at least 90%, 95%, 97%, 98%, or 99% identity thereto. In some embodiments, the nucleic acids described herein include SEQ ID NO: 46 or SEQ ID NO: 48, or a sequence having at least 90%, 95%, 97%, 98%, or 99% identity thereto.

In some embodiments, the nucleic acids described herein include a sequence encoding a furin cleavage site selected from GTGAEDPRPSRKRRSLGDVG (SEQ ID NO: 45) or GTGAEDPRPSRKRR (SEQ ID NO: 47). In some embodiments, the nucleic acids described herein include SEQ ID NO: 46 or SEQ ID NO: 48

In some embodiments, the nucleic acids described herein include a sequence encoding the furin cleavage site of GTGAEDPRPSRKRRSLGDVG (SEQ ID NO: 45).

TABLE 20
Exemplary furin cleavage sites and nucleic acid sequences encoding them.

| | Amino acid sequence | Nucleic acid sequence |
|---|---|---|
| Furin cleavage site0 | RNRR (SEQ ID NO: 39) | cgcaaccgccgc (SEQ ID NO: 19) |
| Furin cleavage site1 | RTKR (SEQ ID NO: 43) | cgtactaaaaga (SEQ ID NO: 44) |
| Furin cleavage site2 | GTGAEDPRPSRKRRSLGDVG | ggaaccggcgcggaagacccccggccctccaggaag |
| | (SEQ ID NO: 45) | cgaaggtccctcggagacgtgggt (SEQ ID NO: |
| | | 46) |
| Furin cleavage site3 | GTGAEDPRPSRKRR (SEQ ID | ggaaccggcgcggaagacccccggccctccaggaag |
| | NO: 47) | cgaagg (SEQ ID NO: 48) |
| Furin cleavage site4 | LQWLEQQVAKRRTKR | ctgcaatggctggagcagcaggtggcgaagcggagaa |
| | (SEQ ID NO: 49) | ctaagcgg (SEQ ID NO: 50) |
| Furin cleavage site5 | GTGAEDPRPSRKRRSLGG | ggcacaggtgccgaggaccctcggccaagccgcaaaa |
| | (SEQ ID NO: 51) | ggaggtcacttggcggc (SEQ ID NO: 52) |
| Furin cleavage site6 | GTGAEDPRPSRKRRSLG | ggaaccggagcagaagatcccagaccaagccggaaa |
| | (SEQ ID NO: 53) | aggcggtccctgggt (SEQ ID NO: 54) |
| Furin cleavage site7 | SLNLTESHNSRKKR | agtctcaatttgactgagtcacacaattccaggaagaaaa |
| | (SEQ ID NO: 55) | gg (SEQ ID NO: 56) |
| Furin cleavage site8 | CKINGYPKRGRKRR | tgcaagatcaacggctaccctaagaggggcagaaagc |
| | (SEQ ID NO: 57) | ggcgg (SEQ ID NO: 58) |

In some embodiments, the nucleic acid sequence comprising a minigene and a transgene, e.g., described herein, can include one or more sequences encoding a peptide cleavage sites (e.g., an self-cleaving peptide or a substrate for an intracellular protease). In embodiments, the sequence encoding a peptide cleavage site is disposed between the minigene and the transgene. Examples of self-cleaving peptide cleavage sites sequences include the following, wherein the GSG residues in parentheses are optional:

TABLE 21
Exemplary self-cleaving peptide sequences and nucleic acid sequences encoding them
(GSG sequence in each is optional).

| | Amino acid sequence | Nucleic acid sequence |
|---|---|---|
| T2A | (GSG) E G R G S L L T C G D | GGCAGCGGCGAAGGCCGCGGCAGCCT |
| | V E E N P G P (SEQ ID NO: | GCTGACCTGCGGCGATGTGGAAGAAAA |
| | 59) | CCCGGGCCCG (SEQ ID NO: 20) |
| T2A (without GSG) | E G R G S L L T C G D V E E | GAAGGCCGCGGCAGCCTGCTGACCTG |
| | N P G P (SEQ ID NO: 61) | CGGCGATGTGGAAGAAAACCCGGGCC |
| | | C (SEQ ID NO: 62) |
| P2A | (GSG) A T N F S L L K Q A G | (ggcagcggc)gccaccaacttcagcctgctgaagcagg |
| | D V E E N P G P (SEQ ID NO: | ccggcgacgtggaggagaaccccggcccc (SEQ ID |
| | 63) | NO: 64) |
| E2A | (GSG) Q C T N Y A L L K L A | (ggcagcggc)cagtgcaccaactacgccctgctgaagc |
| | G D V E S N P G P (SEQ ID | tggccggcgacgtggagagcaaccccggcccc (SEQ |
| | NO: 65) | ID NO: 66) |
| F2A | (GSG) V K Q T L N F D L L K L | (ggcagcggc)gtgaagcagaccctgaacttcgacctgc |
| | A G D V E S N P G P (SEQ ID | tgaagctggccggcgacgtggagagcaaccccggccc |
| | NO: 67) | c (SEQ ID NO: 68) |

In some embodiments, the nucleic acid molecule includes a sequence encoding a protease cleavage site, such as a furin cleavage site, and a sequence encoding a self-cleaving peptide, for example a 2A peptide, for example a T2A peptide. In embodiments, the nucleic acid comprises the sequence encoding the furin cleavage site 5′ to the sequence encoding the 2A-encoding sequence. In embodiments, the furin cleavage site comprises or consists of SEQ ID NO: 39 and the T2A sequence comprises or consists of SEQ ID NO: 59 or SEQ ID NO: 61. In embodiments, the sequence encoding the furin cleavage site is or comprises SEQ ID NO: 19 and the sequence encoding the peptide cleavage site is or comprises SEQ ID NO: 20 or SEQ ID NO: 62. In embodiments, the sequence encoding the 2A sequence is disposed immediately 5′ of the transgene (e.g., the sequence encoding the protein of interest), such that upon cleavage, fewer than 10, 9, 8, 7, 6, 5, 4, 3, 2, or 1 amino acids of the minigene, furin cleavage site and/or 2A peptide are left on the protein of interest.

### Promoters

All cells in the animal or human body contain the same DNA, yet different cells in different tissues express, on the one hand, a set of common genes, and on the other, a set of genes that vary depending on the type of tissue and the stage of development. Without being bound by theory, any promoter that does not contain an intron can be used in the various aspects and embodiments (e.g., in the nucleic acid molecules) described herein. Exemplary promoters that can be used with the various aspects and embodiments described herein include, but are not limited to, the cytomegalovirus (CMV) promoter, the CAG promoter, the SV40 promoter, the JeT promoter, the PGK promoter and the chicken beta-actin promoter (CBA) promoter. In embodiments, the promoter is active in more than one cell type. In other embodiments, the promoter is active in one cell type (e.g., cell-specific) or in cell types of one tissue (e.g., tissue-specific), such as, for example, central nervous tissue (e.g., brain tissue). In embodiments, the promoter is neuron specific. Examples of neuron specific promoters that can be used in the various aspects and embodiments described herein include, but are not limited to, isolated or synthetic neuron-specific promoters and functional fragments thereof used in vectors and other nucleic acids to drive expression of an operatively linked minigene and transgene, e.g., promoters derived from neuron-specific enolase (NSE) (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al., (1987) Cell, 51:7-19; Llewellyn et al. (2010) Nat. Med., 16(10):1161-1166); a serotonin receptor promoter (see, e.g., GenBank S62283); a tyrosine hydroxylase promoter (TH) (see, e.g., Oh et al., (2009) Gene Ther., 16:437; Sasaoka et al., (1992) Mol. Brain Res., 16:274; Boundy et al., (1998) J. Neurosci., 18:9989; and Kaneda et al., (1991) Neuron, 6:583-594); a GnRH promoter (see, e.g., Radovick et al., (1991) Proc. Nat. Acad. Sci. USA, 88:3402-3406); an L7 promoter (see, e.g., Oberdick et al., (1990) Science, 248:223-226); a DNMT promoter (see, e.g., Bartge et al., (1988) Proc. Nat. Acad. Sci. USA, 85:3648-3652); an enkephalin promoter (see, e.g., Comb et al., (1988) EMBO J., 17:3793-3805); a myelin basic protein (MBP) promoter; a Ca2+-calmodulin-dependent protein kinase II-alpha (CamKIM) promoter (see, e.g., Mayford et al., (1996) Proc. Natl. Acad. Sci. USA, 93:13250; and Casanova et al., (2001) Genesis, 31:37); a CMV enhancer/platelet-derived growth factor-p promoter (see, e.g., Liu et al., (2004) Gene Ther., 11:52-60); and the like. In some embodiments, portions or all of the minimal human synapsin 1 promoter (SYN) are used. Kugler et al., (2003) Gene Ther., 10(4): 337-47; Thiel et al, (1991) Proc. Natl. Acad. Sci. USA, 88(8) 3431-5; Castle et al., (2016) Methods Mol. Biol., 1382: 133-49; McLean et al., (2014) Neurosci. Lett., 576: 73-78; Kugler et al., (2003) Virology, 311(1): 89-95.

In some embodiments, a tissue- or cell-specific promoter is configured to provide higher expression of an operatively linked minigene and/or transgene in a neuronal cell or tissue relative to that in a non-neuronal cell. In some embodiments, the neuron specific promoter is configured to provide higher expression of an operatively linked minigene and/or transgene in a neuron relative to that in a non-neuronal cell. Examples of neuronal cells or tissue include those comprising neurons, as well as Schwann cells, glial cells, astrocytes, etc. Examples of non-neuronal cells include, but are not limited to, hepatic cells, cardiomyocytes, red blood cells, epithelial cells etc. Higher levels of expression of an operatively linked minigene and/or transgene may include an increase in the number of RNA transcripts produced from transcription of the minigene and/or transgene. In some embodiments, the number of RNA transcripts produced may be measured by PCR. In some other embodiments, the number of RNA transcripts produced may be measured by RT-PCR, e.g., qPCR. In some embodiments, the number of RNA transcripts produced may be measured by sequencing. In some embodiments, the number of RNA transcripts produced may be measured by single-molecule Fluorescence In-Situ Hybridization (FISH). In some embodiments, the number of RNA transcripts produced may be measured by Northern blot analysis. Higher levels of expression of an operatively linked minigene and/or transgene may alternatively or in addition include an increase in the amount of protein produced, when the minigene and/or transgene encodes a protein of interest. In some embodiments, the amount of protein produced may be measured by an enzyme-linked immunosorbent assay (ELISA). In some embodiments, the amount of protein produced may be measured by Western blot analysis. In some embodiments, the amount of protein produced may be measured by immunostaining. In some embodiments, the amount of protein produced may be measured by time-resolved Forster Resonance Energy Transfer (TR-FRET). In some embodiments, the amount of protein produced may be measured by immunohistochemistry (IHC). In some embodiments, the level of expression is measured by more than one of these or other methods.

In aspects and embodiments, the promoter is a JeT promoter comprising SEQ ID NO: 13. In aspects and embodiments, the promoter is a human synapsin promoter comprising SEQ ID NO: 86.

Poly A Signal Sequence

In various embodiments, the nucleic acids, vectors and other compositions disclosed herein may comprise one or more polyadenylation (PolyA) signal sequences. The polyadenylation signal sequences may comprise a central sequence (e.g., AAUAAA) flanked by auxiliary sequence elements. Without being bound by theory, the sequence may signal the end of the transcript and serve as the site where a homopolymeric A sequence is added on the 3′ end by polyadenylate polymerase.

Polyadenylation signal sequences known in the art are contemplated, including but not limiting to the SV40 polyA, the human growth hormone (HGH) polyA, the bovine growth hormone (BGH) polyA, the beta-globin polyA, the alpha-globin polyA, the ovalbumin polyA, the kappa-light chain polyA, and a synthetic polyA. PolyA signal sequences may be used in the nucleic acids and other compositions disclosed herein. In some embodiments, the polyA sequence in the transgene or nucleic acid sequence consists of SEQ ID NO: 22 or a functional fragment thereof. In some embodiments, the transgene or nucleic acid sequence comprises a sequence having at least about 80, 85, 90, 95, 98, or 99% identity to SEQ ID NO: 22 or a functional fragment thereof. In some embodiments, the polyA sequence in the transgene or nucleic acid consists of a sequence of at least about 80, 85, 90, 95, 98, or 99% identity to SEQ ID NO: 22 or a functional fragment thereof. In some embodiments, the polyA sequence in the transgene or nucleic acid sequence consists of SEQ ID NO: 89 or a functional fragment thereof. In some embodiments, the transgene or nucleic acid sequence comprises a sequence having at least about 80, 85, 90, 95, 98, or 99% identity to SEQ ID NO: 89 or a functional fragment thereof. In some embodiments, the polyA sequence in the transgene or nucleic acid consists of a sequence of at least about 80, 85, 90, 95, 98, or 99% identity to SEQ ID NO: 89 or a functional fragment thereof.

Post-Transcriptional Regulatory Elements

In various embodiments, the nucleic acids, transgenes, and other compositions disclosed herein may comprise one or more post-transcriptional regulatory elements (PREs), e.g., those that can enhance or otherwise improve expression of the transgene. Without being bound by the theory, PREs may enhance expression by enabling stability and 3′ end formation of mRNA, and/or may facilitate the nucleocytoplasmic export of unspliced mRNAs. PREs may also comprise binding sites for RNA-binding proteins (RBPs) or microRNAs.

Exemplary PREs include but are not limited to a PRE from the Hepatitis B virus (HPRE), bat virus (BPRE), ground squirrel virus (GSPRE), arctic squirrel virus (ASPRE), duck virus (DPRE), chimpanzee virus (CPRE) wooly monkey virus (WMPRE) or woodchuck virus (WPRE). In some embodiments, the nucleic acid or transgene comprises a PRE. In certain embodiments, the PRE comprises the HPRE.

In some embodiments, a synthetic PRE is used. An example sequence of a synthetic PRE includes the sequence of the HPRE-NOX SEQ ID NO: 88, or a fragment thereof. In some embodiments, PREs may be disposed downstream (or 3′ to) a promoter element.

Exemplary PREs also include, but are not limited to, a PRE comprising, e.g., consisting of, SEQ ID NO: 72, or a fragment thereof. Exemplary PREs also include, but are not limited to, a PRE comprising, e.g., consisting of, SEQ ID NO: 73, or a fragment thereof.

(SEQ ID NO: 72)
ACAGGCCTATTGATTGGAAAGTATGTCAACGAATTGTGGGTCTTTTGGGG
TTTGCTGCCCCTTTTACGCAATGTGGATATCCTGCTTTAATGCCTTTATA
TGCATGTATACAAGCAAAACAGGCTTTTACTTTCTCGCCAACTTACAAGG
CCTTTCTAAGTAAACAGTATCTGACCCTTTACCCCGTTGCTCGGCAACGG
CCTGGTCTGTGCCAAGTGTTTGCTGACGCAACCCCCACTGGTTGGGGCTT
GGCCATAGGCCATCAGCGCATGCGTGGAACCTTTGTGTCTCCTCTGCCGA
TCCATACTGCGGAACTCCTAGCCGCTTGTTTTGCTCGCAGCAGGTCTGGA
GCGAAACTCATCGGGACTGACAATTCTGTCGTGCTCTCCCGCAAGTATAC
ATCGTTTCCAGGGCTGCTAGGCTGTGCTGCCAACTGGATCCTGCGCGGGA
CGTCCTTTGTTTACGTCCCGTCGGCGCTGAATCCCGCGGACGACCCCTCC
CGGGGCCGCTTGGGGCTCTACCGCCCGCTTCTCCGTCTGCCGTACCGACC
GACCACGGGGCGCACCTCTCTTTACGCGGACTCCCCGTCTGTGCCTTCTC
ATCTGCCGGACCGTGTGCACTTCGCTTCACCTCTGCACGTCGCATGGAGA
CCACCGTGAACGCCCACCGGAACCTGCCCAAGGTCTTGCATAAGAGGACT
CTTGGACTTTCAGCAATGTC
(SEQ ID NO: 73)
AACAGGCCTATTGATTGGAAAGTATGTCAACGAATTGTGGGTCTTTTGGG
GTTTGCTGCCCCTTTTACGCAATGTGGATATCCTGCTTTAATGCCTTTAT
ATGCATGTATACAAGCAAAACAGGCTTTTACTTTCTCGCCAACTTACAAG
GCCTTTCTAAGTAAACAGTATCTGACCCTTTACCCCGTTGCTCGGCAACG
GCCTGGTCTGTGCCAAGTGTTTGCTGACGCAACCCCCACTGGTTGGGGCT
TGGCCATAGGCCATCAGCGCATGCGTGGAACCTTTGTGTCTCCTCTGCCG
ATCCATACTGCGGAACTCCTAGCCGCTTGTTTTGCTCGCAGCAGGTCTGG
AGCGAAACTCATCGGGACTGACAATTCTGTCGTGCTCTCCCGCAAGTATA
CATCGTTTCCAGGGCTGCTAGGCTGTGCTGCCAACTGGATCCTGCGCGGG
ACGTCCTTTGTTTACGTCCCGTCGGCGCTGAATCCCGCGGACGACCCCTC
CCGGGGCCGCTTGGGGCTCTACCGCCCGCTTCTCCGTCTGCCGTACCGAC
CGACCACGGGGCGCACCTCTCTTTACGCGGACTCCCCGTCTGTGCCTTCT
CATCTGCCGGACCGTGTGCACTTCGCTTCACCTCTGCACGTCGCATGGAG
ACCACCGTGAACGCCCACCGGAACCTGCCCAAGGTCTTGCATAAGAGGAC
TCTTGGACTTTCAGCAATGTC

Exemplary PREs also include a PRE comprising or consisting of sequence with at least 85%, at least 90% at least 91%, at least 92%, at least 93%, at least 94%, at least 95%, at least 96%, at least 97%, at least 98%, or at least 99% sequence identity to a PRE described herein, e.g., to SEQ ID NO: 88, SEQ ID NO: 72 or SEQ ID NO: 73.

In some embodiments, PREs may be disposed downstream (or 3′ to) a transgene sequence or protein-coding sequence. In some embodiments, PREs may be disposed upstream of (or 5′ to) a polyA sequence. In some embodiments, PREs may be disposed upstream of (or 5′ to) a transgene sequence or protein-coding sequence.

Transgenes

In various embodiments, the minigenes and other regulatory elements disclosed herein may be used to regulate expression of an operably linked transgene. In some embodiments, the transgene encodes a protein such as an antibody or functional binding fragment, a receptor, an enzyme, etc. In some embodiments, the transgene encodes a therapeutic nucleic acid such as an shRNA, siRNA, gRNA for use in CRISPR, etc. In some embodiments, more than one transgene may be used (e.g., a nucleic acid or vector may encode more than one protein or RNA that provides therapeutic benefits). Examples of methods to increase levels of these functional polypeptides or nucleic acids in cells include transfection or transduction of a nucleic acid sequence encoding the polypeptide of interest, e.g., in a nucleic acid or vector disclosed herein, e.g., an AAV viral vector.

i. Proteins

In various embodiments, the minigenes and other regulatory elements disclosed herein may be used to regulate (e.g., turn on or turn off, in the presence or absence of a splice modulator) expression of polypeptides. Without being bound by theory, increases in the level of polypeptides in may provide therapeutic effects by providing for a polypeptide whose expression is reduced or missing in a subject patient's tissue. Without being bound by theory, controlling the timing or location of expression, e.g., by application or withdrawal of a splice modulator, may improve the effectiveness and/or safety of such therapeutic protein by ensuring expression only when it wanted. Exemplary polypeptides that may regulated by the minigenes described herein include but are not limited to superoxide dismutase, aromatic acid decarboxylase (AADC), survival of motor neuron (SMN) protein, progranulin (PRGN), a Cas9 protein, a zinc finger nuclease or a TALEN), or a therapeutic protein such as, for example, a protein selected from MeCP2, CLN2, CLN3, CLN4, CLN5, CLN6, CLN7, and CLN8, or a protein related to spinacerebella ataxia (SCA), optionally any of SCA1-SCA29.

In various embodiments, the minigenes and other regulatory elements disclosed herein may be used to regulate expression of progranulin (PGRN). Without being bound by theory, increases in the level of functional PRGN polypeptides in neurons may provide therapeutic effects, e.g., in the treatment of FTD. Without being bound by theory, PGRN is typically observed in humans as a ubiquitously expressed, 88 kDa secreted glycoprotein. It is encoded by the human granulin gene (GRN). Exemplary nucleic acids encoding the progranulin protein include NG_007886.1 and NM_002087.3 as defined by RefSeqGene, and NC_000017.11 and NC_000017.10 as defined by NCBI Reference Sequences. Exemplary progranulin polypeptide sequences include NP_002078.1. In some embodiments, the progranulin polypeptide contains seven granulin-like domains, which consist of highly conserved tandem repeats of a rare 12 cysteinyl motif (SEQ ID NO: 102) connected by linker sequences.

In some embodiments, peptide fragments of PGRN and nucleic acids encoding them are encompassed by the term PGRN to the extent they retain one or more function of PGRN. Cleavage of PGRN to form granulins (GRNs) or epithelins may produce proteins with different function and are outside the meaning of fragments of PGRN as used herein. In some embodiments, a nucleic acid, vector, or other composition disclosed herein comprises a transgene sequence encoding a human protein. In some embodiments, the transgene sequence encodes PGRN. In some embodiments, the transgene sequence encodes a human progranulin (hPGRN) protein. In some embodiments, the transgene sequence encodes a codon-optimized version of the hPGRN protein. In some embodiments, the transgene sequence comprises a sequence of SEQ ID NO: 87 or a functional fragment thereof, e.g., a fragment capable of providing detectable changes in one or more of the functions provided by intact PGRN. In some embodiments, the transgene sequence comprises a sequence with at least 99%, 98%, 97%, 96%, 95%, 94%, 93%, 92%, 91%, 90%, 85%, 80%, 75%, or 70% sequence identity (or any percentage in between) to SEQ ID NO: 87. In some embodiments, the hPGRN encoded by the transgene comprises an amino acid sequence of SEQ ID NO: 87. In some embodiments, the hPGRN encoded by the heterologous nucleic acid sequence comprises a sequence with at least 99%, 98%, 97%, 96%, 95%, 94%, 93%, 92%, 91%, 90%, 85%, 80%, 75%, or 70% sequence identity comprises an amino acid sequence of SEQ ID NO: 81.

| | Sequence |
|---|---|
| hPGRN | TGGACCCTGGTGAGCTGGGTGGCCTTAACAGCAGGGCTGGTGGCTGGAAC |
| Nucleic acid | GCGGTGCCCAGATGGTCAGTTCTGCCCTGTGGCCTGCTGCCTGGACCCCG |
| SEQ ID NO: | GAGGAGCCAGCTACAGCTGCTGCCGTCCCCTTCTGGACAAATGGCCCACA |
| 87 | ACACTGAGCAGGCATCTGGGTGGCCCCTGCCAGGTTGATGCCCACTGCTC |
| | TGCCGGCCACTCCTGCATCTTTACCGTCTCAGGGACTTCCAGTTGCTGCCC |
| | CTTCCCAGAGGCCGTGGCATGCGGGGATGGCCATCACTGCTGCCCACGG |
| | GGCTTCCACTGCAGTGCAGACGGGCGATCCTGCTTCCAAAGATCAGGTAA |
| | CAACTCCGTGGGTGCCATCCAGTGCCCTGATAGTCAGTTCGAATGCCCGG |
| | ACTTCTCCACGTGCTGTGTTATGGTCGATGGCTCCTGGGGGTGCTGCCCCA |
| | TGCCCCAGGCTTCCTGCTGTGAAGACAGGGTGCACTGCTGTCCGCACGGT |
| | GCCTTCTGCGACCTGGTTCACACCCGCTGCATCACACCCACGGGCACCCA |
| | CCCCCTGGCAAAGAAGCTCCCTGCCCAGAGGACTAACAGGGCAGTGGCCT |
| | TGTCCAGCTCGGTCATGTGTCCGGACGCACGGTCCCGGTGCCCTGATGGT |
| | TCTACCTGCTGTGAGCTGCCCAGTGGGAAGTATGGCTGCTGCCCAATGCC |
| | CAACGCCACCTGCTGCTCCGATCACCTGCACTGCTGCCCCCAAGACACTGT |
| | GTGTGACCTGATCCAGAGTAAGTGCCTCTCCAAGGAGAACGCTACCACGG |
| | ACCTCCTCACTAAGCTGCCTGCGCACACAGTGGGGGATGTGAAATGTGACA |
| | TGGAGGTGAGCTGCCCAGATGGCTATACCTGCTGCCGTCTACAGTCGGGG |
| | GCCTGGGGCTGCTGCCCTTTTACCCAGGCTGTGTGCTGTGAGGACCACAT |
| | ACACTGCTGTCCCGCGGGGTTTACGTGTGACACGCAGAAGGGTACCTGTG |
| | AACAGGGGCCCCACCAGGTGCCCTGGATGGAGAAGGCCCCAGCTCACCTC |
| | AGCCTGCCAGACCCACAAGCCTTGAAGAGAGATGTCCCCTGTGATAATGTC |
| | AGCAGCTGTCCCTCCTCCGATACCTGCTGCCAACTCACGTCTGGGGAGTG |
| | GGGCTGCTGTCCAATCCCAGAGGCTGTCTGCTGCTCGGACCACCAGCACT |
| | GCTGCCCCCAGGGCTACACGTGTGTAGCTGAGGGGCAGTGTCAGCGAGG |
| | AAGCGAGATCGTGGCTGGACTGGAGAAGATGCCTGCCCGCCGGGCTTCCT |
| | TATCCCACCCCAGAGACATCGGCTGTGACCAGCACACCAGCTGCCCGGTG |
| | GGGCAGACCTGCTGCCCGAGCCTGGGTGGGAGCTGGGCCTGCTGCCAGT |
| | TGCCCCATGCTGTGTGCTGCGAGGATCGCCAGCACTGCTGCCCGGCTGGC |
| | TACACCTGCAACGTGAAGGCTCGATCCTGCGAGAAGGAAGTGGTCTCTGC |
| | CCAGCCTGCCACCTTCCTGGCCCGTAGCCCTCACGTGGGTGTGAAGGACG |
| | TGGAGTGTGGGGAAGGACACTTCTGCCATGATAACCAGACCTGCTGCCGA |
| | GACAACCGACAGGGCTGGGCCTGCTGTCCCTACCGCCAGGGCGTCTGTTG |
| | TGCTGATCGGCGCCACTGCTGTCCTGCTGGCTTCCGCTGCGCAGCCAGGG |
| | GTACCAAGTGTTTGCGCAGGGAGGCCCCGCGCTGGGACGCCCCTTTGAGG |
| | GACCCAGCCTTGAGACAGCTGCTGTAG |
| hPGRN | MWTLVSWVALTAGLVAGTRCPDGQFCPVACCLDPGGASYSCCRPLLDKWPTTLSRHL |
| Amino acid | GGPCQVDAHCSAGHSCIFTVSGTSSCCPFPEAVACGDGHHCCPRGFHCSADGRSCF |
| SEQ ID NO: | QRSGNNSVGAIQCPDSQFECPDFSTCCVMVDGSWGCCPMPQASCCEDRVHCCPHG |
| 81 | AFCDLVHTRCITPTGTHPLAKKLPAQRTNRAVALSSSVMCPDARSRCPDGSTCCELPS |
| | GKYGCCPMPNATCCSDHLHCCPQDTVCDLIQSKCLSKENATTDLLTKLPAHTVGDVKC |
| | DMEVSCPDGYTCCRLQSGAWGCCPFTQAVCCEDHIHCCPAGFTCDTQKGTCEQGPH |
| | QVPWMEKAPAHLSLPDPQALKRDVPCDNVSSCPSSDTCCQLTSGEWGCCPIPEAVCC |
| | SDHQHCCPQGYTCVAEGQCQRGSEIVAGLEKMPARRASLSHPRDIGCDQHTSCPVGQ |
| | TCCPSLGGSWACCQLPHAVCCEDRQHCCPAGYTCNVKARSCEKEVVSAQPATFLARS |
| | PHVGVKDVECGEGHFCHDNQTCCRDNRQGWACCPYRQGVCCADRRHCCPAGFRCA |
| | ARGTKCLRREAPRWDAPLRDPALRQLL |

ii. RNA

In various embodiments, the isolated nucleic acids, vectors, and other compositions disclosed herein may comprise a transgene sequence encoding a sequence that provides neuronal tissue-specific therapeutic effects without requiring protein translation. In some embodiments, the promoters, silencers, regulatory elements, and other nucleic acid elements disclosed herein may be used to regulate neuronal tissue or neuron-specific expression of RNA. In some embodiments, the transgene sequence encodes a ribonucleic acid providing a particular therapeutic function. In some embodiments, the transgene sequence encodes a siRNA. In some embodiments, the transgene sequence encodes a shRNA. In some embodiments, the transgene sequence encodes an miRNA. In some embodiments, the transgene sequence encodes a tRNA.

iii. Antibody

In various embodiments, the promoters, silencers, and other regulatory elements disclosed herein may be used to regulate neuronal tissue or neuron-specific expression of antibodies or fragments thereof. For instance, in some embodiments, the transgene sequence encodes an antibody. In some embodiments, the transgene sequence encodes a fragment of an antibody, e.g., one that retains antigen-binding capabilities. In some embodiments, the transgene sequence encodes a light chain of an antibody. In some embodiments, the transgene sequence encodes a heavy chain of an antibody. In some embodiments, the transgene sequence encodes a V_{H}. In some embodiments, the transgene sequence encodes a V_{L}. In some embodiments, the transgene sequence encodes a V_{H}. In some embodiments, the transgene sequence encodes a Fab. In some embodiments, the transgene sequence encodes a scFv. In some embodiments, the transgene sequence encodes an enzyme with neuron-specific function.

iii. More than One Component

In various embodiments, the promoters, silencers, and other regulatory elements disclosed herein may be used to regulate neuronal tissue or neuron-specific expression of more than one transgene. In some embodiments, the transgene sequences encode both an RNA and a polypeptide. In some embodiments, the transgene sequence encodes components of a CRISPR/Cas system. In some embodiments, the transgene sequence encodes a Cas9 protein. In some embodiments, the transgene sequence encodes a Cpf1 protein. In some embodiments, the transgene sequence encodes a CRISPR RNA (crRNA). In some embodiments, the transgene encodes a transactivating crRNA (tracRNA

In some embodiments, a nucleic acid, vector, or other composition disclosed herein comprises a minigene (e.g., as described herein), a transgene sequence encoding hPGRN, a PRE, and a polyA signal sequence, e.g., present in that order from 5′ to 3′. In some embodiments, the nucleic acid, vector, or other composition comprises, from 5′ to 3′, a promoter, a minigene, a sequence encoding a protease cleavage site (e.g., a furin cleavage site), a sequence encoding a self-cleaving peptide (e.g., a T2A peptide), a transgene sequence encoding hPGRN, a PRE, and a polyA signal sequence. In some embodiments, the nucleic acid, vector, or other composition comprises, from 5′ to 3′, a promoter, a minigene comprising SEQ ID NO: 16 (e.g., a minigene comprising or consisting of SEQ ID NO: 71 or SEQ ID NO: 94), a sequence encoding a furin cleavage site comprising or consisting of SEQ ID NO: 19, a sequence encoding a self cleaving T2A peptide comprising or consisting of SEQ ID NO: 20, a transgene encoding PRGN (e.g., SEQ ID NO: 87), a PRE sequence comprising SEQ ID NO: 88, and a polyA sequence (e.g., a polyA comprising or consisting of SEQ ID NO: 89).

In any of the aforementioned aspects and embodiments, the nucleic acid sequences contemplated may be DNA, RNA, or modified versions thereof. Modified nucleic acids may be distinguished from naturally occurring nucleic acids by modifications to the backbone of the polynucleotide chain, for example, peptide nucleic acids (PNA), morpholinos, locked nucleic acids (LNA), glycol nucleic acids (GNA) and threose nucleic acid (TNA). Modified nucleic acids may also include analogs with modifications to the four nucleobases. In some embodiments, the nucleic acids are PNAs. In some embodiments, the nucleic acids are LNAs. In some embodiments, the nucleic acids are morpholinos. In some embodiments, the nucleic acids are in a single-stranded form. In some embodiments, the nucleic acids are in double-stranded form. In some embodiments, the nucleic acids are linear. In some embodiments, the nucleic acids are circular. In some embodiments, the nucleic acids are plasmids.

Viral Vectors

Also disclosed herein are vectors comprising the nucleic acids (e.g., minigenes, transgenes, other nucleic acid components such as promoters, PREs and polyAs, and combinations thereof) discussed herein. In some embodiments, a vector may serve to deliver a transgene to a target cell and/or to increase expression of that transgene in a target cell. In various embodiments, the vector may be used to regulate expression of proteins, antibodies or functional binding fragments, enzymes, etc., and/or nucleic acids, e.g., shRNA, siRNA, gRNA for use in CRISPR, etc., through use in combination with a splice modulator

For instance, a vector may comprise a “on-switch” minigene linked to a transgene encoding a therapeutic protein and/or RNA and, upon addition of a splice modulator, increase the expression of that transgene. In other embodiments, a vector may comprise an “off-switch” minigene linked to a transgene encoding a therapeutic protein and/or RNA and, upon addition of a splice modulator, decrease the expression of that transgene. In some embodiments, the vector may comprise a DNA or RNA (or a mixture thereof) sequence that comprises an insert (e.g., at least one open reading frame of a transgene sequence) and one or more additional elements. The vector may serve to transfer genetic information to another cell. Vectors may be used for cloning, e.g., as cloning vectors or plasmids. Vectors may also be designed specifically for other purposes, such as cellular infection, e.g., in a human neuronal cell, to drive expression, e.g., therapeutic protein and/or RNA expression. In some embodiments, vectors comprising the nucleic acids disclosed herein are contemplated. The vectors may be a DNA vector, a circular vector, or a plasmid. In some embodiments, the vector is double stranded. In other embodiments the vector is single stranded.

In some embodiments, the vector is a viral vector. In some embodiments, the vector is a viral vector used to deliver transgene sequence(s) to neuronal cells or tissue. Examples of viruses used for vectors include but are not limited to retroviruses, adenoviruses, lentiviruses, adeno-associated viruses, and other hybrid viruses. In some embodiments, the viral vector is an adeno-associated viral (AAV) vector, chimeric AAV vector, adenoviral vector, retroviral vector, lentiviral vector, DNA viral vector, herpes simplex viral vector, baculoviral vector, or any mutant or derivative thereof.

Without being bound by theory, viral vectors disclosed herein may insert their genomes into the host cell that they infect, thus delivering its nucleic acid sequence to the host. The viral genome inserted may be episomal or may be integrated into the chromosomes of the host cell at a site that may be random or targeted. In an embodiment, the vector is a viral vector used to deliver transgene sequences to cells. Examples of viruses used for vectors include but are not limited to retroviruses, adenoviruses, lentiviruses, adeno-associated viruses, and other hybrid viruses. Warnock et al., (2011) Methods Mol. Biol., 737:1-25. Lentivirus is a genus of retroviruses that can integrate significant amounts of viral DNA into a host cell, making them an efficient method of gene delivery. On the other hand, adenoviruses introduce genetic material that is not integrate into the chromosome of the host cell, thus reducing the risk of disrupting the host cell. In some embodiments, the viral vector is an adeno-associated viral (AAV) vector, chimeric AAV vector, adenoviral vector, retroviral vector, lentiviral vector, DNA viral vector, herpes simplex viral vector, baculoviral vector, or any mutant or derivative thereof.

In some embodiments, the vector comprising the transgene is or is derived from an adeno-associated virus (AAV). In some embodiments, the vector is a recombinant adeno-associated viral vector (rAAV). The rAAV genomes may comprise one or more AAV ITRs flanking a minigene and transgene sequence encoding a polypeptide (including, but not limited to, a hPGRN polypeptide) or encoding siRNA, shRNA, antisense, and/or miRNA directed at mutated proteins or control sequences of their genes. The minigene and transgene sequences are operatively linked, and may be linked by sequence encoding one or more protease cleavage sites or sequences encoding one or more self-cleaving peptides, or combinations thereof. In embodiments, the vectors additionally comprise other transcriptional control elements such as those disclosed herein, e.g., promoter, enhancer, PRE, and/or polyA sequences that are functional in target cells to drive expression of the transgene sequence. The transgene sequence may also include intron sequences to facilitate processing of an RNA transcript when expressed in mammalian cells.

In various embodiments, the AAV vector, e.g., the rAAV vector, is a self-complementary AAV vector (scAAV). As used herein, “self-complementary” means the coding region has been designed to form an intra-molecular double-stranded template, e.g., in one or more inverted terminal repeats (ITRs). Without being bound by theory, a rate-limiting step for AAV genome often involves the second-strand synthesis since the typical AAV genome is a single-stranded DNA template. Ferrari et al, (1996) J. Virology, 70(5): 3227-34; Fisher et al, (1996) J. Virology, 70(1): 520-32. However, for scAAV genomes, upon infection, the two complementary halves of scAAV may associate to form one double stranded DNA (dsDNA) unit that is ready for replication and transcription rather than waiting for cell mediated synthesis of the second strand. In some embodiments, the rAAV vector disclosed herein is a scAAV vector and provides for faster and/or increased expression.

In some embodiments, the rAAV vectors disclosed herein lack one or more (e.g., all) AAV rep and/or cap genes. An AAV vector may comprise (e.g., in its ITRs) nucleic acid sequences (e.g., DNA) from any suitable AAV serotype. Suitable AAV serotypes include, but are not limited to, AAV serotypes AAV-1, AAV-2, AAV-3, AAV-4, AAV-5, AAV-6, AAV-7, AAV-8, AAV-9, AAV-10, AAV-11, AAV-12, AAVrh8, AAVrh10, AAV.Anc80, AAV.Anc80L65, AAV-DJ, and AAV-DJ/8, AAVrh37, AAV-DJ, AAV-DJ/8, AAV-PHP.B, AAV-PHP.B2, AAV-PHP.B3, AAV-PHP.A, AAV-PHP.eB, and AAV-PHP.S. For instance, an AAV vector, e.g., an scAAV vector, may comprise nucleic acid sequences from an AAV2, e.g., ITR sequences from an AAV2. An AAV vector, e.g., an scAAV vector, may also comprise nucleic acids from more than one serotype. The nucleotide sequences of the genomes of the AAV serotypes are known in the art. For example, the complete genome of AAV1 is provided in GenBank Accession No. NC_002077; the complete genome of AAV2 is provided in GenBank Accession No. NC 001401 and Srivastava et al., Virol., 45: 555-564 {1983): the complete genome of AAV3 is provided in GenBank Accession No. NC_1829; the complete genome of AAV4 is provided in GenBank Accession No. NC_001829; the AAV5 genome is provided in GenBank Accession No. AF085716; the complete genome of AAV-6 is provided in GenBank Accession No. NC_00 1862; at least portions of AAV7 and AAV8 genomes are provided in GenBank Accession Nos. AX753246 and AX753249, respectively; the AAV9 genome is provided in Gao et al., J. Virol., 78: 6381-6388 (2004); the AAV10 genome is provided in Williams, (2006) Mol. Ther., 13(1): 67-76; and the AAV11 genome is provided in Mori et al., (2004) Virology, 330(2): 375-383.

In some embodiments, functional inverted terminal repeat (ITR) sequences may be used to support, e.g., the rescue, replication and packaging of the AAV virion. Thus, an AAV vector disclosed herein may include sequences that in cis provide for replication and packaging (e.g., functional ITRs) of the virus. The ITRs can be but need not be the wild-type nucleotide sequences, and may be altered, e.g., by the insertion, deletion or substitution of nucleotides, so long as the sequences provide for functional rescue, replication and packaging. The ITRs may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, and AAV-11. The nucleotide sequences of the genomes of the AAV serotypes are known in the art. For example, the complete genome of AAV-1 is provided in GenBank Accession No. NC_002077; the complete genome of AAV-2 is provided in GenBank Accession No. NC 001401 and Srivastava et al., Virol., 45: 555-564 {1983): the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., (2004) J. Virol., 78: 6381-6388; the AAV-10 genome is provided in Williams, (2006) Mol. Ther., 13(1): 67-76; and the AAV-11 genome is provided in Mori et al., (2004) Virology, 330(2): 375-383. In one embodiment, the vector is an AAV-9 vector, with AAV-2 derived ITRs.

In some embodiments, the rAAV vector disclosed herein comprise one or more ITRs, e.g., two ITRs, with one upstream and the other downstream of a transgene (e.g., encoding hPGRN) and/or the other nucleic acid elements discussed above. In some embodiments, a nucleic acid disclosed herein, e.g., in an scAAV vector, comprises a first ITR that is disposed 5′ and a second ITR that is disposed 3′ to the promoter, minigene, transgene, post-transcriptional regulatory element, and/or polyA, e.g., wherein the ITRs are independently 1, 2, 3, 4, 5, 6, 7, 8, 9, 10, 11, 12, 13, 14, 15, 16, 17, 18, 19, 20, 50, 100, 150, 200, 250 nucleotides 5′ and/or 3′ of the other elements. An ITR sequence may be wild-type, or it may comprise one or more mutations, e.g., as long as it retains one or more function of a wild-type ITR. In some embodiments, wild-type ITR may be modified to comprise a deletion of a terminal resolution site. In some embodiments, an scAAV as disclosed herein may comprise two ITR sequences, where both are wild-type, variant, or modified AAV ITR sequences. In some embodiments, at least one ITR sequence is a wild-type, variant or modified AAV ITR sequence. In some embodiments, the two ITR sequences are both wild-type, variant or modified AAV ITR sequences. In some embodiments, the “left” or 5′-ITR is a modified AAV ITR sequence that allows for production of self-complementary genomes, and the “right” or 3′-ITR is a wild-type AAV ITR sequence. In some embodiments, the “right” or 3′-ITR is a modified AAV ITR sequence that allows for the production of self-complementary genomes, and the “left” or 5′-ITR is a wild-type AAV ITR sequence. In some embodiments, the ITR sequences are wild-type, variant, or modified AAV2 ITR sequences. In some embodiments, at least one ITR sequence is a wild-type, variant or modified AAV2 ITR sequence. In some embodiments, the two ITR sequences are both wild-type, variant or modified AAV2 ITR sequences. In some embodiments, the “left” or 5′-ITR is a modified AAV2 ITR sequence that allows for production of self-complementary genomes, and the “right” or 3′-ITR is a wild-type AAV2 ITR sequence. In some embodiments, the “right” or 3′-ITR is a modified AAV2 ITR sequence that allows for the production of self-complementary genomes, and the “left” or 5′-ITR is a wild-type AAV2 ITR sequence. Exemplary sequences that may be used for one or more of the ITRs are described herein. In some embodiments, the AAV vector comprises SEQ ID NO: 12 and SEQ ID NO: 23. In some embodiments, the AAV vector comprises SEQ ID NO: 85 and SEQ ID NO: 90. Embodiments of AAV ITRs provided in WO/2019/094253 (PCT/US2018/058744), which is incorporated herein by reference in its entirety, may also be used for any AAV ITR disclosed herein.

In various embodiments, a vector disclosed herein may comprise a minigene and a nucleic acid sequence encoding a hPGRN disclosed herein. In some embodiments, addition of a splice modulator increases the expression of a functional PRGN polypeptide in a targeted cell. In other embodiments, addition of a splice modulator decreases expression of a functional PRGN polypeptide in a targeted cell. In some embodiments, the vector is a viral vector. In some embodiments, the vector comprising the transgene encoding hPGRN is or is derived from an AAV. In some embodiments, the vector is an rAAV. In various embodiments, the AAV vector comprising the transgene encoding a hPGRN disclosed herein, e.g., the rAAV vector, is an scAAV. The rAAV genomes may comprise one or more AAV ITRs flanking a transgene sequence encoding hPGRN. The transgene sequence may be operatively linked to transcriptional control elements such as those disclosed herein, e.g., promoter, enhancer, PRE, and/or polyA sequences that are functional in target cells to drive expression of the transgene sequence.

In some embodiments, the rAAV vector lacks one or more (e.g., all) AAV rep and/or cap genes. An AAV vector may comprise (e.g., in its ITRs) nucleic acid sequences (e.g., DNA) from any suitable AAV serotype. Suitable AAV serotypes include, but are not limited to, AAV serotypes AAV-1, AAV-2, AAV-3, AAV-4, AAV-5, AAV-6, AAV-7, AAV-8, AAV-9, AAV-10 and AAV-11. For instance, an AAV vector, e.g., an scAAV vector, may comprise nucleic acid sequences from an AAV-2, e.g., ITR sequences from an AAV-2. An AAV vector, e.g., an scAAV vector, may also comprise nucleic acids from more than one serotype. GenBank Accession No. NC 001401 and Srivastava et al., Virol., 45: 555-564 {1983); GenBank Accession No. NC_1829; GenBank Accession No. NC_001829; GenBank Accession No. AF085716; GenBank Accession No. NC_00 1862; GenBank Accession Nos. AX753246 and AX753249; Gao et al., J. Virol., 78: 6381-6388 (2004); Williams, (2006) Mol. Ther., 13(1): 67-76; and Mori et al., (2004) Virology, 330(2): 375-383.

In some embodiments, functional inverted terminal repeat (ITR) sequences in a viral vector comprising the transgene encoding a hPGRN disclosed herein may be used to support, e.g., the rescue, replication and packaging of the AAV virion. Thus, an AAV vector disclosed herein may include sequences that in cis provide for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging. The ITRs may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10 and AAV-11. GenBank Accession No. NC_002077; GenBank Accession No. NC 001401 and Srivastava et al., Virol., 45: 555-564 {1983); GenBank Accession No. NC_1829; GenBank Accession No. NC_001829; GenBank Accession No. AF085716; GenBank Accession No. NC_00 1862; GenBank Accession Nos. AX753246 and AX753249, respectively; Gao et al., (2004) J. Virol., 78: 6381-6388; Williams, (2006) Mol. Ther., 13(1): 67-76; and Mori et al., (2004) Virology, 330(2): 375-383. In one embodiment, the vector is an AAV-9 vector, with AAV-2 derived ITRs.

In some embodiments, the AAV viral vector comprises a sequence of SEQ ID NO: 91. In some embodiments, the AAV viral vector comprises a sequence of SEQ ID NO: 11. In each of these embodiments, the transgene sequence may be replaced with a sequence encoding an alternate molecule of interest, e.g., as described herein.

In some embodiments, a vector or nucleic acid sequence disclosed herein forms a cloning vector or an expression vector. In such embodiments, the vector may comprise other components that facilitate replication or maintenance of the vector. In some embodiments, the vector further comprises a selectable marker for clonal selection. In some embodiments, the selectable marker in the vector comprises a prokaryotic or eukaryotic antibiotic resistance gene. In some embodiments, the selectable marker in the vector comprises a kanamycin resistance gene. In some embodiments, the selectable marker in the vector comprises an ampicillin resistance gene. In some embodiments, the vector further comprises a puromycin resistance gene. In some embodiments, the selectable marker in the vector comprises a hygromycin resistance gene. In some embodiments, the vector (e.g., plasmid) comprises a nucleic acid sequence of SEQ ID NO: 92.

Exemplary AAV vector sequence comprising a minigene and transgene encoding EGFP:

(SEQ ID NO: 11)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGAATTCGGGCGGAGTTAGGGCGGAGCCAATCAGCGTGCGCCGTTC
CGAAAGTTGCCTTTTATGGCTGGGCGGAGAATGGGCGGTGAACGCCGATG
ATTATATAAGGACGCGCCGGGTGTGGCACAGCTAGTTCCGTCGCAGCCGG
GATTTGGGTCGCGGTTCTTGTTTGTGGATCCCTGTGATCGTCACTTGACA
CCGGTCTTCCAGAGGAGATTGGAAAACTTGAAGAAGAAGTGGATTGTGCT
AATATTGCCCTGAAAGCAGCCACCATGGATTGGGAGAGTTGGAAACAAAA
TTTGCAAATTGATATCAAGTTAGCATTTACAGATTTGGCTGAGGAGAATA
TCCATTATTTTGAACAGGTAATTAGTGTTGTTTGATATTGCTTCATTTTA
AAGTTATTTGCTCATTTACTTTTGGTCCGTCCATTGTTGAAAGAGTGTAT
TAAAGAACAAGTGTCACATTCTATTGCCTCTCTGGTAGCTTGGTTTTGTT
GAAGTTGTCAGTTACCATTTGGTTTTGTTTATCCTCAGTTTGTTGTTTTG
GATTTGGATTCTTCAAAAGCATTTGATATTGCTTTCTATTGATTGTCCTA
ACTACTCCTCTTTCCTCTCCCTTCTCCATTTTTGAAGAGTTTGCAAAGGA
AGGAAAGGAGCAGAGACTTGATTGAGCAGAAAATCATTTCAGGGCCTGTT
CTCTATTGTCCTTGCTATCCTGTCTTCTGTAGCTATCTGAAACCATCAAC
AAAGGAGCACACCATTCCATCAGCAAAAGAGTAACAACATCTTTTTTTAA
GTTCATTTTGTTTTTCAGTTGATTGTATTTCAATTTTTTTACAGCTGACT
TTTCTCAGAGAAGTTTTTTTTTTATTGTAAACATACTTTTTCTAGAAAGT
ATATTTTAAAATAACATCTTTAACCTTATCTCTGGCTGAATTATTGAATA
TTTGAAATTATTACATTAACAAAATTTTGTCTTACAGCAGTGGTCCCCAA
CCTTCTTAGCAGTAGCATCCCTCATTAAGAATTAAAATTTGTAGAAATTG
ACAAGGATTCTGACAAGCTGTTGGGAGAGAAGAATAGAGCAGATTGCAGT
AGGAACAGTTGTGTTAGAATTTATTAATCCTTTAACACTGAAAGTAAACT
ATTGTTGATTGCCTCTTGGTGTGTTTCCATTATTCAGTGCTCTTGCTAAG
TGGGAGTCATTCCTTACATCAACCACCAACCTTCACTTGGAAGAAGCTAG
CGAAGATAAACCTCGCAACCGCCGCGGCAGCGGCGAAGGCCGCGGCAGCC
TGCTGACCTGCGGCGATGTGGAAGAAAACCCGGGCCCGGTGAGCAAGGGC
GAGGAGCTGTTCACCGGGGTGGTGCCCATCCTGGTCGAGCTGGACGGCGA
CGTAAACGGCCACAAGTTCAGCGTGTCCGGCGAGGGCGAGGGCGATGCCA
CCTACGGCAAGCTGACCCTGAAGTTCATCTGCACCACCGGCAAGCTGCCC
GTGCCCTGGCCCACCCTCGTGACCACCCTGACCTACGGCGTGCAGTGCTT
CAGCCGCTACCCCGACCACATGAAGCAGCACGACTTCTTCAAGTCCGCCA
TGCCCGAAGGCTACGTCCAGGAGCGCACCATCTTCTTCAAGGACGACGGC
AACTACAAGACCCGCGCCGAGGTGAAGTTCGAGGGCGACACCCTGGTGAA
CCGCATCGAGCTGAAGGGCATCGACTTCAAGGAGGACGGCAACATCCTGG
GGCACAAGCTGGAGTACAACTACAACAGCCACAACGTCTATATCATGGCC
GACAAGCAGAAGAACGGCATCAAGGTGAACTTCAAGATCCGCCACAACATC
GAGGACGGCAGCGTGCAGCTCGCCGACCACTACCAGCAGAACACCCCCATC
GGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAG
TTCGTGACCGCCGCCGGGATCACTCTCGGCATGGACGAGCTGTACAAGTAA
TGCTTTATTTGTGAAATTTGTGATGCTATTGCTTTATTTGTAACCATTATA
AGCTGCAATAAACAAGTTAACAACAACAATTGCATTCATTTTATGTTTCAG
GTTCAGGGGGAGGTGTGGGAGGTTTTTTAAAATCGATCTGAGGAACCCCTA
GTGATGGAGTTGGCCACTCCCTCTCTGCGCGCTCGCTCGCTCACTGAGGCC
GGGCGACCAAAGGTCGCCCGACGCCCGGGCTTTGCCCGGGCGGCCTCAGTG
AGCGAGCGAGCGCGCAGAGAGGGAGTGG

Table 2 and Table 3 describe exemplary sequences of the nucleic acids, vectors, and minigenes.

TABLE 2
Examplary minigene and AAV vector sequences having a SNX7-derived minigene.

| Sequence | | SEQ ID |
|---|---|---|
| description | SEQUENCE | NO: |
| AAV2 5′-ITR with trs | CTGCGCGCTCGCTCGCTCACTGAGGCCGCCCGGGCAAAGCCC | 12 |
| deletion | GGGCGTCGGGCGACCTTTGGTCGCCCGGCCTCAGTGAGCGAG | |
| | CGAGCGCGCAGAGAGGGAGTGG | |
| JeT promoter | AATTCGGGCGGAGTTAGGGCGGAGCCAATCAGCGTGCGCCG | 13 |
| | TTCCGAAAGTTGCCTTTTATGGCTGGGCGGAGAATGGGCGGT | |
| | GAACGCCGATGATTATATAAGGACGCGCCGGGTGTGGCACA | |
| | GCTAGTTCCGTCGCAGCCGGGATTTGGGTCGCGGTTCTTGTTT | |
| | GTGGATCCCTGTGATCGTCACTTGACA | |
| SNX Minigene | CTTCCAGAGGAGATTGGAAAACTTGAAGAAGAAGTGGATTGT | 14 |
| (Version 1) First Exon | GCTAATATTGCCCTGAAAGCAGCCACCATGGATTGGGAGAG | |
| (purine-rich exonic | TTGGAAACAAAATTTGCAAATTGATATCAAGTTAGCATTTAC | |
| splicing enhancer in | AGATTTGGCTGAGGAGAATATCCATTATTTTGAACAG | |
| italics; Kozak in bold; | | |
| start codon | | |
| underlined) | | |
| SNX7 Minigene | GTAATTAGTGTTGTTTGATATTGCTTCATTTTAAAGTTATTTG | 15 |
| (Version 1) first intron | CTCATTTACTTTTGGTCCGTCCATTGTTGAAAGAGTGTATTAA | |
| | AGAACAAGTGTCACATTCTATTGCCTCTCTGGTAGCTTGGTTT | |
| | TGTTGAAGTTGTCAGTTACCATTTGGTTTTGTTTATCCTCAGT | |
| | TTGTTGTTTTGGATTTGGATTCTTCAAAAGCATTTGATATTGC | |
| | TTTCTATTGATTGTCCTAACTACTCCTCTTTCCTCTCCCTTCTC | |
| | CATTTTTGAAG | |
| SNX7 Minigene | AGTTTGCAAAGGAAGGAAAGGAGCAGAGACTTGATTGAGCA | 16 |
| (Version 1) second | GAAAATCATTTCAGGGCCTGTTCTCTATTGTCCTTGCTATCCT | |
| exon | GTCTTCTGTAGCTATCTGAAACCATCAACAAAGGAGCACACC | |
| | ATTCCATCAGCAAAAGA | |
| SNX7 Minigene (both | GTAACAACATCTTTTTTTAAGTTCATTTTGTTTTTCAGTTGATT | 17 |
| Version 1 and Version | GTATTTCAATTTTTTTACAGCTGACTTTTCTCAGAGAAGTTTT | |
| 2) second intron | TTTTTTATTGTAAACATACTTTTTCTAGAAAGTATATTTTAAA | |
| | ATAACATCTTTAACCTTATCTCTGGCTGAATTATTGAATATTT | |
| | GAAATTATTACATTAACAAAATTTTGTCTTACAGCAGTGGTC | |
| | CCCAACCTTCTTAGCAGTAGCATCCCTCATTAAGAATTAAAA | |
| | TTTGTAGAAATTGACAAGGATTCTGACAAGCTGTTGGGAGAG | |
| | AAGAATAGAGCAGATTGCAGTAGGAACAGTTGTGTTAGAAT | |
| | TTATTAATCCTTTAACACTGAAAGTAAACTATTGTTGATTGCC | |
| | TCTTGGTGTGTTTCCATTATTCAG | |
| SNX7 Minigene (Both | TGCTCTTGCTAAGTGGGAGTCATTCCTTACATCAACCACCAA | 18 |
| Version 1 and Version | CCTTCACTTGGAAGAAGCTAGCGAAGATAAACCT | |
| 2) third exon | | |
| Full SNX7 minigene | CTTCCAGAGGAGATTGGAAAACTTGAAGAAGAAGTGGATTG | 71 |
| (Version 1) sequence | TGCTAATATTGCCCTGAAAGCAGCCACCATGGATTGGGAGAG | |
| | TTGGAAACAAAATTTGCAAATTGATATCAAGTTAGCATTTAC | |
| | AGATTTGGCTGAGGAGAATATCCATTATTTTGAACAGGTAAT | |
| | TAGTGTTGTTTGATATTGCTTCATTTTAAAGTTATTTGCTCAT | |
| | TTACTTTTGGTCCGTCCATTGTTGAAAGAGTGTATTAAAGAA | |
| | CAAGTGTCACATTCTATTGCCTCTCTGGTAGCTTGGTTTTGTT | |
| | GAAGTTGTCAGTTACCATTTGGTTTTGTTTATCCTCAGTTTGT | |
| | TGTTTTGGATTTGGATTCTTCAAAAGCATTTGATATTGCTTTC | |
| | TATTGATTGTCCTAACTACTCCTCTTTCCTCTCCCTTCTCCATT | |
| | TTTGAAGAGTTTGCAAAGGAAGGAAAGGAGCAGAGACTTGA | |
| | TTGAGCAGAAAATCATTTCAGGGCCTGTTCTCTATTGTCCTTG | |
| | CTATCCTGTCTTCTGTAGCTATCTGAAACCATCAACAAAGGA | |
| | GCACACCATTCCATCAGCAAAAGAGTAACAACATCTTTTTTT | |
| | AAGTTCATTTTGTTTTTCAGTTGATTGTATTTCAATTTTTTTAC | |
| | AGCTGACTTTTCTCAGAGAAGTTTTTTTTTTATTGTAAACATA | |
| | CTTTTTCTAGAAAGTATATTTTAAAATAACATCTTTAACCTTA | |
| | TCTCTGGCTGAATTATTGAATATTTGAAATTATTACATTAACA | |
| | AAATTTTGTCTTACAGCAGTGGTCCCCAACCTTCTTAGCAGT | |
| | AGCATCCCTCATTAAGAATTAAAATTTGTAGAAATTGACAAG | |
| | GATTCTGACAAGCTGTTGGGAGAGAAGAATAGAGCAGATTG | |
| | CAGTAGGAACAGTTGTGTTAGAATTTATTAATCCTTTAACAC | |
| | TGAAAGTAAACTATTGTTGATTGCCTCTTGGTGTGTTTCCATT | |
| | ATTCAGTGCTCTTGCTAAGTGGGAGTCATTCCTTACATCAAC | |
| | CACCAACCTTCACTTGGAAGAAGCTAGCGAAGATAAACCT | |
| Sequence encoding | CGCAACCGCCGC | 19 |
| furin cleavage | | |
| sequence | | |
| Sequence encoding | GGCAGCGGCGAAGGCCGCGGCAGCCTGCTGACCTGCGGCGA | 20 |
| T2A peptide | TGTGGAAGAAAACCCGGGCCCG | |
| Sequence encoding | GTGAGCAAGGGCGAGGAGCTGTTCACCGGGGTGGTGCCCAT | 21 |
| EGFP (transgene) | CCTGGTCGAGCTGGACGGCGACGTAAACGGCCACAAGTTCA | |
| | GCGTGTCCGGCGAGGGCGAGGGCGATGCCACCTACGGCAAG | |
| | CTGACCCTGAAGTTCATCTGCACCACCGGCAAGCTGCCCGTG | |
| | CCCTGGCCCACCCTCGTGACCACCCTGACCTACGGCGTGCAG | |
| | TGCTTCAGCCGCTACCCCGACCACATGAAGCAGCACGACTTC | |
| | TTCAAGTCCGCCATGCCCGAAGGCTACGTCCAGGAGCGCACC | |
| | ATCTTCTTCAAGGACGACGGCAACTACAAGACCCGCGCCGA | |
| | GGTGAAGTTCGAGGGCGACACCCTGGTGAACCGCATCGAGC | |
| | TGAAGGGCATCGACTTCAAGGAGGACGGCAACATCCTGGGG | |
| | CACAAGCTGGAGTACAACTACAACAGCCACAACGTCTATATC | |
| | ATGGCCGACAAGCAGAAGAACGGCATCAAGGTGAACTTCAA | |
| | GATCCGCCACAACATCGAGGACGGCAGCGTGCAGCTCGCCG | |
| | ACCACTACCAGCAGAACACCCCCATCGGCGACGGCCCCGTG | |
| | CTGCTGCCCGACAACCACTACCTGAGCACCCAGTCCGCCCTG | |
| | AGCAAAGACCCCAACGAGAAGCGCGATCACATGGTCCTGCT | |
| | GGAGTTCGTGACCGCCGCCGGGATCACTCTCGGCATGGACGA | |
| | GCTGTACAAGTAA | |
| SV40 polyA | TGCTTTATTTGTGAAATTTGTGATGCTATTGCTTTATTTGTAA | 22 |
| | CCATTATAAGCTGCAATAAACAAGTTAACAACAACAATTGCA | |
| | TTCATTTTATGTTTCAGGTTCAGGGGGAGGTGTGGGAGGTTT | |
| | TTTAA | |
| AAV2 3′-ITR | AGGAACCCCTAGTGATGGAGTTGGCCACTCCCTCTCTGCGCG | 23 |
| | CTCGCTCGCTCACTGAGGCCGGGCGACCAAAGGTCGCCCGAC | |
| | GCCCGGGCTTTGCCCGGGCGGCCTCAGTGAGCGAGCGAGCG | |
| | CGCAGAGAGGGAGTGG | |
| SNX Minigene | GAAGAAGAAGATATCAAGTTAGCATTTACAGATTTGGCTGAGGAGA | 96 |
| (Version 2) First Exon | AGAACAG | |
| SNX7 Minigene | GTAATTAGTGTTGTTTGATATTGCTTCATTTTAAAGTTATTTGCTCAT | 97 |
| (Version 2) first intron | TTAGCATTTGATATTGCTTTCTATTGATTGTCCTAACTACTCCTCTT | |
| | TCCTCTCCCTTCTCCATTTTTGAAG | |
| SNX7 Minigene | AGTTTGCAAAGGAAGGAAAGGAGCAGAGACTTGATTGAGCAGAAA | 98 |
| (Version 2) second | ATCATTTCAGGGCCTGTTCTCTATTGTCCTTGCTATCCTGTCTTCT | |
| exon (start condon in | GTAGCTATCTGAAACCATCAACAAAGGAGCACACCAT [symbol] GCATCAG | |
| Bold; modified | CAAAAGA | |
| nucleotides in Italic) | | |
| Full SNX7 minigene | GAAGAAGAAGATATCAAGTTAGCATTTACAGATTTGGCTGAGGAG | 94 |
| (Version 2) sequence | AAGAACAGGTAATTAGTGTTGTTTGATATTGCTTCATTTTAAAGTTA | |
| | TTTGCTCATTTAGCATTTGATATTGCTTTCTATTGATTGTCCTAACTA | |
| | CTCCTCTTTCCTCTCCCTTCTCCATTTTTGAAGAGTTTGCAAAGGAA | |
| | GGAAAGGAGCAGAGACTTGATTGAGCAGAAAATCATTTCAGGGCC | |
| | TGTTCTCTATTGTCCTTGCTATCCTGTCTTCTGTAGCTATCTGAAACC | |
| | ATCAACAAAGGAGCACACCATGGCATCAGCAAAAGAGTAACAACA | |
| | TCTTTTTTTAAGTTCATTTTGTTTTTCAGTTGATTGTATTTCAATTTTT | |
| | TTACAGCTGACTTTTCTCAGAGAAGTTTTTTTTTTATTGTAAACATA | |
| | CTTTTTCTAGAAAGTATATTTTAAAATAACATCTTTAACCTTATCTC | |
| | TGGCTGAATTATTGAATATTTGAAATTATTACATTAACAAAATTTTG | |
| | TCTTACAGCAGTGGTCCCCAACCTTCTTAGCAGTAGCATCCCTCATT | |
| | AAGAATTAAAATTTGTAGAAATTGACAAGGATTCTGACAAGCTGTT | |
| | GGGAGAGAAGAATAGAGCAGATTGCAGTAGGAACAGTTGTGTTAG | |
| | AATTTATTAATCCTTTAACACTGAAAGTAAACTATTGTTGATTGCCT | |
| | CTTGGTGTGTTTCCATTATTCAGTGCTCTTGCTAAGTGGGAGTCATT | |
| | CCTTACATCAACCACCAACCTTCACTTGGAAGAAGCTAGCGAAGAT | |
| | AAACCT | |

TABLE 3
Sequence of exemplary components, and plasmid encoding a single-stranded
AAV comprising a minigene and transgene encoding human progranulin

| Sequence | | SEQ ID |
|---|---|---|
| description | SEQUENCE | NO: |
| AAV2 5′-ITR with trs | CCTGCAGGCAGCTGCGCGCTCGCTCGCTCACTGAGGCCGCCC | 85 |
| deletion | GGGCAAAGCCCGGGCGTCGGGCGACCTTTGGTCGCCCGGCC | |
| | TCAGTGAGCGAGCGAGCGCGCAGAGAGGGAGTGGCCAACTC | |
| | CATCACTAGGGGTTCCT | |
| Human Synapsin | AACCGAGTATCTGCAGAGGGCCCTGCGTATGAGTGCAAGTG | 86 |
| promoter | GGTTTTAGGACCAGGATGAGGCGGGGTGGGGGTGCCTACCT | |
| | GACGACCGACCCCGACCCACTGGACAAGCACCCAACCCCCA | |
| | TTCCCCAAATTGCGCATCCCCTATCAGAGAGGGGGAGGGGA | |
| | AACAGGATGCGGCGAGGCGCGTGCGCACTGCCAGCTTCAGC | |
| | ACCGCGGACAGTGCCTTCGCCCCCGCCTGGCGGCGCGCGCCA | |
| | CCGCCGCCTCAGCACTGAAGGCGCGCTGACGTCACTCGCCGG | |
| | TCCCCCGCAAACTCCCCTTCCCGGCCACCTTGGTCGCGTCCG | |
| | CGCCGCCGCCGGCCCAGCCGGACCGCACCACGCGAGGCGCG | |
| | AGATAGGGGGGCACGGGCGCGACCATCTGCGCTGCGGCGCC | |
| | GGCGACTCAGCGCTGCCTCAGTCTGCGGTGGGCAGCGGAGG | |
| | AGTCGTGTCGTGCCTGAGAGCGCAGCTGT | |
| Full SNX7 minigene | CTTCCAGAGGAGATTGGAAAACTTGAAGAAGAAGTGGATTGT | 71 |
| sequence (version 1) | GCTAATATTGCCCTGAAAGCAGCCACCATGGATTGGGAGAG | |
| | TTGGAAACAAAATTTGCAAATTGATATCAAGTTAGCATTTAC | |
| | AGATTTGGCTGAGGAGAATATCCATTATTTTGAACAGGTAAT | |
| | TAGTGTTGTTTGATATTGCTTCATTTTAAAGTTATTTGCTCAT | |
| | TTACTTTTGGTCCGTCCATTGTTGAAAGAGTGTATTAAAGAA | |
| | CAAGTGTCACATTCTATTGCCTCTCTGGTAGCTTGGTTTTGTT | |
| | GAAGTTGTCAGTTACCATTTGGTTTTGTTTATCCTCAGTTTGT | |
| | TGTTTTGGATTTGGATTCTTCAAAAGCATTTGATATTGCTTTC | |
| | TATTGATTGTCCTAACTACTCCTCTTTCCTCTCCCTTCTCCATT | |
| | TTTGAAGAGTTTGCAAAGGAAGGAAAGGAGCAGAGACTTGA | |
| | TTGAGCAGAAAATCATTTCAGGGCCTGTTCTCTATTGTCCTTG | |
| | CTATCCTGTCTTCTGTAGCTATCTGAAACCATCAACAAAGGA | |
| | GCACACCATTCCATCAGCAAAAGAGTAACAACATCTTTTTTT | |
| | AAGTTCATTTTGTTTTTCAGTTGATTGTATTTCAATTTTTTTAC | |
| | AGCTGACTTTTCTCAGAGAAGTTTTTTTTTTATTGTAAACATA | |
| | CTTTTTCTAGAAAGTATATTTTAAAATAACATCTTTAACCTTA | |
| | TCTCTGGCTGAATTATTGAATATTTGAAATTATTACATTAACA | |
| | AAATTTTGTCTTACAGCAGTGGTCCCCAACCTTCTTAGCAGT | |
| | AGCATCCCTCATTAAGAATTAAAATTTGTAGAAATTGACAAG | |
| | GATTCTGACAAGCTGTTGGGAGAGAAGAATAGAGCAGATTG | |
| | CAGTAGGAACAGTTGTGTTAGAATTTATTAATCCTTTAACAC | |
| | TGAAAGTAAACTATTGTTGATTGCCTCTTGGTGTGTTTCCATT | |
| | ATTCAGTGCTCTTGCTAAGTGGGAGTCATTCCTTACATCAAC | |
| | CACCAACCTTCACTTGGAAGAAGCTAGCGAAGATAAACCT | |
| Sequence encoding | CGCAACCGCCGC | 19 |
| furin cleavage | | |
| sequence | | |
| Sequence encoding | GGCAGCGGCGAAGGCCGCGGCAGCCTGCTGACCTGCGGCGA | 20 |
| T2A peptide | TGTGGAAGAAAACCCGGGCCCG | |
| Sequence encoding | TGGACCCTGGTGAGCTGGGTGGCCTTAACAGCAGGGCTGGT | 87 |
| human progranulin | GGCTGGAACGCGGTGCCCAGATGGTCAGTTCTGCCCTGTGGC | |
| (transgene) | CTGCTGCCTGGACCCCGGAGGAGCCAGCTACAGCTGCTGCCG | |
| | TCCCCTTCTGGACAAATGGCCCACAACACTGAGCAGGCATCT | |
| | GGGTGGCCCCTGCCAGGTTGATGCCCACTGCTCTGCCGGCCA | |
| | CTCCTGCATCTTTACCGTCTCAGGGACTTCCAGTTGCTGCCCC | |
| | TTCCCAGAGGCCGTGGCATGCGGGGATGGCCATCACTGCTGC | |
| | CCACGGGGCTTCCACTGCAGTGCAGACGGGCGATCCTGCTTC | |
| | CAAAGATCAGGTAACAACTCCGTGGGTGCCATCCAGTGCCCT | |
| | GATAGTCAGTTCGAATGCCCGGACTTCTCCACGTGCTGTGTT | |
| | ATGGTCGATGGCTCCTGGGGGTGCTGCCCCATGCCCCAGGCT | |
| | TCCTGCTGTGAAGACAGGGTGCACTGCTGTCCGCACGGTGCC | |
| | TTCTGCGACCTGGTTCACACCCGCTGCATCACACCCACGGGC | |
| | ACCCACCCCCTGGCAAAGAAGCTCCCTGCCCAGAGGACTAA | |
| | CAGGGCAGTGGCCTTGTCCAGCTCGGTCATGTGTCCGGACGC | |
| | ACGGTCCCGGTGCCCTGATGGTTCTACCTGCTGTGAGCTGCC | |
| | CAGTGGGAAGTATGGCTGCTGCCCAATGCCCAACGCCACCTG | |
| | CTGCTCCGATCACCTGCACTGCTGCCCCCAAGACACTGTGTG | |
| | TGACCTGATCCAGAGTAAGTGCCTCTCCAAGGAGAACGCTAC | |
| | CACGGACCTCCTCACTAAGCTGCCTGCGCACACAGTGGGGG | |
| | ATGTGAAATGTGACATGGAGGTGAGCTGCCCAGATGGCTAT | |
| | ACCTGCTGCCGTCTACAGTCGGGGGCCTGGGGCTGCTGCCCT | |
| | TTTACCCAGGCTGTGTGCTGTGAGGACCACATACACTGCTGT | |
| | CCCGCGGGGTTTACGTGTGACACGCAGAAGGGTACCTGTGA | |
| | ACAGGGGCCCCACCAGGTGCCCTGGATGGAGAAGGCCCCAG | |
| | CTCACCTCAGCCTGCCAGACCCACAAGCCTTGAAGAGAGAT | |
| | GTCCCCTGTGATAATGTCAGCAGCTGTCCCTCCTCCGATACC | |
| | TGCTGCCAACTCACGTCTGGGGAGTGGGGCTGCTGTCCAATC | |
| | CCAGAGGCTGTCTGCTGCTCGGACCACCAGCACTGCTGCCCC | |
| | CAGGGCTACACGTGTGTAGCTGAGGGGCAGTGTCAGCGAGG | |
| | AAGCGAGATCGTGGCTGGACTGGAGAAGATGCCTGCCCGCC | |
| | GGGCTTCCTTATCCCACCCCAGAGACATCGGCTGTGACCAGC | |
| | ACACCAGCTGCCCGGTGGGGCAGACCTGCTGCCCGAGCCTG | |
| | GGTGGGAGCTGGGCCTGCTGCCAGTTGCCCCATGCTGTGTGC | |
| | TGCGAGGATCGCCAGCACTGCTGCCCGGCTGGCTACACCTGC | |
| | AACGTGAAGGCTCGATCCTGCGAGAAGGAAGTGGTCTCTGC | |
| | CCAGCCTGCCACCTTCCTGGCCCGTAGCCCTCACGTGGGTGT | |
| | GAAGGACGTGGAGTGTGGGGAAGGACACTTCTGCCATGATA | |
| | ACCAGACCTGCTGCCGAGACAACCGACAGGGCTGGGCCTGC | |
| | TGTCCCTACCGCCAGGGCGTCTGTTGTGCTGATCGGCGCCAC | |
| | TGCTGTCCTGCTGGCTTCCGCTGCGCAGCCAGGGGTACCAAG | |
| | TGTTTGCGCAGGGAGGCCCCGCGCTGGGACGCCCCTTTGAGG | |
| | GACCCAGCCTTGAGACAGCTGCTGTAG | |
| HPRE-NOX | AACAGGCCTATTGATTGGAAAGTATGTCAACGAATTGTGGGT | 88 |
| | CTTTTGGGGTTTGCTGCCCCTTTTACGCAATGTGGATATCCTG | |
| | CTTTAATGCCTTTATATGCATGTATACAAGCAAAACAGGCTT | |
| | TTACTTTCTCGCCAACTTACAAGGCCTTTCTAAGTAAACAGT | |
| | ATCTGACCCTTTACCCCGTTGCTCGGCAACGGCCTGGTCTGT | |
| | GCCAAGTGTTTGCTGACGCAACCCCCACTGGTTGGGGCTTGG | |
| | CCATAGGCCATCAGCGCATGCGTGGAACCTTTGTGTCTCCTC | |
| | TGCCGATCCATACTGCGGAACTCCTAGCCGCTTGTTTTGCTC | |
| | GCAGCAGGTCTGGAGCGAAACTCATCGGGACTGACAATTCT | |
| | GTCGTGCTCTCCCGCAAGTATACATCGTTTCCAGGGCTGCTA | |
| | GGCTGTGCTGCCAACTGGATCCTGCGCGGGACGTCCTTTGTT | |
| | TACGTCCCGTCGGCGCTGAATCCCGCGGACGACCCCTCCCGG | |
| | GGCCGCTTGGGGCTCTACCGCCCGCTTCTCCGTCTGCCGTAC | |
| | CGACCGACCACGGGGCGCACCTCTCTTTACGCGGACTCCCCG | |
| | TCTGTGCCTTCTCATCTGCCGGACCGTGTGCACTTCGCTTCAC | |
| | CTCTGCACGTCGCATGGAGACCACCGTGAACGCCCACCGGA | |
| | ACCTGCCCAAGGTCTTGCATAAGAGGACTCTTGGACTTTCAG | |
| | CAATGTC | |
| HGH polyA | ACGGGTGGCATCCCTGTGACCCCTCCCCAGTGCCTCTCCTGG | 89 |
| | CCCTGGAAGTTGCCACTCCAGTGCCCACCAGCCTTGTCCTAA | |
| | TAAAATTAAGTTGCATCATTTTGTCTGACTAGGTGTCCTTCTA | |
| | TAATATTATGGGGTGGAGGGGGGTGGTATGGAGCAAGGGGC | |
| | AAGTTGGGAAGACAACCTGTAGGGCCTGCGGGGTCTATTGG | |
| | GAACCAAGCTGGAGTGCAGTGGCACAATCTTGGCTCACTGC | |
| | AATCTCCGCCTCCTGGGTTCAAGCGATTCTCCTGCCTCAGCCT | |
| | CCCGAGTTGTTGGGATTCCAGGCATGCATGACCAGGCTCAGC | |
| | TAATTTTTGTTTTTTTGGTAGAGACGGGGTTTCACCATATTGG | |
| | CCAGGCTGGTCTCCAACTCCTAATCTCAGGTGATCTACCCAC | |
| | CTTGGCCTCCCAAATTGCTGGGATTACAGGCGTGAACCACTG | |
| | CTCCCTTCCCTGTCCTT | |
| AAV2 3′-ITR | AGGAACCCCTAGTGATGGAGTTGGCCACTCCCTCTCTGCGCG | 90 |
| | CTCGCTCGCTCACTGAGGCCGGGCGACCAAAGGTCGCCCGA | |
| | CGCCCGGGCTTTGCCCGGGCGGCCTCAGTGAGCGAGCGAGC | |
| | GCGCAGCTGCCTGCAGG | |
| Full rAAV genome | CCTGCAGGCAGCTGCGCGCTCGCTCGCTCACTGAGGCCGCCC | 91 |
| sequence | GGGCAAAGCCCGGGCGTCGGGCGACCTTTGGTCGCCCGGCC | |
| | TCAGTGAGCGAGCGAGCGCGCAGAGAGGGAGTGGCCAACTC | |
| | CATCACTAGGGGTTCCTGCGGCCGCACGCGTAACCGAGTATC | |
| | TGCAGAGGGCCCTGCGTATGAGTGCAAGTGGGTTTTAGGACC | |
| | AGGATGAGGCGGGGTGGGGGTGCCTACCTGACGACCGACCC | |
| | CGACCCACTGGACAAGCACCCAACCCCCATTCCCCAAATTGC | |
| | GCATCCCCTATCAGAGAGGGGGAGGGGAAACAGGATGCGGC | |
| | GAGGCGCGTGCGCACTGCCAGCTTCAGCACCGCGGACAGTG | |
| | CCTTCGCCCCCGCCTGGCGGCGCGCGCCACCGCCGCCTCAGC | |
| | ACTGAAGGCGCGCTGACGTCACTCGCCGGTCCCCCGCAAACT | |
| | CCCCTTCCCGGCCACCTTGGTCGCGTCCGCGCCGCCGCCGGC | |
| | CCAGCCGGACCGCACCACGCGAGGCGCGAGATAGGGGGGCA | |
| | CGGGCGCGACCATCTGCGCTGCGGCGCCGGCGACTCAGCGC | |
| | TGCCTCAGTCTGCGGTGGGCAGCGGAGGAGTCGTGTCGTGCC | |
| | TGAGAGCGCAGCTGTGAATTCCTTCCAGAGGAGATTGGAAA | |
| | ACTTGAAGAAGAAGTGGATTGTGCTAATATTGCCCTGAAAGC | |
| | AGCCACCATGGATTGGGAGAGTTGGAAACAAAATTTGCAAA | |
| | TTGATATCAAGTTAGCATTTACAGATTTGGCTGAGGAGAATA | |
| | TCCATTATTTTGAACAGGTAATTAGTGTTGTTTGATATTGCTT | |
| | CATTTTAAAGTTATTTGCTCATTTACTTTTGGTCCGTCCATTG | |
| | TTGAAAGAGTGTATTAAAGAACAAGTGTCACATTCTATTGCC | |
| | TCTCTGGTAGCTTGGTTTTGTTGAAGTTGTCAGTTACCATTTG | |
| | GTTTTGTTTATCCTCAGTTTGTTGTTTTGGATTTGGATTCTTCA | |
| | AAAGCATTTGATATTGCTTTCTATTGATTGTCCTAACTACTCC | |
| | TCTTTCCTCTCCCTTCTCCATTTTTGAAGAGTTTGCAAAGGAA | |
| | GGAAAGGAGCAGAGACTTGATTGAGCAGAAAATCATTTCAG | |
| | GGCCTGTTCTCTATTGTCCTTGCTATCCTGTCTTCTGTAGCTA | |
| | TCTGAAACCATCAACAAAGGAGCACACCATTCCATCAGCAA | |
| | AAGAGTAACAACATCTTTTTTTAAGTTCATTTTGTTTTTCAGT | |
| | TGATTGTATTTCAATTTTTTTACAGCTGACTTTTCTCAGAGAA | |
| | GTTTTTTTTTTATTGTAAACATACTTTTTCTAGAAAGTATATT | |
| | TTAAAATAACATCTTTAACCTTATCTCTGGCTGAATTATTGAA | |
| | TATTTGAAATTATTACATTAACAAAATTTTGTCTTACAGCAGT | |
| | GGTCCCCAACCTTCTTAGCAGTAGCATCCCTCATTAAGAATT | |
| | AAAATTTGTAGAAATTGACAAGGATTCTGACAAGCTGTTGGG | |
| | AGAGAAGAATAGAGCAGATTGCAGTAGGAACAGTTGTGTTA | |
| | GAATTTATTAATCCTTTAACACTGAAAGTAAACTATTGTTGA | |
| | TTGCCTCTTGGTGTGTTTCCATTATTCAGTGCTCTTGCTAAGT | |
| | GGGAGTCATTCCTTACATCAACCACCAACCTTCACTTGGAAG | |
| | AAGCTAGCGAAGATAAACCTCGCAACCGCCGCGGCAGCGGC | |
| | GAAGGCCGCGGCAGCCTGCTGACCTGCGGCGATGTGGAAGA | |
| | AAACCCGGGCCCGTGGACCCTGGTGAGCTGGGTGGCCTTAA | |
| | CAGCAGGGCTGGTGGCTGGAACGCGGTGCCCAGATGGTCAG | |
| | TTCTGCCCTGTGGCCTGCTGCCTGGACCCCGGAGGAGCCAGC | |
| | TACAGCTGCTGCCGTCCCCTTCTGGACAAATGGCCCACAACA | |
| | CTGAGCAGGCATCTGGGTGGCCCCTGCCAGGTTGATGCCCAC | |
| | TGCTCTGCCGGCCACTCCTGCATCTTTACCGTCTCAGGGACTT | |
| | CCAGTTGCTGCCCCTTCCCAGAGGCCGTGGCATGCGGGGATG | |
| | GCCATCACTGCTGCCCACGGGGCTTCCACTGCAGTGCAGACG | |
| | GGCGATCCTGCTTCCAAAGATCAGGTAACAACTCCGTGGGTG | |
| | CCATCCAGTGCCCTGATAGTCAGTTCGAATGCCCGGACTTCT | |
| | CCACGTGCTGTGTTATGGTCGATGGCTCCTGGGGGTGCTGCC | |
| | CCATGCCCCAGGCTTCCTGCTGTGAAGACAGGGTGCACTGCT | |
| | GTCCGCACGGTGCCTTCTGCGACCTGGTTCACACCCGCTGCA | |
| | TCACACCCACGGGCACCCACCCCCTGGCAAAGAAGCTCCCTG | |
| | CCCAGAGGACTAACAGGGCAGTGGCCTTGTCCAGCTCGGTC | |
| | ATGTGTCCGGACGCACGGTCCCGGTGCCCTGATGGTTCTACC | |
| | TGCTGTGAGCTGCCCAGTGGGAAGTATGGCTGCTGCCCAATG | |
| | CCCAACGCCACCTGCTGCTCCGATCACCTGCACTGCTGCCCC | |
| | CAAGACACTGTGTGTGACCTGATCCAGAGTAAGTGCCTCTCC | |
| | AAGGAGAACGCTACCACGGACCTCCTCACTAAGCTGCCTGC | |
| | GCACACAGTGGGGGATGTGAAATGTGACATGGAGGTGAGCT | |
| | GCCCAGATGGCTATACCTGCTGCCGTCTACAGTCGGGGGCCT | |
| | GGGGCTGCTGCCCTTTTACCCAGGCTGTGTGCTGTGAGGACC | |
| | ACATACACTGCTGTCCCGCGGGGTTTACGTGTGACACGCAGA | |
| | AGGGTACCTGTGAACAGGGGCCCCACCAGGTGCCCTGGATG | |
| | GAGAAGGCCCCAGCTCACCTCAGCCTGCCAGACCCACAAGC | |
| | CTTGAAGAGAGATGTCCCCTGTGATAATGTCAGCAGCTGTCC | |
| | CTCCTCCGATACCTGCTGCCAACTCACGTCTGGGGAGTGGGG | |
| | CTGCTGTCCAATCCCAGAGGCTGTCTGCTGCTCGGACCACCA | |
| | GCACTGCTGCCCCCAGGGCTACACGTGTGTAGCTGAGGGGC | |
| | AGTGTCAGCGAGGAAGCGAGATCGTGGCTGGACTGGAGAAG | |
| | ATGCCTGCCCGCCGGGCTTCCTTATCCCACCCCAGAGACATC | |
| | GGCTGTGACCAGCACACCAGCTGCCCGGTGGGGCAGACCTG | |
| | CTGCCCGAGCCTGGGTGGGAGCTGGGCCTGCTGCCAGTTGCC | |
| | CCATGCTGTGTGCTGCGAGGATCGCCAGCACTGCTGCCCGGC | |
| | TGGCTACACCTGCAACGTGAAGGCTCGATCCTGCGAGAAGG | |
| | AAGTGGTCTCTGCCCAGCCTGCCACCTTCCTGGCCCGTAGCC | |
| | CTCACGTGGGTGTGAAGGACGTGGAGTGTGGGGAAGGACAC | |
| | TTCTGCCATGATAACCAGACCTGCTGCCGAGACAACCGACAG | |
| | GGCTGGGCCTGCTGTCCCTACCGCCAGGGCGTCTGTTGTGCT | |
| | GATCGGCGCCACTGCTGTCCTGCTGGCTTCCGCTGCGCAGCC | |
| | AGGGGTACCAAGTGTTTGCGCAGGGAGGCCCCGCGCTGGGA | |
| | CGCCCCTTTGAGGGACCCAGCCTTGAGACAGCTGCTGTAGGT | |
| | CGACTAAACAGGCCTATTGATTGGAAAGTATGTCAACGAATT | |
| | GTGGGTCTTTTGGGGTTTGCTGCCCCTTTTACGCAATGTGGAT | |
| | ATCCTGCTTTAATGCCTTTATATGCATGTATACAAGCAAAAC | |
| | AGGCTTTTACTTTCTCGCCAACTTACAAGGCCTTTCTAAGTAA | |
| | ACAGTATCTGACCCTTTACCCCGTTGCTCGGCAACGGCCTGG | |
| | TCTGTGCCAAGTGTTTGCTGACGCAACCCCCACTGGTTGGGG | |
| | CTTGGCCATAGGCCATCAGCGCATGCGTGGAACCTTTGTGTC | |
| | TCCTCTGCCGATCCATACTGCGGAACTCCTAGCCGCTTGTTTT | |
| | GCTCGCAGCAGGTCTGGAGCGAAACTCATCGGGACTGACAA | |
| | TTCTGTCGTGCTCTCCCGCAAGTATACATCGTTTCCAGGGCTG | |
| | CTAGGCTGTGCTGCCAACTGGATCCTGCGCGGGACGTCCTTT | |
| | GTTTACGTCCCGTCGGCGCTGAATCCCGCGGACGACCCCTCC | |
| | CGGGGCCGCTTGGGGCTCTACCGCCCGCTTCTCCGTCTGCCG | |
| | TACCGACCGACCACGGGGCGCACCTCTCTTTACGCGGACTCC | |
| | CCGTCTGTGCCTTCTCATCTGCCGGACCGTGTGCACTTCGCTT | |
| | CACCTCTGCACGTCGCATGGAGACCACCGTGAACGCCCACCG | |
| | GAACCTGCCCAAGGTCTTGCATAAGAGGACTCTTGGACTTTC | |
| | AGCAATGTCAACTCGAGAGATCTACGGGTGGCATCCCTGTGA | |
| | CCCCTCCCCAGTGCCTCTCCTGGCCCTGGAAGTTGCCACTCC | |
| | AGTGCCCACCAGCCTTGTCCTAATAAAATTAAGTTGCATCAT | |
| | TTTGTCTGACTAGGTGTCCTTCTATAATATTATGGGGTGGAG | |
| | GGGGGTGGTATGGAGCAAGGGGCAAGTTGGGAAGACAACCT | |
| | GTAGGGCCTGCGGGGTCTATTGGGAACCAAGCTGGAGTGCA | |
| | GTGGCACAATCTTGGCTCACTGCAATCTCCGCCTCCTGGGTT | |
| | CAAGCGATTCTCCTGCCTCAGCCTCCCGAGTTGTTGGGATTC | |
| | CAGGCATGCATGACCAGGCTCAGCTAATTTTTGTTTTTTTGGT | |
| | AGAGACGGGGTTTCACCATATTGGCCAGGCTGGTCTCCAACT | |
| | CCTAATCTCAGGTGATCTACCCACCTTGGCCTCCCAAATTGC | |
| | TGGGATTACAGGCGTGAACCACTGCTCCCTTCCCTGTCCTTCT | |
| | GATTTTGTAGGTAACCACGTGCGGACCGAGCGGCCGCAGGA | |
| | ACCCCTAGTGATGGAGTTGGCCACTCCCTCTCTGCGCGCTCG | |
| | CTCGCTCACTGAGGCCGGGCGACCAAAGGTCGCCCGACGCC | |
| | CGGGCTTTGCCCGGGCGGCCTCAGTGAGCGAGCGAGCGCGC | |
| | AGCTGCCTGCAGG | |
| Plasmid Sequence | CCTGCAGGCAGCTGCGCGCTCGCTCGCTCACTGAGGCCGCCC | 92 |
| (beta lactamase | GGGCAAAGCCCGGGCGTCGGGCGACCTTTGGTCGCCCGGCC | |
| coding sequence is | CTAGTGAGCGAGCGAGCGCGCAGAGAGGGAGTGGCCAACTC | |
| highlighted gray; pUC | CATCACTAGGGGTTCCTGCGGCCGCACGCGTAACCGAGTATC | |
| origin of replication is | TGCAGAGGGCCCTGCGTATGAGTGCAAGTGGGTTTTAGGACC | |
| underlined | AGGATGAGGCGGGGTGGGGGTGCCTACCTGACGACCGACCC | |
| | CGACCCACTGGACAAGCACCCAACCCCCATTCCCCAAATTGC | |
| | GCATCCCCTATCAGAGAGGGGGAGGGGAAACAGGATGCGGC | |
| | GAGGCGCGTGCGCACTGCCAGCTTCAGCACCGCGGACAGTG | |
| | CCTTCGCCCCCGCCTGGCGGCGCGCGCCACCGCCGCCTCAGC | |
| | ACTGAAGGCGCGCTGACGTCACTCGCCGGTCCCCCGCAAACT | |
| | CCCCTTCCCGGCCACCTTGGTCGCGTCCGCGCCGCCGCCGGC | |
| | CCAGCCGGACCGCACCACGCGAGGCGCGAGATAGGGGGGCA | |
| | CGGGCGCGACCATCTGCGCTGCGGCGCCGGCGACTCAGCGC | |
| | TGCCTCAGTCTGCGGTGGGCAGCGGAGGAGTCGTGTCGTGCC | |
| | TGAGAGCGCAGCTGTGAATTCCTTCCAGAGGAGATTGGAAA | |
| | ACTTGAAGAAGAAGTGGATTGTGCTAATATTGCCCTGAAAGC | |
| | AGCCACCATGGATTGGGAGAGTTGGAAACAAAATTTGCAAA | |
| | TTGATATCAAGTTAGCATTTACAGATTTGGCTGAGGAGAATA | |
| | TCCATTATTTTGAACAGGTAATTAGTGTTGTTTGATATTGCTT | |
| | CATTTTAAAGTTATTTGCTCATTTACTTTTGGTCCGTCCATTG | |
| | TTGAAAGAGTGTATTAAAGAACAAGTGTCACATTCTATTGCC | |
| | TCTCTGGTAGCTTGGTTTTGTTGAAGTTGTCAGTTACCATTTG | |
| | GTTTTGTTTATCCTCAGTTTGTTGTTTTGGATTTGGATTCTTCA | |
| | AAAGCATTTGATATTGCTTTCTATTGATTGTCCTAACTACTCC | |
| | TCTTTCCTCTCCCTTCTCCATTTTTGAAGAGTTTGCAAAGGAA | |
| | GGAAAGGAGCAGAGACTTGATTGAGCAGAAAATCATTTCAG | |
| | GGCCTGTTCTCTATTGTCCTTGCTATCCTGTCTTCTGTAGCTA | |
| | TCTGAAACCATCAACAAAGGAGCACACCATTCCATCAGCAA | |
| | AAGAGTAACAACATCTTTTTTTAAGTTCATTTTGTTTTTCAGT | |
| | TGATTGTATTTCAATTTTTTTACAGCTGACTTTTCTCAGAGAA | |
| | GTTTTTTTTTTATTGTAAACATACTTTTTCTAGAAAGTATATT | |
| | TTAAAATAACATCTTTAACCTTATCTCTGGCTGAATTATTGAA | |
| | TATTTGAAATTATTACATTAACAAAATTTTGTCTTACAGCAGT | |
| | GGTCCCCAACCTTCTTAGCAGTAGCATCCCTCATTAAGAATT | |
| | AAAATTTGTAGAAATTGACAAGGATTCTGACAAGCTGTTGGG | |
| | AGAGAAGAATAGAGCAGATTGCAGTAGGAACAGTTGTGTTA | |
| | GAATTTATTAATCCTTTAACACTGAAAGTAAACTATTGTTGA | |
| | TTGCCTCTTGGTGTGTTTCCATTATTCAGTGCTCTTGCTAAGT | |
| | GGGAGTCATTCCTTACATCAACCACCAACCTTCACTTGGAAG | |
| | AAGCTAGCGAAGATAAACCTCGCAACCGCCGCGGCAGCGGC | |
| | GAAGGCCGCGGCAGCCTGCTGACCTGCGGCGATGTGGAAGA | |
| | AAACCCGGGCCCGTGGACCCTGGTGAGCTGGGTGGCCTTAA | |
| | CAGCAGGGCTGGTGGCTGGAACGCGGTGCCCAGATGGTCAG | |
| | TTCTGCCCTGTGGCCTGCTGCCTGGACCCCGGAGGAGCCAGC | |
| | TACAGCTGCTGCCGTCCCCTTCTGGACAAATGGCCCACAACA | |
| | CTGAGCAGGCATCTGGGTGGCCCCTGCCAGGTTGATGCCCAC | |
| | TGCTCTGCCGGCCACTCCTGCATCTTTACCGTCTCAGGGACTT | |
| | CCAGTTGCTGCCCCTTCCCAGAGGCCGTGGCATGCGGGGATG | |
| | GCCATCACTGCTGCCCACGGGGCTTCCACTGCAGTGCAGACG | |
| | GGCGATCCTGCTTCCAAAGATCAGGTAACAACTCCGTGGGTG | |
| | CCATCCAGTGCCCTGATAGTCAGTTCGAATGCCCGGACTTCT | |
| | CCACGTGCTGTGTTATGGTCGATGGCTCCTGGGGGTGCTGCC | |
| | CCATGCCCCAGGCTTCCTGCTGTGAAGACAGGGTGCACTGCT | |
| | GTCCGCACGGTGCCTTCTGCGACCTGGTTCACACCCGCTGCA | |
| | TCACACCCACGGGCACCCACCCCCTGGCAAAGAAGCTCCCTG | |
| | CCCAGAGGACTAACAGGGCAGTGGCCTTGTCCAGCTCGGTC | |
| | ATGTGTCCGGACGCACGGTCCCGGTGCCCTGATGGTTCTACC | |
| | TGCTGTGAGCTGCCCAGTGGGAAGTATGGCTGCTGCCCAATG | |
| | CCCAACGCCACCTGCTGCTCCGATCACCTGCACTGCTGCCCC | |
| | CAAGACACTGTGTGTGACCTGATCCAGAGTAAGTGCCTCTCC | |
| | AAGGAGAACGCTACCACGGACCTCCTCACTAAGCTGCCTGC | |
| | GCACACAGTGGGGGATGTGAAATGTGACATGGAGGTGAGCT | |
| | GCCCAGATGGCTATACCTGCTGCCGTCTACAGTCGGGGGCCT | |
| | GGGGCTGCTGCCCTTTTACCCAGGCTGTGTGCTGTGAGGACC | |
| | ACATACACTGCTGTCCCGCGGGGTTTACGTGTGACACGCAGA | |
| | AGGGTACCTGTGAACAGGGGCCCCACCAGGTGCCCTGGATG | |
| | GAGAAGGCCCCAGCTCACCTCAGCCTGCCAGACCCACAAGC | |
| | CTTGAAGAGAGATGTCCCCTGTGATAATGTCAGCAGCTGTCC | |
| | CTCCTCCGATACCTGCTGCCAACTCACGTCTGGGGAGTGGGG | |
| | CTGCTGTCCAATCCCAGAGGCTGTCTGCTGCTCGGACCACCA | |
| | GCACTGCTGCCCCCAGGGCTACACGTGTGTAGCTGAGGGGC | |
| | AGTGTCAGCGAGGAAGCGAGATCGTGGCTGGACTGGAGAAG | |
| | ATGCCTGCCCGCCGGGCTTCCTTATCCCACCCCAGAGACATC | |
| | GGCTGTGACCAGCACACCAGCTGCCCGGTGGGGCAGACCTG | |
| | CTGCCCGAGCCTGGGTGGGAGCTGGGCCTGCTGCCAGTTGCC | |
| | CCATGCTGTGTGCTGCGAGGATCGCCAGCACTGCTGCCCGGC | |
| | TGGCTACACCTGCAACGTGAAGGCTCGATCCTGCGAGAAGG | |
| | AAGTGGTCTCTGCCCAGCCTGCCACCTTCCTGGCCCGTAGCC | |
| | CTCACGTGGGTGTGAAGGACGTGGAGTGTGGGGAAGGACAC | |
| | TTCTGCCATGATAACCAGACCTGCTGCCGAGACAACCGACAG | |
| | GGCTGGGCCTGCTGTCCCTACCGCCAGGGCGTCTGTTGTGCT | |
| | GATCGGCGCCACTGCTGTCCTGCTGGCTTCCGCTGCGCAGCC | |
| | AGGGGTACCAAGTGTTTGCGCAGGGAGGCCCCGCGCTGGGA | |
| | CGCCCCTTTGAGGGACCCAGCCTTGAGACAGCTGCTGTAGGT | |
| | CGACTAAACAGGCCTATTGATTGGAAAGTATGTCAACGAATT | |
| | GTGGGTCTTTTGGGGTTTGCTGCCCCTTTTACGCAATGTGGAT | |
| | ATCCTGCTTTAATGCCTTTATATGCATGTATACAAGCAAAAC | |
| | AGGCTTTTACTTTCTCGCCAACTTACAAGGCCTTTCTAAGTAA | |
| | ACAGTATCTGACCCTTTACCCCGTTGCTCGGCAACGGCCTGG | |
| | TCTGTGCCAAGTGTTTGCTGACGCAACCCCCACTGGTTGGGG | |
| | CTTGGCCATAGGCCATCAGCGCATGCGTGGAACCTTTGTGTC | |
| | TCCTCTGCCGATCCATACTGCGGAACTCCTAGCCGCTTGTTTT | |
| | GCTCGCAGCAGGTCTGGAGCGAAACTCATCGGGACTGACAA | |
| | TTCTGTCGTGCTCTCCCGCAAGTATACATCGTTTCCAGGGCTG | |
| | CTAGGCTGTGCTGCCAACTGGATCCTGCGCGGGACGTCCTTT | |
| | GTTTACGTCCCGTCGGCGCTGAATCCCGCGGACGACCCCTCC | |
| | CGGGGCCGCTTGGGGCTCTACCGCCCGCTTCTCCGTCTGCCG | |
| | TACCGACCGACCACGGGGCGCACCTCTCTTTACGCGGACTCC | |
| | CCGTCTGTGCCTTCTCATCTGCCGGACCGTGTGCACTTCGCTT | |
| | CACCTCTGCACGTCGCATGGAGACCACCGTGAACGCCCACCG | |
| | GAACCTGCCCAAGGTCTTGCATAAGAGGACTCTTGGACTTTC | |
| | AGCAATGTCAACTCGAGAGATCTACGGGTGGCATCCCTGTGA | |
| | CCCCTCCCCAGTGCCTCTCCTGGCCCTGGAAGTTGCCACTCC | |
| | AGTGCCCACCAGCCTTGTCCTAATAAAATTAAGTTGCATCAT | |
| | TTTGTCTGACTAGGTGTCCTTCTATAATATTATGGGGTGGAG | |
| | GGGGGTGGTATGGAGCAAGGGGCAAGTTGGGAAGACAACCT | |
| | GTAGGGCCTGCGGGGTCTATTGGGAACCAAGCTGGAGTGCA | |
| | GTGGCACAATCTTGGCTCACTGCAATCTCCGCCTCCTGGGTT | |
| | CAAGCGATTCTCCTGCCTCAGCCTCCCGAGTTGTTGGGATTC | |
| | CAGGCATGCATGACCAGGCTCAGCTAATTTTTGTTTTTTTGGT | |
| | AGAGACGGGGTTTCACCATATTGGCCAGGCTGGTCTCCAACT | |
| | CCTAATCTCAGGTGATCTACCCACCTTGGCCTCCCAAATTGC | |
| | TGGGATTACAGGCGTGAACCACTGCTCCCTTCCCTGTCCTTCT | |
| | GATTTTGTAGGTAACCACGTGCGGACCGAGCGGCCGCAGGA | |
| | ACCCCTAGTGATGGAGTTGGCCACTCCCTCTCTGCGCGCTCG | |
| | CTCGCTCACTGAGGCCGGGCGACCAAAGGTCGCCCGACGCC | |
| | CGGGCTTTGCCCGGGCGGCCTCAGTGAGCGAGCGAGCGCGC | |
| | AGCTGCCTGCAGGAAGCTGTAAGCTCTAGGAGATCCGAACC | |
| | AGATAAGTGAAATCTAGTTCCAAACTATTTTGTCATTTTTAAT | |
| | TTTCGTATTAGCTTACGACGCTACACCCAGTTCCCATCTATTT | |
| | TGTCACTCTTCCCTAAATAATCCTTAAAAACTCCATTTCCACC | |
| | CCTCCCAGTTCCCAACTATTTTGTCCGCCCACAGCGGGGCAT | |
| | TTTTCTTCCTGTTATGTTTTTAATCAAACATCCTGCCAACTCC | |
| | ATGTGACAAACCGTCATCTTCGGCTACTTTTTCTCTGTCACAG | |
| | AATGAAAATTTTTCTGTCATCTCTTCGTTATTAATGTTTGTAA | |
| | TTGACTGAATATCAACGCTTATTTGCAGCCTGAATGGCGAAT | |
| | GGACGCGCCCTGTAGCGGCGCATTAAGCGCGGCGGGTGTGG | |
| | TGGTTACGCGCAGCGTGACCGCTACACTTGCCAGCGCCCTAG | |
| | CGCCCGCTCCTTTCGCTTTCTTCCCTTCCTTTCTCGCCACGTTC | |
| | GCCGGCTTTCCCCGTCAAGCTCTAAATCGGGGGCTCCCTTTA | |
| | GGGTTCCGATTTAGTGCTTTACGGCACCTCGACCCCAAAAAA | |
| | CTTGATTAGGGTGATGGTTCACGTAGTGGGCCATCGCCCTGA | |
| | TAGACGGTTTTTCGCCCTTTGACGTTGGAGTCCACGTTCTTTA | |
| | ATAGTGGACTCTTGTTCCAAACTGGAACAACACTCAACCCTA | |
| | TCTCGGTCTATTCTTTTGATTTATAAGGGATTTTGCCGATTTC | |
| | GGCCTATTGGTTAAAAAATGAGCTGATTTAACAAAAATTTAA | |
| | CGCGAATTTTAACAAAATATTAACGTTTACAATTTCAGGTGG | |
| | CACTTTTCGGGGAAATGTGCGCGGAACCCCTATTTGTTTATTT | |
| | TTCTAAATACATTCAAATATGTATCCGCTCATGAGACAATAA | |
| | CCCTGATAAATGCTTCAATAATATTGAAAAAGGAAGAGTAT | |
| | GAGTATTCAACATTTCCGTGTCGCCCTTATTCCCTTTTTTGCG | |
| | GCATTTTGCCTTCCTGTTTTTGCTCACCCAGAAACGCTGGTGA | |
| | AAGTAAAAGATGCTGAAGATCAGTTGGGTGCACGAGTGGGT | |
| | TACATCGAACTGGATCTCAACAGCGGTAAGATCCTTGAGAGT | |
| | TTTCGCCCCGAAGAACGTTTTCCAATGATGAGCACTTTTAAA | |
| | GTTCTGCTATGTGGCGCGGTATTATCCCGTATTGACGCCGGG | |
| | CAAGAGCAACTCGGTCGCCGCATACACTATTCTCAGAATGAC | |
| | TTGGTTGAGTACTCACCAGTCACAGAAAAGCATCTTACGGAT | |
| | GGCATGACAGTAAGAGAATTATGCAGTGCTGCCATAACCAT | |
| | GAGTGATAACACTGCGGCCAACTTACTTCTGACAACGATCGG | |
| | AGGACCGAAGGAGCTAACCGCTTTTTTGCACAACATGGGGG | |
| | ATCATGTAACTCGCCTTGATCGTTGGGAACCGGAGCTGAATG | |
| | AAGCCATACCAAACGACGAGCGTGACACCACGATGCCTGTA | |
| | GCAATGGCAACAACGTTGCGCAAACTATTAACTGGCGAACT | |
| | ACTTACTCTAGCTTCCCGGCAACAATTAATAGACTGGATGGA | |
| | GGCGGATAAAGTTGCAGGACCACTTCTGCGCTCGGCCCTTCC | |
| | GGCTGGCTGGTTTATTGCTGATAAATCTGGAGCCGGTGAGCG | |
| | TGGGTCTCGCGGTATCATTGCAGCACTGGGGCCAGATGGTAA | |
| | GCCCTCCCGTATCGTAGTTATCTACACGACGGGGAGTCAGGC | |
| | AACTATGGATGAACGAAATAGACAGATCGCTGAGATAGGTG | |
| | CCTCACTGATTAAGCATTGGTAACTGTCAGACCAAGTTTACT | |
| | CATATATACTTTAGATTGATTTAAAACTTCATTTTTAATTTAA | |
| | AAGGATCTAGGTGAAGATCCTTTTTGATAATCTCATGACCAA | |
| | AATCCCTTAACGTGAGTTTTCGTTCCACTGAGCGTCAGACCC | |
| | CGTAGAAAAGATCAAAGGATCTTCTTGAGATCCTTTTTTTCT | |
| | GCGCGTAATCTGCTGCTTGCAAACAAAAAAACCACCGCTACC | |
| | AGCGGTGGTTTGTTTGCCGGATCAAGAGCTACCAACTCTTTT | |
| | TCCGAAGGTAACTGGCTTCAGCAGAGCGCAGATACCAAATA | |
| | CTGTCCTTCTAGTGTAGCCGTAGTTAGGCCACCACTTCAAGA | |
| | ACTCTGTAGCACCGCCTACATACCTCGCTCTGCTAATCCTGTT | |
| | ACCAGTGGCTGCTGCCAGTGGCGATAAGTCGTGTCTTACCGG | |
| | GTTGGACTCAAGACGATAGTTACCGGATAAGGCGCAGCGGT | |
| | CGGGCTGAACGGGGGGTTCGTGCACACAGCCCAGCTTGGAG | |
| | CGAACGACCTACACCGAACTGAGATACCTACAGCGTGAGCA | |
| | TTGAGAAAGCGCCACGCTTCCCGAAGGGAGAAAGGCGGACA | |
| | GGTATCCGGTAAGCGGCAGGGTCGGAACAGGAGAGCGCACG | |
| | AGGGAGCTTCCAGGGGGAAACGCCTGGTATCTTTATAGTCCT | |
| | GTCGGGTTTCGCCACCTCTGACTTGAGCGTCGATTTTTGTGAT | |
| | GCTCGTCAGGGGGGCGGAGCCTATGGAAAAACGCCAGCAAC | |
| | GCGGCCTTTTTACGGTTCCTGGCCTTTTGCTGGCCTTTTGCTC | |
| | ACATGTTCTTTCCTGCGTTATCCCCTGATTCTGTGGATAACCG | |
| | TATTACCGCCTTTGAGTGAGCTGATACCGCTCGCCGCAGCCG | |
| | AACGACCGAGCGCAGCGAGTCAGTGAGCGAGGAAGCGGAA | |
| | GAGCGCCTGATGCGGTATTTTCTCCTTACGCATCTGTGCGGT | |
| | ATTTCACACCGCAGACCAGCCGCGTAACCTGGCAAAATCGGT | |
| | TACGGTTGAGTAATAAATGGATGCCCTGCGTAAGCGGGTGTG | |
| | GGCGGACAATAAAGTCTTAAACTGAACAAAATAGATCTAAA | |
| | CTATGACAATAAAGTCTTAAACTAGACAGAATAGTTGTAAAC | |
| | TGAAATCAGTCCAGTTATGCTGTGAAAAAGCATACTGGACTT | |
| | TTGTTATGGCTAAAGCAAACTCTTCATTTTCTGAAGTGCAAA | |
| | TTGCCCGTCGTATTAAAGAGGGGCGTGGCCAAGGGCATGGT | |
| | AAAGACTATATTCGCGGCGTTGTGACAATTTACCGAACAACT | |
| | CCGCGGCCGGGAAGCCGATCTCGGCTTGAACGAATTGTTAG | |
| | GTGGCGGTACTTGGGTCGATATCAAAGTGCATCACTTCTTCC | |
| | CGTATGCCCAACTTTGTATAGAGAGCCACTGCGGGATCGTCA | |
| | CCGTAATCTGCTTGCACGTAGATCACATAAGCACCAAGCGCG | |
| | TTGGCCTCATGCTTGAGGAGATTGATGAGCGCGGTGGCAATG | |
| | CCCTGCCTCCGGTGCTCGCCGGAGACTGCGAGATCATAGATA | |
| | TAGATCTCACTACGCGGCTGCTCAAACCTGGGCAGAACGTAA | |
| | GCCGCGAGAGCGCCAACAACCGCTTCTTGGTCGAAGGCAGC | |
| | AAGCGCGATGAATGTCTTACTACGGAGCAAGTTCCCGAGGT | |
| | AATCGGAGTCCGGCTGATGTTGGGAGTAGGTGGCTACGTCTC | |
| | CGAACTCACGACCGAAAAGATCAAGAGCAGCCCGCATGGAT | |
| | TTGACTTGGTCAGGGCCGAGCCTACATGTGCGAATGATGCCC | |
| | ATACTTGAGCCACCTAACTTTGTTTTAGGGCGACTGCCCTGC | |
| | TGCGTAACATCGTTGCTGCTGCGTAACATCGTTGCTGCTCCA | |
| | TAACATCAAACATCGACCCACGGCGTAACGCGCTTGCTGCTT | |
| | GGATGCCCGAGGCATAGACTGTACAAAAAAACAGTCATAAC | |
| | AAGCCATGAAAACCGCCACTGCGCCGTTACCACCGCTGCGTT | |
| | CGGTCAAGGTTCTGGACCAGTTGCGTGAGCGCATACGCTACT | |
| | TGCATTACAGTTTACGAACCGAACAGGCTTATGTCAACTGGG | |
| | TTCGTGCCTTCATCCGTTTCCACGGTGTGCGTCACCCGGCAA | |
| | CCTTGGGCAGCAGCGAAGTCGAGGCATTTCTGTCCTGGCTGG | |
| | CGAACGAGCGCAAGGTTTCGGTCTCCACGCATCGTCAGGCAT | |
| | TGGCGGCCTTGCTGTTCTTCTACGGCAAGGTGCTGTGCACGG | |
| | ATCTGCCCTGGCTTCAGGAGATCGGAAGACCTCGGCCGTCGC | |
| | GGCGCTTGCCGGTGGTGCTGACCCCGGATGAAGTGGTTCGCA | |
| | TCCTCGGTTTTCTGGAAGGCGAGCATCGTTTGTTCGCCCAGG | |
| | ACTCTAGCTATAGTTCTAGTGGTTGGCTACAGCTTGCATG | |
| Plasmid comprising | CTGCGCGCTCGCTCGCTCACTGAGGCCGCCCGGGCAAAGCCCGGGC | 93 |
| SNX7 minigene | GTCGGGCGACCTTTGGTCGCCCGGCCTCAGTGAGCGAGCGAGCGCG | |
| (grey), JeT promoter | CAGAGAGGGAGTGGAATTCGGGCGGAGTTAGGGCGGAGCCAATCA | |
| (underline), Furin/T2A | GCGTGCGCCGTTCCGAAAGTTGCCTTTTATGGCTGGGCGGAGAATG | |
| site (bold), and GFP | GGCGGTGAACGCCGATGATTATATAAGGACGCGCCGGGTGTGGCA | |
| (italicized) | CAGCTAGTTCCGTCGCAGCCGGGATTTGGGTCGCGGTTCTTGTTTGT | |
| | GGATCCCTGTGATCGTCACTTGACACCGGTCTTCCAGAGGAGATTG | |
| | GAAAACTTGAAGAAGAAGTGGATTGTGCTAATATTGCCCTGAAAGC | |
| | AGCCACCATGGATTGGGAGAGTTGGAAACAAAATTTGCAAATTGAT | |
| | ATCAAGTTAGCATTTACAGATTTGGCTGAGGAGAATATCCATTATT | |
| | TTGAACAGGTAATTAGTGTTGTTTGATATTGCTTCATTTTAAAGTTA | |
| | TTTGCTCATTTACTTTTGGTCCGTCCATTGTTGAAAGAGTGTATTAA | |
| | AGAACAAGTGTCACATTCTATTGCCTCTCTGGTAGCTTGGTTTTGTT | |
| | GAAGTTGTCAGTTACCATTTGGTTTTGTTTATCCTCAGTTTGTTGTTT | |
| | TGGATTTGGATTCTTCAAAAGCATTTGATATTGCTTTCTATTGATTG | |
| | TCCTAACTACTCCTCTTTCCTCTCCCTTCTCCATTTTTGAAGAGTTTG | |
| | CAAAGGAAGGAAAGGAGCAGAGACTTGATTGAGCAGAAAATCATT | |
| | TCAGGGCCTGTTCTCTATTGTCCTTGCTATCCTGTCTTCTGTAGCTAT | |
| | CTGAAACCATCAACAAAGGAGCACACCATTCCATCAGCAAAAGAG | |
| | TAACAACATCTTTTTTTAAGTTCATTTTGTTTTTCAGTTGATTGTATT | |
| | TCAATTTTTTTACAGCTGACTTTTCTCAGAGAAGTTTTTTTTTTATTG | |
| | TAAACATACTTTTTCTAGAAAGTATATTTTAAAATAACATCTTTAAC | |
| | CTTATCTCTGGCTGAATTATTGAATATTTGAAATTATTACATTAACA | |
| | AAATTTTGTCTTACAGCAGTGGTCCCCAACCTTCTTAGCAGTAGCAT | |
| | CCCTCATTAAGAATTAAAATTTGTAGAAATTGACAAGGATTCTGAC | |
| | AAGCTGTTGGGAGAGAAGAATAGAGCAGATTGCAGTAGGAACAGT | |
| | TGTGTTAGAATTTATTAATCCTTTAACACTGAAAGTAAACTATTGTT | |
| | GATTGCCTCTTGGTGTGTTTCCATTATTCAGTGCTCTTGCTAAGTGG | |
| | GAGTCATTCCTTACATCAACCACCAACCTTCACTTGGAAGAAGCTA | |
| | GCGAAGATAAACCTCGCAACCGCCGCGGCAGCGGCGAAGGCCG | |
| | CGGCAGCCTGCTGACCTGCGGCGATGTGGAAGAAAACCCGGGC | |
| | CCGGTGAGCAAGGGCGAGGAGCTGTTCACCGGGGTGGTGCCCATCCT | |
| | GGTCGAGCTGGACGGCGACGTAAACGGCCACAAGTTCAGCGTGTCCGG | |
| | CGAGGGCGAGGGCGATGCCACCTACGGCAAGCTGACCCTGAAGTTCAT | |
| | CTGCACCACCGGCAAGCTGCCCGTGCCCTGGCCCACCCTCGTGACCAC | |
| | CCTGACCTACGGCGTGCAGTGCTTCAGCCGCTACCCCGACCACATGAA | |
| | GCAGCACGACTTCTTCAAGTCCGCCATGCCCGAAGGCTACGTCCAGGA | |
| | GCGCACCATCTTCTTCAAGGACGACGGCAACTACAAGACCCGCGCCGA | |
| | GGTGAAGTTCGAGGGCGACACCCTGGTGAACCGCATCGAGCTGAAGGG | |
| | CATCGACTTCAAGGAGGACGGCAACATCCTGGGGCACAAGCTGGAGTA | |
| | CAACTACAACAGCCACAACGTCTATATCATGGCCGACAAGCAGAAGAAC | |
| | GGCATCAAGGTGAACTTCAAGATCCGCCACAACATCGAGGACGGCAGC | |
| | GTGCAGCTCGCCGACCACTACCAGCAGAACACCCCCATCGGCGACGGC | |
| | CCCGTGCTGCTGCCCGACAACCACTACCTGAGCACCCAGTCCGCCCTG | |
| | AGCAAAGACCCCAACGAGGCGCGATCACATGGTCCTGCTGGAGTTC | |
| | GTGACCGCCGCCGGGATCACTCTCGGCATGGACGAGCTGTACAAGTAA | |
| | TGCTTTATTTGTGAAATTTGTGATGCTATTGCTTTATTTGTAACCATT | |
| | ATAAGCTGCAATAAACAAGTTAACAACAACAATTGCATTCATTTTA | |
| | TGTTTCAGGTTCAGGGGGAGGTGTGGGAGGTTTTTTAAAATCGATC | |
| | TGAGGAACCCCTAGTGATGGAGTTGGCCACTCCCCCCCCCCCCCGG | |
| | CGATTCTCTTGTTTGCTCCAGACTCTCAGGCAATGACCTGATAGCCT | |
| | TTGTAGAGACCTCTCAAAAATAGCTACCCTCTCCGGCATGAATTTA | |
| | TCAGCTAGAACGGTTGAATATCATATTGATGGTGATTTGACTGTCTC | |
| | CGGCCTTTCTCACCCGTTTGAATCTTTACCTACACATTACTCAGGCA | |
| | TTGCATTTAAAATATATGAGGGTTCTAAAAATTTTTATCCTTGCGTT | |
| | GAAATAAAGGCTTCTCCCGCAAAAGTATTACAGGGTCATAATGTTT | |
| | TTGGTACAACCGATTTAGCTTTATGCTCTGAGGCTTTATTGCTTAAT | |
| | TTTGCTAATTCTTTGCCTTGCCTGTATGATTTATTGGATGTTGGAAT | |
| | CGCCTGATGCGGTATTTTCTCCTTACGCATCTGTGCGGTATTTCACA | |
| | CCGCATATGGTGCACTCTCAGTACAATCTGCTCTGATGCCGCATAG | |
| | TTAAGCCAGCCCCGACACCCGCCAACACTATGGTGCACTCTCAGTA | |
| | CAATCTGCTCTGATGCCGCATAGTTAAGCCAGCCCCGACACCCGCC | |
| | AACACCCGCTGACGCGCCCTGACGGGCTTGTCTGCTCCCGGCATCC | |
| | GCTTACAGACAAGCTGTGACCGTCTCCGGGAGCTGCATGTGTCAGA | |
| | GGTTTTCACCGTCATCACCGAAACGCGCGAGACGAAAGGGCCTCGT | |
| | GATACGCCTATTTTTATAGGTTAATGTCATGATAATAATGGTTTCTT | |
| | AGACGTCAGGTGGCACTTTTCGGGGAAATGTGCGCGGAACCCCTAT | |
| | TTGTTTATTTTTCTAAATACATTCAAATATGTATCCGCTCATGAGAC | |
| | AATAACCCTGATAAATGCTTCAATAATATTGAAAAAGGAAGAGTAT | |
| | GAGCCATATTCAACGGGAAACGTCGAGGCCGCGATTAAATTCCAAC | |
| | ATGGATGCTGATTTATATGGGTATAAATGGGCTCGCGATAATGTCG | |
| | GGCAATCAGGTGCGACAATCTATCGCTTGTATGGGAAGCCCGATGC | |
| | GCCAGAGTTGTTTCTGAAACATGGCAAAGGTAGCGTTGCCAATGAT | |
| | GTTACAGATGAGATGGTCAGACTAAACTGGCTGACGGAATTTATGC | |
| | CACTTCCGACCATCAAGCATTTTATCCGTACTCCTGATGATGCATGG | |
| | TTACTCACCACTGCGATCCCCGGAAAAACAGCGTTCCAGGTATTAG | |
| | AAGAATATCCTGATTCAGGTGAAAATATTGTTGATGCGCTGGCAGT | |
| | GTTCCTGCGCCGGTTGCACTCGATTCCTGTTTGTAATTGTCCTTTTA | |
| | ACAGCGATCGCGTATTTCGCCTCGCTCAGGCGCAATCACGAATGAA | |
| | TAACGGTTTGGTTGATGCGAGTGATTTTGATGACGAGCGTAATGGC | |
| | TGGCCTGTTGAACAAGTCTGGAAAGAAATGCATAAACTTTTGCCAT | |
| | TCTCACCGGATTCAGTCGTCACTCATGGTGATTTCTCACTTGATAAC | |
| | CTTATTTTTGACGAGGGGAAATTAATAGGTTGTATTGATGTTGGAC | |
| | GAGTCGGAATCGCAGACCGATACCAGGATCTTGCCATCCTATGGAA | |
| | CTGCCTCGGTGAGTTTTCTCCTTCATTACAGAAACGGCTTTTTCAAA | |
| | AATATGGTATTGATAATCCTGATATGAATAAATTGCAGTTTCATTTG | |
| | ATGCTCGATGAGTTTTTCTAACTGTCAGACCAAGTTTACTCATATAT | |
| | ACTTTAGATTGATTTAAAACTTCATTTTTAATTTAAAAGGATCTAGG | |
| | TGAAGATCCTTTTTGATAATCTCATGACCAAAATCCCTTAACGTGA | |
| | GTTTTCGTTCCACTGAGCGTCAGACCCCGTAGAAAAGATCAAAGGA | |
| | TCTTCTTGAGATCCTTTTTTTCTGCGCGTAATCTGCTGCTTGCAAAC | |
| | AAAAAAACCACCGCTACCAGCGGTGGTTTGTTTGCCGGATCAAGAG | |
| | CTACCAACTCTTTTTCCGAAGGTAACTGGCTTCAGCAGAGCGCAGA | |
| | TACCAAATACTGTTCTTCTAGTGTAGCCGTAGTTAGGCCACCACTTC | |
| | AAGAACTCTGTAGCACCGCCTACATACCTCGCTCTGCTAATCCTGTT | |
| | ACCAGTGGCTGCTGCCAGTGGCGATAAGTCGTGTCTTACCGGGTTG | |
| | GACTCAAGACGATAGTTACCGGATAAGGCGCAGCGGTCGGGCTGA | |
| | ACGGGGGGTTCGTGCACACAGCCCAGCTTGGAGCGAACGACCTAC | |
| | ACCGAACTGAGATACCTACAGCGTGAGCTATGAGAAAGCGCCACG | |
| | CTTCCCGAAGGGAGAAAGGCGGACAGGTATCCGGTAAGCGGCAGG | |
| | GTCGGAACAGGAGAGCGCACGAGGGAGCTTCCAGGGGGAAACGCC | |
| | TGGTATCTTTATAGTCCTGTCGGGTTTCGCCACCTCTGACTTGAGCG | |
| | TCGATTTTTGTGATGCTCGTCAGGGGGGCGGAGCCTATGGAAAAAC | |
| | GCCAGCAACGCGGCCTTTTTACGGTTCCTGGCCTTTTGCTGGCCTTT | |
| | TGCTCACATGTTCTTTCCTGCGTTATCCCCTGATTCTGTGGATAACC | |
| | GTATTACCGCCTTTGAGTGAGCTGATACCGCTCGCCGCAGCCGAAC | |
| | GACCGAGCGCAGCGAGTCAGTGAGCGAGGAAGCGGAAGAGCGCCC | |
| | AATACGCAAACCGCCTCTCCCCGCGCGTTGGCCGATTCATTAATGC | |
| | AGCTGGCGTAATAGCGAAGAGGCCCGCACCGATCGCCCTTCCCAAC | |
| | AGTTGCGCAGCCTGAATGGCGAATGGCGATTCCGTTGCAATGGCTG | |
| | GCGGTAATATTGTTCTGGATATTACCAGCAAGGCCGATAGTTTGAG | |
| | TTCTTCTACTCAGGCAAGTGATGTTATTACTAATCAAAGAAGTATTG | |
| | CGACAACGGTTAATTTGCGTGATGGACAGACTCTTTTACTCGGTGG | |
| | CCTCACTGATTATAAAAACACTTCTCAGGATTCTGGCGTACCGTTCC | |
| | TGTCTAAAATCCCTTTAATCGGCCTCCTGTTTAGCTCCCGCTCTGAT | |
| | TCTAACGAGGAAAGCACGTTATACGTGCTCGTCAAAGCAACCATAG | |
| | TACGCGCCCTGTAGCGGCGCATTAAGCGCGGCGGGTGTGGTGGTTA | |
| | CGCGCAGCGTGACCGCTACACTTGCCAGCGCCCTAGCGCCCGCTCC | |
| | TTTCGCTTTCTTCCCTTCCTTTCTCGCCACGTTCGCCGGCTTTCCCCG | |
| | TCAAGCTCTAAATCGGGGGCTCCCTTTAGGGTTCCGATTTAGTGCTT | |
| | TACGGCACCTCGACCCCAAAAAACTTGATTAGGGTGATGGTTCACG | |
| | TAGTGGGCCATCGCCCTGATAGACGGTTTTTCGCCCTTTGACGTTGG | |
| | AGTCCACGTTCTTTAATAGTGGACTCTTGTTCCAAACTGGAACAAC | |
| | ACTCAACCCTATCTCGGTCTATTCTTTTGATTTATAAGGGATTTTGC | |
| | CGATTTCGGCCTATTGGTTAAAAAATGAGCTGATTTAACAAAAATT | |
| | TAACGCGAATTTTAACAAAATATTAACGCTTACAATTTAAATATTT | |
| | GCTTATACAATCTTCCTGTTTTTGGGGCTTTTCTGATTATCAACCGG | |
| | GGTACATATGATTGACATGCTAGTTTTACGATTACCGTTCATCGCC | |
| Full SNX7 minigene | GAAGAAGAAGATATCAAGTTAGCATTTACAGATTTGGCTGAGGAG | 94 |
| (version 2) | AAGAACAGGTAATTAGTGTTGTTTGATATTGCTTCATTTTAAAGTTA | |
| | TTTGCTCATTTAGCATTTGATATTGCTTTCTATTGATTGTCCTAACTA | |
| | CTCCTCTTTCCTCTCCCTTCTCCATTTTTGAAGAGTTTGCAAAGGAA | |
| | GGAAAGGAGCAGAGACTTGATTGAGCAGAAAATCATTTCAGGGCC | |
| | TGTTCTCTATTGTCCTTGCTATCCTGTCTTCTGTAGCTATCTGAAACC | |
| | ATCAACAAAGGAGCACACCATGGCATCAGCAAAAGAGTAACAACA | |
| | TCTTTTTTTAAGTTCATTTTGTTTTTCAGTTGATTGTATTTCAATTTTT | |
| | TTACAGCTGACTTTTCTCAGAGAAGTTTTTTTTTTATTGTAAACATA | |
| | CTTTTTCTAGAAAGTATATTTTAAAATAACATCTTTAACCTTATCTC | |
| | TGGCTGAATTATTGAATATTTGAAATTATTACATTAACAAAATTTTG | |
| | TCTTACAGCAGTGGTCCCCAACCTTCTTAGCAGTAGCATCCCTCATT | |
| | AAGAATTAAAATTTGTAGAAATTGACAAGGATTCTGACAAGCTGTT | |
| | GGGAGAGAAGAATAGAGCAGATTGCAGTAGGAACAGTTGTGTTAG | |
| | AATTTATTAATCCTTTAACACTGAAAGTAAACTATTGTTGATTGCCT | |
| | CTTGGTGTGTTTCCATTATTCAGTGCTCTTGCTAAGTGGGAGTCATT | |
| | CCTTACATCAACCACCAACCTTCACTTGGAAGAAGCTAGCGAAGAT | |
| | AAACCT | |
| Plasmid comprising | GCCCAATACGCAAACCGCCTCTCCCCGCGCGTTGGCCGATTCATTA | 95 |
| modttted SNX7 | ATGCAGCTGATTCTAACGAGGAAAGCACGTTATACGTGCTCGTCAA | |
| minigene version 2 | AGCAACCATAGTACGCGCCCTGTAGCGGCGCATTAAGCGCGGCGG | |
| (grey), JeT promoter | GTGTGGTGGTTACGCGCAGCGTGACCGCTACACTTGCCAGCGCCCT | |
| (underline), Furin/T2A | AGCGCCCGCTCCTTTCGCTTTCTTCCCTTCCTTTCTCGCCACGTTCGC | |
| site (bold), and | CGGCTTTCCCCGTCAAGCTCTAAATCGGGGGCTCCCTTTAGGGTTCC | |
| luciferase (italicized) | GATTTAGTGCTTTACGGCACCTCGACCCCAAAAAACTTGATTAGGG | |
| | TGATGGTTCACGTAGTGGGCCATCGCCCTGATAGACGGTTTTTCGC | |
| | CCTTTGACGTTGGAGTCCACGTTCTTTAATAGTGGACTCTTGTTCCA | |
| | AACTGGAACAACACTCAACCCTATCTCGGTCTATTCTTTTGATTTAT | |
| | AAGGGATTTTGCCGATTTCGGCCTATTGGTTAAAAAATGAGCTGAT | |
| | TTAACAAAAATTTAACGCGAATTTTAACAAAATATTAACGCTTACA | |
| | ATTTAAATATTTGCTTATACAATCTTCCTGTTTTTGGGGCTTTTCTGA | |
| | TTATCAACCGGGGTACATATGATTGACATGCTAGTTTTACGATTACC | |
| | GTTCATCGCCCTGCGCGCTCGCTCGCTCACTGAGGCCGCCCGGGCA | |
| | AAGCCCGGGCGTCGGGCGACCTTTGGTCGCCCGGCCTCAGTGAGCG | |
| | AGCGAGCGCGCAGAGAGGGAGTGGAATTCAATTCGGGCGGAGTTA | |
| | GGGCGGAGCCAATCAGCGTGCGCCGTTCCGAAAGTTGCCTTTTATG | |
| | GCTGGGCGGAGAATGGGCGGTGAACGCCGATGATTATATAAGGAC | |
| | GCGCCGGGTGTGGCACAGCTAGTTCCGTCGCAGCCGGGATTTGGGT | |
| | CGCGGTTCTTGTTTGTGGATCCCTGTGATCGTCACTTGACACCGGTG | |
| | AAGAAGAAGATATCAAGTTAGCATTTACAGATTTGGCTGAGGAGA | |
| | AGAACAGGTAATTAGTGTTGTTTGATATTGCTTCATTTTAAAGTTAT | |
| | TTGCTCATTTAGCATTTGATATTGCTTTCTATTGATTGTCCTAACTAC | |
| | TCCTCTTTCCTCTCCCTTCTCCATTTTTGAAGAGTTTGCAAAGGAAG | |
| | GAAAGGAGCAGAGACTTGATTGAGCAGAAAATCATTTCAGGGCCT | |
| | GTTCTCTATTGTCCTTGCTATCCTGTCTTCTGTAGCTATCTGAAACC | |
| | ATCAACAAAGGAGCACACCATGGCATCAGCAAAAGAGTAACAACA | |
| | TCTTTTTTTAAGTTCATTTTGTTTTTCAGTTGATTGTATTTCAATTTTT | |
| | TTACAGCTGACTTTTCTCAGAGAAGTTTTTTTTTTATTGTAAACATA | |
| | CTTTTTCTAGAAAGTATATTTTAAAATAACATCTTTAACCTTATCTC | |
| | TGGCTGAATTATTGAATATTTGAAATTATTACATTAACAAAATTTTG | |
| | TCTTACAGCAGTGGTCCCCAACCTTCTTAGCAGTAGCATCCCTCATT | |
| | AAGAATTAAAATTTGTAGAAATTGACAAGGATTCTGACAAGCTGTT | |
| | GGGAGAGAAGAATAGAGCAGATTGCAGTAGGAACAGTTGTGTTAG | |
| | AATTTATTAATCCTTTAACACTGAAAGTAAACTATTGTTGATTGCCT | |
| | CTTGGTGTGTTTCCATTATTCAGTGCTCTTGCTAAGTGGGAGTCATT | |
| | CCTTACATCAACCACCAACCTTCACTTGGAAGAAGCTAGCGAAGAT | |
| | AAACCTCGCAACCGCCGCGGCAGCGGCGAAGGCCGCGGCAGCC | |
| | TGCTGACCTGCGGCGATGTGGAAGAAAACCCGGGCCCGGTCTTC | |
| | ACACTCGAAGATTTCGTTGGGGACTGGCGACAGACAGCCGGCTACAAC | |
| | CTGGACCAAGTCCTTGAACAGGGAGGTGTGTCCAGTTTGTTTCAGAATC | |
| | TCGGGGTGTCCGTAACTCCGATCCAAAGGATTGTCCTGAGCGGTGAAAA | |
| | TGGGCTGAAGATCGACATCCATGTCATCATCCCGTATGAAGGTCTGAGC | |
| | GGCGACCAAATGGGCCAGATCGAAAAAATTTTTAAGGTGGTGTACCCTG | |
| | TGGATGATCATCACTTTAAGGTGATCCTGCACTATGGCACACTGGTAATC | |
| | GACGGGGTTACGCCGAACATGATCGACTATTTCGGACGGCCGTATGAA | |
| | GGCATCGCCGTGTTCGACGGCAAAAAGATCACTGTAACAGGGACCCTG | |
| | TGGAACGGCAACAAAATTATCGACGAGCGCCTGATCAACCCCGACGGC | |
| | TCCCTGCTGTTCCGAGTAACCATCAACGGAGTGACCGGCTGGCGGCTG | |
| | TGCGAACGCATTCTGGCGTAACCTGCCCGCTGGGCCTCCCAACGGGC | |
| | CCTCCTCCCCTCCTTGCACCAAGCTTATCGATACCGTCGACTAGAGC | |
| | TCGCTGATCAGCCTCGACTGTGCCTTCTAGTTGCCAGCCATCTGTTG | |
| | TTTGCCCCTCCCCCGTGCCTTCCTTGACCCTGGAAGGTGCCACTCCC | |
| | ACTGTCCTTTCCTAATAAAATGAGGAAATTGCATCGCATTGTCTGA | |
| | GTAGGTGTCATTCTATTCTGGGGGGTGGGGTGGGGCAGGACAGCAA | |
| | GGGGGAGGATTGGGAAGACAATAGCAGGCATGCTGGGGAGAGATC | |
| | GATCTGAGGAACCCCTAGTGATGGAGTTGGCCACTCCCTCTCTGCG | |
| | CGCTCGCTCGCTCACTGAGGCCGGGCGACCAAAGGTCGCCCGACGC | |
| | CCGGGCTTTGCCCGGGCGGCCTCAGTGAGCGAGCGAGCGCGCAGA | |
| | GAGGGAGTGGCCCCCCCCCCCCCCCCCCCGGCGATTCTCTTGTTTGC | |
| | TCCAGACTCTCAGGCAATGACCTGATAGCCTTTGTAGAGACCTCTC | |
| | AAAAATAGCTACCCTCTCCGGCATGAATTTATCAGCTAGAACGGTT | |
| | GAATATCATATTGATGGTGATTTGACTGTCTCCGGCCTTTCTCACCC | |
| | GTTTGAATCTTTACCTACACATTACTCAGGCATTGCATTTAAAATAT | |
| | ATGAGGGTTCTAAAAATTTTTATCCTTGCGTTGAAATAAAGGCTTCT | |
| | CCCGCAAAAGTATTACAGGGTCATAATGTTTTTGGTACAACCGATT | |
| | TAGCTTTATGCTCTGAGGCTTTATTGCTTAATTTTGCTAATTCTTTGC | |
| | CTTGCCTGTATGATTTATTGGATGTTGGAATCGCCTGATGCGGTATT | |
| | TTCTCCTTACGCATCTGTGCGGTATTTCACACCGCATATGGTGCACT | |
| | CTCAGTACAATCTGCTCTGATGCCGCATAGTTAAGCCAGCCCCGAC | |
| | ACCCGCCAACACCCGCTGACGCGCCCTGACGGGCTTGTCTGCTCCC | |
| | GGCATCCGCTTACAGACAAGCTGTGACCGTCTCCGGGAGCTGCATG | |
| | TGTCAGAGGTTTTCACCGTCATCACCGAAACGCGCGAGACGAAAGG | |
| | GCCTCGTGATACGCCTATTTTTATAGGTTAATGTCATGATAATAATG | |
| | GTTTCTTAGACGTCAGGTGGCACTTTTCGGGGAAATGTGCGCGGAA | |
| | CCCCTATTTGTTTATTTTTCTAAATACATTCAAATATGTATCCGCTC | |
| | ATGAGACAATAACCCTGATAAATGCTTCAATAATATTGAAAAAGGA | |
| | AGAGTATGAGTATTCAACATTTCCGTGTCGCCCTTATTCCCTTTTTT | |
| | GCGGCATTTTGCCTTCCTGTTTTTGCTCACCCAGAAACGCTGGTGAA | |
| | AGTAAAAGATGCTGAAGATCAGTTGGGTGCACGAGTGGGTTACATC | |
| | GAACTGGATCTCAACAGCGGTAAGATCCTTGAGAGTTTTCGCCCCG | |
| | AAGAACGTTTTCCAATGATGAGCACTTTTAAAGTTCTGCTATGTGG | |
| | CGCGGTATTATCCCGTATTGACGCCGGGCAAGAGCAACTCGGTCGC | |
| | CGCATACACTATTCTCAGAATGACTTGGTTGAGTACTCACCAGTCA | |
| | CAGAAAAGCATCTTACGGATGGCATGACAGTAAGAGAATTATGCA | |
| | GTGCTGCCATAACCATGAGTGATAACACTGCGGCCAACTTACTTCT | |
| | GACAACGATCGGAGGACCGAAGGAGCTAACCGCTTTTTTGCACAAC | |
| | ATGGGGGATCATGTAACTCGCCTTGATCGTTGGGAACCGGAGCTGA | |
| | ATGAAGCCATACCAAACGACGAGCGTGACACCACGATGCCTGTAG | |
| | CAATGGCAACAACGTTGCGCAAACTATTAACTGGCGAACTACTTAC | |
| | TCTAGCTTCCCGGCAACAATTAATAGACTGGATGGAGGCGGATAAA | |
| | GTTGCAGGACCACTTCTGCGCTCGGCCCTTCCGGCTGGCTGGTTTAT | |
| | TGCTGATAAATCTGGAGCCGGTGAGCGTGGGTCTCGCGGTATCATT | |
| | GCAGCACTGGGGCCAGATGGTAAGCCCTCCCGTATCGTAGTTATCT | |
| | ACACGACGGGGAGTCAGGCAACTATGGATGAACGAAATAGACAGA | |
| | TCGCTGAGATAGGTGCCTCACTGATTAAGCATTGGTAACTGTCAGA | |
| | CCAAGTTTACTCATATATACTTTAGATTGATTTAAAACTTCATTTTT | |
| | AATTTAAAAGGATCTAGGTGAAGATCCTTTTTGATAATCTCATGAC | |
| | CAAAATCCCTTAACGTGAGTTTTCGTTCCACTGAGCGTCAGACCCC | |
| | GTAGAAAAGATCAAAGGATCTTCTTGAGATCCTTTTTTTCTGCGCGT | |
| | AATCTGCTGCTTGCAAACAAAAAAACCACCGCTACCAGCGGTGGTT | |
| | TGTTTGCCGGATCAAGAGCTACCAACTCTTTTTCCGAAGGTAACTG | |
| | GCTTCAGCAGAGCGCAGATACCAAATACTGTCCTTCTAGTGTAGCC | |
| | GTAGTTAGGCCACCACTTCAAGAACTCTGTAGCACCGCCTACATAC | |
| | CTCGCTCTGCTAATCCTGTTACCAGTGGCTGCTGCCAGTGGCGATA | |
| | AGTCGTGTCTTACCGGGTTGGACTCAAGACGATAGTTACCGGATAA | |
| | GGCGCAGCGGTCGGGCTGAACGGGGGGTTCGTGCACACAGCCCAG | |
| | CTTGGAGCGAACGACCTACACCGAACTGAGATACCTACAGCGTGA | |
| | GCTATGAGAAAGCGCCACGCTTCCCGAAGGGAGAAAGGCGGACAG | |
| | GTATCCGGTAAGCGGCAGGGTCGGAACAGGAGAGCGCACGAGGGA | |
| | GCTTCCAGGGGGAAACGCCTGGTATCTTTATAGTCCTGTCGGGTTTC | |
| | GCCACCTCTGACTTGAGCGTCGATTTTTGTGATGCTCGTCAGGGGG | |
| | GCGGAGCCTATGGAAAAACGCCAGCAACGCGGCCTTTTTACGGTTC | |
| | CTGGCCTTTTGCTGGCCTTTTGCTCACATGTTCTTTCCTGCGTTATCC | |
| | CCTGATTCTGTGGATAACCGTATTACCGCCTTTGAGTGAGCTGATAC | |
| | CGCTCGCCGCAGCCGAACGACCGAGCGCAGCGAGTCAGTGAGCGA | |
| | GGAAGCGGAAGAGC | |

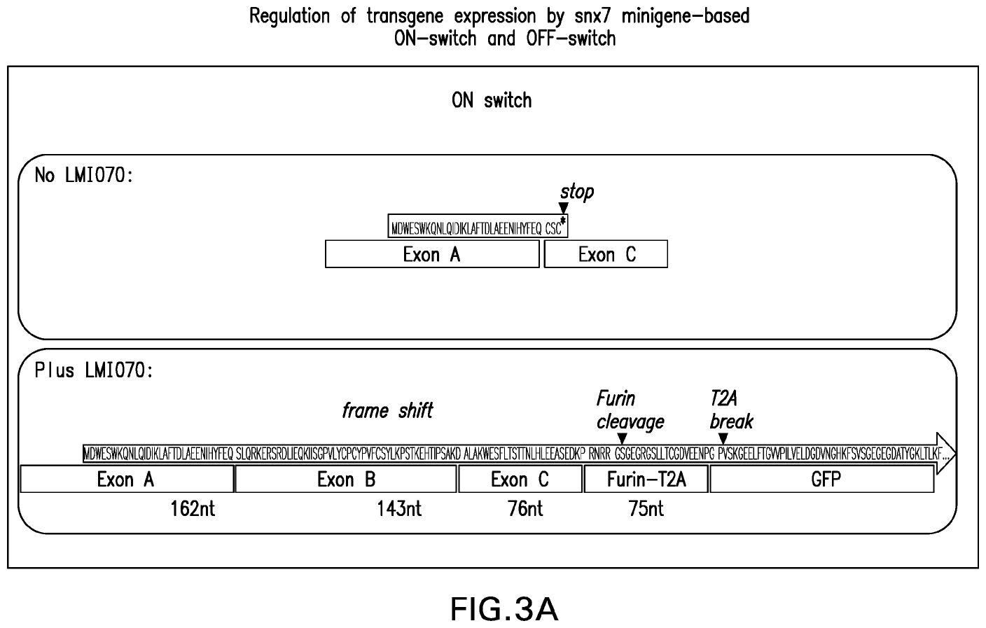

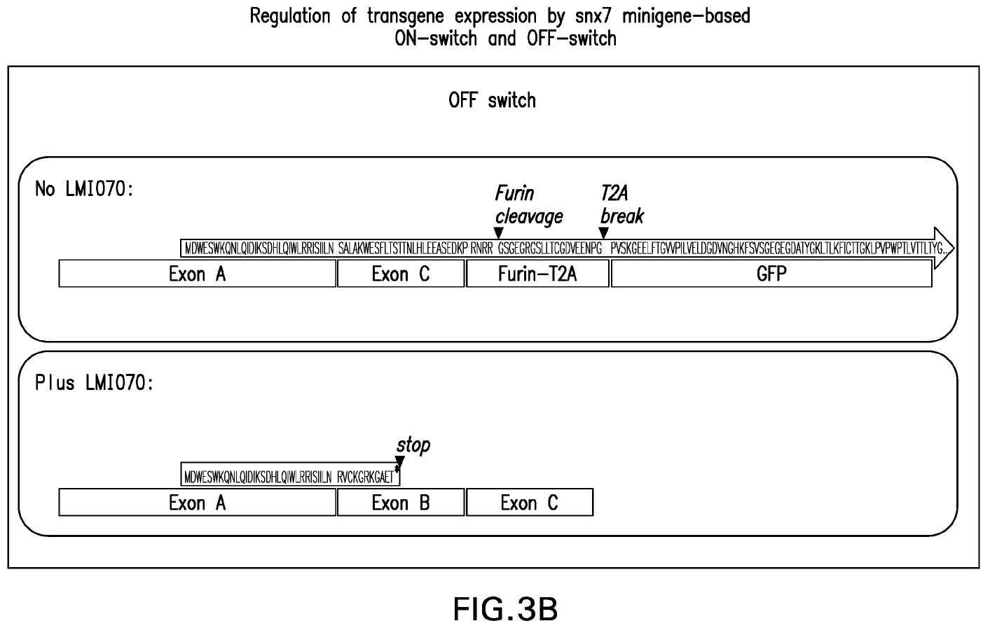

In various embodiments, a minigene or vector disclosed herein may be used to increase in the levels of functional polypeptide, e.g., the level of hPGRN, in response to the presence or absence of splice modulator. In some embodiments, a vector disclosed herein exhibits higher expression of the transgene sequence in the presence of a splice modulator compared to the expression of the same vector in the absence of the splice modulator. In some embodiments, the level of expression of the molecule of interest in the presence of the splice modulator is greater, e.g., 2, 3, 4, 5, 6, 7, 8, 9, 10, 20, 30, 40, 50, 60, 70, 80, 90, or 100 fold greater, than the level of expression of the molecule of interest in the absence of the splice modulator. In some embodiments, the level of expression of the molecule of interest in the absence of the splice modulator is greater, e.g., 2, 3, 4, 5, 6, 7, 8, 9, 10, 20, 30, 40, 50, 60, 70, 80, 90, or 100 fold greater, than the level of expression of the molecule of interest in the presence of the splice modulator. In some embodiments, the increase in expression of the transgene sequence is measured by an increase in the number of RNA transcripts of the transgene sequence. In some embodiments, the increase in expression of the transgene sequence is measured by PCR. In some embodiments, the increase in expression of the transgene sequence is measured by RT-PCR. In some embodiments, the increase in expression of the transgene sequence is measured by qPCR. In some embodiments, the increase in expression of the transgene sequence is measured by qRT-PCR. In some embodiments, the increase in expression of the transgene sequence is measured by sequencing. In some embodiments, the increase in expression of the transgene sequence is measured by Northern blot analysis. In some embodiments, the increase in expression of the transgene sequence is measured by single-molecule Fluorescence In-Situ Hybridization (FISH). In some embodiments, the increase in expression of the transgene sequence is measured by an increase in the amount of protein encoded by the transgene produced. In some embodiments, the increase in expression of the transgene sequence is measured by an enzyme-linked immunosorbent assay (ELISA). In some embodiments, the increase in expression of the transgene sequence is measured by Western blot analysis. In some embodiments, the increase in expression of the transgene sequence is measured by immunostaining. In some embodiments, the increase in expression of the transgene sequence is measured by more than one of the above listed methods. In some embodiments, the increase in expression of the transgene sequence is measured by the amount of mRNA which includes the second exon. In some embodiments, the increase in expression of the transgene sequence is measured by the amount of mRNA which includes a direct first exon to third exon splice. Exemplary polypeptides produced in the presence or absence of splice modulator from vectors incorporating either an on-switch minigene or an off-switch minigene are depicted in FIG. 3 (in each case, prior to cleavage of the protease cleavage site and/or self-cleaving peptide sequence).

Recombinant Virus

In various embodiments, the nucleic acids and vectors discussed herein may be present in one or more virus particle, such as a recombinant virus particle. Recombinant viruses are viruses generated by recombinant means. Various different viral types may be used, e.g., retroviruses, adenovirus, lentivirus, AAV, murine leukemia viruses, etc. Without being bound by theory, vectors delivered from retroviruses such as the lentivirus may provide for long-term gene transfer since they allow long-term, stable integration of a transgene and its propagation in daughter cells and may also provide low immunogenicity. Other suitable retroviruses include gammaretroviruses. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June, 3(6): 677-713. In some embodiments, the virus is a recombinant adenovirus comprising a nucleic acid or vector disclosed herein. In some embodiments, the virus is a recombinant AAV comprising a nucleic acid or vector disclosed herein.

In some embodiments, the nucleic acids or vectors disclosed herein are for use in the manufacture of a recombinant virus. In some embodiments, the nucleic acids or vectors disclosed herein are for use in the manufacture of an rAAV. Thus, also disclosed herein, in various embodiments, are virus compositions (also referred to as virions), e.g., rAAV virus compositions comprising a viral vector or nucleic acid disclosed above. In some embodiments, the recombinant virus is an adeno-associated virus (AAV) or any mutant or derivative thereof. In some embodiments, the recombinant virus is a chimeric AAV or any mutant or derivative thereof. In some embodiments, the recombinant virus is an adenovirus or any mutant or derivative thereof. In some embodiments, the recombinant virus is a retrovirus or any mutant or derivative thereof. In some embodiments, the recombinant virus is a lentivirus or any mutant or derivative thereof. In some embodiments, the recombinant virus is a DNA virus or any mutant or derivative thereof. In some embodiments, the recombinant virus is a herpes simplex virus or any mutant or derivative thereof. In some embodiments, the recombinant virus is a baculovirus or any mutant or derivative thereof.

In some embodiments, an AAV disclosed herein may comprise one or more AAV capsid proteins. AAV capsid proteins may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAVrh8, AAVfh10, AAV-DJ, AAV-DJ/8, AAV-PHP.B, AAV-PHP.B2, AAV-PHP.B3, AAV-PHP.A, AAV-PHP.eB, and AAV-PHP.S. In some embodiments, one or more capsid protein in an AAV is from an AAV-9. Without being bound by theory, typically in AAV, three capsid proteins, VP1, VP2 and VP3 multimerize to form the capsid. The polypeptide sequences of capsid proteins are known in the art, and can also be derived from the genome of the AAV. These can be used as exemplary capsids in the AAV virus compositions disclosed herein. For example, the complete genome of AAV-1 is provided in GenBank Accession No. NC_002077; the complete genome of AAV-2 is provided in GenBank Accession No. NC 001401 and Srivastava et al., Virol., 45: 555-564 {1983): the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Williams, (2006) Mol. Ther., 13(1): 67-76; and the AAV-11 genome is provided in Mori et al., (2004) Virology, 330(2): 375-383. Capsid proteins AAV-PHP.B, AAV-PHP.B2, AAV-PHP.B3, AAV-PHP.A, AAV-PHP.eB, or AAV-PHP.S are provided in Deverman et al., (2016) Nat. Biotech., 34: 204-209 and Chan et al., (2017) Nat. Neurosci., 20: 1172-1179. In some embodiments, the recombinant virus is an AAV comprising one or more AAV1, AAV2, AAV3, AAV4, AAV5, AAV6, AAV7, AAV 8, AAV9, AAV10, and AAV11, AAV 12, AAVrh8, AAVrh10, AAV-DJ, AAV-DJ/8, AAV-PHP.B, AAV-PHP.B2, AAV-PHP.B3, AAV-PHP.A, AAV-PHP.eB, or AAV-PHP.S capsid serotype, or a functional variant thereof. In some embodiments, the recombinant virus is an AAV comprising a combination of capsids from more than one AAV serotype.

In some embodiments, AAV compositions disclosed herein comprise one or more cis-acting sequences directing viral DNA replication (rep), encapsidation/packaging and host cell chromosome integration are contained within the ITRs. In some embodiments, one or more of these sequences may also be present in trans rather than cis, e.g., on a separate plasmid during the virus manufacturing process in a host cell. Typically, three AAV promoters (named p5, p19, and p40 for their relative map locations) drive the expression of the two AAV internal open reading frames encoding rep and cap genes in wild-type virus. In some embodiments, one or more of these promoters and/or open reading frames are present in cis in an AAV vector and/or AAV virion disclosed herein, or are present on separate plasmids during the AAV virus manufacturing process, e.g., in a host cell producing the virus. The two rep promoters (p5 and p19), coupled with the differential splicing of the single AAV intron (at nucleotides 2107 and 2227), may result in the production of four rep proteins (rep 78, rep 68, rep 52, and rep 40) from the rep gene. Rep proteins possess multiple enzymatic properties that are ultimately responsible for replicating the viral genome. The cap gene is typically expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, (1992) Curr. Topics Microbiol. Imm., 158: 97-129.

In some embodiments, the AAV capsid proteins VP1, VP2, VP3 used in the AAV disclosed herein are encoded by or comprise the following sequences:

VP1 nucleic acid (SEQ ID NO: 74):
atggctgccgatggttatcttccagattggctcgaggacaaccttagtgaaggaattcgcgagtggtgggctttgaaacctggagcccctcaacccaa
ggcaaatcaacaacatcaagacaacgctcgaggtcttgtgcttccgggttacaaataccttggacccggcaacggactcgacaagggggagccg
gtcaacgcagcagacgcggcggccctcgagcacgacaaggcctacgaccagcagctcaaggccggagacaacccgtacctcaagtacaacc
acgccgacgccgagttccaggagcggctcaaagaagatacgtcttttgggggcaacctcgggcgagcagtcttccaggccaaaaagaggcttctt
gaacctcttggtctggttgaggaagcggctaagacggctcctggaaagaagaggcctgtagagcagtctcctcaggaaccggactcctccgcggg
tattggcaaatcgggtgcacagcccgctaaaaagagactcaatttcggtcagactggcgacacagagtcagtcccagaccctcaaccaatcgga
gaacctcccgcagccccctcaggtgtgggatctcttacaatggcttcaggtggtggcgcaccagtggcagacaataacgaaggtgccgatggagt
gggtagttcctcgggaaattggcattgcgattcccaatggctgggggacagagtcatcaccaccagcacccgaacctgggccctgcccacctaca
acaatcacctctacaagcaaatctccaacagcacatctggaggatcttcaaatgacaacgcctacttcggctacagcaccccctgggggtattttga
cttcaacagattccactgccacttctcaccacgtgactggcagcgactcatcaacaacaactggggattccggcctaagcgactcaacttcaagctct
tcaacattcaggtcaaagaggttacggacaacaatggagtcaagaccatcgccaataaccttaccagcacggtccaggtcttcacggactcagac
tatcagctcccgtacgtgctcgggtcggctcacgagggctgcctcccgccgttcccagcggacgttttcatgattcctcagtacgggtatctgacgctta
atgatggaagccaggccgtgggtcgttcgtccttttactgcctggaatatttcccgtcgcaaatgctaagaacgggtaacaacttccagttcagctacg
agtttgagaacgtacctttccatagcagctacgctcacagccaaagcctggaccgactaatgaatccactcatcgaccaatacttgtactatctctcaa
agactattaacggttctggacagaatcaacaaacgctaaaattcagtgtggccggacccagcaacatggctgtccagggaagaaactacatacct
ggacccagctaccgacaacaacgtgtctcaaccactgtgactcaaaacaacaacagcgaatttgcttggcctggagcttcttcttgggctctcaatgg
acgtaatagcttgatgaatcctggacctgctatggccagccacaaagaaggagaggaccgtttctttcctttgtctggatctttaatttttggcaaacaag
gaactggaagagacaacgtggatgcggacaaagtcatgataaccaacgaagaagaaattaaaactactaacccggtagcaacggagtcctat
ggacaagtggccacaaaccaccagagtgcccaagcacaggcgcagaccggctgggttcaaaaccaaggaatacttccgggtatggtttggcag
gacagagatgtgtacctgcaaggacccatttgggccaaaattcctcacacggacggcaactttcacccttctccgctgatgggagggtttggaatga
agcacccgcctcctcagatcctcatcaaaaacacacctgtacctgcggatcctccaacggccttcaacaaggacaagctgaactctttcatcaccc
agtattctactggccaagtcagcgtggagatcgagtgggagctgcagaaggaaaacagcaagcgctggaacccggagatccagtacacttcca
actattacaagtctaataatgttgaatttgctgttaatactgaaggtgtatatagtgaaccccgccccattggcaccagatacctgactcgtaatctgtaa
VP2 nucleic acid (SEQ ID NO: 75):
acggctcctggaaagaagaggcctgtagagcagtctcctcaggaaccggactcctccgcgggtattggcaaatcgggtgcacagcccgctaaaa
agagactcaatttcggtcagactggcgacacagagtcagtcccagaccctcaaccaatcggagaacctcccgcagccccctcaggtgtgggatct
cttacaatggcttcaggtggtggcgcaccagtggcagacaataacgaaggtgccgatggagtgggtagttcctcgggaaattggcattgcgattccc
aatggctgggggacagagtcatcaccaccagcacccgaacctgggccctgcccacctacaacaatcacctctacaagcaaatctccaacagca
catctggaggatcttcaaatgacaacgcctacttcggctacagcaccccctgggggtattttgacttcaacagattccactgccacttctcaccacgtg
actggcagcgactcatcaacaacaactggggattccggcctaagcgactcaacttcaagctcttcaacattcaggtcaaagaggttacggacaac
aatggagtcaagaccatcgccaataaccttaccagcacggtccaggtcttcacggactcagactatcagctcccgtacgtgctcgggtcggctcac
gagggctgcctcccgccgttcccagcggacgttttcatgattcctcagtacgggtatctgacgcttaatgatggaagccaggccgtgggtcgttcgtcct
tttactgcctggaatatttcccgtcgcaaatgctaagaacgggtaacaacttccagttcagctacgagtttgagaacgtacctttccatagcagctacgc
tcacagccaaagcctggaccgactaatgaatccactcatcgaccaatacttgtactatctctcaaagactattaacggttctggacagaatcaacaa
acgctaaaattcagtgtggccggacccagcaacatggctgtccagggaagaaactacatacctggacccagctaccgacaacaacgtgtctcaa
ccactgtgactcaaaacaacaacagcgaatttgcttggcctggagcttcttcttgggctctcaatggacgtaatagcttgatgaatcctggacctgctat
ggccagccacaaagaaggagaggaccgtttctttcctttgtctggatctttaatttttggcaaacaaggaactggaagagacaacgtggatgcggac
aaagtcatgataaccaacgaagaagaaattaaaactactaacccggtagcaacggagtcctatggacaagtggccacaaaccaccagagtgc
ccaagcacaggcgcagaccggctgggttcaaaaccaaggaatacttccgggtatggtttggcaggacagagatgtgtacctgcaaggacccattt
gggccaaaattcctcacacggacggcaactttcacccttctccgctgatgggagggtttggaatgaagcacccgcctcctcagatcctcatcaaaaa
cacacctgtacctgcggatcctccaacggccttcaacaaggacaagctgaactctttcatcacccagtattctactggccaagtcagcgtggagatc
gagtgggagctgcagaaggaaaacagcaagcgctggaacccggagatccagtacacttccaactattacaagtctaataatgttgaatttgctgtta
atactgaaggtgtatatagtgaaccccgccccattggcaccagatacctgactcgtaatctgtaa
VP3 nucleic acid (SEQ ID NO: 76):
atggcttcaggtggtggcgcaccagtggcagacaataacgaaggtgccgatggagtgggtagttcctcgggaaattggcattgcgattcccaatgg
ctgggggacagagtcatcaccaccagcacccgaacctgggccctgcccacctacaacaatcacctctacaagcaaatctccaacagcacatctg
gaggatcttcaaatgacaacgcctacttcggctacagcaccccctgggggtattttgacttcaacagattccactgccacttctcaccacgtgactggc
agcgactcatcaacaacaactggggattccggcctaagcgactcaacttcaagctcttcaacattcaggtcaaagaggttacggacaacaatgga
gtcaagaccatcgccaataaccttaccagcacggtccaggtcttcacggactcagactatcagctcccgtacgtgctcgggtcggctcacgagggc
tgcctcccgccgttcccagcggacgttttcatgattcctcagtacgggtatctgacgcttaatgatggaagccaggccgtgggtcgttcgtccttttactgc
ctggaatatttcccgtcgcaaatgctaagaacgggtaacaacttccagttcagctacgagtttgagaacgtacctttccatagcagctacgctcacag
ccaaagcctggaccgactaatgaatccactcatcgaccaatacttgtactatctctcaaagactattaacggttctggacagaatcaacaaacgcta
aaattcagtgtggccggacccagcaacatggctgtccagggaagaaactacatacctggacccagctaccgacaacaacgtgtctcaaccactgt
gactcaaaacaacaacagcgaatttgcttggcctggagcttcttcttgggctctcaatggacgtaatagcttgatgaatcctggacctgctatggccag
ccacaaagaaggagaggaccgtttctttcctttgtctggatctttaatttttggcaaacaaggaactggaagagacaacgtggatgcggacaaagtc
atgataaccaacgaagaagaaattaaaactactaacccggtagcaacggagtcctatggacaagtggccacaaaccaccagagtgcccaagc
acaggcgcagaccggctgggttcaaaaccaaggaatacttccgggtatggtttggcaggacagagatgtgtacctgcaaggacccatttgggcca
aaattcctcacacggacggcaactttcacccttctccgctgatgggagggtttggaatgaagcacccgcctcctcagatcctcatcaaaaacacacc
tgtacctgcggatcctccaacggccttcaacaaggacaagctgaactctttcatcacccagtattctactggccaagtcagcgtggagatcgagtgg
gagctgcagaaggaaaacagcaagcgctggaacccggagatccagtacacttccaactattacaagtctaataatgttgaatttgctgttaatactg
aaggtgtatatagtgaaccccgccccattggcaccagatacctgactcgtaatctgtaa
VP1 Protein (SEQ ID NO: 77):
MAADGYLPDWLEDNLSEGIREWWALKPGAPQPKANQQHQDNARGLVLPGYKYLGPGNGLDKGEPVNA
ADAAALEHDKAYDQQLKAGDNPYLKYNHADAEFQERLKEDTSFGGNLGRAVFQAKKRLLEPLGLVEEAA
KTAPGKKRPVEQSPQEPDSSAGIGKSGAQPAKKRLNFGQTGDTESVPDPQPIGEPPAAPSGVGSLTMAS
GGGAPVADNNEGADGVGSSSGNWHCDSQWLGDRVITTSTRTWALPTYNNHLYKQISNSTSGGSSNDN
AYFGYSTPWGYFDFNRFHCHFSPRDWQRLINNNWGFRPKRLNFKLFNIQVKEVTDNNGVKTIANNLTST
VQVFTDSDYQLPYVLGSAHEGCLPPFPADVFMIPQYGYLTLNDGSQAVGRSSFYCLEYFPSQMLRTGNN
FQFSYEFENVPFHSSYAHSQSLDRLMNPLIDQYLYYLSKTINGSGQNQQTLKFSVAGPSNMAVQGRNYIP
GPSYRQQRVSTTVTQNNNSEFAWPGASSWALNGRNSLMNPGPAMASHKEGEDRFFPLSGSLIFGKQG
TGRDNVDADKVMITNEEEIKTTNPVATESYGQVATNHQSAQAQAQTGWVQNQGILPGMVWQDRDVYLQ
GPIWAKIPHTDGNFHPSPLMGGFGMKHPPPQILIKNTPVPADPPTAFNKDKLNSFITQYSTGQVSVEIEWE
LQKENSKRWNPEIQYTSNYYKSNNVEFAVNTEGVYSEPRPIGTRYLTRNL
VP2 Protein (SEQ ID NO: 78):
TAPGKKRPVEQSPQEPDSSAGIGKSGAQPAKKRLNFGQTGDTESVPDPQPIGEPPAAPSGVGSLTMAS
GGGAPVADNNEGADGVGSSSGNWHCDSQWLGDRVITTSTRTWALPTYNNHLYKQISNSTSGGSSNDN
AYFGYSTPWGYFDFNRFHCHFSPRDWQRLINNNWGFRPKRLNFKLFNIQVKEVTDNNGVKTIANNLTST
VQVFTDSDYQLPYVLGSAHEGCLPPFPADVFMIPQYGYLTLNDGSQAVGRSSFYCLEYFPSQMLRTGNN
FQFSYEFENVPFHSSYAHSQSLDRLMNPLIDQYLYYLSKTINGSGQNQQTLKFSVAGPSNMAVQGRNYIP
GPSYRQQRVSTTVTQNNNSEFAWPGASSWALNGRNSLMNPGPAMASHKEGEDRFFPLSGSLIFGKQG
TGRDNVDADKVMITNEEEIKTTNPVATESYGQVATNHQSAQAQAQTGWVQNQGILPGMVWQDRDVYLQ
GPIWAKIPHTDGNFHPSPLMGGFGMKHPPPQILIKNTPVPADPPTAFNKDKLNSFITQYSTGQVSVEIEWE
LQKENSKRWNPEIQYTSNYYKSNNVEFAVNTEGVYSEPRPIGTRYLTRNL
VP3 Protein (SEQ ID NO: 79):
MASGGGAPVADNNEGADGVGSSSGNWHCDSQWLGDRVITTSTRTWALPTYNNHLYKQISNSTSGGSS
NDNAYFGYSTPWGYFDFNRFHCHFSPRDWQRLINNNWGFRPKRLNFKLFNIQVKEVTDNNGVKTIANNL
TSTVQVFTDSDYQLPYVLGSAHEGCLPPFPADVFMIPQYGYLTLNDGSQAVGRSSFYCLEYFPSQMLRT
GNNFQFSYEFENVPFHSSYAHSQSLDRLMNPLIDQYLYYLSKTINGSGQNQQTLKFSVAGPSNMAVQGR
NYIPGPSYRQQRVSTTVTQNNNSEFAWPGASSWALNGRNSLMNPGPAMASHKEGEDRFFPLSGSLIFG
KQGTGRDNVDADKVMITNEEEIKTTNPVATESYGQVATNHQSAQAQAQTGWVQNQGILPGMVWQDRD
VYLQGPIWAKIPHTDGNFHPSPLMGGFGMKHPPPQILIKNTPVPADPPTAFNKDKLNSFITQYSTGQVSVE
IEWELQKENSKRWNPEIQYTSNYYKSNNVEFAVNTEGVYSEPRPIGTRYLTRNL

In one embodiment, the recombinant virus is an AAV comprising an AAV9 capsid serotype or any mutant or derivative thereof. In some embodiments, the recombinant virus comprises AAV9 capsid proteins VP1, VP2 and VP3. In some embodiments, the recombinant virus is a scAAV.

In some embodiments, a recombinant virus may be used to increase the levels of functional polypeptides in specific cell types. In some embodiments, the virus disclosed herein exhibits higher expression of the transgene sequence in a specific tissue type as compared to the expression of the same virus in a different tissue type. In some embodiments, the virus exhibits higher expression of the transgene sequence in a neuronal tissue, fluid or cell as compared to the expression of the same virus in a non-neuronal tissue, fluid or cell. In some embodiments, a vector disclosed herein exhibits higher expression of the transgene sequence in the presence of a splice modulator compared to the expression of the same vector in the absence of the splice modulator. In some embodiments, the level of expression of the molecule of interest from the recombinant virus in the presence of the splice modulator is greater, e.g., 2, 3, 4, 5, 6, 7, 8, 9, 10, 20, 30, 40, 50, 60, 70, 80, 90, or 100 fold greater, than the level of expression of the molecule of interest from the recombinant virus in the absence of the splice modulator. In some embodiments, the level of expression of the molecule of interest from the recombinant virus in the absence of the splice modulator is greater, e.g., 2, 3, 4, 5, 6, 7, 8, 9, 10, 20, 30, 40, 50, 60, 70, 80, 90, or 100 fold greater, than the level of expression of the molecule of interest from the recombinant virus in the presence of the splice modulator. In some embodiments, the increase in expression of the transgene sequence is measured by an increase in the number of RNA transcripts of the transgene sequence. In some embodiments, the increase in expression of the transgene sequence is measured by PCR. In some embodiments, the increase in expression of the transgene sequence is measured by RT-PCR. In some embodiments, the increase in expression of the transgene sequence is measured by qPCR. In some embodiments, the increase in expression of the transgene sequence is measured by qRT-PCR. In some embodiments, the increase in expression of the transgene sequence is measured by sequencing. In some embodiments, the increase in expression of the transgene sequence is measured by Northern blot analysis. In some embodiments, the increase in expression of the transgene sequence is measured by single-molecule Fluorescence In-Situ Hybridization (FISH). In some embodiments, the increase in expression of the transgene sequence is measured by an increase in the amount of protein encoded by the transgene produced. In some embodiments, the increase in expression of the transgene sequence is measured by an enzyme-linked immunosorbent assay (ELISA). In some embodiments, the increase in expression of the transgene sequence is measured by Western blot analysis. In some embodiments, the increase in expression of the transgene sequence is measured by immunostaining. In some embodiments, the increase in expression of the transgene sequence is measured by more than one of the above listed methods. In some embodiments, the increase in expression of the transgene sequence is measured by the amount of mRNA which includes the second exon. In some embodiments, the increase in expression of the transgene sequence is measured by the amount of mRNA which includes a direct first exon to third exon splice. Exemplary polypeptides produced in the presence or absence of splice modulator from vectors incorporating either an on-switch minigene or an off-switch minigene are depicted in FIG. 3 (in each case, prior to cleavage of the protease cleavage site and/or self-cleaving peptide sequence). It is contemplated that once the polypeptide comprising the protease cleavage site and/or self-cleaving peptide sequence, the sequence(s) are cleaved such that the protein of interest is produced without (or with fewer than 1, 2, 3, 4, 5, 6, 7, 8, 9, or 10 amino acids of) heterologous sequence derived from the minigene or cleavage sequences.

In various embodiments, the target cells of this disclosure may be any mammalian cell type. In some aspects of this disclosure, the nucleic acids and vectors regulate expression in a neuronal tissue or fluid or cell. In some embodiments, the neuronal tissue is the brain. In some embodiments, the neuronal tissue is the frontal lobe of the brain. In some embodiments, the neuronal tissue is the temporal lobe of the brain. In some embodiments, the neuronal tissue is the central nervous system. In some embodiments, the neuronal tissue is the spinal cord. In some embodiments, the neuronal cell is a human neuronal cell. In some embodiments, the neuronal cell is a neuron. In some embodiments, the neuronal cell is an astrocyte. In some embodiments, the neuronal fluid is cerebrospinal fluid. In some embodiments, a non-neuronal tissue is the liver. In some embodiments, the non-neuronal fluid is plasma. In some embodiments, a non-neuronal cell is a hepatocyte. In some embodiments, a non-neuronal cell is a stellate fat storing cell. In some embodiments, a non-neuronal cell is a Kupffer cell. In some embodiments, a non-neuronal cell is a liver endothelial cell. In some embodiments, the non-neuronal fluid is plasma. In some embodiments, the non-neuronal fluid is serum. In some embodiments, the non-neuronal fluid is blood.

Methods of Producing Recombinant Virus

Also disclosed herein, in various embodiments, are methods of producing recombinant virus comprising neuron specific promoters. In some embodiments, nucleic acid sequences, e.g., plasmids encoding an AAV or other viral genome, are used to produce the recombinant virus. In some embodiments, nucleic acid sequences, e.g., plasmids, comprising an AAV rep gene and/or an AAV cap gene are also used in preparing the AAV or other virus. Also disclosed herein are nucleic acid sequences, e.g., plasmids, comprising an adenovirus helper function gene. In some embodiments, the nucleic acids encoding the AAV rep, AAV cap, and/or adenovirus helper genes may be present in the same structure, e.g., a single plasmid, or they may be present in separate structures. In some embodiments, the one or more plasmids are cotransfected with the nucleic acid encoding the AAV vector into competent cells, and the cells are cultured to produce the recombinant virus. In some cases, the plasmids encoding AAV viral genome and AAV rep and/or cap genes are transferred to cells permissible for infection with a helper virus of AAV (e.g., adenovirus, E1-deleted adenovirus or herpesvirus). In some embodiments, the rAAV genome is assembled into infectious viral particles with AAV capsid proteins in the cells after transfection. Techniques to produce rAAV particles, in which an AAV genome to be packaged, rep and cap genes, and helper virus functions are provided to a cell are known in the art and may include, e.g., electroporation. In some embodiments, production of rAAV involves the following components present within a single cell (denoted herein as a packaging cell): a rAAV vector, AAV rep and cap genes separate from (i.e., not in) the rAAV vector, and helper virus functions. Production of pseudotyped rAAV is disclosed in, for example, WO 01/83692 which is incorporated by reference herein in its entirety. In various embodiments, AAV capsid proteins may be modified to enhance delivery of the recombinant vector. Modifications to capsid proteins are generally known in the art. See, for example, US 2005/0053922 and US 2009/0202490, the disclosures of which are incorporated by reference herein in their entirety.

In various embodiments, general principles of viral vector production may be utilized to produce the vectors and virus, e.g., rAAV, disclosed herein. Carter, (1992) Curr. Opinions Biotech., 1533-539; Muzyczka, (1992) Curr. Topics Microbial. Immunol., 158:97-129. Various approaches are disclosed in Ratschin et al., (1984) Mol. Cell. Biol., 4: 2072; Hennonat et al., (1984) Proc. Natl. Acad. Sci. USA, 81: 6466; Tratschin et al., (1985) Mol. Cell. Biol., 5: 3251; McLaughlin et al., (1988) J. Virol., 62: 1963; Lebkowski et al., (1988) Mol. Cell. Biol., 7:349; Samulski et al. (1989) J. Virol., 63:3822-3828; U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., (1995) Vaccine, 13: 1244-1250; Paul et al., (1993) Hum. Gene Ther., 4: 609-615; Clark et al. (1996) Gene Therapy, 3: 1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

An exemplary method of generating a packaging cell is to create a cell line that stably expresses all the necessary components for AAV particle production. For example, a plasmid (or multiple plasmids) encoding a rAAV vector lacking AAV rep and cap genes, AAV rep and cap genes separate from the rAAV vector, and a selectable marker, such as a neomycin resistance gene, are integrated into the genome of a cell. AAV genomes have been introduced into bacterial plasmids by procedures such as GC tailing (Samulski et al., (1982) Proc. Natl. Acad. Sci. USA, 79: 2077-2081), addition of synthetic linkers containing restriction endonuclease cleavage sites (Laughlin et al., (1983) Gene, 23:65-73) or by direct, blunt-end ligation (Senapathy et al., (1984) J. Biol. Chem., 259: 4661-4666). The packaging cell line is then infected with a helper virus such as adenovirus and/or a plasmid encoding a helper virus. The advantages of this method are that the cells are selectable and are suitable for large-scale production of rAAV. Other examples of suitable methods employ adenovirus or baculovirus rather than plasmids to introduce rAAV vectors and/or rep and cap genes into packaging cells.

In some embodiments, a method of producing recombinant virus comprises providing a nucleic acid to be packaged. In some embodiments, the nucleic acid is a plasmid. In other embodiments, the nucleic acid comprises a transgene sequence interposed between a first AAV terminal repeat and a second AAV terminal repeat. In some embodiments, the transgene encodes human progranulin (hPGRN). In some embodiments, the method of producing recombinant virus comprises providing one or more additional nucleic acids. In some embodiments, the one or more additional nucleic acids comprises an AAV rep gene and/or an AAV cap gene. In some embodiments, the one or more additional nucleic acids comprises an AAV rep gene derived from an AAV serotype 1, AAV serotype 2, AAV serotype 3, AAV serotype 4, AAV serotype 5, AAV serotype 6, AAV serotype 7, AAV serotype 8, or AAV serotype 9. In some embodiments, the one or more additional nucleic acids comprises an AAV cap gene derived from an AAV serotype 1, AAV serotype 2, AAV serotype 3, AAV serotype 4, AAV serotype 5, AAV serotype 6, AAV serotype 7, AAV serotype 8, or AAV serotype 9. In some embodiments, the one or more additional nucleic acids comprises one or more of an adenovirus helper function gene.

In some embodiments, the nucleic acids are co-transfected into competent cells or packaging cells. Methods of co-transfection are known in the art, and include, but are not limited to, transfection by lipofectamine, electroporation, and polyethylenimine. Competent cells or packaging cells may be non-adherent cells cultured in suspension or adherent cells. In one embodiment any suitable packaging cell line may be used, such as HeLa cells, HEK 293 cells and PerC.6 cells (a cognate 293 line). In one embodiment, the packaging cells are human cells. In one embodiment, the packaging cells are HEK 293 cells. In one embodiment, the packaging cells are insect cells. In one embodiment, the packaging cells are Sf9 cells. In some embodiments, the method comprises culturing the transfected cells to produce recombinant virus. In some embodiments, the method comprises recovering the recombinant virus. Methods of recovering recombinant virus include, e.g., those disclosed in U.S. Pat. Nos. 6,143,548 and 9,408,904. In some embodiments, recombinant virus is secreted into cell culture media and purified from the media. In some embodiments, packaging cells are lysed, and the contents purified to recover the recombinant virus. In some embodiments, the virus is recovered from the packaging cell by filtration or centrifugation. In some embodiments, the virus is recovered from the packaging cell by chromatography.

In various embodiments, disclosed herein are cells comprising the nucleic acids disclosed herein, cells comprising the vectors disclosed herein, or cells comprising the viruses disclosed herein. The cells comprising the nucleic acids disclosed herein, cells comprising the vectors disclosed herein, or cells comprising the viruses disclosed herein, may be human cells. The cells comprising the nucleic acids disclosed herein, cells comprising the vectors disclosed herein, or cells comprising the viruses disclosed herein, may also be insect cells. In some embodiments, the cells comprising the nucleic acids disclosed herein, cells comprising the vectors disclosed herein, or cells comprising the viruses disclosed herein are HEK293 cells. In some other embodiments, the cells comprising the nucleic acids disclosed herein, cells comprising the vectors disclosed herein, or cells comprising the viruses disclosed herein are Sf9 cells.

In some embodiments, the method of producing recombinant virus comprises transfecting an insect cell. In some embodiments, the method comprises transfecting an insect cell with a baculovirus comprising the nucleic acids as disclosed herein. In some embodiments, the method comprises transfecting an insect cell with baculovirus comprising a nucleic acid comprising a transgene sequence interposed between a first AAV terminal repeat and a second AAV terminal repeat. In some embodiments, the method comprises transfecting an insect cell with a baculovirus comprising one or more additional nucleic acids. In some embodiments, the one or more additional nucleic acids comprises an AAV rep gene and/or an AAV cap gene. In some embodiments, the one or more additional nucleic acids comprises an AAV rep gene derived from an AAV serotype 1, AAV serotype 2, AAV serotype 3, AAV serotype 4, AAV serotype 5, AAV serotype 6, AAV serotype 7, AAV serotype 8, or AAV serotype 9. In some embodiments, the one or more additional nucleic acids comprises an AAV cap gene derived from an AAV serotype 1, AAV serotype 2, AAV serotype 3, AAV serotype 4, AAV serotype 5, AAV serotype 6, AAV serotype 7, AAV serotype 8, or AAV serotype 9.c. In some embodiments, the one or more additional nucleic acids comprises one or more of an adenovirus helper function gene. In some embodiments, the insect cells are cultivated under conditions suitable to produce recombinant virus. In some embodiments, the virus is recovered from the insect cell. In some embodiments, the virus is recovered from the insect cell by filtration or centrifugation. In some embodiments, the virus is recovered from the insect cell by chromatography.

Pharmaceutical Compositions

In various embodiments, pharmaceutical compositions are disclosed. In some embodiments, a pharmaceutical composition comprises one or more nucleic acids, vectors and/or viruses disclosed herein. In some embodiments, the pharmaceutical composition comprises a pharmaceutically acceptable carrier.

The nucleic acids, vectors, and/or recombinant virus according to the present disclosure (e.g., viral particles) can be formulated to prepare pharmaceutically useful compositions. Exemplary formulations include, for example, those disclosed in U.S. Pat. Nos. 9,051,542 and 6,703,237, which are incorporated by reference in their entirety. The compositions of the disclosure can be formulated for administration to a mammalian subject, e.g., a human. In some embodiments, delivery systems may be formulated for intramuscular, intradermal, mucosal, subcutaneous, intravenous, intrathecal, injectable depot type devices, or topical administration.

In some embodiments, when the delivery system is formulated as a solution or suspension, the delivery system is in an acceptable carrier, e.g., an aqueous carrier. A variety of aqueous carriers may be used, e.g., water, buffered water, 0.8% saline, 0.3% glycine, hyaluronic acid and the like. These compositions may be sterilized and/or sterile filtered. The resulting aqueous solutions may be packaged for use as is, or lyophilized. In some embodiments, the lyophilized preparation is combined with a sterile solution prior to administration.

In some embodiments, the compositions, e.g., pharmaceutical compositions, may contain pharmaceutically acceptable auxiliary substances to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. In some embodiments, the pharmaceutical composition comprises a preservative. In some other embodiments, the pharmaceutical composition does not comprise a preservative.

Method of Use and Treatment

Without being bound by theory, the nucleic acids and other embodiments described herein are used in a method of conditionally expressing a molecule (e.g., protein) of interest, said method comprising: contacting an expression system, e.g. a cell comprising the nucleic acid molecule described herein, a vector described herein or a recombinant virus described herein, with a splice modulator, e.g., LMI070, wherein: a) in the presence of said splice modulator, expression of said protein of interest is increased, e.g., 2, 3, 4, 5, 6, 7, 8, 9, 10, 20, 30, 50, or 100 fold greater, relative to the level of expression of said protein of interest in the absence of said splice modulator; and b) in the absence of said splice modulator, expression of said protein of interest is substantially decreased, e.g., e.g., 2, 3, 4, 5, 6, 7, 8, 9, 10, 20, 30, 50 or 100 fold less, relative to the level of expression of said protein of interest in the presence of the splice modulator.

In embodiments the nucleic acids and other embodiments described herein are used in a method of conditionally expressing a protein of interest, said method comprising: contacting an expression system, e.g. a cell comprising the nucleic acid molecule described herein, a vector described herein or a recombinant virus described herein, with a splice modulator, e.g., LMI070, wherein: a) in the absence of said splice modulator, expression of said protein of interest is increased, e.g., 2, 3, 4, 5, 6, 7, 8, 9, 10, 20, 30, 50 or 100 fold greater, relative to the level of expression of said protein of interest in the presence of said splice modulator; and b) in the presence of said splice modulator, expression of said protein of interest is substantially decreased, e.g., e.g., 2, 3, 4, 5, 6, 7, 8, 9, 10, 20, 30, 50 or 100 fold less, relative to the level of expression of said protein of interest in the absence of the splice modulator.

In embodiments, provided is a method of treating a subject in need of a gene therapy, said method comprising administering to said subject a nucleic acid molecule described herein, a vector described herein a recombinant virus described herein, or a pharmaceutical composition described herein. In embodiments, the method further comprises administering to the subject a splice modulator. In embodiments, the splice modulator is administered periodically (e.g., for a time, separated by times of no administration). In embodiments, the method further comprises administering to the subject an amount of a splice modulator, e.g., LMI070, effective to cause at least a 2, 3, 4, 5, 6, 7, 8, 9, 10, 20, 30, 50 or 100 fold increase or decrease in expression of the protein of interest, relative to the expression level of the protein of interest in the absence of the splice modulator.

Without being bound by theory, mutations in the gene encoding neuron-specific proteins such as progranulin may be implicated in neurodegenerative diseases. In some embodiments, the nucleic acids, vectors, and viruses disclosed herein may be administered to increase neuron-specific expression of a wild-type gene whose loss has been implicated in a neurodegenerative disease. For instance, administration may be used to increase levels of functional progranulin polypeptides. Co-treatment with a splice modulator allows for the expression level to be controlled (modulated). In some embodiments, administering a nucleic acid, vector, and/or virus disclosed herein may serve to treat, prevent, delay, slow, a disease, such as for example, frontotemporal dementia. In some embodiments, the nucleic acids, vectors, and viruses disclosed herein are used in conjunction with a splice modulator to modulate expression of the transgene.

As used herein, “frontotemporal dementia” (FTD) is an umbrella term for a diverse group of disorders that primarily affect the frontal and temporal lobes of the brain—the areas generally associated with personality, behavior and language. FTD is typically driven by degeneration of the frontotemporal lobar regions of the brain. In frontotemporal dementia, portions of these lobes shrink (atrophy). Signs and symptoms may vary, depending upon the portion of the brain affected. The most common signs and symptoms of frontotemporal dementia involve extreme changes in behavior and personality. These include increasingly inappropriate actions, loss of empathy and other interpersonal skills, lack of judgment and inhibition, apathy, repetitive compulsive behavior, a decline in personal hygiene, changes in eating habits, predominantly overeating, oral exploration and consumption of inedible objects, and a lack of awareness of thinking or behavioral changes. Rarer subtypes of FTD are characterized by problems with movement, similar to those associated with Parkinson's disease or amyotrophic lateral sclerosis. Since the discovery that several gene mutations can cause both FTD and amyotrophic lateral sclerosis (ALS), it is increasingly being recognized that FTD and ALS share neurodegenerative pathways and may be part of a common spectrum. Mutations in the progranulin gene (GRN) have recently been identified as a major cause of FTD, with majority of the mutations leading to loss of functional hPGRN polypeptide. Babykumari et al., (2017) Brain, 140(12): 3081-3104; Baker et al., (2006) Nature, 442: 916-19; Cruts et al., (2006) Nature, 442: 920-4; Gaweda-Walerych et al, (2018) Neurobiol. Aging, 72:186.e9-186.e12; Galimerti et al., (2018) Expert Opin. Ther. Targets, 22(7):579-585; Wauters et al., (2018) Neurobiol. Aging, 67:84-94; and Mendez, (2018) Neuropsychiatr. Dis. Treat., 26:14:657-662. Methods for detecting mutations in PGRN include, e.g., those disclosed in WO2008/019187.

In various embodiments, the nucleic acids, vectors, and/or viruses disclosed herein may be used in methods of treating a disorder caused by one or more mutations in the gene encoding progranulin. In one embodiment, the term “treating” comprises the step of administering an effective dose, or effective multiple doses, of a composition comprising a nucleic acid, a vector, a recombinant virus, or a pharmaceutical composition as disclosed herein, to an animal (including a human being) in need thereof. If the dose is administered prior to development of a disorder/disease, the administration is prophylactic. If the dose is administered after the development of a disorder/disease, the administration is therapeutic. In embodiments, an effective dose is a dose that detectably alleviates (either eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. The term encompasses but does not require complete treatment (i.e., curing) and/or prevention. In some embodiments, an effective dose comprises 1×10¹⁰ to 1×10¹⁵ vector genome per milliliter (vg/ml) of a virus as disclosed herein. In some embodiments, an effective dose comprises 1×10⁶ to 1×10¹⁰ plaque forming units per milliliter (pfu/ml) of a virus as disclosed herein. In some embodiments, an effective dose comprises 1×10⁶ to 1×10⁹ transducing units per milliliter (TU/ml) of a virus as disclosed herein. Examples of disease states contemplated for treatment are set out herein.

In some embodiments, the mutations in the gene encoding progranulin are deletion mutations. In some embodiments, the mutations in the gene encoding progranulin are null mutations. In some embodiments, the mutations in the gene encoding progranulin are indels. In some embodiments, the mutations in the gene encoding progranulin are loss-of-function mutations. In some embodiments, the mutations in the gene encoding progranulin are knock-out mutations. In some embodiments, the mutations in the gene encoding progranulin results in loss of expression and/or function of the progranulin protein. In some embodiments, a patient in need of treatment with the nucleic acids, vectors, and/or viruses disclosed herein is identified by screening for a progranulin mutation prior to administration. In some embodiments, screening comprises obtaining a sample of cells or tissue from a subject and sequencing or genotyping one or more genetic loci in the sample to check for the presence of a progranulin mutation. In some embodiments, the screening is performed on genetic material from samples such as (but not limited to) saliva, blood, and/or skin cells.

In some embodiments, a method of treating comprises delivering to a subject in need thereof a therapeutically effective amount of a nucleic acid disclosed herein. In some embodiments, a method of treating comprises delivering to a subject in need thereof a therapeutically effective amount of a vector disclosed herein. In some embodiments, a method of treating comprises delivering to a subject in need thereof a therapeutically effective amount of a recombinant virus disclosed herein. In some embodiments, a method of treating comprises delivering to a subject in need thereof a therapeutically effective amount of a pharmaceutical composition disclosed herein. In some embodiments, the disorder is a neurodegenerative disorder. In some embodiments, the disorder is a frontotemporal dementia. In some embodiments, the disorder is Alzheimer's disease. In some embodiments, the disorder is Parkinson's disease. In some embodiments, the disorder is amyotrophic lateral sclerosis (ALS).

In some embodiments, a nucleic acid, vector, recombinant virus, or pharmaceutical composition disclosed herein is used in treating a disorder caused by one or more mutations in the gene encoding progranulin, e.g., a mutation which results in loss of expression and/or function of the progranulin protein. In some embodiments, the disorder is a neurodegenerative disorder. In some embodiments, the disorder is a frontotemporal dementia. In some embodiments, the disorder is Alzheimer's disease. In some embodiments, the disorder is Parkinson's disease. In some embodiments, the disorder is amyotrophic lateral sclerosis (ALS).

In some embodiments, a nucleic acid, vector, recombinant virus, or pharmaceutical compositions disclosed herein is used in the manufacture of a medicament, for treating a subject in need thereof. In embodiments, the subject suffers from a disorder caused by one or more mutations in the gene encoding progranulin, e.g., a mutation which results in loss of expression and/or function of the progranulin protein.

In various embodiments, the nucleic acid, vector, recombinant virus, or pharmaceutical composition disclosed herein may be delivered to the subject in need thereof by an intravenous administration, direct brain administration (e.g., intrathecal, intracerebral, and/or intraventricular administration), intranasal administration, intra-aural administration, or intra-ocular route administration, or any combination thereof. In some embodiments, the nucleic acid, vector, recombinant virus, or pharmaceutical composition is delivered by intrathecal administration. In some embodiments, the nucleic acid, vector, recombinant virus, or pharmaceutical composition is delivered by an intracerebral or intraventricular route of administration. In some embodiments, the administered nucleic acid, vector, recombinant virus, or pharmaceutical composition is ultimately delivered to the brain, spinal cord, peripheral nervous system, and/or CNS, either directly or by transfer after administration to a separate tissue or fluid, e.g., blood.

Without being bound by theory, in some embodiments the methods disclosed herein may rescue cells that carry mutations on a gene coding for a polypeptide, e.g., progranulin, that result in a non-functioning polypeptide. In some embodiments, a method of expressing a molecule, for example a protein or ribonucleic acid (e.g., an siRNA), comprises delivering to a cell a nucleic acid, viral vector, virus, or pharmaceutical composition disclosed herein. In some embodiments, the cell is a neuronal cell. In some embodiments, the cell is a mammalian cell. In some embodiments, the cell is a human cell. In some embodiments, the neuronal cell is a neuron. In some embodiments, delivery is done in vitro. In some embodiments, delivery is done ex vivo. In some embodiments, the delivery is by systemic administration. In some embodiments, the delivery is local. In some embodiments, the delivery is by direct application to the target tissue. In some embodiments, the target tissue is the brain. In some embodiments, the delivery is by injection into the brain. In some embodiments, the delivery is by intrathecal administration. Without being bound by theory, the methods disclosed herein may reduce lipofuscin deposition, astrocyte and microglia activation, and/or inflammation in the brain of a human or mouse with a mutation in the PGRN protein, thus providing potential benefits to subjects in need thereof.

In various embodiments, the nucleic acids, vectors, viruses, and pharmaceutical compositions disclosed herein may be used to treat a disorder, e.g., FTD. In some embodiments, a nucleic acid, vector, virus, and/or pharmaceutical composition disclosed herein may be used in the manufacture of a medicament for treating a disorder, e.g., FTD. In some embodiments, the disorder is caused by one or more mutations in the gene encoding progranulin. In some embodiments, the mutation in the progranulin gene results in a loss of expression of the progranulin protein. In some embodiments, the mutation in the progranulin gene results in loss of function of the progranulin protein. In some embodiments, the use comprises delivering to a subject in need thereof a therapeutically effective amount of a nucleic acid encoding hPGRN, e.g., in a vector, virus, and/or pharmaceutical composition disclosed herein.

Also provided herein is a kit comprising a nucleic acid molecule described herein, a vector described herein, a recombinant virus described herein, a cell described herein, or a pharmaceutical composition described herein; and a splice modulator.

The present disclosure is further illustrated by the following examples that should not be construed as limiting. The contents of all references, patents, and published patent applications cited throughout this application, as well as the Figures, are incorporated herein by reference in their entirety for all purposes.

### EXAMPLES

The following examples are to be considered illustrative and not limiting on the scope of the disclosure disclosed above.

### Example 1. Identification of Splice-Modulator Binding Sites from the Human Genome

A normal Human Fibroblast line (HD1994) was treated with active (LMI070 and splice modulator 2) and inactive analogs (splice modulator 3) or DMSO for 24 hours. The following compound doses were used in both the NSC34 and HD1994 cell line 1:
LMI070 was tested at a concentration of (100 nM) and a high dose of (5 uM)
Splice modulator 2 was tested at 750 nM
Splice modulator 3 was tested at 5 uM

DMSO treatment control was included for both cell lines. There were 3 biological replicates per group.

Total RNA was isolated using the Qiagen RNeasy Mini isolation kit. RNA-Seq libraries were prepared using the Illumina TruSeq RNA Sample Prep kit v2 and sequenced using the Illumina HiSeq2500 platform. Each sample was sequenced on four different lanes belonging to the same flow cells to a length of 2×76 base-pairs (bp). The quality of the generated reads was assessed by running FastQC (version 0.10.1) on the FASTQ files provided by the sequencing lab (data release file for DM00012.txt). The average quality per base in Phred score was computed for each sample. The reads are of excellent quality (mean Phred score >28 for all base positions). A similar quality trend that decreases to the 5′ and 3 ends, was observed, as expected by Illumina chemistry.

A total of 847 million 76-base-pair (bp) paired-end reads were mapped to the Homo sapiens genome (hg19), the human RefSeq (Pruitt et al., 2007) transcripts (release 59, May 3, 2013) using TopHat (2.0.3)

TopHat (2.0.3) alignments against the human genome (hg19) were computed for each of the 15 replicates separately. In order to increase the ability to detect exons, three alignment files (bam files) were pooled for each of the five conditions (DMSO, LMI070 at 5 uM, LMI070 at 100 nM, splice modulator 2 at 750 nM and splice modulator 3 at 5 uM) before the transcript assembly by Cufflinks (2.1.1). After transcript assembly, the exon coordinates were extracted from the transcript gtf files. Exons on alternative chromosomes and on chromosome M were excluded and the strand information was ignored. That yielded 273866 putative exons. Exons that do not intersect any RefSeq exon (release 59, May 3, 2013) are considered as candidates for non annotated splice in events. That results in 19474 candidates. To gain further confidence, overlapping exons were merged in the full set of all RefSeq exons plus the initial 19474 candidates resulting in 229665 non overlapping exons. For this set of exons all possible exon-exon junctions within each RefSeq gene were considered. A junction database was created using R (2.15.2) scripts and bedtools (2.15.0). The first mate of each paired end read was then mapped against the database. Only non annotated exons supported by at least one junction alignment were retained. This excludes in particular candidates not attached to a RefSeq gene. That leaves 10898 final candidates. Sequences for these candidates were extracted from hg19 using bedtools. To assess variability separate Cufflinks assemblies were computed for each replicate and checked whether each candidate is seen in such an assembly. In addition the alignments against the junction database was used to determine the number of junctions that skip over a new exon. The information was used to estimate a splice-in fraction. Further the read coverage for the 10898 candidates was determined for each replicate using bedtools on the TopHat alignments (bam files) and then aggregated within each of the five conditions. The original fastq files were reprocessed with STAR (020201) and aligned against the human genome (hg38). The 10898 candidate exons were lifted over to hg38 using the UCSC genome browser tools—7 candidates could not be lifted. The junctions detected by STAR were mapped to the remaining 10891 candidates and provide an alternative source of junction counts. The final 10 candidates (described in Table 1) for validation were selected from the 10898 putative not annotated exons found in the human SMA RNA Seq dataset as follows 1) STAR aggregated junction counts & TopHat exon coverage=0 for all samples for splice modulator 3 and DMSO (no leakiness); 2) LMI070 and splice modulator 2 STAR aggregated junction counts & TopHat exon coverage >60 (dynamic range); 3) Exon length <100 bp (for feasibility); and 3′end AGA/GTAAG (to confirm presence of splice modulator binding site)

### Example 2. Construction of Minigene Switch

With the design concepts in mind (FIGS. 1A and 1B), a specific minigine ON-switch was designed using the SNX7 gene sequences identified in Example 1. FIG. 2A shows a schematic diagram of the SNX7 locus identified in Example 1 containing a splice modulator (LMI070) exonic target binding site at chromosome: GRCh37:1:99204216:99204359:1 (AGTTTGCAAAGGAAGGAAAGGAGCAGAGACTTGAATGAGCAGAAAATCATTTCAGGGCCTGTTCTC TATGTCCTTGCTATCCCTGTCTTCTGTAGCTATTCTGAAACCATCAACAAAGGAGCACACCATTCCAT CAGCAAAAGA (SEQ ID NO: 80)), as well as an intronic sequence downstream of exon 8 at chromosome:GRCh37:1:99203793:99203946:1 (CTTCCAGAGGAGATTGGAAAACTTGAAGATAAAGTGGAATGTGCTAATAATGCCCTGAAAGCAGATT GGGAGAGATGGAAACAAAATATGCAAAATGATATCAAGTTAGCATTTACAGATATGGCTGAGGAGAA TATCCATTATTATGAACAG (SEQ ID NO: 99)), and 21,251 nucleotides upstream of exon 9 at chromosome:GRCh37:1:99225610:99225687:1 (TGCCTTGCTACGTGGGAGTCATTCCTTACATCACAGACCAACCTTCACTTGGAAGAAGCCTCTGAAG ATAAACCTTAA (SEQ ID NO: 100)). Using these sequence the non-naturally occurring SNX7 minigene was constructed (version 1) (FIG. 2) using exon 8 (called exon A), the 270 nucleotide intron located between exon 8 and the identified cryptic exon comprising the splice modulator binding site (AB), an exon comprising a splice modulator (e.g., LMI070) binding site at its 3′ end (called exon B), and a 407 nucleotide intron fragment between the cryptic exon and exon 9 (shortened from 21,251 nt; BC), and exon 9 (called exon C). Additional modifications were made to the minigene to improve its performance, such as: 1) a Kozak consensus sequence and ATG codon (GCCACCATG) was inserted at position 65 in exon A; 2) All other ATG sequences in the minigene were replaced with TTG; 3) a TA at position 20 of exon A was replaced with AG to make GAAGAAGAA sequence (SEQ ID NO: 69); 4) 1 nt was removed from exon B to create frame shift (number of nucleotides=3n−1) in ORF; 5) T was inserted at position 4 of exon C to create frame shift in ORF resulting in multiple stop codons; 6) TAC at position 9 of exon C was changed to TAA to create earlier termination codon; 7) CAG at position 34 of exon C was changed to ACC to mutate a potential cryptic splice site; 8) CTCT at position 60 of exon C was changed to TAGC to create a Nhe I restriction site; and 9) TAA at the end of exon C was removed to create continuous ORF. This sequence was then inserted into a scAAV vector using molecular cloning techniques. The scAAV was created by combining, AAV2 ITR containing a deletion of trs, followed by a JeT promoter, followed by the SNX7 minigene (see FIG. 2B), followed by a coding sequence for a furin cleavage site (RNRR (SEQ ID NO: 39)) added to the end of exon C, followed by coding sequence for a T2A peptide, followed by a transgene sequence (here, a coding sequence for EGFP without the first ATG); followed by a SV40 late polyadenylation signal, followed by an AAV2 ITR (See FIG. 2C). FIG. 3 shows the predicted mRNA products of the scAAV in the presence or absence of splice modulator.

### Example 3. In Vitro Performance of ON-Switch in HEK293 Cells

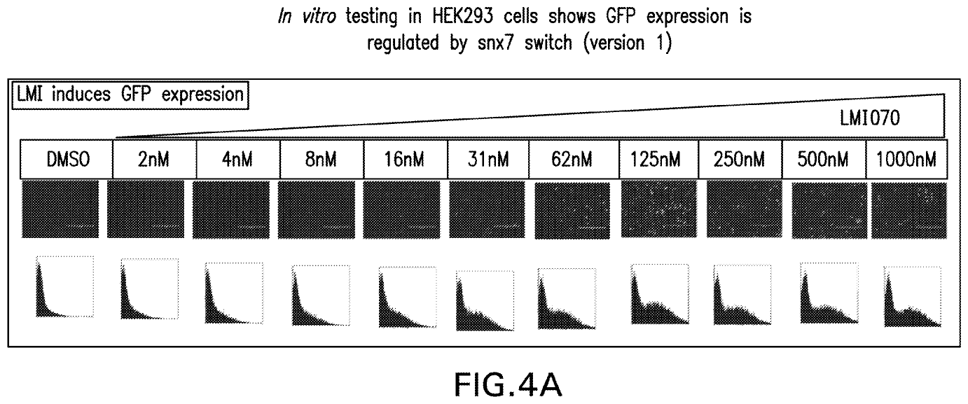

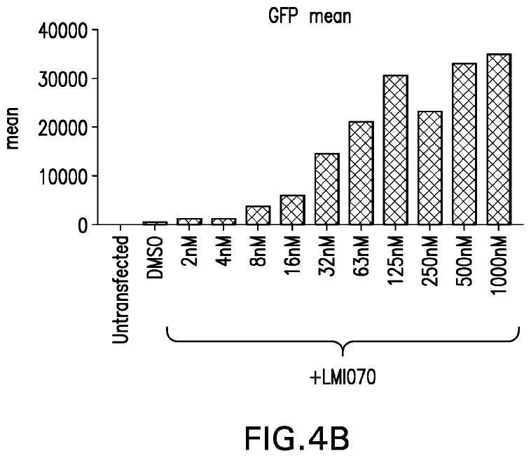

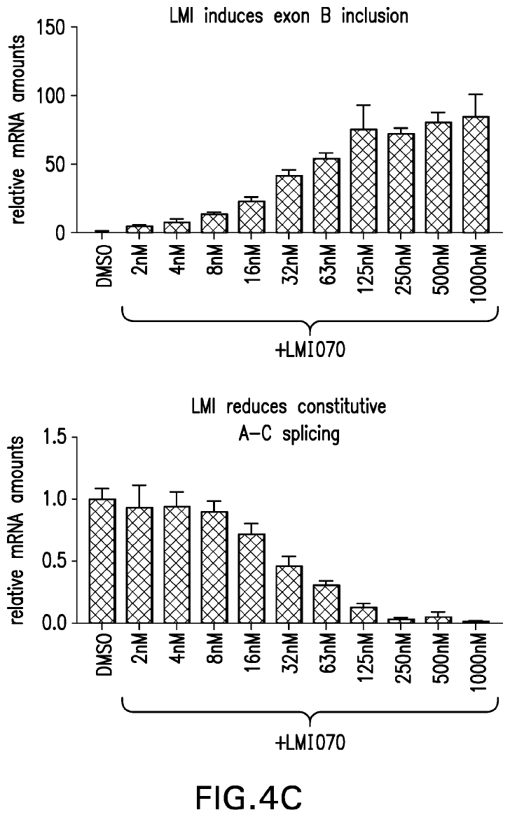

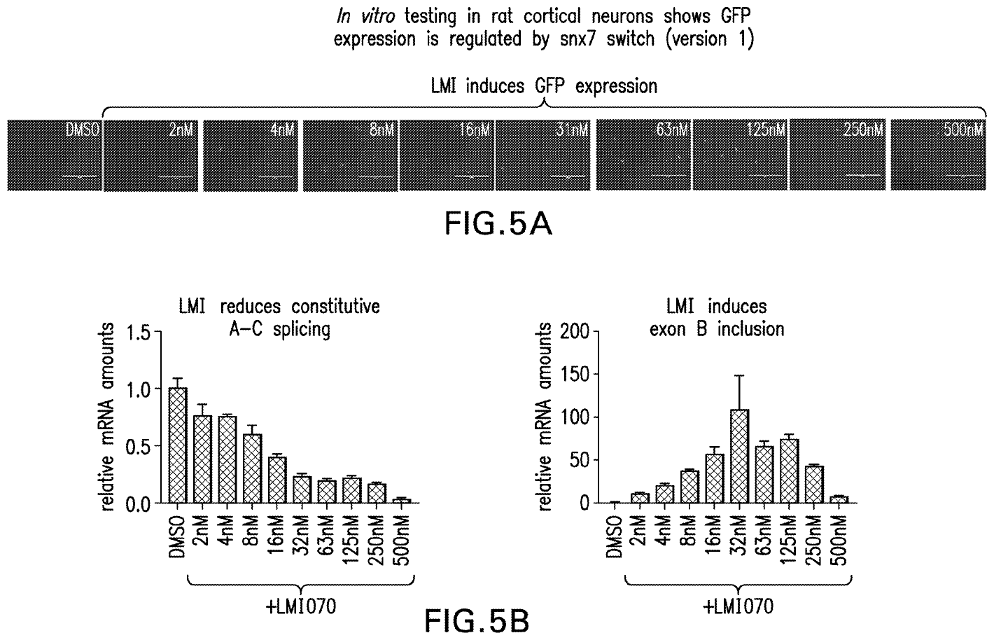

HEK293 cells were maintained in complete DMEM media and seeded in 24-well plate at 100,000 cells per well densite day before transfection. Each well was transfected with 2 ug of pJSNX-GFP plasmid DNA using Lipofectamine2000 (Invitrogen) according to manufacturer's protocol. Transfection media was replaced with complete DMEM 4 hours later. Initial 1 mM stock of LMI070 in DMSO was diluted in 1/1,000-1/500,000 in DMEM to achieve concentrations 2 nm-1 uM when added to the cells in 24 hours later. Control cells received 0.1% DMSO. GFP expression was evaluated 48 hours post transfection using fluorescent microscope. No GFP expression was observed in control DMSO-treated cells (FIG. 4A). For quantitative analysis of GFP expression, cells were trypsinized and analyzed by FACS using SONY SH-800 flow cytometer. Mean fluorescence intensity was used for relative measurement of GFP expression. Control DMSO-treated cells showed no detectable GFP expression, while dose-dependent increase in GFP expression was observed in LMI070-treated samples (FIG. 4B). For RNA splicing analysis, total RNA was extracted from cells using Trizol (Invitrogen) according to manufacturer's protocol. cDNA was synthesized using Superscript III 1st strand supermix for qRT-PCR (Invitrogen). Inclusion of exon B was evaluated using qPCR by measuring amounts of exonB-exonC amplified by CAACAAAGGAGCACACCATTC (SEQ ID NO: 103) and GCGGTTGCGAGGTTTATCT (SEQ ID NO: 104) primers pair as compared to total transgene mRNA amplified by primers specific to exon C, GCGGTTGCGAGGTTTATCT (SEQ ID NO: 104) and CTCTTGCTAAGTGGGAGTCATT (SEQ ID NO: 105). Inclusion of exon B in 125 nM LMI070-treated cells was found to be upregulated 75 times as compared to DMSO-treated cells (FIG. 4C). Amounts of constitutively spliced RNA (i.e. exonA-exonC) was measured using GTGCTAATAATGCCCTGAAAGC (SEQ ID NO: 106) and CCACTTAGCAAGAGCACTGT (SEQ ID NO: 107) primers pair. 1 uM LMI070-treated cells demonstrated 60 times lower exonA-exonC splicing as compared to control DMSO-treated cells.

### Example 4. Regulation of GFP Expression by SNX7 Switch in Rat Cortical Neurons

Primary rat neurons were prepared from dissected rat embryo cortices digested with papain and cultured in complete Neurobasal media in 24-well poly-D-Lysine plates (Corning) at density 150,000 cells/well for 7 days. Half of the media was replaced with fresh media day before transfection. Each well was transfected with 2 ug of pJSNX-GFP plasmid DNA using Lipofectamine2000 (Invitrogen) according to manufacturer's protocol, except cells were washed three times with optiMEM before adding DNA-liposomes cocktails. Transfection media was replaced with conditioned media containing 50% fresh complete Neurobasal media 4 hours later. Next day, 1 mM stock of LMI070 in DMSO was diluted in 1/1,000-1/500,000 in DMEM to achieve concentrations 2 nm-1 uM when added to the cells. Control cells received 0.1% DMSO. GFP expression was evaluated 6 days post transfection using fluorescent microscope. No GFP expression was observed in control DMSO-treated cells (FIG. 4A). For RNA splicing analysis, total RNA was extracted from cells using Trizol (Invitrogen) according to manufacturer's protocol. cDNA was synthesized using Superscript III 1st strand supermix for qRT-PCR (Invitrogen). Inclusion of exon B was evaluated using qPCR by measuring amounts of exonB-exonC amplified by CAACAAAGGAGCACACCATTC (SEQ ID NO: 103) and GCGGTTGCGAGGTTTATCT (SEQ ID NO: 104) primers pair as compared to total transgene mRNA amplified by primers specific to exon C, GCGGTTGCGAGGTTTATCT (SEQ ID NO: 104) and CTCTTGCTAAGTGGGAGTCATT (SEQ ID NO: 105). Inclusion of exon B in 31 nM LMI070-treated cells was found to be upregulated more than 100 times as compared to DMSO-treated cells (FIG. 4B). Amounts of constitutively spliced RNA (i.e. exonA-exonC) was measured using GTGCTAATAATGCCCTGAAAGC (SEQ ID NO: 106) and CCACTTAGCAAGAGCACTGT (SEQ ID NO: 107) primers pair. 500 nM LMI070-treated cells demonstrated 30 times lower exonA-exonC splicing as compared to control DMSO-treated cells.

### Example 5. Regulatable Expression of Human Progranulin in Rat Cortical Neurons by SNX7 Switch

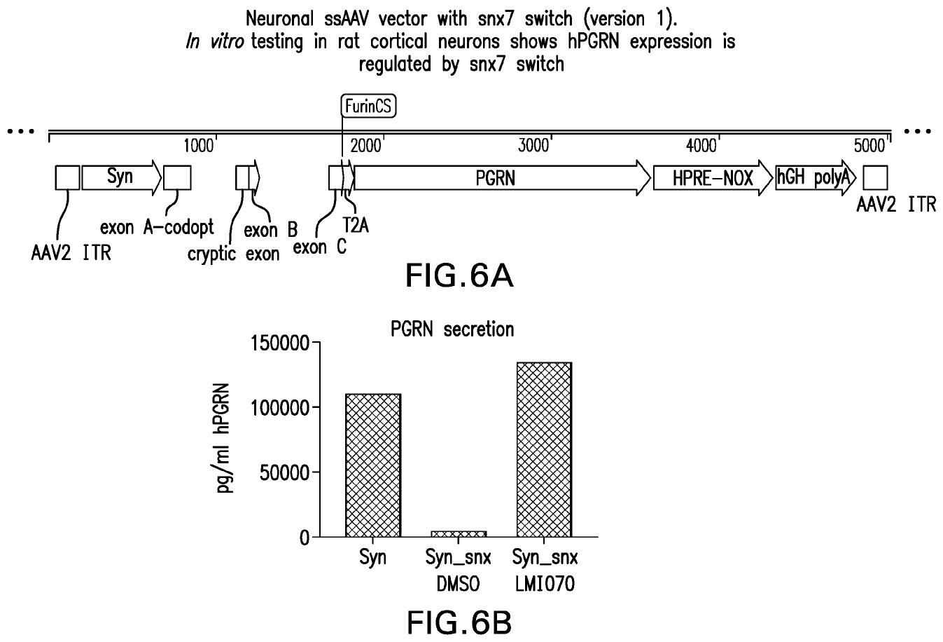

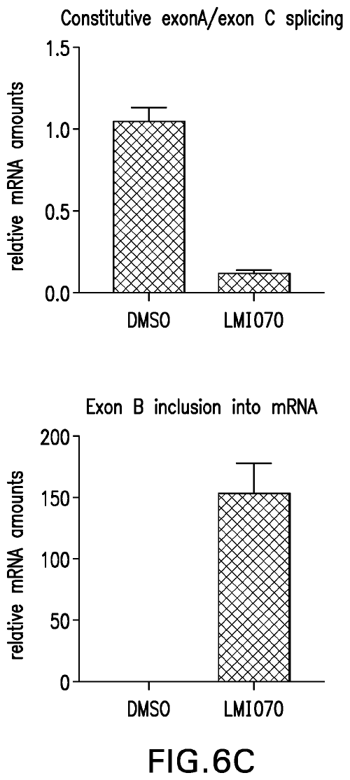

Primary rat neurons were prepared from dissected rat embryo cortices digested with papain and cultured in complete Neurobasal media in 24-well poly-D-Lysine plates (Corning) at density 150,000 cells/well for 7 days. Half of the media was replaced with fresh media day before transfection. Each well was transfected with 2 ug of pSyn-snx7-PGRN (FIG. 6A) or control pSyn-PGRN plasmids, which do not contain snx7 minigene, using Lipofectamine2000 (Invitrogen) according to manufacturer's protocol, except cells were washed three times with optiMEM before adding DNA-liposomes cocktails. Transfection media was replaced with conditioned media containing 50% fresh complete Neurobasal media 4 hours later. Next day, 1 mM stock of LMI070 in DMSO was diluted in 1/10,000 in DMEM to achieve concentrations 100 nm when added to the cells. Control cells received 0.01% DMSO. hPGRN expression was measured 6 days post transfection using TR-FRET assay. In pSyn-snx7-PGRN transfected cells, expression of hPGRN was induced by LMI070 more than 30 times comparing to DMSO-treated control (FIG. 6B). For RNA splicing analysis, total RNA was extracted from cells using Trizol (Invitrogen) according to manufacturer's protocol. cDNA was synthesized using Superscript III 1st strand supermix for qRT-PCR (Invitrogen). Inclusion of exon B was evaluated using qPCR by measuring amounts of exonB-exonC junction amplified by CAACAAAGGAGCACACCATTC (SEQ ID NO: 103) and GCGGTTGCGAGGTTTATCT (SEQ ID NO: 104) primers pair as compared to total transgene mRNA amplified by primers specific to exon C, GCGGTTGCGAGGTTTATCT (SEQ ID NO: 104) and CTCTTGCTAAGTGGGAGTCATT (SEQ ID NO: 105). Inclusion of exon B in LMI070-treated cells was found to be upregulated more than 150 times as compared to DMSO-treated cells for pSyn-snx7-PGRN transfected cells (FIG. 6C). Amounts of constitutively spliced RNA (i.e. exonA-exonC) was measured using GTGCTAATAATGCCCTGAAAGC (SEQ ID NO: 106) and CCACTTAGCAAGAGCACTGT (SEQ ID NO: 107) primers pair. LMI070-treated cells demonstrated 10 times lower exonA-exonC splicing in pSyn-snx7-PGRN transfected cells as compared to DMSO-treated cells.

### Example 6. Modification of Minigene to Reduce Size and Eliminate Peptide Expression in the Absence of LMI070

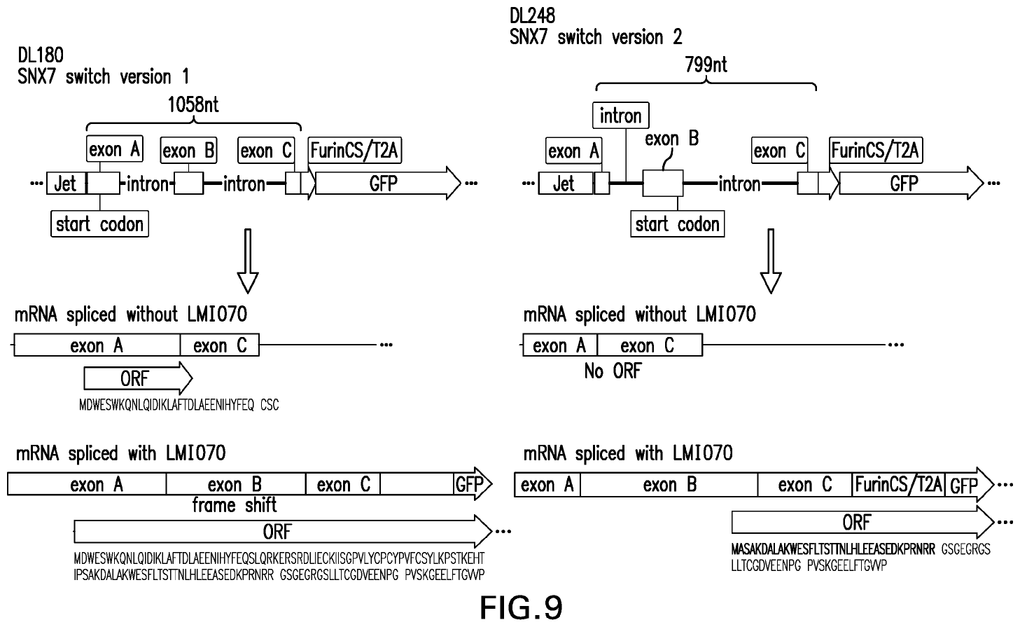

The SNX7 minigene was further modified to reduce the overall size and eliminate peptide expression in the absence LMI070. In particular, exon A was shortened 109 nt to 53 nt while the region adjacent to the 3′ splice site was kept, the resulting exon A has the sequence of SEQ ID NO: 96. First intron was shorted from 150 nt to 120 nt while splice sites and branch point were preserved. The resulting first intron has the sequence of SEQ ID NO: 97. The region containing the start codon in exon A of the first version of SNX7 minigene was deleted, and a start codon was constructed by changing TC to GG in exon B of the new version of SNX7 minigene. The resulting exon B has the sequence of SEQ ID NO:98. By switching start codon to Exon B, protein expression occurs only in the presence of LMI070. The second intron was kept the same, as it was found that this sequence contains essential cis elements. The sequence of the modified SNX minigene (version 2) is shown in SEQ ID NO: 94. FIG. 9 shows schematic diagram of the new version (version 2) of minigene as compared to the previous version of SNX7 minigene (version 1).

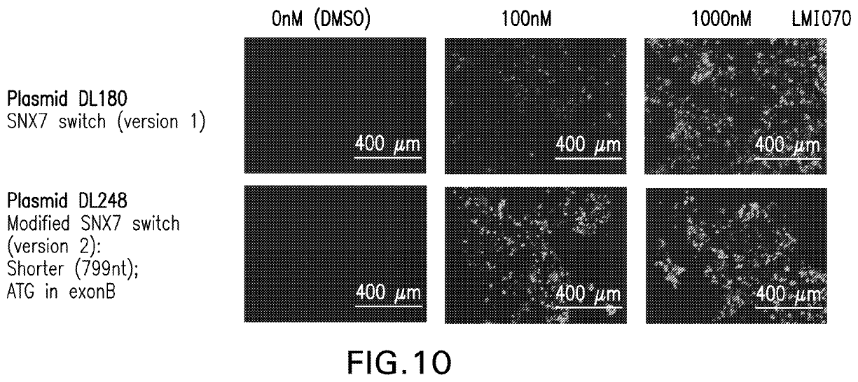

The modified SNX7 minigene (version 2) was inserted into a scAAV vector using molecular cloning techniques. The sequence of the vector comprising the modified SNX7 minigene (version 2) is shown in SEQ ID NO: 95. HEK293 cells were maintained in complete DMEM media and seeded in 24-well plate at 100,000 cells per well densite day before transfection. Each well was transfected with 2 ug of plasmid DNA DL180 containing SNX7 switch version 1 or plasmid DL182 containing SNX7 version 2 using Lipofectamine2000 (Invitrogen) according to manufacturer's protocol. Transfection media was replaced with complete DMEM 4 hours later. Initial 1 mM stock of LMI070 in DMSO was diluted in 1/1,000-1/500,000 in DMEM to achieve concentrations 100 nm-1 uM when added to the cells in 24 hours later. Control cells received 0.1% DMSO. GFP expression was evaluated 48 hours post transfection using fluorescent microscope. FIG. 10 shows that the modified SNX7 minigene (version 2) is more sensitive than the previous version.

### Example 7. Oral Administration of LMI070 Switches on Transgene Expression in Mouse Brain in Time Dependent Manner

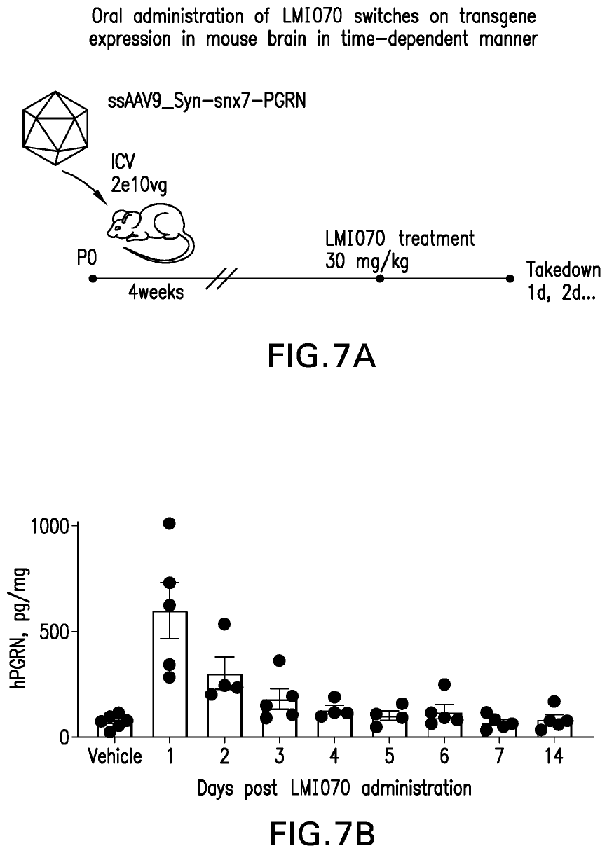

ssAAV9 viral vector encoding hPGRN under control of synapsin promoter with SNX7 switch (version 1) was produced in HEK293 cells and purified by iodixanol. 2e10vg of AAV vector in 2 uL was injected ICV in C57Bl/6 neonatal mice at P0. At 4 weeks of age, 30 mg/kg of LMI070 or vehicle control was administered orally through gavage. 4-6 mice per group were taken down at specified time points (FIG. 7A). After transcardial perfusion with PBS posterior half of the left hemisphere was homogenyzed in Precellys tube. TR-FRET assay was used for measurement of human PGRN expressed from AAV vector. Results indicate rapid and transient induction of hPGRN expression in the brain after 24 hours post LMI070 administration. The transgenic protein expression returned to untreated levels after 4 days post LMI070 administration (FIG. 7B).

### Example 8. LMI070 Switch on Transgene Expression In Vivo in Dose Dependent Manner

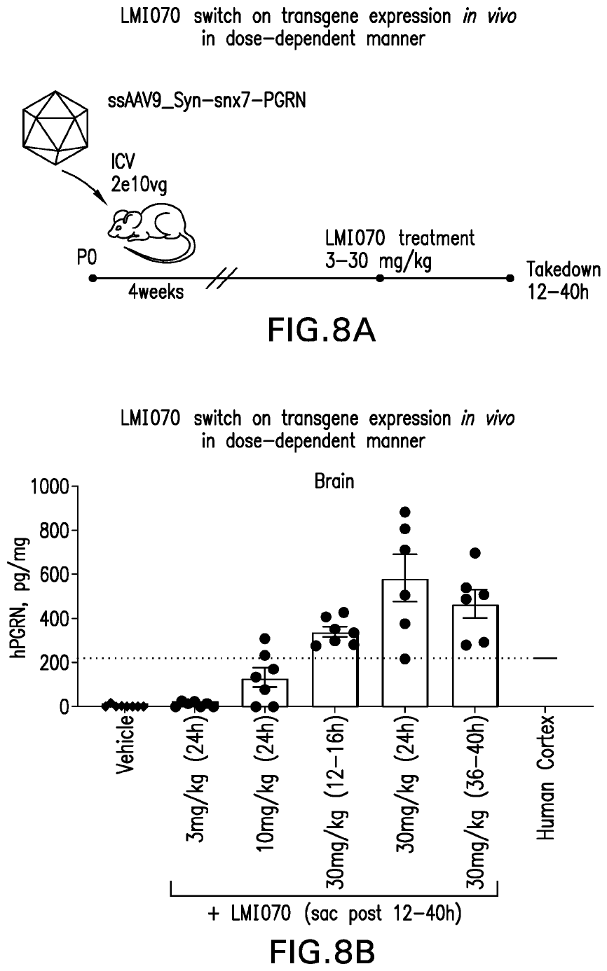

ssAAV9 viral vector encoding hPGRN under control of synapsin promoter with SNX7 switch (version 1) was produced in HEK293 cells and purified by iodixanol. 2e10vg of AAV vector in 2 uL was injected ICV in FVB neonatal mice at P0. At 4 weeks of age, 3, 10 or 30 mg/kg of LMI070 or vehicle control was administered orally through gavage. 6-7 mice per group were taken down at specified time points (FIG. 8A). After transcardial perfusion with PBS posterior half of the left hemisphere was homogenyzed in Precellys tube. TR-FRET assay was used for measurement of human PGRN expressed from AAV vector. Sample of human cortex was used as a control for physiological PGRN levels (˜200 pg/mg). Results indicate rapid (12 hours post LMI070 administration) accumulation of transgenic hPGRN in the brain, which starts to decline at 24 hour point. Transgene expression demonstrated dose response to LMI070 administration.

1. A nucleic acid molecule comprising a minigene linked to a transgene encoding a protein of interest, wherein the minigene comprises:
a. A first exon;
b. A first intron;
c. A second exon;
d. A second intron; and
e. A third exon;
wherein said second exon comprises a splice modulator binding sequence and wherein, in the presence of a splice modulator, said second exon is included in an mRNA product of the nucleic acid, and in the absence of said splice modulator, said second exon is not included in an mRNA product of the nucleic acid.

2. The nucleic acid molecule of claim 1, wherein the third exon comprises a stop codon that is in frame in the mRNA product of the nucleic acid produced in the absence of the splice modulator and which is not in frame in the mRNA product of the nucleic acid produced in the presence of the splice modulator.

3. The nucleic acid molecule of claim 1, wherein the second exon comprises a stop codon that is in frame in the mRNA product of the nucleic acid produced in the presence of the splice modulator.

4. The nucleic acid molecule of claim 1, wherein the first and the third exons do not comprise a start codon, and wherein the second exon comprises a start codon.

5. The nucleic acid molecule of any one of claims 1-4, comprising a sequence encoding a protease cleavage site disposed between the minigene and the transgene.

6. The nucleic acid molecule of claim 5, wherein said protease cleavage site is cleaved by a mammalian protease.

7. The nucleic acid molecule of claim 6, wherein the mammalian protease is furin, PCSK1, PCSK5, PCSK6, PCSK7, cathepsin B, Granzyme B, Factor XA, Enterokinase, genenase, sortase, precission protease, thrombin, TEV protease, or elastase 1.

8. The nucleic acid molecule of any one of claims 4-7, wherein the protease cleavage site comprises a polypeptide having an cleavage motif selected from the group consisting of RX(K/R)R consensus motif, RXXX[KR]R consensus motif, RRX consensus motif, RNRR (SEQ ID NO: 39), I-E-P-D-X consensus motif (SEQ ID NO: 35), Glu/Asp-Gly-Arg, Asp-Asp-Asp-Asp-Lys (SEQ ID NO: 36), Pro-Gly-Ala-Ala-His-Tyr (SEQ ID NO: 37), LPXTG/A consensus motif, Leu-Glu-Val-Phe-Gln-Gly-Pro (SEQ ID NO: 38), Leu-Val-Pro-Arg-Gly-Ser (SEQ ID NO: 40), E-N-L-Y-F-Q-G (SEQ ID NO: 41), and [AGSV]-x (SEQ ID NO: 42).

9. The nucleic acid molecule of any one of claims 4-8, wherein said cleavage site is cleaved by furin.

10. The nucleic acid molecule of claim 9, wherein the protease cleavage site cleaved by furin is
(SEQ ID NO: 39)
RNRR;
(SEQ ID NO: 43)
RTKR;
(SEQ ID NO: 45)
GTGAEDPRPSRKRRSLGDVG;
(SEQ ID NO: 47)
GTGAEDPRPSRKRR;
(SEQ ID NO: 49)
LQWLEQQVAKRRTKR;
(SEQ ID NO: 51)
GTGAEDPRPSRKRRSLGG;
(SEQ ID NO: 53)
GTGAEDPRPSRKRRSLG;
(SEQ ID NO: 55)
SLNLTESHNSRKKR;
or
(SEQ ID NO: 57)
CKINGYPKRGRKRR.

11. The nucleic acid molecule of claim 10, wherein the protease cleavage site cleaved by furin comprises RNRR (SEQ ID NO: 39).

12. The nucleic acid molecule of claim 11, wherein the sequence encoding the protease cleave site comprises, e.g., consists of, CGCAACCGCCGC (SEQ ID NO: 19).

13. The nucleic acid molecule of any one of claims 1-12, comprising a sequence encoding a self-cleaving peptide disposed between the minigene and the transgene, optionally wherein the self-cleaving peptide cleaves within 1, 2, 3, 4, 5, 6, 7, 8, 9, or 10 amino acids of the N-terminus of the protein of interest.

14. The nucleic acid molecule of claim 13, wherein the self-cleaving peptide is a 2A peptide, optionally selected from a T2A peptide, a P2A peptide, a E2A peptide and a F2A peptide.

15. The nucleic acid molecule of any one of claims 13-14, wherein the self-cleaving peptide comprises a T2A peptide.

16. The nucleic acid molecule of any one of claims 13-15, wherein the self-cleaving peptide comprises EGRGSLLTCGDVEENPGP (SEQ ID NO: 61), optionally wherein the self-cleaving peptide comprises (GSG)EGRGSLLTCGDVEENPGP (SEQ ID NO: 59).

17. The nucleic acid molecule of any one of claims 1-16, wherein the splice modulator binding sequence is located at the 3′ terminus of the second exon.

18. The nucleic acid molecule of any one of claims 1-17, wherein the splice modulator binding sequence comprises, e.g., consists of, AGA and the splice modulator is 5-(1H-Pyrazol-4-yl)-2-(6-((2,2,6,6-tetramethylpiperidin-4-yl)oxy)pyridazin-3-yl)phenol (LMI070).

19. The nucleic acid molecule of any one of claims 1-18, wherein the second exon comprises, e.g., consists of a sequence selected from:
a.
(SEQ ID NO: 1)
CCTTGCTATCCCTGTCTTCTGTAGCTATTCTGAAACCATCAACAAAGGAGC
ACACCATTCCATCAGCAAAAGA;
b.
(SEQ ID NO: 2)
GTAATTAGCTGAGAAGGAAGATCTGAAGGTTTAACGAGAGAGGGCGAGAGA
TACAAAATATCTGCTAGGAGA;
c.
(SEQ ID NO: 3)
GGATTGTTTGTATTCCTGCCAATGATTTGTGAGACAGTCTGTTCCCCACAT
CCTCGTCAACAGA;
d.
(SEQ ID NO: 4)
CTTTCTGACATCTTAACGAGGCAATACAGAGAGACGAATTTTCATCAGTTT
GTTCAGGGAGACACATATAACAAAAGA;
e.
(SEQ ID NO: 5)
ATCCATACATACTTAATGCTGAAATGTGAAGGGCTGAGAAAAAAGAAAAG
A;
f.
(SEQ ID NO: 6)
AATTGGAAACATCGAGGGAAAATGGGCTTTTTATTATTAAAACAAAACCTC
AGTATTATCACTTAGAAACCTGAAATTGAACTCCAAAAGCCAAAGA;
g.
(SEQ ID NO: 7)
AAGAATGTTCCTTTTGTGAAGAATGACTTAAGGAAGATTCATGATGACTGA
GTGTGCCCGTGTGGAACTTTAGGACATAGATGCACTCCTACAGA;
h.
(SEQ ID NO: 8)
TTGTCCTTCACTCCGTACTCCAGTTGGCCAAGCATAGGTCGCATGCCAGGG
TCAAGGAGACTAAGGGAGA;
i.
(SEQ ID NO: 9)
GACATACAGACATGGCAGCCCCTAGCATGTGTATCCTAAGA;
j.
(SEQ ID NO: 10)
ACATACAGACATGGCAGCCCCTAGCATGTGTATCCTAAGA;
k.
(SEQ ID NO: 80)
AGTTTGCAAAGGAAGGAAAGGAGCAGAGACTTGAATGAGCAGAAAATCATT
TCAGGGCCTGTTCTCTATGTCCTTGCTATCCCTGTCTTCTGTAGCTATTCT
GAAACCATCAACAAAGGAGCACACCATTCCATCAGCAAAAGA
and
l. A fragment or mutant of any of (a) to (k) having at least 90%, at least 95% at least 96%, at least 97%, at least 98% or at least 99% identity thereto.

20. The nucleic acid molecule of any one of claims 1-19, wherein the second exon comprises a sequence derived from an exon of SNX7, optionally wherein the sequence is derived a cryptic exon of SNX7.

21. The nucleic acid molecule of any one of claims 1-20, wherein the second exon comprises, e.g., consists of,
a.
(SEQ ID NO: 16)
AGTTTGCAAAGGAAGGAAAGGAGCAGAGACTTGATTGAGCAGAAAATCATT
TCAGGGCCTGTTCTCTATTGTCCTTGCTATCCTGTCTTCTGTAGCTATCTG
AAACCATCAACAAAGGAGCACACCATTCCATCAGCAAAAGA;
b. a fragment of SEQ ID NO: 16; or
c. a mutant sequence of SEQ ID NO: 16 or a fragment thereof having at least 90%, at least 95% at least 96%, at least 97%, at least 98% or at least 99% identity thereto.

22. The nucleic acid molecule of any one of claims 1-20, wherein the second exon comprises, e.g. consists of,
a.
(SEQ ID NO: 98)
AGTTTGCAAAGGAAGGAAAGGAGCAGAGACTTGATTGAGCAGAAAATCATT
TCAGGGCCTGTTCTCTATTGTCCTTGCTATCCTGTCTTCTGTAGCTATCTG
AAACCATCAACAAAGGAGCACACCATGGCATCAGCAAAAGA;
b. a fragment of SEQ ID NO: 98; or
c. a mutant sequence of SEQ ID NO: 98 or a fragment thereof having at least 90%, at least 95% at least 96%, at least 97%, at least 98% or at least 99% identity thereto.

23. The nucleic acid molecule of any one of claims 1-2 and 4-22, wherein the second exon consists of 3n−1 nucleotides, where n is an integer.

24. The nucleic acid molecule of any one of claims 1-21, wherein the first exon comprises:
a. One or more, e.g., three, GAA repeats (SEQ ID NO: 69) (for example, comprises GAAGAAGAA (SEQ ID NO: 69));
b. A Kozak sequence (e.g., a Kozak sequence comprising GCCACC (SEQ ID NO: 70)); or
c. Both (a) and (b).

25. The nucleic acid molecule of any one of claims 1-23, wherein the minigene has been modified to:
a. Remove or mutate all but a single start codon, e.g., an ATG start codon;
b. Remove or mutate all cryptic splice donor and splice acceptor sequences other than those at the termini of the first exon, the second exon and the third exon.

26. The nucleic acid molecule of claim 25, wherein the single start codon is disposed within the first exon.

27. The nucleic acid molecule of claim 25, wherein the single start codon is disposed within the second exon.

28. The nucleic acid molecule of any one of claims 1-27, wherein the minigene comprises fewer than 2000, fewer than 1900, fewer than 1800, fewer than 1700, fewer than 1600, fewer than 1500, fewer than 1400, fewer than 1300, fewer than 1200, fewer than 1100, fewer than 1000, fewer than 900, fewer than 800, fewer than 700, fewer than 600 or fewer than 500 nucleotides.

29. The nucleic acid molecule of any one of claims 1-27, wherein the minigene comprises between about 2500 and about 500 nucleotides, e.g., between about 2000 and about 500 nucleotides, e.g., between about 1500 and about 600 nucleotides, e.g., between about 1200 and about 700 nucleotides, e.g., between about 1100 and about 800 nucleotides, e.g. between about 800 and about 500 nucleotides, e.g. between 800 and about 600 nucleotides, e.g. between about 800 and about 700 nucleotides.

30. The nucleic acid molecule of any one of claims 1-2 and 4-29, wherein the minigene comprises, e.g., consists of, SEQ ID NO: 71 or SEQ ID NO: 94, or a sequence with at least 90, 91, 92, 93, 94, 95, 96, 97, 98, or 99% identity thereto, or a functional fragment thereof.

31. A nucleic acid molecule, comprising (a) a transgene encoding a protein of interest, and (b) a minigene comprising, e.g., consisting of, SEQ ID NO: 71 or SEQ ID NO: 94, or a sequence with at least 90, 91, 92, 93, 94, 95, 96, 97, 98, or 99% identity thereto, or a functional fragment thereof.

32. The nucleic acid molecule of claim 31, further comprising a sequence encoding a furin cleavage site, said sequence comprising SEQ ID NO: 19, and a sequence encoding a self-cleaving peptide, said sequence comprising SEQ ID NO: 20, optionally wherein the minigene is disposed 5′ to the sequence encoding the furin cleavage site (e.g., immediately 5′ to the sequence encoding the furin cleavage site), the sequence encoding the furin cleavage site is disposed 5′ to the sequence encoding the self-cleaving peptide (e.g., immediately 5′ to the sequence encoding the self-cleaving peptide), and the sequence encoding the self-cleaving peptide is disposed 5′ to the transgene (e.g., immediately 5′ to the transgene).

33. The nucleic acid molecule of any one of claims 1-32, further comprising a promoter operably linked to the minigene and transgene, optionally wherein said promoter is disposed 5′ to the minigene.

34. The nucleic acid molecule of claim 33, wherein the promoter is a JeT promoter, a CBA promoter, a PGK promoter, or a synapsin promoter, or any promoter that does not comprise an intron.

35. The nucleic acid molecule of any one of claims 1-34, further comprising a post-transcriptional regulatory element.

36. The nucleic acid molecule of claim 35, wherein the post-transcriptional regulatory element (PRE) comprises a PRE derived from hepatitis B (HPRE), bat (BPRE), ground squirrel (GSPRE), arctic squirrel (ASPRE), duck (DPRE), chimpanzee (CPRE) and wooly monkey (WMPRE) or woodchuck (WPRE), optionally wherein said post-transcriptional regulatory element is disposed 3′ to the transgene.

37. The nucleic acid molecule of claim 35, wherein the post-transcriptional regulatory element comprises SEQ ID NO: 72, SEQ ID NO: 73, or SEQ ID NO: 88.

38. The nucleic acid molecule of any one of claims 1-37, wherein said construct further comprises a polyadenylation signal (polyA), optionally wherein said polyA is disposed 3′ to the transgene.

39. The nucleic acid molecule of claim 38, wherein the poly A signal is an SV40 polyA, human growth hormone (HGH) polyA, or bovine growth hormone (BGH) polyA, a beta-globin polyA, an alpha-globin polyA, an ovalbumin polyA, a kappa-light chain polyA, and a synthetic polyA.

40. The nucleic acid molecule of any one of claims 38-39, wherein the polyA comprises, e.g., consists of, SEQ ID NO: 22.

41. A vector comprising a nucleic acid according to any one of claims 1-40.

42. The vector of claim 41, wherein the vector is a DNA vector, optionally a circular vector, optionally a plasmid.

43. The vector of claim 41 or 42, wherein the vector is double stranded or single stranded.

44. The vector of any one of claims 41-43, wherein the vector is double stranded.

45. The vector of any one of claims 41-44, wherein the vector is a viral vector.

46. The vector of claim 45, wherein the viral vector is an adeno-associated viral (AAV) vector, chimeric AAV vector, adenoviral vector, retroviral vector, lentiviral vector, DNA viral vector, herpes simplex viral vector, baculoviral vector, or any mutant or derivative thereof.

47. The vector of claim 46, wherein the viral vector is a recombinant AAV vector, optionally a self-complementary AAV (scAAV) vector.

48. The vector of claim 47, wherein the recombinant AAV vector comprises one or more inverted terminal repeats (ITRs), optionally wherein the ITRs are AAV2 ITRs, optionally wherein the AAV vector comprises two ITRs, optionally wherein the two ITRs comprise SEQ ID NO: 12 and SEQ ID NO: 23.

49. The vector of any one of claims 41-48, wherein the vector comprises, e.g. from 5′ to 3′:
a. an ITR, optionally an AAV2 ITR, optionally, wherein the ITR has been modified to comprise a deletion of a terminal resolution site, optionally comprising SEQ ID NO: 12;
b. a promoter, optionally a JeT promoter comprising or consisting of SEQ ID NO: 13;
c. a nucleic acid molecule of any one of claims 1-32;
d. a polyA signal, optionally comprising or consisting of SEQ ID NO: 22; and
e. an ITR, optionally an AAV2 ITR, optionally comprising or consisting of SEQ ID NO: 23.

50. A recombinant virus comprising the nucleic acid of any one of claims 1-40, or the vector of any one of claims 41-49.

51. The recombinant virus of claim 50, wherein the recombinant virus is an adeno-associated virus (AAV), chimeric AAV, adenovirus, retrovirus, lentivirus, DNA virus, herpes simplex virus, baculovirus, or any mutant or derivative thereof.

52. The recombinant virus of claim 51, wherein the virus is an AAV.

53. The recombinant virus of claim 52, wherein the AAV comprises one or more of an AAV1, AAV2, AAV3, AAV4, AAV5, AAV6, AAV7, AAV 8, AAV9, AAV10, and AAV11, AAV12, AAVrh8, AAVrh10, AAVrh36, AAVrh37, AAV-DJ, AAV-DJ/8, AAV.Anc80, AAV.Anc80L65, AAV-PHP.B, AAV-PHP.B2, AAV-PHP.B3, AAV-PHP.A, AAV-PHP.eB, and AAV-PHP.S capsid serotype, or a variant thereof, e.g., a combination of capsids from more than one AAV serotype.

54. The recombinant virus of claim 52, wherein the AAV comprises an AAV9 capsid serotype or any mutant or derivative thereof.

55. The recombinant virus of claim 54, comprising AAV9 capsid proteins VP1, VP2, and VP3, e.g., as encoded by SEQ ID NO: 74, SEQ ID NO: 75, and SEQ ID NO: 76, respectively, or comprising an amino acid sequence of SEQ ID NO: 77, SEQ ID NO: 78, SEQ and ID NO: 79, respectively.

56. The recombinant virus of any one of claims 50-55, wherein the AAV comprises a self-complementary AAV (scAAV) vector or a single-stranded AAV(ssAAV) vector.

57. A cell comprising the nucleic acid molecule of any one of claims 1-40, the vector of any one of claims 41-49 or the recombinant virus of any one of claims 50-56.

58. The cell of claim 57, wherein the cell is a human cell.

59. The cell of any one of claims 57-58, wherein the cell is a neuron or astrocyte.

60. The cell of any one of claims 57-59, wherein when the cell comprises a splice modulator, e.g., LMI070, the level of expression of the protein of interest is greater, e.g., 2, 3, 4, 5, 6, 7, 8, 9, 10, 20, 30, 50 or 100 fold greater, than the level of expression of the protein of interest when the cell does not comprise said splice modulator, optionally wherein the level of expression when the cell does not comprise said splice modulator is undetectable.

61. The cell of any one of claims 57-59, wherein when the cell does not comprise a splice modulator, e.g., LMI070, the level of expression of the protein of interest is greater, e.g., 2, 3, 4, 5, 6, 7, 8, 9, 10, 20, 30, 50 or 100 fold greater, than the level of expression of the protein of interest when the cell comprises said splice modulator, optionally wherein the level of expression when the cell comprises said splice modulator is undetectable.

62. A method of conditionally expressing a protein of interest, said method comprising: contacting an expression system (e.g. a cell, e.g., a cell of any one of claims 57-61) comprising the nucleic acid molecule of any one of claims 1-2 and 4-40, the vector of any one of claims 41-49 or the recombinant virus of any one of claims 50-56, with a splice modulator, e.g., LMI070, wherein:
a. in the presence of said splice modulator, expression of said protein of interest is increased, e.g., 2, 3, 4, 5, 6, 7, 8, 9, 10, 20, 30, 50 or 100 fold greater, relative to the level of expression of said protein of interest in the absence of said splice modulator; and
b. in the absence of said splice modulator, expression of said protein of interest is substantially decreased, e.g., e.g., 2, 3, 4, 5, 6, 7, 8, 9, 10, 20, 30, 50 or 100 fold less, relative to the level of expression of said protein of interest in the presence of the splice modulator.

63. A method of conditionally expressing a protein of interest, said method comprising: contacting an expression system (e.g. a cell, e.g., a cell of any one of claims 57-61) comprising the nucleic acid molecule of any one of claims 1 or 3-36, the vector of any one of claims 41-49 or the recombinant virus of any one of claims 50-56, with a splice modulator, e.g., LMI070, wherein:
a. in the absence of said splice modulator, expression of said protein of interest is increased, e.g., 2, 3, 4, 5, 6, 7, 8, 9, 10, 20, 30, 50 or 100 fold greater, relative to the level of expression of said protein of interest in the presence of said splice modulator; and
b. in the presence of said splice modulator, expression of said protein of interest is substantially decreased, e.g., e.g., 2, 3, 4, 5, 6, 7, 8, 9, 10, 20, 30, 50 or 100 fold less, relative to the level of expression of said protein of interest in the absence of the splice modulator.

64. A pharmaceutical composition comprising the nucleic acid molecule of any one of claims 1-40, the vector of any one of claims 41-49, the recombinant virus of any one of claims 50-56, or the cell of any one of claims 57-61.

65. A method of treating a subject in need of a gene therapy, said method comprising administering to said subject the nucleic acid molecule of any one of claims 1-40, the vector of any one of claims 41-49, the recombinant virus of any one of claims 50-56, the cell of any one of claims 57-61, or the pharmaceutical composition of claim 64.

66. The method of claim 65, wherein the method further comprises administering to the subject an amount of a splice modulator, e.g., LMI070, effective to cause at least a 2, 3, 4, 5, 6, 7, 8, 9, 10, 20, 30, 50 or 100 fold increase or decrease in expression of the protein of interest, relative to the expression level of the protein of interest in the absence of the splice modulator.

67. A kit comprising the nucleic acid molecule of any one of claims 1-40, the vector of any one of claims 41-49, the recombinant virus of any one of claims 50-56, the cell of any one of claims 57-61, or the pharmaceutical composition of claim 64; and a splice modulator.

68. The nucleic acid molecule of any one of claims 1-40, the vector of any one of claims 41-49, the recombinant virus of any one of claims 50-56, the cell of any one of claims 57-61, or the pharmaceutical composition of claim 60, for use in a method of conditionally expressing a protein of interest, said method comprising: contacting an expression system (e.g. a cell, e.g., a cell of any one of claims 57-61) comprising the nucleic acid molecule of any one of claims 1-2 and 4-40, the vector of any one of claims 41-49 or the recombinant virus of any one of claims 50-62, with a splice modulator, e.g., LMI070, wherein:
a. in the presence of said splice modulator, expression of said protein of interest is increased, e.g., is at least 2, 3, 4, 5, 6, 7, 8, 9, 10, 20, 30, 50 or 100 fold greater, relative to the level of expression of said protein of interest in the absence of said splice modulator; and
b. in the absence of said splice modulator, expression of said protein of interest is substantially decreased, e.g., is at least 2, 3, 4, 5, 6, 7, 8, 9, 10, 20, 30, 50 or 100 fold less, relative to the level of expression of said protein of interest in the presence of the splice modulator.

69. The nucleic acid molecule of any one of claims 1-40, the vector of any one of claims 41-49, the recombinant virus of any one of claims 50-56, the cell of any one of claims 57-61, or the pharmaceutical composition of claim 64, for use in a method of conditionally expressing a protein of interest, said method comprising: contacting an expression system (e.g. a cell, e.g., a cell of any one of claims 57-61) comprising the nucleic acid molecule of any one of claims 1 or 3-40, the vector of any one of claims 41-49 or the recombinant virus of any one of claims 50-56, with a splice modulator, e.g., LMI070, wherein:
a. in the absence of said splice modulator, expression of said protein of interest is increased, e.g., is at least 2, 3, 4, 5, 6, 7, 8, 9, 10, 20, 30, 50 or 100 fold greater, relative to the level of expression of said protein of interest in the presence of said splice modulator; and
b. in the presence of said splice modulator, expression of said protein of interest is substantially decreased, e.g., is at least 2, 3, 4, 5, 6, 7, 8, 9, 10, 20, 30, 50 or 100 fold less, relative to the level of expression of said protein of interest in the absence of the splice modulator.

70. The nucleic acid molecule of any one of claims 1-40, the vector of any one of claims 41-49, the recombinant virus of any one of claims 50-56, the cell of any one of claims 57-61, or the pharmaceutical composition of claim 64, for use in a method of treating a subject in need of a gene therapy.

71. The nucleic acid molecule of any one of claims 1-40, the vector of any one of claims 41-49, the recombinant virus of any one of claims 50-56, the cell of any one of claims 57-61, the method of any one of claims 62-63 and 65-66, the pharmaceutical composition of claim 64, or the nucleic acid, vector, recombinant virus, cell, or pharmaceutical composition for use according to any one of claims 64-66, wherein the transgene encodes a protein of a genome editing system (for example, an RNA-guided nuclease such as a Cas9 protein, a zinc finger nuclease or a TALEN), an RNA (for example, a shRNA, or miRNA), an antibody or antibody fragment, or a therapeutic protein (for example, protein selected from progranulin, SMN, MeCP2, CLN2, CLN3, CLN4, CLN5, CLN6, CLN7, CLN8).


SEQUENCE LISTING (113 sequences)

SEQ ID NO: 1 - DNA, 73 bp, Homo sapiens
ccttgctatccctgtcttctgtagctattctgaaaccatcaacaaaggagcacaccattccatcagcaaaaga

SEQ ID NO: 2 - DNA, 72 bp, Unknown
  source: /note=“Description of Unknown: Exonic target sequence of LMI070”
gtaattagctgagaaggaagatctgaaggtttaacgagagagggcgagagatacaaaatatctgctaggaga

SEQ ID NO: 3 - DNA, 64 bp, Unknown
  source: /note=“Description of Unknown: Exonic target sequence of LMI070”
ggattgtttgtattcctgccaatgatttgtgagacagtctgttccccacatcctcgtcaacaga

SEQ ID NO: 4 - DNA, 78 bp, Unknown
  source: /note=“Description of Unknown: Exonic target sequence of LMI070”
ctttctgacatcttaacgaggcaatacagagagacgaattttcatcagtttgttcagggagacacatataacaaaaga

SEQ ID NO: 5 - DNA, 51 bp, Unknown
  source: /note=“Description of Unknown: Exonic target sequence of LMI070”
atccatacatacttaatgctgaaatgtgaagggctgagaaaaaagaaaaga

SEQ ID NO: 6 - DNA, 97 bp, Unknown
  source: /note=“Description of Unknown: Exonic target sequence of LMI070”
aattggaaacatcgagggaaaatgggctttttattattaaaacaaaacctcagtattatcacttagaaacctgaaattgaactccaaaagccaaaga

SEQ ID NO: 7 - DNA, 95 bp, Unknown
  source: /note=“Description of Unknown: Exonic target sequence of LMI070”
aagaatgttccttttgtgaagaatgacttaaggaagattcatgatgactgagtgtgcccgtgtggaactttaggacatagatgcactcctacaga

SEQ ID NO: 8 - DNA, 70 bp, Unknown
  source: /note=“Description of Unknown: Exonic target sequence of LMI070”
ttgtccttcactccgtactccagttggccaagcataggtcgcatgccagggtcaaggagactaagggaga

SEQ ID NO: 9 - DNA, 41 bp, Unknown
  source: /note=“Description of Unknown: Exonic target sequence of LMI070”
gacatacagacatggcagcccctagcatgtgtatcctaaga

SEQ ID NO: 10 - DNA, 40 bp, Unknown
  source: /note=“Description of Unknown: Exonic target sequence of LMI070”
acatacagacatggcagcccctagcatgtgtatcctaaga

SEQ ID NO: 11 - DNA, 2438 bp, Artificial Sequence
  source: /note=“Description of Artificial Sequence: Synthetic polynucleotide”
ctgcgcgctcgctcgctcactgaggccgcccgggcaaagcccgggcgtcgggcgacctttggtcgcccggcctcagtgagcgagcgagcgcgcagagagggagtggaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacaccggtcttccagaggagattggaaaacttgaagaagaagtggattgtgctaatattgccctgaaagcagccaccatggattgggagagttggaaacaaaatttgcaaattgatatcaagttagcatttacagatttggctgaggagaatatccattattttgaacaggtaattagtgttgtttgatattgcttcattttaaagttatttgctcatttacttttggtccgtccattgttgaaagagtgtattaaagaacaagtgtcacattctattgcctctctggtagcttggttttgttgaagttgtcagttaccatttggttttgtttatcctcagtttgttgttttggatttggattcttcaaaagcatttgatattgctttctattgattgtcctaactactcctctttcctctcccttctccatttttgaagagtttgcaaaggaaggaaaggagcagagacttgattgagcagaaaatcatttcagggcctgttctctattgtccttgctatcctgtcttctgtagctatctgaaaccatcaacaaaggagcacaccattccatcagcaaaagagtaacaacatctttttttaagttcattttgtttttcagttgattgtatttcaatttttttacagctgacttttctcagagaagttttttttttattgtaaacatactttttctagaaagtatattttaaaataacatctttaaccttatctctggctgaattattgaatatttgaaattattacattaacaaaattttgtcttacagcagtggtccccaaccttcttagcagtagcatccctcattaagaattaaaatttgtagaaattgacaaggattctgacaagctgttgggagagaagaatagagcagattgcagtaggaacagttgtgttagaatttattaatcctttaacactgaaagtaaactattgttgattgcctcttggtgtgtttccattattcagtgctcttgctaagtgggagtcattccttacatcaaccaccaaccttcacttggaagaagctagcgaagataaacctcgcaaccgccgcggcagcggcgaaggccgcggcagcctgctgacctgcggcgatgtggaagaaaacccgggcccggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgctttatttgtgaaatttgtgatgctattgctttatttgtaaccattataagctgcaataaacaagttaacaacaacaattgcattcattttatgtttcaggttcagggggaggtgtgggaggttttttaaaatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 12 - DNA, 106 bp, Artificial Sequence
  source: /note=“Description of Artificial Sequence: Synthetic polynucleotide”
ctgcgcgctcgctcgctcactgaggccgcccgggcaaagcccgggcgtcgggcgacctttggtcgcccggcctcagtgagcgagcgagcgcgcagagagggagtgg

SEQ ID NO: 13 - DNA, 194 bp, Artificial Sequence
  source: /note=“Description of Artificial Sequence: Synthetic polynucleotide”
aattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgaca

SEQ ID NO: 14 - DNA, 162 bp, Artificial Sequence
  source: /note=“Description of Artificial Sequence: Synthetic polynucleotide”
cttccagaggagattggaaaacttgaagaagaagtggattgtgctaatattgccctgaaagcagccaccatggattgggagagttggaaacaaaatttgcaaattgatatcaagttagcatttacagatttggctgaggagaatatccattattttgaacag

SEQ ID NO: 15 - DNA, 270 bp, Artificial Sequence
  source: /note=“Description of Artificial Sequence: Synthetic polynucleotide”
gtaattagtgttgtttgatattgcttcattttaaagttatttgctcatttacttttggtccgtccattgttgaaagagtgtattaaagaacaagtgtcacattctattgcctctctggtagcttggttttgttgaagttgtcagttaccatttggttttgtttatcctcagtttgttgttttggatttggattcttcaaaagcatttgatattgctttctattgattgtcctaactactcctctttcctctcccttctccatttttgaag

SEQ ID NO: 16 - DNA, 143 bp, Artificial Sequence
  source: /note=“Description of Artificial Sequence: Synthetic polynucleotide”
agtttgcaaaggaaggaaaggagcagagacttgattgagcagaaaatcatttcagggcctgttctctattgtccttgctatcctgtcttctgtagctatctgaaaccatcaacaaaggagcacaccattccatcagcaaaaga

SEQ ID NO: 17 - DNA, 407 bp, Artificial Sequence
  source: /note=“Description of Artificial Sequence: Synthetic polynucleotide”
gtaacaacatctttttttaagttcattttgtttttcagttgattgtatttcaatttttttacagctgacttttctcagagaagttttttttttattgtaaacatactttttctagaaagtatattttaaaataacatctttaaccttatctctggctgaattattgaatatttgaaattattacattaacaaaattttgtcttacagcagtggtccccaaccttcttagcagtagcatccctcattaagaattaaaatttgtagaaattgacaaggattctgacaagctgttgggagagaagaatagagcagattgcagtaggaacagttgtgttagaatttattaatcctttaacactgaaagtaaactattgttgattgcctcttggtgtgtttccattattcag

SEQ ID NO: 18 - DNA, 76 bp, Artificial Sequence
  source: /note=“Description of Artificial Sequence: Synthetic oligonucleotide”
tgctcttgctaagtgggagtcattccttacatcaaccaccaaccttcacttggaagaagctagcgaagataaacct

SEQ ID NO: 19 - DNA, 12 bp, Artificial Sequence
  source: /note=“Description of Artificial Sequence: Synthetic oligonucleotide”
cgcaaccgccgc

SEQ ID NO: 20 - DNA, 63 bp, Artificial Sequence
  source: /note=“Description of Artificial Sequence: Synthetic oligonucleotide”
ggcagcggcgaaggccgcggcagcctgctgacctgcggcgatgtggaagaaaacccgggcccg

SEQ ID NO: 21 - DNA, 717 bp, Artificial Sequence
  source: /note=“Description of Artificial Sequence: Synthetic polynucleotide”
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 22 - DNA, 132 bp, Artificial Sequence
  source: /note=“Description of Artificial Sequence: Synthetic polynucleotide”
tgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 23 - DNA, 141 bp, Artificial Sequence
  source: /note=“Description of Artificial Sequence: Synthetic polynucleotide”
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 24-34 - skipped (11 entries)

SEQ ID NO: 35 - protein, 5 aa, Artificial Sequence
  source: /note=“Description of Artificial Sequence: Synthetic peptide”
  MOD_RES (5)..(5): Any amino acid
IEPDX

SEQ ID NO: 36 - protein, 5 aa, Artificial Sequence
  source: /note=“Description of Artificial Sequence: Synthetic peptide”
DDDDK

SEQ ID NO: 37 - protein, 6 aa, Artificial Sequence
  source: /note=“Description of Artificial Sequence: Synthetic peptide”
PGAAHY

SEQ ID NO: 38 - protein, 7 aa, Artificial Sequence
  source: /note=“Description of Artificial Sequence: Synthetic peptide”
LEVFQGP

SEQ ID NO: 39 - protein, 4 aa, Artificial Sequence
  source: /note=“Description of Artificial Sequence: Synthetic peptide”
RNRR

SEQ ID NO: 40 - protein, 6 aa, Artificial Sequence
  source: /note=“Description of Artificial Sequence: Synthetic peptide”
LVPRGS

SEQ ID NO: 41 - protein, 7 aa, Artificial Sequence
  source: /note=“Description of Artificial Sequence: Synthetic peptide”
ENLYFQG

SEQ ID NO: 42 - protein, 2 aa, Artificial Sequence
  source: /note=“Description of Artificial Sequence: Synthetic peptide”
  VARIANT (1)..(1): /replace=“Gly” or “Ser” or “Val”
  MOD_RES (2)..(2): Any amino acid
  SITE (1)..(2): /note=“Variant residues given in the sequence have no preference with respect to those in the annotations for variant positions”
AX

SEQ ID NO: 43 - protein, 4 aa, Artificial Sequence
  source: /note=“Description of Artificial Sequence: Synthetic peptide”
RTKR

SEQ ID NO: 44 - DNA, 12 bp, Artificial Sequence
  source: /note=“Description of Artificial Sequence: Synthetic oligonucleotide”
cgtactaaaaga

SEQ ID NO: 45 - protein, 20 aa, Artificial Sequence
  source: /note=“Description of Artificial Sequence: Synthetic peptide”
GTGAEDPRPSRKRRSLGDVG

SEQ ID NO: 46 - DNA, 60 bp, Artificial Sequence
  source: /note=“Description of Artificial Sequence: Synthetic oligonucleotide”
ggaaccggcgcggaagacccccggccctccaggaagcgaaggtccctcggagacgtgggt

SEQ ID NO: 47 - protein, 14 aa, Artificial Sequence
  source: /note=“Description of Artificial Sequence: Synthetic peptide”
GTGAEDPRPSRKRR

SEQ ID NO: 48 - DNA, 42 bp, Artificial Sequence
  source: /note=“Description of Artificial Sequence: Synthetic oligonucleotide”
ggaaccggcgcggaagacccccggccctccaggaagcgaagg

SEQ ID NO: 49 - protein, 15 aa, Artificial Sequence
  source: /note=“Description of Artificial Sequence: Synthetic peptide”
LQWLEQQVAKRRTKR

SEQ ID NO: 50 - DNA, 45 bp, Artificial Sequence
  source: /note=“Description of Artificial Sequence: Synthetic oligonucleotide”
ctgcaatggctggagcagcaggtggcgaagcggagaactaagcgg

SEQ ID NO: 51 - protein, 18 aa, Artificial Sequence
  source: /note=“Description of Artificial Sequence: Synthetic peptide”
GTGAEDPRPSRKRRSLGG

SEQ ID NO: 52 - DNA, 54 bp, Artificial Sequence
  source: /note=“Description of Artificial Sequence: Synthetic oligonucleotide”
ggcacaggtgccgaggaccctcggccaagccgcaaaaggaggtcacttggcggc

SEQ ID NO: 53 - protein, 17 aa, Artificial Sequence
  source: /note=“Description of Artificial Sequence: Synthetic peptide”
GTGAEDPRPSRKRRSLG

SEQ ID NO: 54 - DNA, 51 bp, Artificial Sequence
  source: /note=“Description of Artificial Sequence: Synthetic oligonucleotide”
ggaaccggagcagaagatcccagaccaagccggaaaaggcggtccctgggt

SEQ ID NO: 55 - protein, 14 aa, Artificial Sequence
  source: /note=“Description of Artificial Sequence: Synthetic peptide”
SLNLTESHNSRKKR

SEQ ID NO: 56 - DNA, 42 bp, Artificial Sequence
  source: /note=“Description of Artificial Sequence: Synthetic oligonucleotide”
agtctcaatttgactgagtcacacaattccaggaagaaaagg

SEQ ID NO: 57 - protein, 14 aa, Artificial Sequence
  source: /note=“Description of Artificial Sequence: Synthetic peptide”
CKINGYPKRGRKRR

SEQ ID NO: 58 - DNA, 42 bp, Artificial Sequence
  source: /note=“Description of Artificial Sequence: Synthetic oligonucleotide”
tgcaagatcaacggctaccctaagaggggcagaaagcggcgg

SEQ ID NO: 59 - protein, 21 aa, Artificial Sequence
  source: /note=“Description of Artificial Sequence: Synthetic peptide”
  VARIANT (1)..(3): /replace=“ ”
  SITE (1)..(21): /note=“Variant residues given in the sequence have no preference with respect to those in the annotations for variant positions”
GSGEGRGSLLTCGDVEENPGP

SEQ ID NO: 60 - skipped

SEQ ID NO: 61 - protein, 18 aa, Thosea asigna virus 2A
EGRGSLLTCGDVEENPGP

SEQ ID NO: 62 - DNA, 53 bp, Thosea asigna virus 2A
gaaggccgcggcagcctgctgacctgcggcgatgtggaagaaaacccgggccc

SEQ ID NO: 63 - protein, 22 aa, Artificial Sequence
  source: /note=“Description of Artificial Sequence: Synthetic peptide”
  VARIANT (1)..(3): /replace=“ ”
  SITE (1)..(22): /note=“Variant residues given in the sequence have no preference with respect to those in the annotations for variant positions”
GSGATNFSLLKQAGDVEENPGP

SEQ ID NO: 64 - DNA, 66 bp, Artificial Sequence
  source: /note=“Description of Artificial Sequence: Synthetic oligonucleotide”
  variation (1)..(9): /replace=“ ”
  misc_feature (1)..(66): /note=“Variant nucleotides given in the sequence have no preference with respect to those in the annotations for variant positions”
ggcagcggcgccaccaacttcagcctgctgaagcaggccggcgacgtggaggagaaccccggcccc

SEQ ID NO: 65 - protein, 23 aa, Artificial Sequence
  source: /note=“Description of Artificial Sequence: Synthetic peptide”
  VARIANT (1)..(3): /replace=“ ”
  SITE (1)..(23): /note=“Variant residues given in the sequence have no preference with respect to those in the annotations for variant positions”
GSGQCTNYALLKLAGDVESNPGP

SEQ ID NO: 66 - DNA, 69 bp, Artificial Sequence
  source: /note=“Description of Artificial Sequence: Synthetic oligonucleotide”
  variation (1)..(9): /replace=“ ”
  misc_feature (1)..(69): /note=“Variant nucleotides given in the sequence have no preference with respect to those in the annotations for variant positions”
ggcagcggccagtgcaccaactacgccctgctgaagctggccggcgacgtggagagcaaccccggcccc

SEQ ID NO: 67 - protein, 25 aa, Artificial Sequence
  source: /note=“Description of Artificial Sequence: Synthetic peptide”
  VARIANT (1)..(3): /replace=“ ”
  SITE (1)..(25): /note=“Variant residues given in the sequence have no preference with respect to those in the annotations for variant positions”
GSGVKQTLNFDLLKLAGDVESNPGP

SEQ ID NO: 68 - DNA, 75 bp, Artificial Sequence
  source: /note=“Description of Artificial Sequence: Synthetic oligonucleotide”
  variation (1)..(9): /replace=“ ”
  misc_feature (1)..(75): /note=“Variant nucleotides given in the sequence have no preference with respect to those in the annotations for variant positions”
ggcagcggcgtgaagcagaccctgaacttcgacctgctgaagctggccggcgacgtggagagcaaccccggcccc

SEQ ID NO: 69 - DNA, 9 bp, Artificial Sequence
  source: /note=“Description of Artificial Sequence: Synthetic oligonucleotide”
gaagaagaa

SEQ ID NO: 70 - DNA, 6 bp, Artificial Sequence
  source: /note=“Description of Artificial Sequence: Synthetic oligonucleotide”
gccacc

SEQ ID NO: 71 - DNA, 1058 bp, Artificial Sequence
  source: /note=“Description of Artificial Sequence: Synthetic polynucleotide”
cttccagaggagattggaaaacttgaagaagaagtggattgtgctaatattgccctgaaagcagccaccatggattgggagagttggaaacaaaatttgcaaattgatatcaagttagcatttacagatttggctgaggagaatatccattattttgaacaggtaattagtgttgtttgatattgcttcattttaaagttatttgctcatttacttttggtccgtccattgttgaaagagtgtattaaagaacaagtgtcacattctattgcctctctggtagcttggttttgttgaagttgtcagttaccatttggttttgtttatcctcagtttgttgttttggatttggattcttcaaaagcatttgatattgctttctattgattgtcctaactactcctctttcctctcccttctccatttttgaagagtttgcaaaggaaggaaaggagcagagacttgattgagcagaaaatcatttcagggcctgttctctattgtccttgctatcctgtcttctgtagctatctgaaaccatcaacaaaggagcacaccattccatcagcaaaagagtaacaacatctttttttaagttcattttgtttttcagttgattgtatttcaatttttttacagctgacttttctcagagaagttttttttttattgtaaacatactttttctagaaagtatattttaaaataacatctttaaccttatctctggctgaattattgaatatttgaaattattacattaacaaaattttgtcttacagcagtggtccccaaccttcttagcagtagcatccctcattaagaattaaaatttgtagaaattgacaaggattctgacaagctgttgggagagaagaatagagcagattgcagtaggaacagttgtgttagaatttattaatcctttaacactgaaagtaaactattgttgattgcctcttggtgtgtttccattattcagtgctcttgctaagtgggagtcattccttacatcaaccaccaaccttcacttggaagaagctagcgaagataaacct

SEQ ID NO: 72 - DNA, 720 bp, Unknown
  source: /note=“Description of Unknown: PRE sequence”
acaggcctattgattggaaagtatgtcaacgaattgtgggtcttttggggtttgctgccccttttacgcaatgtggatatcctgctttaatgcctttatatgcatgtatacaagcaaaacaggcttttactttctcgccaacttacaaggcctttctaagtaaacagtatctgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcgaaactcatcgggactgacaattctgtcgtgctctcccgcaagtatacatcgtttccagggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgaccgaccacggggcgcacctctctttacgcggactccccgtctgtgccttctcatctgccggaccgtgtgcacttcgcttcacctctgcacgtcgcatggagaccaccgtgaacgcccaccggaacctgcccaaggtcttgcataagaggactcttggactttcagcaatgtc

SEQ ID NO: 73 - DNA, 721 bp, Unknown
  source: /note=“Description of Unknown: PRE sequence”
aacaggcctattgattggaaagtatgtcaacgaattgtgggtcttttggggtttgctgccccttttacgcaatgtggatatcctgctttaatgcctttatatgcatgtatacaagcaaaacaggcttttactttctcgccaacttacaaggcctttctaagtaaacagtatctgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcgaaactcatcgggactgacaattctgtcgtgctctcccgcaagtatacatcgtttccagggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgaccgaccacggggcgcacctctctttacgcggactccccgtctgtgccttctcatctgccggaccgtgtgcacttcgcttcacctctgcacgtcgcatggagaccaccgtgaacgcccaccggaacctgcccaaggtcttgcataagaggactcttggactttcagcaatgtc

SEQ ID NO: 74 - DNA, 2211 bp, Adeno-associated virus
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 75 - DNA, 1800 bp, Adeno-associated virus
acggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 76 - DNA, 1605 bp, Adeno-associated virus
atggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 77 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 78 - protein, 599 aa, Adeno-associated virus
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 79 - protein, 534 aa, Adeno-associated virus
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 80 - DNA, 144 bp, Homo sapiens
agtttgcaaaggaaggaaaggagcagagacttgaatgagcagaaaatcatttcagggcctgttctctatgtccttgctatccctgtcttctgtagctattctgaaaccatcaacaaaggagcacaccattccatcagcaaaaga

SEQ ID NO: 81 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 82-84 - skipped (3 entries)

SEQ ID NO: 85 - DNA, 141 bp, Artificial Sequence
  source: /note=“Description of Artificial Sequence: Synthetic polynucleotide”
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 86 - DNA, 483 bp, Homo sapiens
aaccgagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgt

SEQ ID NO: 87 - DNA, 1779 bp, Homo sapiens
tggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtag

SEQ ID NO: 88 - DNA, 721 bp, Artificial Sequence; identical to SEQ ID NO: 73
  source: /note=“Description of Artificial Sequence: Synthetic polynucleotide”

SEQ ID NO: 89 - DNA, 479 bp, Artificial Sequence
  source: /note=“Description of Artificial Sequence: Synthetic polynucleotide”
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 90 - DNA, 141 bp, Artificial Sequence
  source: /note=“Description of Artificial Sequence: Synthetic polynucleotide”
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 91 - DNA, 4958 bp, Artificial Sequence
  source: /note=“Description of Artificial Sequence: Synthetic polynucleotide”
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaaccgagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgaattccttccagaggagattggaaaacttgaagaagaagtggattgtgctaatattgccctgaaagcagccaccatggattgggagagttggaaacaaaatttgcaaattgatatcaagttagcatttacagatttggctgaggagaatatccattattttgaacaggtaattagtgttgtttgatattgcttcattttaaagttatttgctcatttacttttggtccgtccattgttgaaagagtgtattaaagaacaagtgtcacattctattgcctctctggtagcttggttttgttgaagttgtcagttaccatttggttttgtttatcctcagtttgttgttttggatttggattcttcaaaagcatttgatattgctttctattgattgtcctaactactcctctttcctctcccttctccatttttgaagagtttgcaaaggaaggaaaggagcagagacttgattgagcagaaaatcatttcagggcctgttctctattgtccttgctatcctgtcttctgtagctatctgaaaccatcaacaaaggagcacaccattccatcagcaaaagagtaacaacatctttttttaagttcattttgtttttcagttgattgtatttcaatttttttacagctgacttttctcagagaagttttttttttattgtaaacatactttttctagaaagtatattttaaaataacatctttaaccttatctctggctgaattattgaatatttgaaattattacattaacaaaattttgtcttacagcagtggtccccaaccttcttagcagtagcatccctcattaagaattaaaatttgtagaaattgacaaggattctgacaagctgttgggagagaagaatagagcagattgcagtaggaacagttgtgttagaatttattaatcctttaacactgaaagtaaactattgttgattgcctcttggtgtgtttccattattcagtgctcttgctaagtgggagtcattccttacatcaaccaccaaccttcacttggaagaagctagcgaagataaacctcgcaaccgccgcggcagcggcgaaggccgcggcagcctgctgacctgcggcgatgtggaagaaaacccgggcccgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtaggtcgactaaacaggcctattgattggaaagtatgtcaacgaattgtgggtcttttggggtttgctgccccttttacgcaatgtggatatcctgctttaatgcctttatatgcatgtatacaagcaaaacaggcttttactttctcgccaacttacaaggcctttctaagtaaacagtatctgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcgaaactcatcgggactgacaattctgtcgtgctctcccgcaagtatacatcgtttccagggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgaccgaccacggggcgcacctctctttacgcggactccccgtctgtgccttctcatctgccggaccgtgtgcacttcgcttcacctctgcacgtcgcatggagaccaccgtgaacgcccaccggaacctgcccaaggtcttgcataagaggactcttggactttcagcaatgtcaactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 92 - DNA, 9220 bp, Artificial Sequence
  source: /note=“Description of Artificial Sequence: Synthetic polynucleotide”
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtaaccgagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgaattccttccagaggagattggaaaacttgaagaagaagtggattgtgctaatattgccctgaaagcagccaccatggattgggagagttggaaacaaaatttgcaaattgatatcaagttagcatttacagatttggctgaggagaatatccattattttgaacaggtaattagtgttgtttgatattgcttcattttaaagttatttgctcatttacttttggtccgtccattgttgaaagagtgtattaaagaacaagtgtcacattctattgcctctctggtagcttggttttgttgaagttgtcagttaccatttggttttgtttatcctcagtttgttgttttggatttggattcttcaaaagcatttgatattgctttctattgattgtcctaactactcctctttcctctcccttctccatttttgaagagtttgcaaaggaaggaaaggagcagagacttgattgagcagaaaatcatttcagggcctgttctctattgtccttgctatcctgtcttctgtagctatctgaaaccatcaacaaaggagcacaccattccatcagcaaaagagtaacaacatctttttttaagttcattttgtttttcagttgattgtatttcaatttttttacagctgacttttctcagagaagttttttttttattgtaaacatactttttctagaaagtatattttaaaataacatctttaaccttatctctggctgaattattgaatatttgaaattattacattaacaaaattttgtcttacagcagtggtccccaaccttcttagcagtagcatccctcattaagaattaaaatttgtagaaattgacaaggattctgacaagctgttgggagagaagaatagagcagattgcagtaggaacagttgtgttagaatttattaatcctttaacactgaaagtaaactattgttgattgcctcttggtgtgtttccattattcagtgctcttgctaagtgggagtcattccttacatcaaccaccaaccttcacttggaagaagctagcgaagataaacctcgcaaccgccgcggcagcggcgaaggccgcggcagcctgctgacctgcggcgatgtggaagaaaacccgggcccgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtaggtcgactaaacaggcctattgattggaaagtatgtcaacgaattgtgggtcttttggggtttgctgccccttttacgcaatgtggatatcctgctttaatgcctttatatgcatgtatacaagcaaaacaggcttttactttctcgccaacttacaaggcctttctaagtaaacagtatctgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcgaaactcatcgggactgacaattctgtcgtgctctcccgcaagtatacatcgtttccagggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgaccgaccacggggcgcacctctctttacgcggactccccgtctgtgccttctcatctgccggaccgtgtgcacttcgcttcacctctgcacgtcgcatggagaccaccgtgaacgcccaccggaacctgcccaaggtcttgcataagaggactcttggactttcagcaatgtcaactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggaagctgtaagctctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgcatg

SEQ ID NO: 93 - DNA, 5954 bp, Artificial Sequence
  source: /note=“Description of Artificial Sequence: Synthetic polynucleotide”
ctgcgcgctcgctcgctcactgaggccgcccgggcaaagcccgggcgtcgggcgacctttggtcgcccggcctcagtgagcgagcgagcgcgcagagagggagtggaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacaccggtcttccagaggagattggaaaacttgaagaagaagtggattgtgctaatattgccctgaaagcagccaccatggattgggagagttggaaacaaaatttgcaaattgatatcaagttagcatttacagatttggctgaggagaatatccattattttgaacaggtaattagtgttgtttgatattgcttcattttaaagttatttgctcatttacttttggtccgtccattgttgaaagagtgtattaaagaacaagtgtcacattctattgcctctctggtagcttggttttgttgaagttgtcagttaccatttggttttgtttatcctcagtttgttgttttggatttggattcttcaaaagcatttgatattgctttctattgattgtcctaactactcctctttcctctcccttctccatttttgaagagtttgcaaaggaaggaaaggagcagagacttgattgagcagaaaatcatttcagggcctgttctctattgtccttgctatcctgtcttctgtagctatctgaaaccatcaacaaaggagcacaccattccatcagcaaaagagtaacaacatctttttttaagttcattttgtttttcagttgattgtatttcaatttttttacagctgacttttctcagagaagttttttttttattgtaaacatactttttctagaaagtatattttaaaataacatctttaaccttatctctggctgaattattgaatatttgaaattattacattaacaaaattttgtcttacagcagtggtccccaaccttcttagcagtagcatccctcattaagaattaaaatttgtagaaattgacaaggattctgacaagctgttgggagagaagaatagagcagattgcagtaggaacagttgtgttagaatttattaatcctttaacactgaaagtaaactattgttgattgcctcttggtgtgtttccattattcagtgctcttgctaagtgggagtcattccttacatcaaccaccaaccttcacttggaagaagctagcgaagataaacctcgcaaccgccgcggcagcggcgaaggccgcggcagcctgctgacctgcggcgatgtggaagaaaacccgggcccggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgctttatttgtgaaatttgtgatgctattgctttatttgtaaccattataagctgcaataaacaagttaacaacaacaattgcattcattttatgtttcaggttcagggggaggtgtgggaggttttttaaaatcgatctgaggaacccctagtgatggagttggccactcccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgcc

SEQ ID NO: 94 - DNA, 799 bp, Artificial Sequence
  source: /note=“Description of Artificial Sequence: Synthetic polynucleotide”
gaagaagaagatatcaagttagcatttacagatttggctgaggagaagaacaggtaattagtgttgtttgatattgcttcattttaaagttatttgctcatttagcatttgatattgctttctattgattgtcctaactactcctctttcctctcccttctccatttttgaagagtttgcaaaggaaggaaaggagcagagacttgattgagcagaaaatcatttcagggcctgttctctattgtccttgctatcctgtcttctgtagctatctgaaaccatcaacaaaggagcacaccatggcatcagcaaaagagtaacaacatctttttttaagttcattttgtttttcagttgattgtatttcaatttttttacagctgacttttctcagagaagttttttttttattgtaaacatactttttctagaaagtatattttaaaataacatctttaaccttatctctggctgaattattgaatatttgaaattattacattaacaaaattttgtcttacagcagtggtccccaaccttcttagcagtagcatccctcattaagaattaaaatttgtagaaattgacaaggattctgacaagctgttgggagagaagaatagagcagattgcagtaggaacagttgtgttagaatttattaatcctttaacactgaaagtaaactattgttgattgcctcttggtgtgtttccattattcagtgctcttgctaagtgggagtcattccttacatcaaccaccaaccttcacttggaagaagctagcgaagataaacct

SEQ ID NO: 95 - DNA, 5446 bp, Artificial Sequence
  source: /note=“Description of Artificial Sequence: Synthetic polynucleotide”
gcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacaccggtgaagaagaagatatcaagttagcatttacagatttggctgaggagaagaacaggtaattagtgttgtttgatattgcttcattttaaagttatttgctcatttagcatttgatattgctttctattgattgtcctaactactcctctttcctctcccttctccatttttgaagagtttgcaaaggaaggaaaggagcagagacttgattgagcagaaaatcatttcagggcctgttctctattgtccttgctatcctgtcttctgtagctatctgaaaccatcaacaaaggagcacaccatggcatcagcaaaagagtaacaacatctttttttaagttcattttgtttttcagttgattgtatttcaatttttttacagctgacttttctcagagaagttttttttttattgtaaacatactttttctagaaagtatattttaaaataacatctttaaccttatctctggctgaattattgaatatttgaaattattacattaacaaaattttgtcttacagcagtggtccccaaccttcttagcagtagcatccctcattaagaattaaaatttgtagaaattgacaaggattctgacaagctgttgggagagaagaatagagcagattgcagtaggaacagttgtgttagaatttattaatcctttaacactgaaagtaaactattgttgattgcctcttggtgtgtttccattattcagtgctcttgctaagtgggagtcattccttacatcaaccaccaaccttcacttggaagaagctagcgaagataaacctcgcaaccgccgcggcagcggcgaaggccgcggcagcctgctgacctgcggcgatgtggaagaaaacccgggcccggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaacctgcccgctgggcctcccaacgggccctcctcccctccttgcaccaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 96 - DNA, 53 bp, Artificial Sequence
  source: /note=“Description of Artificial Sequence: Synthetic oligonucleotide”
gaagaagaagatatcaagttagcatttacagatttggctgaggagaagaacag

SEQ ID NO: 97 - DNA, 120 bp, Artificial Sequence
  source: /note=“Description of Artificial Sequence: Synthetic polynucleotide”
gtaattagtgttgtttgatattgcttcattttaaagttatttgctcatttagcatttgatattgctttctattgattgtcctaactactcctctttcctctcccttctccatttttgaag

SEQ ID NO: 98 - DNA, 143 bp, Artificial Sequence
  source: /note=“Description of Artificial Sequence: Synthetic polynucleotide”
agtttgcaaaggaaggaaaggagcagagacttgattgagcagaaaatcatttcagggcctgttctctattgtccttgctatcctgtcttctgtagctatctgaaaccatcaacaaaggagcacaccatggcatcagcaaaaga

SEQ ID NO: 99 - DNA, 153 bp, Homo sapiens
cttccagaggagattggaaaacttgaagataaagtggaatgtgctaataatgccctgaaagcagattgggagagatggaaacaaaatatgcaaaatgatatcaagttagcatttacagatatggctgaggagaatatccattattatgaacag

SEQ ID NO: 100 - DNA, 78 bp, Homo sapiens
tgccttgctacgtgggagtcattccttacatcacagaccaaccttcacttggaagaagcctctgaagataaaccttaa

SEQ ID NO: 101 - DNA, 18 bp, Artificial Sequence
  source: /note=“Description of Artificial Sequence: Synthetic oligonucleotide”
  misc_feature (1)..(18): /note=“This sequence may encompass 1-6 ′gaa′ repeating units”
gaagaagaagaagaagaa

SEQ ID NO: 102 - protein, 12 aa, Artificial Sequence
  source: /note=“Description of Artificial Sequence: Synthetic peptide”
CCCCCCCCCCCC

SEQ ID NO: 103 - DNA, 21 bp, Artificial Sequence
  source: /note=“Description of Artificial Sequence: Synthetic primer”
caacaaaggagcacaccattc

SEQ ID NO: 104 - DNA, 19 bp, Artificial Sequence
  source: /note=“Description of Artificial Sequence: Synthetic primer”
gcggttgcgaggtttatct

SEQ ID NO: 105 - DNA, 22 bp, Artificial Sequence
  source: /note=“Description of Artificial Sequence: Synthetic primer”
ctcttgctaagtgggagtcatt

SEQ ID NO: 106 - DNA, 22 bp, Artificial Sequence
  source: /note=“Description of Artificial Sequence: Synthetic primer”
gtgctaataatgccctgaaagc

SEQ ID NO: 107 - DNA, 20 bp, Artificial Sequence
  source: /note=“Description of Artificial Sequence: Synthetic primer”
ccacttagcaagagcactgt

SEQ ID NO: 108 - protein, 34 aa, Artificial Sequence
  source: /note=“Description of Artificial Sequence: Synthetic polypeptide”
MDWESWKQNLQIDIKLAFTDLAEENIHYFEQCSC

SEQ ID NO: 109 - protein, 149 aa, Artificial Sequence
  source: /note=“Description of Artificial Sequence: Synthetic polypeptide”
MDWESWKQNLQIDIKSDHLQIWLRRISIILNSALAKWESFLTSTTNLHLEEASEDKPRNRRGSGEGRGSLLTCGDVEENPGPVSKGEELFTGVVPILVELDGDVNGHKFSVSGEGEGDATYGKLTLKFICTTGKLPVPWPTLVTTLTYG

SEQ ID NO: 110 - protein, 175 aa, Artificial Sequence
  source: /note=“Description of Artificial Sequence: Synthetic polypeptide”
MDWESWKQNLQIDIKLAFTDLAEENIHYFEQSLQRKERSRDLIEQKIISGPVLYCPCYPVFCSYLKPSTKEHTIPSAKDALAKWESFLTSTTNLHLEEASEDKPRNRRGSGEGRGSLLTCGDVEENPGPVSKGEELFTGVVPILVELDGDVNGHKFSVSGEGEGDATYGKLTLKF

SEQ ID NO: 111 - protein, 42 aa, Artificial Sequence
  source: /note=“Description of Artificial Sequence: Synthetic polypeptide”
MDWESWKQNLQIDIKSDHLQIWLRRISIILNRVCKGRKGAET

SEQ ID NO: 112 - protein, 142 aa, Artificial Sequence
  source: /note=“Description of Artificial Sequence: Synthetic polypeptide”
MDWESWKQNLQIDIKLAFTDLAEENIHYFEQSLQRKERSRDLIEQKIISGPVLYCPCYPVFCSYLKPSTKEHTIPSAKDALAKWESFLTSTTNLHLEEASEDKPRNRRGSGEGRGSLLTCGDVEENPGPVSKGEELFTGVVP

SEQ ID NO: 113 - protein, 69 aa, Artificial Sequence
  source: /note=“Description of Artificial Sequence: Synthetic polypeptide”
MASAKDALAKWESFLTSTTNLHLEEASEDKPRNRRGSGEGRGSLLTCGDVEENPGPVSKGEELFTGVVP